SEQUENCE LISTING (146 sequences)

SEQ ID NO: 1 - DNA, 873 bp, Pseudomonas cichorii
gtgaacaaagttggcatgttctacacctactggtcgactgagtggatggtcgactttccggcgactgcgaagcgcattgccgggctcggcttcgacttaatggaaatctcgctcggcgagtttcacaatctttccgacgcgaagaagcgtgagctaaaagccgtggctgatgatctggggctcacggtgatgtgctgtatcggactgaagtctgagtacgactttgcctcgccggacaagagcgttcgtgatgccggcacggaatatgtgaagcgcttgctcgacgactgtcacctcctcggcgcgccggtctttgctggccttacgttctgcgcgtggccccaatctccgccgctggacatgaaggataagcgcccttacgtcgaccgtgcaatcgaaagcgttcgtcgtgttatcaaggtagctgaagactacggcattatttatgcactggaagtggtgaaccgattcgagcagtggctttgcaatgacgccaaggaagcaattgcgtttgccgacgcggttgacagtccggcgtgcaaggtccagctcgacacattccacatgaatatcgaagagacttccttccgcgatgcaatccttgcctgcaagggcaagatgggccatttccatttgggcgaagcgaaccgtctgccgccgggcgagggtcgcctgccgtgggatgaaatattcggggcgctgaaggaaatcggatatgacggcaccatcgttatggaaccgttcatgcgcaagggcggctcggtcagccgcgcggtgggcgtatggcgggatatgtcgaacggtgcgacggacgaagagatggacgagcgcgctcgccgctcgttgcagtttgttcgtgacaagctggcctga

SEQ ID NO: 2 - DNA, 873 bp, Artificial Sequence
  Pseudomonas cichorii D-tagatose 3-epimerase DTE codon optimized sequence
atgaacaaagtgggtatgttctatacgtactggtccacggaatggatggttgactttccggcaaccgcgaaacgtattgcgggcctgggcttcgacctgatggaaatttctctgggcgaatttcacaacctgtccgatgcgaaaaagcgtgaactgaaagccgttgccgacgatctgggtctgactgtgatgtgctgtatcggcctgaaatctgaatacgatttcgcgagcccggataaaagcgttcgcgacgccggtactgaatatgtcaaacgtctgctggatgactgtcacctgctgggcgcaccagtgttcgcgggtctgaccttctgtgcgtggccgcagtccccaccgctggacatgaaggataaacgtccgtacgtggaccgtgccatcgaaagcgtgcgccgcgtaatcaaagtcgctgaagattatggcattatttacgctctggaagttgttaaccgtttcgaacagtggctgtgcaacgacgcgaaagaggccattgccttcgctgacgcggtggattctccggcttgcaaagttcagctggacactttccatatgaacatcgaggaaacctccttccgtgacgcgatcctggcttgcaagggtaaaatgggccatttccatctgggcgaagcaaaccgcctgccgccgggcgaaggtcgtctgccgtgggacgaaatttttggcgctctgaaggaaatcggctacgatggcacgattgttatggagccgttcatgcgcaaaggtggctccgtttcccgtgcagttggtgtttggcgtgatatgtctaacggtgccaccgatgaagaaatggacgaacgtgcacgtcgctccctgcaattcgttcgcgataaactggcgtaa

SEQ ID NO: 3 - protein, 290 aa, Pseudomonas cichorii
MNKVGMFYTYWSTEWMVDFPATAKRIAGLGFDLMEISLGEFHNLSDAKKRELKAVADDLGLTVMCCIGLKSEYDFASPDKSVRDAGTEYVKRLLDDCHLLGAPVFAGLTFCAWPQSPPLDMKDKRPYVDRAIESVRRVIKVAEDYGIIYALEVVNRFEQWLCNDAKEAIAFADAVDSPACKVQLDTFHMNIEETSFRDAILACKGKMGHFHLGEANRLPPGEGRLPWDEIFGALKEIGYDGTIVMEPFMRKGGSVSRAVGVWRDMSNGATDEEMDERARRSLQFVRDKLA

SEQ ID NO: 4 - DNA, 888 bp, Rhodobacter sphaeroides
gtgaaaaatcctgtcggcatcatctcgatgcagttcatccggcccttcacctcggagtcgctgcatttcctgaagaagtcccgggccctgggcttcgatttcatcgagcttctcgtgcccgagcccgaagacgggctcgacgcggccgaggtgcggcgcatctgcgagggcgaggggctgggcctcgttctggccgcgcgcgtgaacctccagcgctcgatcgcgagcgaggaggccgcggcgcgggccggcgggcgcgactatctgaaatactgcatcgaggccgccgaggcgctcggcgcgaccatcgtcggcggcccgctctatggcgagccgctggtcttcgccggccgcccgcccttcccctggacggccgagcagatcgccacccgcgccgcccgcaccgtcgaggggctggccgaagtggccccgctcgccgcgagcgcgggcaaggtcttcgggctcgagccgctgaaccgcttcgagaccgacatcgtgaacacgaccgcacaggccatcgaggtggtggatgcggtgggctcgcccggtctcggcgtcatgctcgacacgttccacatgaacatggaggaacgctcgatccccgatgcgatccgcgccacaggcgcgcgcctcgtccattttcaggccaacgagaaccaccgcggcttccccggcaccggcaccatggactggacggccatcgcgcgggcgctggggcaggcgggctacgcgggtccggtctcgctcgagcctttccggcgcgacgacgagcgcgtggcgctgcccatcgcccactggcgcgccccgcacgaggacgaggacgagaagctgcgcgcggggctgggtctcatccgctccgcgatcaccctggcggaggtgacccactga

SEQ ID NO: 5 - protein, 295 aa, Rhodobacter sphaeroides
MKNPVGIISMQFIRPFTSESLHFLKKSRALGFDFIELLVPEPEDGLDAAEVRRICEGEGLGLVLAARVNLQRSIASEEAAARAGGRDYLKYCIEAAEALGATIVGGPLYGEPLVFAGRPPFPWTAEQIATRAARTVEGLAEVAPLAASAGKVFGLEPLNRFETDIVNTTAQAIEVVDAVGSPGLGVMLDTFHMNMEERSIPDAIRATGARLVHFQANENHRGFPGTGTMDWTAIARALGQAGYAGPVSLEPFRRDDERVALPIAHWRAPHEDEDEKLRAGLGLIRSAITLAEVTH

SEQ ID NO: 6 - DNA, 1422 bp, Escherichia coli
atgatgaaacaagaagttatcctggtactcgactgtggcgcgaccaatgtcagggccatcgcggttaatcggcagggcaaaattgttgcccgcgcctcaacgcctaatgccagcgatatcgcgatggaaaacaacacctggcaccagtggtctttagacgccattttgcaacgctttgctgattgctgtcggcaaatcaatagtgaactgactgaatgccacatccgcggtatcgccgtcaccacctttggtgtggatggcgctctggtagataagcaaggcaatctgctctatccgattattagctggaaatgtccgcgaacagcagcggttatggacaatattgaacggttaatctccgcacagcggttgcaggctatttctggcgtcggagcctttagtttcaatacgttatataagttggtgtggttgaaagaaaatcatccacaactgctggaacgcgcgcacgcctggctctttatttcgtcgctgattaaccaccgtttaaccggcgaattcactactgatatcacgatggccggaaccagccagatgctggatatccagcaacgcgatttcagtccgcaaattttacaagccaccggtattccacgccgactcttccctcgtctggtggaagcgggtgaacagattggtacgctacagaacagcgccgcagcaatgctcggcttacccgttggcataccggtgatttccgcaggtcacgatacccagttcgccctttttggcgctggtgctgaacaaaatgaacccgtgctctcttccggtacatgggaaattttaatggttcgcagcgcccaggttgatacttcgctgttaagtcagtacgccggttccacctgcgaactggatagccaggcagggttgtataacccaggtatgcaatggctggcatccggcgtgctggaatgggtgagaaaactgttctggacggctgaaacaccctggcaaatgttgattgaagaagctcgtctgatcgcgcctggcgcggatggcgtaaaaatgcagtgtgatttattgtcgtgtcagaacgctggctggcaaggagtgacgcttaataccacgcgggggcatttctatcgcgcggcgctggaagggttaactgcgcaattacagcgcaatctacagatgctggaaaaaatcgggcactttaaggcctctgaattattgttagtcggtggaggaagtcgcaacacattgtggaatcagattaaagccaatatgcttgatattccggtaaaagttctcgacgacgccgaaacgaccgtcgcaggagctgcgctgttcggttggtatggcgtaggggaatttaacagcccggaagaagcccgcgcacagattcattatcagtaccgttatttctacccgcaaactgaacctgaatttatagaggaagtgtga

SEQ ID NO: 7 - DNA, 1422 bp, Artificial Sequence; identical to SEQ ID NO: 6
  Escherichia coli L-fuculokinase FucK codon optimized sequence

SEQ ID NO: 8 - protein, 473 aa, Escherichia coli
MMKQEVILVLDCGATNVRAIAVNRQGKIVARASTPNASDIAMENNTWHQWSLDAILQRFADCCRQINSELTECHIRGIAVTTFGVDGALVDKQGNLLYPIISWKCPRTAAVMDNIERLISAQRLQAISGVGAFSFNTLYKLVWLKENHPQLLERAHAWLFISSLINHRLTGEFTTDITMAGTSQMLDIQQRDFSPQILQATGIPRRLFPRLVEAGEQIGTLQNSAAAMLGLPVGIPVISAGHDTQFALFGAGAEQNEPVLSSGTWEILMVRSAQVDTSLLSQYAGSTCELDSQAGLYNPGMQWLASGVLEWVRKLFWTAETPWQMLIEEARLIAPGADGVKMQCDLLSCQNAGWQGVTLNTTRGHFYRAALEGLTAQLQRNLQMLEKIGHFKASELLLVGGGSRNTLWNQIKANMLDIPVKVLDDAETTVAGAALFGWYGVGEFNSPEEARAQIHYQYRYFYPQTEPEFIEEV

SEQ ID NO: 9 - DNA, 648 bp, Escherichia coli
atggaacgaaataaacttgctcgtcagattattgacacttgcctggaaatgacccgcctgggactgaaccaggggacagcggggaacgtcagtgtacgttatcaggatgggatgctgattacgcctacaggcattccatatgaaaaactgacggagtcgcatattgtctttattgatggcaacggtaaacatgaggaaggaaagctcccctcaagcgaatggcgtttccatatggcagcctatcaaagcagaccggatgccaacgcggttgttcacaatcatgccgttcattgcacggcagtttccattcttaaccgatcgatccccgctattcactacatgattgcggcggctggcggtaattctattccttgcgcgccttatgcgacctttggaacacgcgaactttctgaacatgttgcgctggctctcaaaaatcgtaaggcaactttgttacaacatcatgggcttatcgcttgtgaggtgaatctggaaaaagcgttatggctggcgcatgaagttgaagtgctggcgcaactttacctgacgaccctggcgattacggacccggtgccagtgctgagcgatgaagagattgccgtagtgctggagaaattcaaaacctatgggttacgaattgaagagtaa

SEQ ID NO: 10 - DNA, 648 bp, Escherichia coli; identical to SEQ ID NO: 9

SEQ ID NO: 11 - protein, 215 aa, Escherichia coli
MERNKLARQIIDTCLEMTRLGLNQGTAGNVSVRYQDGMLITPTGIPYEKLTESHIVFIDGNGKHEEGKLPSSEWRFHMAAYQSRPDANAVVHNHAVHCTAVSILNRSIPAIHYMIAAAGGNSIPCAPYATFGTRELSEHVALALKNRKATLLQHHGLIACEVNLEKALWLAHEVEVLAQLYLTTLAITDPVPVLSDEEIAVVLEKFKTYGLRIEE

SEQ ID NO: 12 - DNA, 1104 bp, Escherichia coli
atggaccgcattattcaatcaccgggtaaatacatccagggcgctgatgtgattaatcgtctgggcgaatacctgaagccgctggcagaacgctggttagtggtgggtgacaaatttgttttaggttttgctcaatccactgtcgagaaaagctttaaagatgctggactggtagtagaaattgcgccgtttggcggtgaatgttcgcaaaatgagatcgaccgtctgcgtggcatcgcggagactgcgcagtgtggcgcaattctcggtatcggtggcggaaaaaccctcgatactgccaaagcactggcacatttcatgggtgttccggtagcgatcgcaccgactatcgcctctaccgatgcaccgtgcagcgcattgtctgttatctacaccgatgagggtgagtttgaccgctatctgctgttgccaaataacccgaatatggtcattgtcgacaccaaaatcgtcgctggcgcacctgcacgtctgttagcggcgggtatcggcgatgcgctggcaacctggtttgaagcgcgtgcctgctctcgtagcggcgcgaccaccatggcgggcggcaagtgcacccaggctgcgctggcactggctgaactgtgctacaacaccctgctggaagaaggcgaaaaagcgatgcttgctgccgaacagcatgtagtgactccggcgctggagcgcgtgattgaagcgaacacctatttgagcggtgttggttttgaaagtggtggtctggctgcggcgcacgcagtgcataacggcctgaccgctatcccggacgcgcatcactattatcacggtgaaaaagtggcattcggtacgctgacgcagctggttctggaaaatgcgccggtggaggaaatcgaaaccgtagctgcccttagccatgcggtaggtttgccaataactctcgctcaactggatattaaagaagatgtcccggcgaaaatgcgaattgtggcagaagcggcatgtgcagaaggtgaaaccattcacaacatgcctggcggcgcgacgccagatcaggtttacgccgctctgctggtagccgaccagtacggtcagcgtttcctgcaagagtgggaataa

SEQ ID NO: 13 - protein, 367 aa, Escherichia coli
MDRIIQSPGKYIQGADVINRLGEYLKPLAERWLVVGDKFVLGFAQSTVEKSFKDAGLVVEIAPFGGECSQNEIDRLRGIAETAQCGAILGIGGGKTLDTAKALAHFMGVPVAIAPTIASTDAPCSALSVIYTDEGEFDRYLLLPNNPNMVIVDTKIVAGAPARLLAAGIGDALATWFEARACSRSGATTMAGGKCTQAALALAELCYNTLLEEGEKAMLAAEQHVVTPALERVIEANTYLSGVGFESGGLAAAHAVHNGLTAIPDAHHYYHGEKVAFGTLTQLVLENAPVEEIETVAALSHAVGLPITLAQLDIKEDVPAKMRIVAEAACAEGETIHNMPGGATPDQVYAALLVADQYGQRFLQEWE

SEQ ID NO: 14 - DNA, 1029 bp, Saccharomyces cerevisiae
atgtcagttttcgtttcaggtgctaacgggttcattgcccaacacattgtcgatctcctgttgaaggaagactataaggtcatcggttctgccagaagtcaagaaaaggccgagaatttaacggaggcctttggtaacaacccaaaattctccatggaagttgtcccagacatatctaagctggacgcatttgaccatgttttccaaaagcacggcaaggatatcaagatagttctacatacggcctctccattctgctttgatatcactgacagtgaacgcgatttattaattcctgctgtgaacggtgttaagggaattctccactcaattaaaaaatacgccgctgattctgtagaacgtgtagttctcacctcttcttatgcagctgtgttcgatatggcaaaagaaaacgataagtctttaacatttaacgaagaatcctggaacccagctacctgggagagttgccaaagtgacccagttaacgcctactgtggttctaagaagtttgctgaaaaagcagcttgggaatttctagaggagaatagagactctgtaaaattcgaattaactgccgttaacccagtttacgtttttggtccgcaaatgtttgacaaagatgtgaaaaaacacttgaacacatcttgcgaactcgtcaacagcttgatgcatttatcaccagaggacaagataccggaactatttggtggatacattgatgttcgtgatgttgcaaaggctcatttagttgccttccaaaagagggaaacaattggtcaaagactaatcgtatcggaggccagatttactatgcaggatgttctcgatatccttaacgaagacttccctgttctaaaaggcaatattccagtggggaaaccaggttctggtgctacccataacacccttggtgctactcttgataataaaaagagtaagaaattgttaggtttcaagttcaggaacttgaaagagaccattgacgacactgcctcccaaattttaaaatttgagggcagaatataa

SEQ ID NO: 15 - protein, 342 aa, Saccharomyces cerevisiae
MSVFVSGANGFIAQHIVDLLLKEDYKVIGSARSQEKAENLTEAFGNNPKFSMEVVPDISKLDAFDHVFQKHGKDIKIVLHTASPFCFDITDSERDLLIPAVNGVKGILHSIKKYAADSVERVVLTSSYAAVFDMAKENDKSLTFNEESWNPATWESCQSDPVNAYCGSKKFAEKAAWEFLEENRDSVKFELTAVNPVYVFGPQMFDKDVKKHLNTSCELVNSLMHLSPEDKIPELFGGYIDVRDVAKAHLVAFQKRETIGQRLIVSEARFTMQDVLDILNEDFPVLKGNIPVGKPGSGATHNTLGATLDNKKSKKLLGFKFRNLKETIDDTASQILKFEGRI

SEQ ID NO: 16 - DNA, 984 bp, Saccharomyces cerevisiae
atgtcttcactggttactcttaataacggtctgaaaatgcccctagtcggcttagggtgctggaaaattgacaaaaaagtctgtgcgaatcaaatttatgaagctatcaaattaggctaccgtttattcgatggtgcttgcgactacggcaacgaaaaggaagttggtgaaggtatcaggaaagccatctccgaaggtcttgtttctagaaaggatatatttgttgtttcaaagttatggaacaattttcaccatcctgatcatgtaaaattagctttaaagaagaccttaagcgatatgggacttgattatttagacctgtattatattcacttcccaatcgccttcaaatatgttccatttgaagagaaataccctccaggattctatacgggcgcagatgacgagaagaaaggtcacatcaccgaagcacatgtaccaatcatagatacgtaccgggctctggaagaatgtgttgatgaaggcttgattaagtctattggtgtttccaactttcagggaagcttgattcaagatttattacgtggttgtagaatcaagcccgtggctttgcaaattgaacaccatccttatttgactcaagaacacctagttgagttttgtaaattacacgatatccaagtagttgcttactcctccttcggtcctcaatcattcattgagatggacttacagttggcaaaaaccacgccaactctgttcgagaatgatgtaatcaagaaggtctcacaaaaccatccaggcagtaccacttcccaagtattgcttagatgggcaactcagagaggcattgccgtcattccaaaatcttccaagaaggaaaggttacttggcaacctagaaatcgaaaaaaagttcactttaacggagcaagaattgaaggatatttctgcactaaatgccaacatcagatttaatgatccatggacctggttggatggtaaattccccacttttgcctga

SEQ ID NO: 17 - protein, 327 aa, Saccharomyces cerevisiae
MSSLVTLNNGLKMPLVGLGCWKIDKKVCANQIYEAIKLGYRLFDGACDYGNEKEVGEGIRKAISEGLVSRKDIFVVSKLWNNFHHPDHVKLALKKTLSDMGLDYLDLYYIHFPIAFKYVPFEEKYPPGFYTGADDEKKGHITEAHVPIIDTYRALEECVDEGLIKSIGVSNFQGSLIQDLLRGCRIKPVALQIEHHPYLTQEHLVEFCKLHDIQVVAYSSFGPQSFIEMDLQLAKTTPTLFENDVIKKVSQNHPGSTTSQVLLRWATQRGIAVIPKSSKKERLLGNLEIEKKFTLTEQELKDISALNANIRFNDPWTWLDGKFPTFA

SEQ ID NO: 18 - DNA, 1164 bp, Escherichia coli
atgaacaactttaatctgcacaccccaacccgcattctgtttggtaaaggcgcaatcgctggtttacgcgaacaaattcctcacgatgctcgcgtattgattacctacggcggcggcagcgtgaaaaaaaccggcgttctcgatcaagttctggatgccctgaaaggcatggacgtgctggaatttggcggtattgagccaaacccggcttatgaaacgctgatgaacgccgtgaaactggttcgcgaacagaaagtgactttcctgctggcggttggcggcggttctgtactggacggcaccaaatttatcgccgcagcggctaactatccggaaaatatcgatccgtggcacattctgcaaacgggcggtaaagagattaaaagcgccatcccgatgggctgtgtgctgacgctgccagcaaccggttcagaatccaacgcagaagcggtgatctcccgtaaaaccacaggcgacaagcaggcgttccattctgcccatgttcagccggtatttgccgtgctcgatccggtttatacctacaccctgccgccgcgtcaggtggctaacggcgtagtggacgcctttgtacacaccgtggaacagtatgttaccaaaccggttgatgccaaaattcaggaccgtttcgcagaaggcattttgctgacgctaatcgaagatggtccgaaagccctgaaagagccagaaaactacgatgtgcgcgccaacgtcatgtgggcggcgactcaggcgctgaacggtttgattggcgctggcgtaccgcaggactgggcaacgcatatgctgggccacgaactgactgcgatgcacggtctggatcacgcgcaaacactggctatcgtcctgcctgcactgtggaatgaaaaacgcgataccaagcgcgctaagctgctgcaatatgctgaacgcgtctggaacatcactgaaggttccgatgatgagcgtattgacgccgcgattgccgcaacccgcaatttctttgagcaattaggcgtgccgacccacctctccgactacggtctggacggcagctccatcccggctttgctgaaaaaactggaagagcacggcatgacccaactgggcgaaaatcatgacattacgttggatgtcagccgccgtatatacgaagccgcccgctaa

SEQ ID NO: 19 - DNA, 1167 bp, Artificial Sequence
  Escherichia coli alcohol dehydrogenase yqhD* codon optimized sequence
atgaacaattttaatttgcatactccaactagaatattatttggaaaaggtgcaattgcaggtttaagggaacaaataccacatgatgcaagggtattaatcacatacggtggtggttctgtcaagaaaactggtgtattggatcaagtattggatgctttaaagggtatggatgtcttggaatttggaggaatcgaaccaaaccctgcttacgagactttaatgaatgctgtcaaattggtcagagaacaaaaggtaacattcttattggctgttggaggtggatcagtattagatggtacaaagttcattgctgctgcagcaaattatccagaaaacattgatccatggcatatattgcaaactggtggtaaggaaataaagtcagctatcccaatgggatgtgttttgacattgcctgcaacaggatcagaatcaaacgctgaagcagtcatctcaagaaagactacaggtgacaaacaggcattccattctgcccatgtccaacctgtatttgctgttttagaccctgtatacacttacacattaccaccaaggcaagtcgcaaatggagttgtcgatgcctttgttcacactgtagaacagtacgtcaccaaaccagtcgatgcaaagatccaggacaggtttgcagaaggtattttattgacattaatcgaagatggaccaaaagcattgaaagagccagagaactatgacgttagggcaaatgttatgtgggctgctacccaggcattgaacggtttaattggtgcaggagttccacaagattgggctacacacatgttgggtcacgagttgaccgccatgcacggtttggaccatgcacagactttagccattgttttgcctgccttatggaacgagaaaagagatactaagagggctaagttattacaatacgctgaaagggtttggaatatcaccgagggatctgatgatgaaaggattgatgccgctattgcagccactagaaacttctttgaacaattaggtgttccaactcacttgtctgactatggtttagatggatcatctattccagctttgttgaagaaattggaagagcacggtatgacccagttgggtgagaatcatgatataaccttagatgtatctaggagaatctacgaggctgctagataatga

SEQ ID NO: 20 - protein, 387 aa, Escherichia coli
MNNFNLHTPTRILFGKGAIAGLREQIPHDARVLITYGGGSVKKTGVLDQVLDALKGMDVLEFGGIEPNPAYETLMNAVKLVREQKVTFLLAVGGGSVLDGTKFIAAAANYPENIDPWHILQTGGKEIKSAIPMGCVLTLPATGSESNAEAVISRKTTGDKQAFHSAHVQPVFAVLDPVYTYTLPPRQVANGVVDAFVHTVEQYVTKPVDAKIQDRFAEGILLTLIEDGPKALKEPENYDVRANVMWAATQALNGLIGAGVPQDWATHMLGHELTAMHGLDHAQTLAIVLPALWNEKRDTKRAKLLQYAERVWNITEGSDDERIDAAIAATRNFFEQLGVPTHLSDYGLDGSSIPALLKKLEEHGMTQLGENHDITLDVSRRIYEAAR

SEQ ID NO: 21 - DNA, 1164 bp, Escherichia coli
atgaacaactttaatctgcacaccccaacccgcattctgtttggtaaaggcgcaatcgctggtttacgcgaacaaattcctcacgatgctcgcgtattgattacctacggcggcggcagcgtgaaaaaaaccggcgttctcgatcaagttctggatgccctgaaaggcatggacgtgctggaatttggcggtattgagccaaacccggcttatgaaacgctgatgaacgccgtgaaactggttcgcgaacagaaagtgactttcctgctggcggttggcggcggttctgtactggacggcaccaaatttatcgccgcagcggctaactatccggaaaatatcgatccgtggcacattctgcaaacgggcggtaaagagattaaaagcgccatcccgatgggctgtgtgctgacgctgccagcaaccggttcagaatccaacgcaggcgcggtgatctcccgtaaaaccacaggcgacaagcaggcgttccattctgcccatgttcagccggtatttgccgtgctcgatccggtttatacctacaccctgccgccgcgtcaggtggctaacggcgtagtggacgcctttgtacacaccgtggaacagtatgttaccaaaccggttgatgccaaaattcaggaccgtttcgcagaaggcattttgctgacgctaatcgaagatggtccgaaagccctgaaagagccagaaaactacgatgtgcgcgccaacgtcatgtgggcggcgactcaggcgctgaacggtttgattggcgctggcgtaccgcaggactgggcaacgcatatgctgggccacgaactgactgcgatgcacggtctggatcacgcgcaaacactggctatcgtcctgcctgcactgtggaatgaaaaacgcgataccaagcgcgctaagctgctgcaatatgctgaacgcgtctggaacatcactgaaggttccgatgatgagcgtattgacgccgcgattgccgcaacccgcaatttctttgagcaattaggcgtgccgacccacctctccgactacggtctggacggcagctccatcccggctttgctgaaaaaactggaagagcacggcatgacccaactgggcgaaaatcatgacattacgttggatgtcagccgccgtatatacgaagccgcccgctaa

SEQ ID NO: 22 - DNA, 1164 bp, Artificial Sequence; identical to SEQ ID NO: 21
  Escherichia coli alcohol dehydrogenase yqhD codon optimized sequence

SEQ ID NO: 23 - protein, 387 aa, Escherichia coli
MNNFNLHTPTRILFGKGAIAGLREQIPHDARVLITYGGGSVKKTGVLDQVLDALKGMDVLEFGGIEPNPAYETLMNAVKLVREQKVTFLLAVGGGSVLDGTKFIAAAANYPENIDPWHILQTGGKEIKSAIPMGCVLTLPATGSESNAGAVISRKTTGDKQAFHSAHVQPVFAVLDPVYTYTLPPRQVANGVVDAFVHTVEQYVTKPVDAKIQDRFAEGILLTLIEDGPKALKEPENYDVRANVMWAATQALNGLIGAGVPQDWATHMLGHELTAMHGLDHAQTLAIVLPALWNEKRDTKRAKLLQYAERVWNITEGSDDERIDAAIAATRNFFEQLGVPTHLSDYGLDGSSIPALLKKLEEHGMTQLGENHDITLDVSRRIYEAAR

SEQ ID NO: 24 - DNA, 981 bp, Escherichia coli
atgaaaaagatacctttaggcacaacggatattacgctttcgcgaatggggttggggacatgggccattggcggcggtcctgcatggaatggcgatctcgatcggcaaatatgtattgatacgattcttgaagcccatcgttgtggcattaatctgattgatactgcgccaggatataactttggcaatagtgaagttatcgtcggtcaggcgttaaaaaaactgccccgtgaacaggttgtagtagaaaccaaatgcggcattgtctgggaacgaaaaggaagtttattcaacaaagttggcgatcggcagttgtataaaaacctttccccggaatctatccgcgaagaggtagcagcgagcttgcaacgtctgggtattgattacatcgatatctacatgacgcactggcagtcggtgccgccattttttacgccgatcgctgaaactgtcgcagtgcttaatgagttaaagtctgaagggaaaattcgcgctataggcgctgctaacgtcgatgctgaccatatccgcgagtatctgcaatatggtgaactggatattattcaggcgaaatacagtatcctcgaccgggcaatggaaaacgaactgctgccactatgtcgtgataatggcattgtggttcaggtttattccccgctagagcagggattgttgaccggcaccatcactcgtgattacgttccgggcggcgctcgggcaaataaagtctggttccagcgtgaaaacatgctgaaagtgattgatatgcttgaacagtggcagccactttgtgctcgttatcagtgcacaattcccactctggcactggcgtggatattaaaacagagtgatttaatctccattcttagtggggctactgcaccggaacaggtacgcgaaaatgtcgcggcactgaatatcaacttatcggatgcagacgcaacattgatgagggaaatggcagaggccctggagcgttaa

SEQ ID NO: 25 - protein, 326 aa, Escherichia coli
MKKIPLGTTDITLSRMGLGTWAIGGGPAWNGDLDRQICIDTILEAHRCGINLIDTAPGYNFGNSEVIVGQALKKLPREQVVVETKCGIVWERKGSLFNKVGDRQLYKNLSPESIREEVAASLQRLGIDYIDIYMTHWQSVPPFFTPIAETVAVLNELKSEGKIRAIGAANVDADHIREYLQYGELDIIQAKYSILDRAMENELLPLCRDNGIVVQVYSPLEQGLLTGTITRDYVPGGARANKVWFQRENMLKVIDMLEQWQPLCARYQCTIPTLALAWILKQSDLISILSGATAPEQVRENVAALNINLSDADATLMREMAEALER

SEQ ID NO: 26 - DNA, 1149 bp, Escherichia coli
atggctaacagaatgattctgaacgaaacggcatggtttggtcggggtgctgttggggctttaaccgatgaggtgaaacgccgtggttatcagaaggcgctgatcgtcaccgataaaacgctggtgcaatgcggcgtggtggcgaaagtgaccgataagatggatgctgcagggctggcatgggcgatttacgacggcgtagtgcccaacccaacaattactgtcgtcaaagaagggctcggtgtattccagaatagcggcgcggattacctgatcgctattggtggtggttctccacaggatacttgtaaagcgattggcattatcagcaacaacccggagtttgccgatgtgcgtagcctggaagggctttccccgaccaataaacccagtgtaccgattctggcaattcctaccacagcaggtactgcggcagaagtgaccattaactacgtgatcactgacgaagagaaacggcgcaagtttgtttgcgttgatccgcatgatatcccgcaggtggcgtttattgacgctgacatgatggatggtatgcctccagcgctgaaagctgcgacgggtgtcgatgcgctcactcatgctattgaggggtatattacccgtggcgcgtgggcgctaaccgatgcactgcacattaaagcgattgaaatcattgctggggcgctgcgaggatcggttgctggtgataaggatgccggagaagaaatggcgctcgggcagtatgttgcgggtatgggcttctcgaatgttgggttagggttggtgcatggtatggcgcatccactgggcgcgttttataacactccacacggtgttgcgaacgccatcctgttaccgcatgtcatgcgttataacgctgactttaccggtgagaagtaccgcgatatcgcgcgcgttatgggcgtgaaagtggaaggtatgagcctggaagaggcgcgtaatgccgctgttgaagcggtgtttgctctcaaccgtgatgtcggtattccgccacatttgcgtgatgttggtgtacgcaaggaagacattccggcactggcgcaggcggcactggatgatgtttgtaccggtggcaacccgcgtgaagcaacgcttgaggatattgtagagctttaccataccgcctggtaa

SEQ ID NO: 27 - DNA, 1149 bp, Artificial Sequence; identical to SEQ ID NO: 26
  Escherichia coli lactaldehyde reductase fucO codon optimized sequence

SEQ ID NO: 28 - protein, 382 aa, Escherichia coli
MANRMILNETAWFGRGAVGALTDEVKRRGYQKALIVTDKTLVQCGVVAKVTDKMDAAGLAWAIYDGVVPNPTITVVKEGLGVFQNSGADYLIAIGGGSPQDTCKAIGIISNNPEFADVRSLEGLSPTNKPSVPILAIPTTAGTAAEVTINYVITDEEKRRKFVCVDPHDIPQVAFIDADMMDGMPPALKAATGVDALTHAIEGYITRGAWALTDALHIKAIEIIAGALRGSVAGDKDAGEEMALGQYVAGMGFSNVGLGLVHGMAHPLGAFYNTPHGVANAILLPHVMRYNADFTGEKYRDIARVMGVKVEGMSLEEARNAAVEAVFALNRDVGIPPHLRDVGVRKEDIPALAQAALDDVCTGGNPREATLEDIVELYHTAW

SEQ ID NO: 29 - DNA, 804 bp, Escherichia coli
atggctatccctgcatttggtttaggtactttccgtctgaaagacgacgttgttatttcatctgtgataacggcgcttgaacttggttatcgcgcaattgataccgcacaaatctatgataacgaagccgcagtaggtcaggcgattgcagaaagtggcgtgccacgtcatgaactctacatcaccactaaaatctggattgaaaatctcagcaaagacaaattgatcccaagtctgaaagagagcctgcaaaaattgcgtaccgattatgttgatctgacgctaatccactggccgtcaccaaacgatgaagtctctgttgaagagtttatgcaggcgctgctggaagccaaaaaacaagggctgacgcgtgagatcggtatttccaacttcacgatcccgttgatggaaaaagcgattgctgctgttggtgctgaaaacatcgctactaaccagattgaactctctccttatctgcaaaaccgtaaagtggttgcctgggctaaacagcacggcatccatattacttcctatatgacgctggcgtatggtaaggccctgaaagatgaggttattgctcgtatcgcagctaaacacaatgcgactccggcacaagtgattctggcgtgggctatgggggaaggttactcagtaattccttcttctactaaacgtaaaaacctggaaagtaatcttaaggcacaaaatttacagcttgatgccgaagataaaaaagcgatcgccgcactggattgcaacgaccgcctggttagcccggaaggtctggctcctgaatgggattaa

SEQ ID NO: 30 - protein, 267 aa, Escherichia coli
MAIPAFGLGTFRLKDDVVISSVITALELGYRAIDTAQIYDNEAAVGQAIAESGVPRHELYITTKIWIENLSKDKLIPSLKESLQKLRTDYVDLTLIHWPSPNDEVSVEEFMQALLEAKKQGLTREIGISNFTIPLMEKAIAAVGAENIATNQIELSPYLQNRKVVAWAKQHGIHITSYMTLAYGKALKDEVIARIAAKHNATPAQVILAWAMGEGYSVIPSSTKRKNLESNLKAQNLQLDAEDKKAIAALDCNDRLVSPEGLAPEWD

SEQ ID NO: 31 - DNA, 828 bp, Escherichia coli
atggctaatccaaccgttattaagctacaggatggcaatgtcatgccccagctgggactgggcgtctggcaagcaagtaatgaggaagtaatcaccgccattcaaaaagcgttagaagtgggttatcgctcgattgataccgccgcggcctacaagaacgaagaaggtgtcggcaaagccctgaaaaatgcctcagtcaacagagaagaactgttcatcaccactaagctgtggaacgacgaccacaagcgcccccgcgaagccctgctcgacagcctgaaaaaactccagcttgattatatcgacctctacttaatgcactggcccgttcccgctatcgaccattatgtcgaagcatggaaaggcatgatcgaattgcaaaaagagggattaatcaaaagcatcggcgtgtgcaacttccagatccatcacctgcaacgcctgattgatgaaactggcgtgacgcctgtgataaaccagatcgaacttcatccgctgatgcaacaacgccagctacacgcctggaacgcgacacacaaaatccagaccgaatcctggagcccattagcgcaaggagggaaaggcgttttcgatcagaaagtcattcgcgatctggcagataaatacggcaaaaccccggcgcagattgttatccgctggcatctggatagcggcctggtggtgatcccgaaatcggtcacaccttcacgtattgccgaaaactttgatgtctgggatttccgtctcgacaaagacgaactcggcgaaattgcaaaactcgatcagggcaagcgtctcggtcccgatcctgaccagttcggcggctaa

SEQ ID NO: 32 - protein, 275 aa, Escherichia coli
MANPTVIKLQDGNVMPQLGLGVWQASNEEVITAIQKALEVGYRSIDTAAAYKNEEGVGKALKNASVNREELFITTKLWNDDHKRPREALLDSLKKLQLDYIDLYLMHWPVPAIDHYVEAWKGMIELQKEGLIKSIGVCNFQIHHLQRLIDETGVTPVINQIELHPLMQQRQLHAWNATHKIQTESWSPLAQGGKGVFDQKVIRDLADKYGKTPAQIVIRWHLDSGLVVIPKSVTPSRIAENFDVWDFRLDKDELGEIAKLDQGKRLGPDPDQFGG

SEQ ID NO: 33 - DNA, 1179 bp, Clostridium acetobutylicum
atgaaagaagttgtaatagctagtgcagtaagaacagcgattggatcttatggaaagtctcttaaggatgtaccagcagtagatttaggagctacagctataaaggaagcagttaaaaaagcaggaataaaaccagaggatgttaatgaagtcattttaggaaatgttcttcaagcaggtttaggacagaatccagcaagacaggcatcttttaaagcaggattaccagttgaaattccagctatgactattaataaggtttgtggttcaggacttagaacagttagcttagcagcacaaattataaaagcaggagatgctgacgtaataatagcaggtggtatggaaaatatgtctagagctccttacttagcgaataacgctagatggggatatagaatgggaaacgctaaatttgttgatgaaatgatcactgacggattgtgggatgcatttaatgattaccacatgggaataacagcagaaaacatagctgagagatggaacatttcaagagaagaacaagatgagtttgctcttgcatcacaaaaaaaagctgaagaagctataaaatcaggtcaatttaaagatgaaatagttcctgtagtaattaaaggcagaaagggagaaactgtagttgatacagatgagcaccctagatttggatcaactatagaaggacttgcaaaattaaaacctgccttcaaaaaagatggaacagttacagctggtaatgcatcaggattaaatgactgtgcagcagtacttgtaatcatgagtgcagaaaaagctaaagagcttggagtaaaaccacttgctaagatagtttcttatggttcagcaggagttgacccagcaataatgggatatggacctttctatgcaacaaaagcagctattgaaaaagcaggttggacagttgatgaattagatttaatagaatcaaatgaagcttttgcagctcaaagtttagcagtagcaaaagatttaaaatttgatatgaataaagtaaatgtaaatggaggagctattgcccttggtcatccaattggagcatcaggtgcaagaatactcgttactcttgtacacgcaatgcaaaaaagagatgcaaaaaaaggcttagcaactttatgtataggtggcggacaaggaacagcaatattgctagaaaagtgctag

SEQ ID NO: 34 - DNA, 1179 bp, Artificial Sequence
  Clostridium acetobutylicum acetyl coenzyme A acetyltransferase thlA codon optimized sequence
atgaaagaagttgttattgcgagcgcggttcgtaccgcgattggcagctatggcaagagcctgaaggatgttccggcggtggacctgggtgcgaccgcgatcaaagaggcggttaagaaagcgggcattaaaccggaggatgtgaacgaagttatcctgggtaacgtgctgcaagcgggtctgggccaaaacccggcgcgtcaggcgagcttcaaggcgggcctgccggttgaaatcccggcgatgaccattaacaaagtttgcggtagcggcctgcgtaccgtgagcctggcggcgcaaatcattaaggcgggtgacgcggatgttatcattgcgggtggcatggagaacatgagccgtgcgccgtacctggcgaacaacgcgcgttggggttatcgtatgggcaacgcgaaattcgtggacgaaatgattaccgacggtctgtgggatgcgtttaacgactaccacatgggcatcaccgcggagaacattgcggaacgttggaacattagccgtgaggaacaagatgagttcgcgctggcgagccagaagaaagcggaggaagcgatcaagagcggccagtttaaagacgaaatcgttccggtggttattaagggtcgtaagggtgaaaccgtggtggacaccgatgaacacccgcgtttcggtagcaccattgagggcctggcgaagctgaaaccggcgtttaagaaagatggcaccgtgaccgcgggtaacgcgagcggcctgaacgactgcgcggcggtgctggttatcatgagcgcggagaaggcgaaagaactgggtgtgaagccgctggcgaaaattgttagctacggtagcgcgggtgtggacccggcgatcatgggttacggcccgttttatgcgaccaaggcggcgattgagaaagcgggttggaccgtggacgaactggatctgatcgagagcaacgaagcgttcgcggcgcaaagcctggcggtggcgaaggatctgaaatttgacatgaacaaggtgaacgtgaacggtggtgcgattgcgctgggtcacccgattggtgcgagcggcgcgcgtatcctggtgaccctggttcacgcgatgcagaaacgtgacgcgaagaaaggtctggcgaccctgtgcattggtggtggtcaaggcaccgcgattctgctggaaaagtgctaa

SEQ ID NO: 35 - protein, 392 aa, Clostridium acetobutylicum
MKEVVIASAVRTAIGSYGKSLKDVPAVDLGATAIKEAVKKAGIKPEDVNEVILGNVLQAGLGQNPARQASFKAGLPVEIPAMTINKVCGSGLRTVSLAAQIIKAGDADVIIAGGMENMSRAPYLANNARWGYRMGNAKFVDEMITDGLWDAFNDYHMGITAENIAERWNISREEQDEFALASQKKAEEAIKSGQFKDEIVPVVIKGRKGETVVDTDEHPRFGSTIEGLAKLKPAFKKDGTVTAGNASGLNDCAAVLVIMSAEKAKELGVKPLAKIVSYGSAGVDPAIMGYGPFYATKAAIEKAGWTVDELDLIESNEAFAAQSLAVAKDLKFDMNKVNVNGGAIALGHPIGASGARILVTLVHAMQKRDAKKGLATLCIGGGQGTAILLEKC

SEQ ID NO: 36 - DNA, 1185 bp, Escherichia coli
atgaaaaattgtgtcatcgtcagtgcggtacgtactgctatcggtagttttaacggttcactcgcttccaccagcgccatcgacctgggggcgacagtaattaaagccgccattgaacgtgcaaaaatcgattcacaacacgttgatgaagtgattatgggtaacgtgttacaagccgggctggggcaaaatccggcgcgtcaggcactgttaaaaagcgggctggcagaaacggtgtgcggattcacggtcaataaagtatgtggttcgggtcttaaaagtgtggcgcttgccgcccaggccattcaggcaggtcaggcgcagagcattgtggcggggggtatggaaaatatgagtttagccccctacttactcgatgcaaaagcacgctctggttatcgtcttggagacggacaggtttatgacgtaatcctgcgcgatggcctgatgtgcgccacccatggttatcatatggggattaccgccgaaaacgtggctaaagagtacggaattacccgtgaaatgcaggatgaactggcgctacattcacagcgtaaagcggcagccgcaattgagtccggtgcttttacagccgaaatcgtcccggtaaatgttgtcactcgaaagaaaaccttcgtcttcagtcaagacgaattcccgaaagcgaattcaacggctgaagcgttaggtgcattgcgcccggccttcgataaagcaggaacagtcaccgctgggaacgcgtctggtattaacgacggtgctgccgctctggtgattatggaagaatctgcggcgctggcagcaggccttacccccctggctcgcattaaaagttatgccagcggtggcgtgccccccgcattgatgggtatggggccagtacctgccacgcaaaaagcgttacaactggcggggctgcaactggcggatattgatctcattgaggctaatgaagcatttgctgcacagttccttgccgttgggaaaaacctgggctttgattctgagaaagtgaatgtcaacggcggggccatcgcgctcgggcatcctatcggtgccagtggtgctcgtattctggtcacactattacatgccatgcaggcacgcgataaaacgctggggctggcaacactgtgcattggcggcggtcagggaattgcgatggtgattgaacggttgaattaa

SEQ ID NO: 37 - protein, 394 aa, Escherichia coli
MKNCVIVSAVRTAIGSFNGSLASTSAIDLGATVIKAAIERAKIDSQHVDEVIMGNVLQAGLGQNPARQALLKSGLAETVCGFTVNKVCGSGLKSVALAAQAIQAGQAQSIVAGGMENMSLAPYLLDAKARSGYRLGDGQVYDVILRDGLMCATHGYHMGITAENVAKEYGITREMQDELALHSQRKAAAAIESGAFTAEIVPVNVVTRKKTFVFSQDEFPKANSTAEALGALRPAFDKAGTVTAGNASGINDGAAALVIMEESAALAAGLTPLARIKSYASGGVPPALMGMGPVPATQKALQLAGLQLADIDLIEANEAFAAQFLAVGKNLGFDSEKVNVNGGAIALGHPIGASGARILVTLLHAMQARDKTLGLATLCIGGGQGIAMVIERLN

SEQ ID NO: 38 - DNA, 1197 bp, Saccharomyces cerevisiae
atgtctcagaacgtttacattgtatcgactgccagaaccccaattggttcattccagggttctctatcctccaagacagcagtggaattgggtgctgttgctttaaaaggcgccttggctaaggttccagaattggatgcatccaaggattttgacgaaattatttttggtaacgttctttctgccaatttgggccaagctccggccagacaagttgctttggctgccggtttgagtaatcatatcgttgcaagcacagttaacaaggtctgtgcatccgctatgaaggcaatcattttgggtgctcaatccatcaaatgtggtaatgctgatgttgtcgtagctggtggttgtgaatctatgactaacgcaccatactacatgccagcagcccgtgcgggtgccaaatttggccaaactgttcttgttgatggtgtcgaaagagatgggttgaacgatgcgtacgatggtctagccatgggtgtacacgcagaaaagtgtgcccgtgattgggatattactagagaacaacaagacaattttgccatcgaatcctaccaaaaatctcaaaaatctcaaaaggaaggtaaattcgacaatgaaattgtacctgttaccattaagggatttagaggtaagcctgatactcaagtcacgaaggacgaggaacctgctagattacacgttgaaaaattgagatctgcaaggactgttttccaaaaagaaaacggtactgttactgccgctaacgcttctccaatcaacgatggtgctgcagccgtcatcttggtttccgaaaaagttttgaaggaaaagaatttgaagcctttggctattatcaaaggttggggtgaggccgctcatcaaccagctgattttacatgggctccatctcttgcagttccaaaggctttgaaacatgctggcatcgaagacatcaattctgttgattactttgaattcaatgaagccttttcggttgtcggtttggtgaacactaagattttgaagctagacccatctaaggttaatgtatatggtggtgctgttgctctaggtcacccattgggttgttctggtgctagagtggttgttacactgctatccatcttacagcaagaaggaggtaagatcggtgttgccgccatttgtaatggtggtggtggtgcttcctctattgtcattgaaaagatatga

SEQ ID NO: 39 - DNA, 1197 bp, Artificial Sequence; identical to SEQ ID NO: 38
  Saccharomyces cerevisiae acetyl coenzyme A acetyltransferase ERG10 codon optimized NT sequence

SEQ ID NO: 40 - protein, 398 aa, Saccharomyces cerevisiae
MSQNVYIVSTARTPIGSFQGSLSSKTAVELGAVALKGALAKVPELDASKDFDEIIFGNVLSANLGQAPARQVALAAGLSNHIVASTVNKVCASAMKAIILGAQSIKCGNADVVVAGGCESMTNAPYYMPAARAGAKFGQTVLVDGVERDGLNDAYDGLAMGVHAEKCARDWDITREQQDNFAIESYQKSQKSQKEGKFDNEIVPVTIKGFRGKPDTQVTKDEEPARLHVEKLRSARTVFQKENGTVTAANASPINDGAAAVILVSEKVLKEKNLKPLAIIKGWGEAAHQPADFTWAPSLAVPKALKHAGIEDINSVDYFEFNEAFSVVGLVNTKILKLDPSKVNVYGGAVALGHPLGCSGARVVVTLLSILQQEGGKIGVAAICNGGGGASSIVIEKI

SEQ ID NO: 41 - DNA, 651 bp, Escherichia coli
atggatgcgaaacaacgtattgcgcgccgtgtggcgcaagagcttcgtgatggtgacatcgttaacttagggatcggtttacccacaatggtcgccaattatttaccggagggtattcatatcactctgcaatcggaaaacggcttcctcggtttaggcccggtcacgacagcgcatccagatctggtgaacgctggcgggcaaccgtgcggtgttttacccggtgcagccatgtttgatagcgccatgtcatttgcgctaatccgtggcggtcatattgatgcctgcgtgctcggcggtttgcaagtagacgaagaagcaaacctcgcgaactgggtagtgcctgggaaaatggtgcccggtatgggtggcgcgatggatctggtgaccgggtcgcgcaaagtgatcatcgccatggaacattgcgccaaagatggttcagcaaaaattttgcgccgctgcaccatgccactcactgcgcaacatgcggtgcatatgctggttactgaactggctgtctttcgttttattgacggcaaaatgtggctcaccgaaattgccgacgggtgtgatttagccaccgtgcgtgccaaaacagaagctcggtttgaagtcgccgccgatctgaatacgcaacggggtgatttatga

SEQ ID NO: 42 - DNA, 651 bp, Artificial Sequence; identical to SEQ ID NO: 41
  Escherichia coli Acetyl-CoAacetoacetate-CoA transferase subunit atoA codon optimized sequence

SEQ ID NO: 43 - protein, 216 aa, Escherichia coli
MDAKQRIARRVAQELRDGDIVNLGIGLPTMVANYLPEGIHITLQSENGFLGLGPVTTAHPDLVNAGGQPCGVLPGAAMFDSAMSFALIRGGHIDACVLGGLQVDEEANLANWVVPGKMVPGMGGAMDLVTGSRKVIIAMEHCAKDGSAKILRRCTMPLTAQHAVHMLVTELAVFRFIDGKMWLTEIADGCDLATVRAKTEARFEVAADLNTQRGDL

SEQ ID NO: 44 - DNA, 663 bp, Escherichia coli
atgaaaacaaaattgatgacattacaagacgccaccggcttctttcgtgacggcatgaccatcatggtgggcggatttatggggattggcactccatcccgcctggttgaagcattactggaatctggtgttcgcgacctgacattgatagccaatgataccgcgtttgttgataccggcatcggtccgctcatcgtcaatggtcgagtccgcaaagtgattgcttcacatatcggcaccaacccggaaacaggtcggcgcatgatatctggtgagatggacgtcgttctggtgccgcaaggtacgctaatcgagcaaattcgctgtggtggagctggacttggtggttttctcaccccaacgggtgtcggcaccgtcgtagaggaaggcaaacagacactgacactcgacggtaaaacctggctgctcgaacgcccactgcgcgccgacctggcgctaattcgcgctcatcgttgcgacacacttggcaacctgacctatcaacttagcgcccgcaactttaaccccctgatagcccttgcggctgatatcacgctggtagagccagatgaactggtcgaaaccggcgagctgcaacctgaccatattgtcacccctggtgccgttatcgaccacatcatcgtttcacaggagagcaaataa

SEQ ID NO: 45 - DNA, 663 bp, Artificial Sequence; identical to SEQ ID NO: 44
  Escherichia coli Acetyl-CoAacetoacetate-CoA transferase subunit atoD codon optimized sequence

SEQ ID NO: 46 - protein, 220 aa, Escherichia coli
MKTKLMTLQDATGFFRDGMTIMVGGFMGIGTPSRLVEALLESGVRDLTLIANDTAFVDTGIGPLIVNGRVRKVIASHIGTNPETGRRMISGEMDVVLVPQGTLIEQIRCGGAGLGGFLTPTGVGTVVEEGKQTLTLDGKTWLLERPLRADLALIRAHRCDTLGNLTYQLSARNFNPLIALAADITLVEPDELVETGELQPDHIVTPGAVIDHIIVSQESK

SEQ ID NO: 47 - DNA, 735 bp, Clostridium acetobutylicum
atgttaaaggatgaagtaattaaacaaattagcacgccattaacttcgcctgcatttcctagaggaccctataaatttcataatcgtgagtattttaacattgtatatcgtacagatatggatgcacttcgtaaagttgtgccagagcctttagaaattgatgagcccttagtcaggtttgaaattatggcaatgcatgatacgagtggacttggttgttatacagaaagcggacaggctattcccgtaagctttaatggagttaagggagattatcttcatatgatgtatttagataatgagcctgcaattgcagtaggaagggaattaagtgcatatcctaaaaagctcgggtatccaaagctttttgtggattcagatactttagtaggaactttagactatggaaaacttagagttgcgacagctacaatggggtacaaacataaagccttagatgctaatgaagcaaaggatcaaatttgtcgccctaattatatgttgaaaataatacccaattatgatggaagccctagaatatgtgagcttataaatgcgaaaatcacagatgttaccgtacatgaagcttggacaggaccaactcgactgcagttatttgatcacgctatggcgccacttaatgatttgccagtaaaagagattgtttctagctctcacattcttgcagatataatattgcctagagctgaagttatatatgattatcttaagtaa

SEQ ID NO: 48 - DNA, 735 bp, Artificial Sequence
  Clostridium acetobutylicum acetoacetate decarboxylase adc codon optimized sequence
atgctgaaggacgaggttattaagcagattagcaccccgctgaccagcccggcgttcccgcgtggtccgtacaagttccataatcgcgaatacttcaacattgtgtatcgtaccgacatggatgcgctgcgtaaggtggttccggagccgctggaaattgacgagccgctggttcgtttcgaaatcatggcgatgcacgataccagcggtctgggctgctacaccgagagcggtcaggcgattccggtgagctttaacggtgttaaaggcgactacctgcacatgatgtatctggataacgaaccggcgattgcggtgggtcgtgagctgagcgcgtacccgaagaaactgggctatccgaagctgttcgtggacagcgataccctggtgggcaccctggactacggcaaactgcgtgttgcgaccgcgaccatgggctataagcacaaagcgctggacgcgaacgaagcgaaggatcagatttgccgtccgaactacatgctgaaaatcattccgaactatgacggtagcccgcgtatctgcgaactgattaacgcgaagatcaccgatgttaccgttcatgaggcgtggaccggcccgacccgtctgcaactgtttgaccacgcgatggcgccgctgaacgatctgccggtgaaagagatcgttagcagcagccacatcctggcggacatcatcctgccgcgtgcggaagttatctacgattacctgaagtaa

SEQ ID NO: 49 - protein, 244 aa, Clostridium acetobutylicum
MLKDEVIKQISTPLTSPAFPRGPYKFHNREYFNIVYRTDMDALRKVVPEPLEIDEPLVRFEIMAMHDTSGLGCYTESGQAIPVSFNGVKGDYLHMMYLDNEPAIAVGRELSAYPKKLGYPKLFVDSDTLVGTLDYGKLRVATATMGYKHKALDANEAKDQICRPNYMLKIIPNYDGSPRICELINAKITDVTVHEAWTGPTRLQLFDHAMAPLNDLPVKEIVSSSHILADIILPRAEVIYDYLK

SEQ ID NO: 50 - DNA, 741 bp, Clostridium beijerinckii
atgttagaaagtgaagtatctaaacaaattacaactccacttgctgctccagcgtttcctagaggaccatataggtttcacaatagagaatatctaaacattatttatcgaactgatttagatgctcttcgaaaaatagtaccagagccacttgaattagatagagcatatgttagatttgaaatgatggctatgcctgatacaaccggactaggctcatatacagaatgtggtcaagctattccagtaaaatataatggtgttaagggtgactacttgcatatgatgtatctagataatgaacctgctattgctgttggaagagaaagtagcgcttatccaaaaaagcttggctatccaaagctatttgttgattcagatactttagttgggacacttaaatatggtacattaccagtagctactgcaacaatgggatataagcacgagcctctagatcttaaagaagcctatgctcaaattgcaagacccaattttatgctaaaaatcattcaaggttacgatggtaagccaagaatttgtgaactaatatgtgcagaaaatactgatataactattcacggtgcttggactggaagtgcacgtctacaattatttagccatgcactagctcctcttgctgatttacctgtattagagattgtatcagcatctcatatcctcacagatttaactcttggaacacctaaggttgtacatgattatctttcagtaaaataa

SEQ ID NO: 51 - DNA, 741 bp, Artificial Sequence
  Clostridium beijerinckii acetoacetate decarboxylase adc codon optimized sequence
atgctggagagcgaagttagcaaacaaatcaccaccccgctggcggcgccggcgttcccgcgtggcccgtaccgttttcataaccgtgagtacctgaacatcatttatcgtaccgacctggatgcgctgcgtaagattgtgccggagccgctggaactggaccgtgcgtacgttcgtttcgagatgatggcgatgccggataccaccggtctgggcagctacaccgaatgcggtcaggcgatcccggtgaagtataacggtgttaaaggcgactacctgcacatgatgtatctggataacgagccggcgattgcggtgggtcgtgaaagcagcgcgtacccgaagaaactgggctatccgaagctgtttgtggacagcgataccctggtgggcaccctgaaatatggcaccctgccggttgcgaccgcgaccatgggctacaagcacgagccgctggacctgaaagaagcgtatgcgcagattgcgcgtccgaacttcatgctgaagatcattcaaggttatgacggcaaaccgcgtatctgcgagctgatttgcgcggaaaacaccgatatcaccatccatggtgcgtggaccggcagcgcgcgtctgcaactgtttagccatgcgctggcgccgctggcggatctgccggtgctggaaatcgttagcgcgagccacattctgaccgatctgaccctgggcaccccgaaggttgtgcatgactatctgagcgtgaagtaa

SEQ ID NO: 52 - protein, 246 aa, Clostridium beijerinckii
MLESEVSKQITTPLAAPAFPRGPYRFHNREYLNIIYRTDLDALRKIVPEPLELDRAYVRFEMMAMPDTTGLGSYTECGQAIPVKYNGVKGDYLHMMYLDNEPAIAVGRESSAYPKKLGYPKLFVDSDTLVGTLKYGTLPVATATMGYKHEPLDLKEAYAQIARPNFMLKIIQGYDGKPRICELICAENTDITIHGAWTGSARLQLFSHALAPLADLPVLEIVSASHILTDLTLGTPKVVHDYLSVK

SEQ ID NO: 53 - DNA, 897 bp, Homo sapiens
atggaagagaagcagatcctgtgcgtggggctagtggtgctggacgtcatcagcctggtggacaagtaccctaaggaggactcggagataaggtgtttgtcccagagatggcagcgcggaggcaacgcgtccaactcctgcaccgttctctccctgctcggagccccctgtgccttcatgggctcaatggctcctggccatgttgctgattttgtcctggatgacctccgccgctattctgtggacctacgctacacagtctttcagaccacaggctccgtccccatcgccacggtcatcatcaacgaggccagtggtagccgcaccatcctatactatgacaggagcctgccagatgtgtctgctacagactttgagaaggttgatctgacccagttcaagtggatccacattgagggccggaacgcatcggagcaggtgaagatgctgcagcggatagacgcacacaacaccaggcagcctccagagcagaagatccgggtgtccgtggaggtggagaagccacgagaggagctcttccagctgtttggctacggagacgtggtgtttgtcagcaaagatgtggccaagcacttggggttccagtcagcagaggaagccttgaggggcttgtatggtcgtgtgaggaaaggggctgtgcttgtctgtgcctgggctgaggagggcgccgacgccctgggccctgatggcaaattgctccactcggatgctttcccgccaccccgcgtggtggatacactgggagctggagacaccttcaatgcctccgtcatcttcagcctctcccaggggaggagcgtgcaggaagcactgagattcgggtgccaggtggccggcaagaagtgtggcctgcagggctttgatggcatcgtttaa

SEQ ID NO: 54 - DNA, 897 bp, Artificial Sequence
  Homo sapiens ketohexokinase C khk-C codon optimized cDNA sequence
atggaggaaaagcaaattctgtgcgttggtctggtggttctggacgtgattagcctggttgataagtacccgaaagaggatagcgaaatccgttgcctgagccagcgttggcaacgtggtggcaacgcgagcaatagctgcaccgttctgagcctgctgggtgcgccgtgcgcgttcatgggtagcatggcgccgggtcatgttgcggacttcctggtggcggattttcgtcgtcgtggtgtggacgttagccaggttgcgtggcaaagcaagggcgataccccgagctcctgctgcatcattaacaacagcaacggtaaccgtaccattgtgctgcacgacaccagcctgccggatgttagcgcgaccgacttcgagaaggtggatctgacccagtttaaatggattcacattgagggccgtaacgcgagcgaacaggttaaaatgctgcaacgtattgatgcgcacaacacccgtcagccgccggaacaaaagattcgtgtgagcgttgaggtggaaaaaccgcgtgaggaactgttccaactgtttggttacggcgacgtggttttcgttagcaaggatgtggcgaaacacctgggttttcaaagcgcggaggaagcgctgcgtggtctgtatggccgtgtgcgtaaaggcgcggttctggtgtgcgcgtgggcggaggaaggcgcggatgcgctgggtccggatggcaaactgctgcacagcgatgcgttcccgccgccgcgtgtggttgacaccctgggtgcgggcgataccttcaacgcgagcgttatctttagcctgagccagggccgtagcgtgcaagaggcgctgcgtttcggctgccaagttgcgggtaaaaaatgcggtctgcaaggctttgacggtatcgtgtaa

SEQ ID NO: 55 - protein, 298 aa, Homo sapiens
MEEKQILCVGLVVLDVISLVDKYPKEDSEIRCLSQRWQRGGNASNSCTVLSLLGAPCAFMGSMAPGHVADFLVADFRRRGVDVSQVAWQSKGDTPSSCCIINNSNGNRTIVLHDTSLPDVSATDFEKVDLTQFKWIHIEGRNASEQVKMLQRIDAHNTRQPPEQKIRVSVEVEKPREELFQLFGYGDVVFVSKDVAKHLGFQSAEEALRGLYGRVRKGAVLVCAWAEEGADALGPDGKLLHSDAFPPPRVVDTLGAGDTFNASVIFSLSQGRSVQEALRFGCQVAGKKCGLQGFDGIV

SEQ ID NO: 56 - DNA, 1095 bp, Homo sapiens
atggcccaccgatttccagccctcacccaggagcagaagaaggagctctcagaaattgcccagagcattgttgccaatggaaaggggatcctggctgcagatgaatctgtaggtaccatggggaaccgcctgcagaggatcaaggtggaaaacactgaagagaaccgccggcagttccgagaaatcctcttctctgtggacagttccatcaaccagagcatcgggggtgtgatccttttccacgagaccctctaccagaaggacagccagggaaagctgttcagaaacatcctcaaggaaaaggggatcgtggtgggaatcaagttagaccaaggaggtgctcctcttgcaggaacaaacaaagaaaccaccattcaagggcttgatggcctctcagagcgctgtgctcagtacaagaaagatggtgttgactttgggaagtggcgtgctgtgctgaggattgccgaccagtgtccatccagcctcgctatccaggaaaacgccaacgccctggctcgctacgccagcatctgtcagcagaatggactggtacctattgttgaaccagaggtaattcctgatggagaccatgacctggaacactgccagtatgttactgagaaggtcctggctgctgtctacaaggccctgaatgaccatcatgtttacctggagggcaccctgctaaagcccaacatggtgactgctggacatgcctgcaccaagaagtatactccagaacaagtagctatggccaccgtaacagctctccaccgtactgttcctgcagctgttcctggcatctgctttttgtctggtggcatgagtgaagaggatgccactctcaacctcaatgctatcaacctttgccctctaccaaagccctggaaactaagtttctcttatggacgggccctgcaggccagtgcactggctgcctggggtggcaaggctgcaaacaaggaggcaacccaggaggcttttatgaagcgggccatggctaactgccaggcggccaaaggacagtatgttcacacgggttcttctggggctgcttccacccagtcgctcttcacagcctgctatacctactag

SEQ ID NO: 57 - DNA, 1095 bp, Artificial Sequence
  Homo sapiens Fructose-bisphosphate aldolase B aldoB codon optimized cDNA sequence
atggcgcaccgttttccggcgctgacccaagagcagaagaaggagctgagcgagattgcgcagagcatcgtggcgaatggtaaaggtattctggcggcggatgagagcgttggtaccatgggcaaccgtctgcagcgtattaaggtggagaacaccgaggaaaaccgtcgtcaattccgtgaaatcctgtttagcgttgatagcagcatcaaccagagcattggtggcgtgatcctgttccacgaaaccctgtaccagaaggacagccaaggtaaactgtttcgtaacattctgaaggaaaaaggtattgtggttggcatcaagctggatcaaggtggcgcgccgctggcgggcaccaacaaggaaaccaccatccagggtctggacggcctgagcgaacgttgcgcgcaatataagaaagatggtgttgacttcggcaagtggcgtgcggtgctgcgtattgcggaccagtgcccgagcagcctggcgatccaagaaaacgcgaacgcgctggcgcgttacgcgagcatctgccagcaaaacggtctggtgccgattgttgagccggaagttatcccggacggcgatcacgacctggagcactgccagtatgtgaccgaaaaggttctggcggcggtgtacaaagcgctgaacgatcaccacgtttatctggagggtaccctgctgaaaccgaacatggtgaccgcgggccatgcgtgcaccaagaaatacaccccggaacaggtggcgatggcgaccgtgaccgcgctgcaccgtaccgttccggcggcggtgccgggtatttgctttctgagcggtggcatgagcgaagaggacgcgaccctgaacctgaacgcgatcaacctgtgcccgctgccgaagccgtggaaactgagcttcagctacggccgtgcgctgcaggcgagcgcgctggcggcgtggggtggcaaggcggcgaacaaagaggcgacccaagaagcgtttatgaagcgtgcgatggcgaactgccaggcggcgaaaggtcaatatgtgcataccggcagcagcggtgcggcgagcacccagagcctgtttaccgcgtgctatacctattaa

SEQ ID NO: 58 - protein, 364 aa, Homo sapiens
MAHRFPALTQEQKKELSEIAQSIVANGKGILAADESVGTMGNRLQRIKVENTEENRRQFREILFSVDSSINQSIGGVILFHETLYQKDSQGKLFRNILKEKGIVVGIKLDQGGAPLAGTNKETTIQGLDGLSERCAQYKKDGVDFGKWRAVLRIADQCPSSLAIQENANALARYASICQQNGLVPIVEPEVIPDGDHDLEHCQYVTEKVLAAVYKALNDHHVYLEGTLLKPNMVTAGHACTKKYTPEQVAMATVTALHRTVPAAVPGICFLSGGMSEEDATLNLNAINLCPLPKPWKLSFSYGRALQASALAAWGGKAANKEATQEAFMKRAMANCQAAKGQYVHTGSSGAASTQSLFTACYTY

SEQ ID NO: 59 - DNA, 747 bp, Caulobacter crescentus
atgtcctcagccatctatcccagcctgaagggcaagcgcgtcgtcatcaccggcggcggctcgggcatcggggccggcctcaccgccggcttcgcccgtcagggcgcggaggtgatcttcctcgacatcgccgacgaggactccagggctcttgaggccgagctggccggctcgccgatcccgccggtctacaagcgctgcgacctgatgaacctcgaggcgatcaaggcggtcttcgccgagatcggcgacgtcgacgtgctggtcaacaacgccggcaatgacgaccgccacaagctggccgacgtgaccggcgcctattgggacgagcggatcaacgtcaacctgcgccacatgctgttctgcacccaggccgtcgcgccgggcatgaagaagcgtggcggcggggcggtgatcaacttcggttcgatcagctggcacctggggcttgaggacctcgtcctctacgaaaccgccaaggccggcatcgaaggcatgacccgcgcgctggcccgggagctgggtcccgacgacatccgcgtcacctgcgtggtgccgggcaacgtcaagaccaagcgccaggagaagtggtacacgcccgaaggcgaggcccagatcgtggcggcccaatgcctgaagggccgcatcgtcccggagaacgtcgccgcgctggtgctgttcctggcctcggatgacgcgtcgctctgcaccggccacgaatactggatcgacgccggctggcgttga

SEQ ID NO: 60 - DNA, 747 bp, Artificial Sequence
  Caulobacter crescentus D-xylose 1-dehydrogenase xylB codon optimized sequence
atgagcagcgcgatctacccgagcctgaaaggtaaacgtgtggtgattaccggcggcggcagcggcattggtgcgggcctgaccgcgggcttcgcgcgtcagggtgcggaagtgatctttctggacattgcggacgaagatagccgtgcgctggaggcggaactggcgggcagcccgatcccgccggtgtacaagcgttgcgatctgatgaacctggaggcgatcaaagcggttttcgcggaaattggcgacgtggatgttctggtgaacaacgcgggtaacgacgaccgtcacaagctggcggatgtgaccggtgcgtattgggatgagcgtattaacgttaacctgcgtcacatgctgttctgcacccaggcggtggcgccgggtatgaagaaacgtggtggcggtgcggttatcaactttggcagcattagctggcacctgggtctggaggacctggtgctgtacgaaaccgcgaaagcgggcatcgagggtatgacccgtgcgctggcgcgtgaactgggtccggacgatattcgtgtgacctgcgtggttccgggtaacgttaagaccaaacgtcaagagaagtggtataccccggagggtgaagcgcagattgttgcggcgcaatgcctgaaaggtcgtattgttccggaaaacgtggcggcgctggttctgtttctggcgagcgatgatgcgagcctgtgcaccggccatgagtattggattgatgcgggctggcgttaa

SEQ ID NO: 61 - protein, 248 aa, Caulobacter crescentus
MSSAIYPSLKGKRVVITGGGSGIGAGLTAGFARQGAEVIFLDIADEDSRALEAELAGSPIPPVYKRCDLMNLEAIKAVFAEIGDVDVLVNNAGNDDRHKLADVTGAYWDERINVNLRHMLFCTQAVAPGMKKRGGGAVINFGSISWHLGLEDLVLYETAKAGIEGMTRALARELGPDDIRVTCVVPGNVKTKRQEKWYTPEGEAQIVAAQCLKGRIVPENVAALVLFLASDDASLCTGHEYWIDAGWR

SEQ ID NO: 62 - DNA, 1173 bp, Haloferax volcanii
atgagccccgcccccaccgacatcgtcgaggagttcacgcgccgcgactggcagggagacgacgtgacgggcaccgtgcgggtcgccatgatcggcctcggctggtggacccgcgacgaggcgattcccgcggtcgaggcgtccgagttctgcgagacgacggtcgtcgtcagcagttcgaaggagaaagccgagggcgcgacggcgttgaccgagtcgataacccacggcctcacctacgacgagttccacgagggggtcgccgccgacgcctacgacgcggtgtacgtcgtcacgccgaacggtctgcatctcccgtacgtcgagaccgccgccgagttggggaaggcggtcctctgcgagaaaccgctggaagcgtcggtcgagcgggccgaaaagctcgtcgccgcctgcgaccgcgccgacgtgcccctgatggtcgcctatcggatgcagaccgagccggccgtccggcgcgcccgcgaactcgtcgaggccggcgtcatcggcgagccggtgttcgtccacggccacatgtcccagcgcctgctcgacgaggtcgtccccgaccccgaccagtggcggctcgaccccgaactctccggcggcgcgaccgtcatggacatcgggctctacccgctgaacaccgcccggttcgtcctcgacgccgaccccgtccgcgtcagggcgaccgcccgcgtcgacgacgaggcgttcgaggccgtcggcgacgagcacgtcagtttcggcgtcgacttcgacgacggcacgctcgcggtctgcaccgccagccagtcggcttaccagttgagccacctccgggtgaccggcaccgagggcgaactcgaaatcgagcccgcgttctacaaccgccaaaagcggggattccgactgtcgtggggggaccagtccgccgactacgacttcgagcaggtaaaccagatgacggaggagttcgactacttcgcgtcccggctcctgtcggattccgaccccgcgcccgacggcgaccacgcgctcgtggacatgcgcgcgatggacgcgatttacgccgcggcggagcgcgggaccgatgtcgccgtcgacgccgccgactccgattccgccgactccgattccgccgacgctgccgccgccaaccacgacgccgaccccgattccgacgggacgtag

SEQ ID NO: 63 - protein, 390 aa, Haloferax volcanii
MSPAPTDIVEEFTRRDWQGDDVTGTVRVAMIGLGWWTRDEAIPAVEASEFCETTVVVSSSKEKAEGATALTESITHGLTYDEFHEGVAADAYDAVYVVTPNGLHLPYVETAAELGKAVLCEKPLEASVERAEKLVAACDRADVPLMVAYRMQTEPAVRRARELVEAGVIGEPVFVHGHMSQRLLDEVVPDPDQWRLDPELSGGATVMDIGLYPLNTARFVLDADPVRVRATARVDDEAFEAVGDEHVSFGVDFDDGTLAVCTASQSAYQLSHLRVTGTEGELEIEPAFYNRQKRGFRLSWGDQSADYDFEQVNQMTEEFDYFASRLLSDSDPAPDGDHALVDMRAMDAIYAAAERGTDVAVDAADSDSADSDSADAAAANHDADPDSDGT

SEQ ID NO: 64 - DNA, 1176 bp, Trichoderma reesei
atggcgtctggaaacccttacaccctgaaatggggcatcatggccaccggcggaatcgcagagaccttctgcaaggatctcctgtgcaaccccgcgattcgaggcgccgatgatgtgcgccacgagattgtggccgtggcctcttccagcagcagcaagagagcagaggagttcctccagagaatcgacggtgcctttgacgccaagacgtacggatcatacccggaacttgtggcagaccccaacgtcgacatcgtctatgtggcaactccccacagccaccacttccagaacaccatgctggcgctggaagccggcaagaacgtcttgtgcgaaaaggctttcaccgtgacggccgcgcaggcccgaaagctggttgagacggccaaggccaagaagctcttcctgatggaagctgtgtggacacggtactttccgctgagtatcaagattcgagagctcattgccgccggcgagattggcactgtctttcgaacaatcgccgacttgtccatcaacgcaaactcagagcagggtcaagccctgaaattcgcagactcacatcgaatggtcaacccggacctcgcaggcggtgccaccttggatctcggagtctatcccttgacctgggtgttccagaccctgtatcatttgcaaccggaggaagacaaggaggctcccaccgtggttgcttccagcaacaagtacaccactggcgcagacgagaataccgccatcatctgcagcttccctcgccacaacagcattggaattgcttcgacgacgatgagggcggacaccgaccccgagaaggacaccattccggcggtccgaattcaaggatccaagggagaaatccaagtcttcttcccgacctaccgaccgctcaagtacaaggtggtgaagacgaacggcgaggcgcagacggttgactgccccatccccggagaccccgcgcgcaagggctcgggccacggaatgttctgggaggcggacgagtgtgctcgatgccttcgcgatggcaagttggagagtgccacgttgccatggaaggagagcattgtcattatggaaacgatggaggaggcgctgaggcagggtggcgtcacgtatccggagctgattaccacggatgtctatgatcccaagagccctctcaacacggggaatcagtag

SEQ ID NO: 65 - protein, 391 aa, Trichoderma reesei
MASGNPYTLKWGIMATGGIAETFCKDLLCNPAIRGADDVRHEIVAVASSSSSKRAEEFLQRIDGAFDAKTYGSYPELVADPNVDIVYVATPHSHHFQNTMLALEAGKNVLCEKAFTVTAAQARKLVETAKAKKLFLMEAVWTRYFPLSIKIRELIAAGEIGTVFRTIADLSINANSEQGQALKFADSHRMVNPDLAGGATLDLGVYPLTWVFQTLYHLQPEEDKEAPTVVASSNKYTTGADENTAIICSFPRHNSIGIASTTMRADTDPEKDTIPAVRIQGSKGEIQVFFPTYRPLKYKVVKTNGEAQTVDCPIPGDPARKGSGHGMFWEADECARCLRDGKLESATLPWKESIVIMETMEEALRQGGVTYPELITTDVYDPKSPLNTGNQ

SEQ ID NO: 66 - DNA, 870 bp, Caulobacter crescentus
atgaccgctcaagtcacttgcgtatgggatctgaaggccacgttgggcgaaggcccgatctggcatggcgacaccctgtggttcgtcgacatcaagcagcgtaaaatccacaactaccaccccgccaccggcgagcgcttcagcttcgacgcgccggatcaggtgaccttcctcgcgccgatcgtcggcgcgaccggctttgtcgtcggtctgaagaccgggattcaccgcttccacccggccacgggcttcagcctgctgctcgaggtcgaggacgcggcgctgaacaaccgccccaacgacgccacggtcgacgcgcaaggccgtctgtggttcggcaccatgcacgacggggaagagaacaatagcggctcgctctatcggatggacctcaccggcgtcgcccggatggaccgcgacatctgcatcaccaacggcccgtgcgtctcgcccgacggcaagaccttctaccacaccgacaccctggaaaagacgatctacgccttcgacctggccgaggacggcctgctgtcgaacaagcgcgtcttcgtgcagttcgccctgggcgacgatgtctatccggacggttcggtcgtcgattccgaaggctatctgtggaccgccctgtggggcggtttcggcgcggtccgcttctcgccgcaaggcgacgccgtgacgcgcatcgaactgcccgcccccaacgtcaccaagccctgcttcggcgggcctgacctgaagaccctctatttcaccaccgcccgcaagggcctgagcgacgagaccctggcccagtacccgctggccggcggtgtgttcgccgttccggtcgatgtggccggccaaccccagcatgaggtccgccttgtctaa

SEQ ID NO: 67 - protein, 289 aa, Caulobacter crescentus
MTAQVTCVWDLKATLGEGPIWHGDTLWFVDIKQRKIHNYHPATGERFSFDAPDQVTFLAPIVGATGFVVGLKTGIHRFHPATGFSLLLEVEDAALNNRPNDATVDAQGRLWFGTMHDGEENNSGSLYRMDLTGVARMDRDICITNGPCVSPDGKTFYHTDTLEKTIYAFDLAEDGLLSNKRVFVQFALGDDVYPDGSVVDSEGYLWTALWGGFGAVRFSPQGDAVTRIELPAPNVTKPCFGGPDLKTLYFTTARKGLSDETLAQYPLAGGVFAVPVDVAGQPQHEVRLV

SEQ ID NO: 68 - DNA, 1776 bp, Caulobacter crescentus
ttgtctaaccgcacgccccgccggttccggtcccgcgattggttcgataaccccgaccatatcgacatgaccgcgctctatctggagcgcttcatgaactacgggatcacgccggaggagctgcgcagcggcaagccgatcatcggcatcgcccagaccggcagcgacatctcgccctgcaaccgcatccacctggacctggtccagcgggtgcgggacgggatccgcgacgccgggggcatccccatggagttcccggtccatccgatcttcgagaactgccgtcgcccgacggcggcgctggaccggaacctctcgtacctgggtctcgtcgagaccctgcacggctatccgatcgacgccgtggttctgaccaccggctgcgacaagaccaccccggccgggatcatggccgccaccacggtcaatatcccggccatcgtgctgtcgggcggcccgatgctggacggctggcacgagaacgagctcgtgggctcgggcaccgtgatctggcgctcgcgccgcaagctggcggccggcgagatcaccgaggaagagttcatcgaccgcgccgccagctcggcgccgtcggcgggccactgcaacaccatgggcacggcctcgaccatgaacgccgtggccgaggcgctgggcctgtcgctgaccggctgcgcggccatccccgccccctaccgcgagcgcggccagatggcctacaagaccggccagcgcatcgtcgatctggcctatgacgacgtcaaaccgctcgacatcctgaccaagcaagccttcgagaacgccatcgccctggtggcggcggccggcggctcgaccaacgcccagccgcacatcgtggccatggcccgtcacgccggcgtcgagatcaccgccgacgactggcgcgcggcctatgacatcccgctgatcgtcaacatgcagccggccggcaagtatctgggcgagcgcttccaccgagccggcggcgcgccggcggtgctgtgggagctgttgcagcaaggccgcctgcacggcgacgtgctgaccgtcaccggcaagacgatgagcgagaacctgcaaggccgcgaaaccagcgaccgcgaggtgatcttcccgtaccacgagccgctggccgagaaggccgggttcctggttctcaagggcaacctcttcgacttcgcgatcatgaagtccagcgtgatcggcgaggagttccgcaagcgctacctgtcgcagcccggccaggaaggcgtgttcgaagcccgcgccatcgtgttcgacggctcggacgactatcacaagcggatcaacgatccggccctggagatcgacgagcgctgcatcctggtgatccgcggcgcgggtccgatcggctggcccggctcggccgaggtcgtcaacatgcagccgccggatcaccttctgaagaaggggatcatgagcctgcccaccctgggcgatggccgtcagtcgggcaccgccgacagcccctcgatcctgaacgcctcgcccgaaagcgcgatcggcggcggcctgtcgtggctgcgcaccggcgacaccatccgcatcgacctcaacaccggccgctgcgacgccctggtcgacgaggcgacgatcgccgcgcgcaagcaggacggcatcccggcggttcccgccaccatgacgccctggcaggaaatctaccgcgcccacgccagtcagctcgacaccggcggcgtgctggagttcgcggtcaagtaccaggacctggcggccaagctgccccgccacaaccactga

SEQ ID NO: 69 - protein, 591 aa, Caulobacter crescentus
MSNRTPRRFRSRDWFDNPDHIDMTALYLERFMNYGITPEELRSGKPIIGIAQTGSDISPCNRIHLDLVQRVRDGIRDAGGIPMEFPVHPIFENCRRPTAALDRNLSYLGLVETLHGYPIDAVVLTTGCDKTTPAGIMAATTVNIPAIVLSGGPMLDGWHENELVGSGTVIWRSRRKLAAGEITEEEFIDRAASSAPSAGHCNTMGTASTMNAVAEALGLSLTGCAAIPAPYRERGQMAYKTGQRIVDLAYDDVKPLDILTKQAFENAIALVAAAGGSTNAQPHIVAMARHAGVEITADDWRAAYDIPLIVNMQPAGKYLGERFHRAGGAPAVLWELLQQGRLHGDVLTVTGKTMSENLQGRETSDREVIFPYHEPLAEKAGFLVLKGNLFDFAIMKSSVIGEEFRKRYLSQPGQEGVFEARAIVFDGSDDYHKRINDPALEIDERCILVIRGAGPIGWPGSAEVVNMQPPDHLLKKGIMSLPTLGDGRQSGTADSPSILNASPESAIGGGLSWLRTGDTIRIDLNTGRCDALVDEATIAARKQDGIPAVPATMTPWQEIYRAHASQLDTGGVLEFAVKYQDLAAKLPRHNH

SEQ ID NO: 70 - DNA, 1968 bp, Escherichia coli
atgtctgttcgcaatatttttgctgacgagagccacgatatttacaccgtcagaacgcacgccgatggcccggacggcgaactcccattaaccgcagagatgcttatcaaccgcccgagcggggatctgttcggtatgaccatgaatgccggaatgggttggtctccggacgagctggatcgggacggtattttactgctcagtacactcggtggcttacgcggcgcagacggtaaacccgtggcgctggcgttgcaccaggggcattacgaactggacatccagatgaaagcggcggccgaggttattaaagccaaccatgccctgccctatgccgtgtacgtctccgatccttgtgacgggcgtactcagggtacaacggggatgtttgattcgctaccataccgaaatgacgcatcgatggtaatgcgccgccttattcgctctctgcccgacgcgaaagcagttattggtgtggcgagttgcgataaggggcttccggccaccatgatggcactcgccgcgcagcacaacatcgcaaccgtgctggtccccggcggcgcgacgctgcccgcaaaggatggagaagacaacggcaaggtgcaaaccattggcgcacgcttcgccaatggcgaattatctctacaggacgcacgccgtgcgggctgtaaagcctgtgcctcttccggcggcggctgtcaatttttgggcactgccgggacatctcaggtggtggccgaaggattgggactggcaatcccacattcagccctggccccttccggtgagcctgtgtggcgggagatcgccagagcttccgcgcgagctgcgctgaacctgagtcaaaaaggcatcaccacccgggaaattctcaccgataaagcgatagagaatgcgatgacggtccatgccgcgttcggtggttcaacaaacctgctgttacacatcccggcaattgctcaccaggcaggttgccatatcccgaccgttgatgactggatccgcatcaacaagcgcgtgccccgactggtgagcgtactgcctaatggcccggtttatcatccaacggtcaatgcctttatggcaggtggtgtgccggaagtcatgttgcatctgcgcagcctcggattgttgcatgaagacgttatgacggttaccggcagcacgctgaaagaaaacctcgactggtgggagcactccgaacggcgtcagcggttcaagcaactcctgctcgatcaggaacaaatcaacgctgacgaagtgatcatgtctccgcagcaagcaaaagcgcgcggattaacctcaactatcaccttcccggtgggcaatattgcgccagaaggttcggtgatcaaatccaccgccattgacccctcgatgattgatgagcaaggtatctattaccataaaggtgtggcgaaggtttatctgtccgagaaaagtgcgatttacgatatcaaacatgacaagatcaaggcgggcgatattctggtcattattggcgttggaccttcaggtacagggatggaagaaacctaccaggttaccagtgccctgaagcatctgtcatacggtaagcatgtttcgttaatcaccgatgcacgtttctcgggcgtttctactggcgcgtgcatcggccatgtggggccagaagcgctggccggaggccccatcggtaaattacgcaccggggatttaattgaaattaaaattgattgtcgcgagcttcacggcgaagtcaatttcctcggaacccgtagcgatgaacaattaccttcacaggaggaggcaactgcaatattaaatgccagacccagccatcaggatttacttcccgatcctgaattgccagatgatacccggctatgggcaatgcttcaggccgtgagtggtgggacatggaccggttgtatttatgatgtaaacaaaattggcgcggctttgcgcgattttatgaataaaaactga

SEQ ID NO: 71 - DNA, 1968 bp, Artificial Sequence; identical to SEQ ID NO: 70
  Escherichia coli xylonate dehydratase yjhG codon optimized sequence

SEQ ID NO: 72 - protein, 655 aa, Escherichia coli [sequence omitted]

SEQ ID NO: 73 - DNA, 1968 bp, Escherichia coli
atgaccattgagaaaattttcaccccgcaggacgacgcgttttatgcggtgatcacccacgcggcggggccgcagggcgctctgccgctgaccccgcagatgctgatggaatctcccagcggcaacctgttcggcatgacgcagaacgccgggatgggctgggacgccaacaagctcaccggcaaagaggtgctgattatcggcactcagggcggcatccgcgccggagacggacgcccaatcgcgctgggctaccacaccgggcattgggagatcggcatgcagatgcaggcggcggcgaaggagatcacccgcaatggcgggatcccgttcgcggccttcgtcagcgatccgtgcgacgggcgctcgcagggcacgcacggtatgttcgattccctgccgtaccgcaacgacgcggcgatcgtgtttcgccgcctgatccgctccctgccgacgcggcgggcggtgatcggcgtagcgacctgcgataaagggctgcccgccaccatgattgcgctggccgcgatgcacgacctgccgactattctggtgccgggcggggcgacgctgccgccgaccgtcggggaagacgcgggcaaggtgcagaccatcggcgcgcgtttcgccaaccacgaactctccctgcaggaggccgccgaactgggctgtcgcgcctgcgcctcgccgggcggcgggtgtcagttcctcggcacggcgggcacctcgcaggtggtcgcggaggcgctgggtctggcgctgccgcactccgcgctggcgccgtccgggcaggcggtgtggctggagatcgcccgccagtcggcgcgcgcggtcagcgagctggatagccgcggcatcaccacgcgggatatcctctccgataaagccatcgaaaacgcgatggtgatccacgcggcgttcggcggctccaccaatttactgctgcacattccggccatcgcccacgcggcgggctgcacgatcccggacgttgagcactggacgcgcatcaaccgtaaagtgccgcgtctggtgagcgtgctgcccaacggcccggactatcacccgaccgtgcgcgccttcctcgcgggcggcgtgccggaggtgatgctccacctgcgcgacctcggcctgctgcatctggacgccatgaccgtgaccggccagacggtgggcgagaaccttgaatggtggcaggcgtccgagcgccgggcgcgcttccgccagtgcctgcgcgagcaggacggcgtagagccggatgacgtgatcctgccgccggagaaggcaaaagcgaaagggctgacctcgacggtctgcttcccgacgggcaacatcgctccggaaggttcggtgatcaaggccacggcgatcgacccgtcggtggtgggcgaagatggcgtataccaccacaccggccgggtgcgggtgtttgtctcggaagcgcaggcgatcaaggcgatcaagcgggaagagattgtgcagggcgatatcatggtggtgatcggcggcgggccgtccggcaccggcatggaagagacctaccagctcacctccgcgctaaagcatatctcgtggggcaagacggtgtcgctcatcaccgatgcgcgcttctcgggcgtgtcgacgggcgcctgcttcggccacgtgtcgccggaggcgctggcgggcgggccgattggcaagctgcgcgataacgacatcatcgagattgccgtggatcgtctgacgttaactggcagcgtgaacttcatcggcaccgcggacaacccgctgacgccggaagagggcgcgcgcgagctggcgcggcggcagacgcacccggacctgcacgcccacgactttttgccggacgacacccggctgtgggcggcactgcagtcggtgagcggcggcacctggaaaggctgtatttatgacaccgataaaattatcgaggtaattaacgccggtaaaaaagcgctcggaatttaa

SEQ ID NO: 74 - DNA, 1968 bp, Artificial Sequence; identical to SEQ ID NO: 73
  Escherichia coli xylonate dehydratase yagF codon optimized sequence

SEQ ID NO: 75 - protein, 655 aa, Escherichia coli
MTIEKIFTPQDDAFYAVITHAAGPQGALPLTPQMLMESPSGNLFGMTQNAGMGWDANKLTGKEVLIIGTQGGIRAGDGRPIALGYHTGHWEIGMQMQAAAKEITRNGGIPFAAFVSDPCDGRSQGTHGMFDSLPYRNDAAIVFRRLIRSLPTRRAVIGVATCDKGLPATMIALAAMHDLPTILVPGGATLPPTVGEDAGKVQTIGARFANHELSLQEAAELGCRACASPGGGCQFLGTAGTSQVVAEALGLALPHSALAPSGQAVWLEIARQSARAVSELDSRGITTRDILSDKAIENAMVIHAAFGGSTNLLLHIPAIAHAAGCTIPDVEHWTRINRKVPRLVSVLPNGPDYHPTVRAFLAGGVPEVMLHLRDLGLLHLDAMTVTGQTVGENLEWWQASERRARFRQCLREQDGVEPDDVILPPEKAKAKGLTSTVCFPTGNIAPEGSVIKATAIDPSVVGEDGVYHHTGRVRVFVSEAQAIKAIKREEIVQGDIMVVIGGGPSGTGMEETYQLTSALKHISWGKTVSLITDARFSGVSTGACFGHVSPEALAGGPIGKLRDNDIIEIAVDRLTLTGSVNFIGTADNPLTPEEGARELARRQTHPDLHAHDFLPDDTRLWAALQSVSGGTWKGCIYDTDKIIEVINAGKKALGI

SEQ ID NO: 76 - DNA, 906 bp, Escherichia coli
atgaaaaaattcagcggcattattccaccggtatccagcacgtttcatcgtgacggaacccttgataaaaaggcaatgcgcgaagttgccgacttcctgattaataaaggggtcgacgggctgttttatctgggtaccggtggtgaatttagccaaatgaatacagcccagcgcatggcactcgccgaagaagctgtaaccattgtcgacgggcgagtgccggtattgattggcgtcggttccccttccactgacgaagcggtcaaactggcgcagcatgcgcaagcctacggcgctgatggtatcgtcgccatcaacccctactactggaaagtcgcaccacgaaatcttgacgactattaccagcagatcgcccgtagcgtcaccctaccggtgatcctgtacaactttccggatctgacgggtcaggacttaaccccggaaaccgtgacgcgtctggctctgcaaaacgagaatatcgttggcatcaaagacaccatcgacagcgttggtcacttgcgtacgatgatcaacacagttaagtcggtacgcccgtcgttttcggtattctgcggttacgatgatcatttgctgaatacgatgctgctgggcggcgacggtgcgataaccgccagcgctaactttgctccggaactctccgtcggcatctaccgcgcctggcgtgaaggcgatctggcgaccgctgcgacgctgaataaaaaactactacaactgcccgctatttacgccctcgaaacaccgtttgtctcactgatcaaatacagcatgcagtgtgtcgggctgcctgtagagacatattgcttaccaccgattcttgaagcatctgaagaagcaaaagataaagtccacgtgctgcttaccgcgcagggcattttaccagtctga

SEQ ID NO: 77 - DNA, 906 bp, Artificial Sequence; identical to SEQ ID NO: 76
  Escherichia coli Uncharacterized lyase yjhH codon optimized sequence

SEQ ID NO: 78 - protein, 301 aa, Escherichia coli
MKKFSGIIPPVSSTFHRDGTLDKKAMREVADFLINKGVDGLFYLGTGGEFSQMNTAQRMALAEEAVTIVDGRVPVLIGVGSPSTDEAVKLAQHAQAYGADGIVAINPYYWKVAPRNLDDYYQQIARSVTLPVILYNFPDLTGQDLTPETVTRLALQNENIVGIKDTIDSVGHLRTMINTVKSVRPSFSVFCGYDDHLLNTMLLGGDGAITASANFAPELSVGIYRAWREGDLATAATLNKKLLQLPAIYALETPFVSLIKYSMQCVGLPVETYCLPPILEASEEAKDKVHVLLTAQGILPV

SEQ ID NO: 79 - DNA, 909 bp, Escherichia coli
atgccgcagtccgcgttgttcacgggaatcattccccctgtctccaccatttttaccgccgacggccagctcgataagccgggcaccgccgcgctgatcgacgatctgatcaaagcaggcgttgacggcctgttcttcctgggcagcggtggcgagttctcccagctcggcgccgaagagcgtaaagccattgcccgctttgctatcgatcatgtcgatcgtcgcgtgccggtgctgatcggcaccggcggcaccaacgcccgggaaaccatcgaactcagccagcacgcgcagcaggcgggcgcggacggcatcgtggtgatcaacccctactactggaaagtgtcggaagcgaacctgatccgctatttcgagcaggtggccgacagcgtcacgctgccggtgatgctctataacttcccggcgctgaccgggcaggatctgactccggcgctggtgaaaaccctcgccgactcgcgcagcaatattatcggcatcaaagacaccatcgactccgtcgcccacctgcgcagcatgatccataccgtcaaaggtgcccatccgcacttcaccgtgctctgcggctacgacgatcatctgttcaataccctgctgctcggcggcgacggggcgatatcggcgagcggcaactttgccccgcaggtgtcggtgaatcttctgaaagcctggcgcgacggggacgtggcgaaagcggccgggtatcatcagaccttgctgcaaattccgcagatgtatcagctggatacgccgtttgtgaacgtgattaaagaggcgatcgtgctctgcggtcgtcctgtctccacgcacgtgctgccgcccgcctcgccgctggacgagccgcgcaaggcgcagctgaaaaccctgctgcaacagctcaagctttgctga

SEQ ID NO: 80 - DNA, 909 bp, Artificial Sequence; identical to SEQ ID NO: 79
  Escherichia coli Probable 2-keto-3-deoxy- galactonate aldolase yagE codon optimized sequence

SEQ ID NO: 81 - protein, 302 aa, Escherichia coli
MPQSALFTGIIPPVSTIFTADGQLDKPGTAALIDDLIKAGVDGLFFLGSGGEFSQLGAEERKAIARFAIDHVDRRVPVLIGTGGTNARETIELSQHAQQAGADGIVVINPYYWKVSEANLIRYFEQVADSVTLPVMLYNFPALTGQDLTPALVKTLADSRSNIIGIKDTIDSVAHLRSMIHTVKGAHPHFTVLCGYDDHLFNTLLLGGDGAISASGNFAPQVSVNLLKAWRDGDVAKAAGYHQTLLQIPQMYQLDTPFVNVIKEAIVLCGRPVSTHVLPPASPLDEPRKAQLKTLLQQLKLC

SEQ ID NO: 82 - DNA, 957 bp, Scheffersomyces stipitis
atgccttctattaagttgaactctggttacgacatgccagccgtcggtttcggctgttggaaagtcgacgtcgacacctgttctgaacagatctaccgtgctatcaagaccggttacagattgttcgacggtgccgaagattacgccaacgaaaagttagttggtgccggtgtcaagaaggccattgacgaaggtatcgtcaagcgtgaagatttgttccttacctccaagttgtggaacaactaccaccacccagacaacgtcgaaaaggccttgaacagaaccctttctgacttgcaagttgactacgttgacttgttcttgatccacttcccagtcaccttcaagttcgttccattagaagaaaagtacccaccaggattctactgtggtaagggtgacaacttcgactacgaagatgttccaattttagagacttggaaggctcttgaaaagttggtcaaggccggtaagatcagatctatcggtgtttctaacttcccaggtgctttgctcttggacttgttgagaggtgctaccatcaagccatctgtcttgcaagttgaacaccacccatacttgcaacaaccaagattgatcgaattcgctcaatcccgtggtattgctgtcaccgcttactcttcgttcggtcctcaatctttcgttgaattgaaccaaggtagagctttgaacacttctccattgttcgagaacgaaactatcaaggctatcgctgctaagcacggtaagtctccagctcaagtcttgttgagatggtcatcccaaagaggcattgccatcattccaaagtccaacactgtcccaagattgttggaaaacaaggacgtcaacagcttcgacttggacgaacaagatttcgctgacattgccaagttggacatcaacttgagattcaacgacccatgggactgggacaagattcctatcttcgtctaa

SEQ ID NO: 83 - DNA, 957 bp, Artificial Sequence
  Scheffersomyces stipitis D-xylose reductase xyl1 codon optimized sequence
atgccatctatcaagttaaattccggttacgacatgcctgctgttggtttcggttgctggaaggttgatgtcgatacttgttccgagcaaatttaccgtgctatcaagactggttacagattgttcgatggtgctgaagactacgccaacgaaaagttagtcggtgctggtgttaaaaaggctatcgacgaaggtattgttaaaagagaagacttgttcttgacttctaagttgtggaacaactaccaccatcctgataacgtcgaaaaagctttgaaccgtaccttgtccgatttgcaagtcgattacgttgatttgttcttgattcatttcccagttaccttcaagttcgttccattggaagagaagtatccaccaggtttctactgtggtaagggtgataacttcgattacgaagatgtcccaatcttagaaacctggaaggctttagaaaagttggttaaggctggtaagatcagatccatcggtgtttctaacttcccaggtgccttattgttagacttattgagaggtgctaccattaagccttccgttttgcaagttgaacatcatccttacttgcaacaaccaagattgatcgaattcgctcaatctagaggtatcgctgttactgcctactcttccttcggtccacaatctttcgttgagttgaaccaaggtagagctttgaacacctctccattgttcgaaaacgaaactattaaggccattgctgctaagcatggtaagtctccagcccaagttttgttgagatggtcttctcaaagaggtatcgctattatcccaaagtctaatactgtcccaagattgttggaaaacaaggacgttaactcctttgatttggatgaacaagactttgctgacatcgctaaattggacatcaacttgagattcaacgacccatgggactgggacaagattccaatttttgtttaa

SEQ ID NO: 84 - protein, 318 aa, Scheffersomyces stipitis
MPSIKLNSGYDMPAVGFGCWKVDVDTCSEQIYRAIKTGYRLFDGAEDYANEKLVGAGVKKAIDEGIVKREDLFLTSKLWNNYHHPDNVEKALNRTLSDLQVDYVDLFLIHFPVTFKFVPLEEKYPPGFYCGKGDNFDYEDVPILETWKALEKLVKAGKIRSIGVSNFPGALLLDLLRGATIKPSVLQVEHHPYLQQPRLIEFAQSRGIAVTAYSSFGPQSFVELNQGRALNTSPLFENETIKAIAAKHGKSPAQVLLRWSSQRGIAIIPKSNTVPRLLENKDVNSFDLDEQDFADIAKLDINLRFNDPWDWDKIPIFV

SEQ ID NO: 85 - DNA, 984 bp, Saccharomyces cerevisiae; identical to SEQ ID NO: 16

SEQ ID NO: 86 - DNA, 984 bp, Artificial Sequence; identical to SEQ ID NO: 16
  Saccharomyces cerevisiae aldose reductase GRE3 codon optimized sequence

SEQ ID NO: 87 - protein, 327 aa, Saccharomyces cerevisiae; identical to SEQ ID NO: 17

SEQ ID NO: 88 - DNA, 1092 bp, Scheffersomyces stipitis
atgactgctaacccttccttggtgttgaacaagatcgacgacatttcgttcgaaacttacgatgccccagaaatctctgaacctaccgatgtcctcgtccaggtcaagaaaaccggtatctgtggttccgacatccacttctacgcccatggtagaatcggtaacttcgttttgaccaagccaatggtcttgggtcacgaatccgccggtactgttgtccaggttggtaagggtgtcacctctcttaaggttggtgacaacgtcgctatcgaaccaggtattccatccagattctccgacgaatacaagagcggtcactacaacttgtgtcctcacatggccttcgccgctactcctaactccaaggaaggcgaaccaaacccaccaggtaccttatgtaagtacttcaagtcgccagaagacttcttggtcaagttgccagaccacgtcagcttggaactcggtgctcttgttgagccattgtctgttggtgtccacgcctctaagttgggttccgttgctttcggcgactacgttgccgtctttggtgctggtcctgttggtcttttggctgctgctgtcgccaagaccttcggtgctaagggtgtcatcgtcgttgacattttcgacaacaagttgaagatggccaaggacattggtgctgctactcacaccttcaactccaagaccggtggttctgaagaattgatcaaggctttcggtggtaacgtgccaaacgtcgttttggaatgtactggtgctgaaccttgtatcaagttgggtgttgacgccattgccccaggtggtcgtttcgttcaagtcggtaacgctgctggtccagtcagcttcccaatcaccgttttcgccatgaaggaattgactttgttcggttctttcagatacggattcaacgactacaagactgctgttggaatctttgacactaactaccaaaacggtagagaaaatgctccaattgactttgaacaattgatcacccacagatacaagttcaaggacgctattgaagcctacgacttggtcagagccggtaagggtgctgtcaagtgtctcattgacggccctgagtaa

SEQ ID NO: 89 - DNA, 1092 bp, Artificial Sequence
  Scheffersomyces stipitis D-xylulose reductase xyl2 codon optimized sequence
atgactgctaacccttccttggtgttgaacaagatcgacgacatttcgttcgaaacttacgatgccccagaaatctctgaacctaccgatgtcctcgtccaggtcaagaaaaccggtatctgtggttccgacatccacttctacgcccatggtagaatcggtaacttcgttttgaccaagccaatggtcttgggtcacgaatccgccggtactgttgtccaggttggtaagggtgtcacctctcttaaggttggtgacaacgtcgctatcgaaccaggtattccatccagattctccgacgaatacaagagcggtcactacaacttgtgtcctcacatggccttcgccgctactcctaactccaaggaaggcgaaccaaacccaccaggtaccttatgtaagtacttcaagtcgccagaagatttcttggtcaagttgccagaccacgtcagcttggaactcggtgctcttgttgagccattgtctgttggtgtccacgcctctaagttgggttccgttgctttcggcgactacgttgccgtctttggagcaggtcctgttggtcttttggctgctgctgtcgccaagaccttcggtgctaagggtgtcatcgtcgttgacattttcgacaacaagttgaagatggccaaggacattggagctgctactcacaccttcaactccaagaccggtggttctgaagaattgatcaaggctttcggtggtaacgtgccaaacgtcgttttggaatgtacaggtgcagaaccttgtatcaagttgggtgttgacgccattgccccaggtggtcgtttcgttcaagtcggtaacgctgctggtccagtcagcttcccaatcaccgttttcgccatgaaggaattgactttgttcggttctttcagatacggattcaacgactacaagactgctgttggaatctttgacactaactaccaaaacggtagagaaaatgctccaattgactttgaacaattgatcacccacagatacaagttcaaggacgctattgaagcctacgacttggtcagagccggtaagggtgctgtcaagtgtctcattgacggccctgagtaa

SEQ ID NO: 90 - protein, 363 aa, Scheffersomyces stipitis
MTANPSLVLNKIDDISFETYDAPEISEPTDVLVQVKKTGICGSDIHFYAHGRIGNFVLTKPMVLGHESAGTVVQVGKGVTSLKVGDNVAIEPGIPSRFSDEYKSGHYNLCPHMAFAATPNSKEGEPNPPGTLCKYFKSPEDFLVKLPDHVSLELGALVEPLSVGVHASKLGSVAFGDYVAVFGAGPVGLLAAAVAKTFGAKGVIVVDIFDNKLKMAKDIGAATHTFNSKTGGSEELIKAFGGNVPNVVLECTGAEPCIKLGVDAIAPGGRFVQVGNAAGPVSFPITVFAMKELTLFGSFRYGFNDYKTAVGIFDTNYQNGRENAPIDFEQLITHRYKFKDAIEAYDLVRAGKGAVKCLIDGPE

SEQ ID NO: 91 - DNA, 1092 bp, Trichoderma reesei
atggcgactcaaacgatcaacaaggatgcgatcagcaacctctccttcgtcctcaacaagcccggcgacgtgacctttgaggagcggccgaagccgaccatcacggaccccaacgacgtcctcgtcgccgtcaactacacgggcatctgcggctccgacgtgcactactgggtgcacggcgccatcgggcacttcgtcgtcaaggacccgatggtgctgggccacgagtcggccggcaccgtcgtcgaggtcggcccggccgtcaagagcctcaagcccggcgaccgcgtcgccctcgagcccggctacccgtgccggcggtgctccttctgccgcgccggcaaatacaacctgtgcccggacatggtcttcgccgccacgccgccgtaccacggcaccctgacgggcctgtgggcggcgcccgccgacttctgctacaagctgccggacggcgtgtcgctgcaggagggcgcgctgatcgagccgctggccgtggccgtccacattgtcaagcaggcccgcgtccagccgggccagtccgtcgtcgtcatgggcgccggccccgtcggcctgctgtgcgccgccgtggccaaggcgtacggcgcctccaccattgtcagcgtcgacatcgtgcagtccaagctcgactttgcgcgcggcttctgctcgacgcacacgtacgtctcgcagcgcatctcggctgaggacaacgcaaaggccatcaaggagctggcgggcctgcccggcggcgccgacgtcgtgattgacgccagcggcgcggagccgtcgatccagacgagcattcacgtcgtccgcatgggcggcacgtacgtccagggcggcatgggcaagagcgacatcacgttccccatcatggccatgtgcctcaaggaggtgacggtccggggctcgttccgctacggcgccggcgactacgagctggcggtcgagctggtccggacggggcgggtggacgtcaagaagctgattacgggcaccgtcagcttcaagcaggcggaggaggcgttccaaaaggtcaagtctggggaggccatcaagattctgattgccgggcccaacgagaaggtgtaa

SEQ ID NO: 92 - protein, 363 aa, Trichoderma reesei
MATQTINKDAISNLSFVLNKPGDVTFEERPKPTITDPNDVLVAVNYTGICGSDVHYWVHGAIGHFVVKDPMVLGHESAGTVVEVGPAVKSLKPGDRVALEPGYPCRRCSFCRAGKYNLCPDMVFAATPPYHGTLTGLWAAPADFCYKLPDGVSLQEGALIEPLAVAVHIVKQARVQPGQSVVVMGAGPVGLLCAAVAKAYGASTIVSVDIVQSKLDFARGFCSTHTYVSQRISAEDNAKAIKELAGLPGGADVVIDASGAEPSIQTSIHVVRMGGTYVQGGMGKSDITFPIMAMCLKEVTVRGSFRYGAGDYELAVELVRTGRVDVKKLITGTVSFKQAEEAFQKVKSGEAIKILIAGPNEKV

SEQ ID NO: 93 - DNA, 1314 bp, Pyromyces sp.
atggctaaggaatatttcccacaaattcaaaagattaagttcgaaggtaaggattctaagaatccattagccttccactactacgatgctgaaaaggaagtcatgggtaagaaaatgaaggattggttacgtttcgccatggcctggtggcacactctttgcgccgaaggtgctgaccaattcggtggaggtacaaagtctttcccatggaacgaaggtactgatgctattgaaattgccaagcaaaaggttgatgctggtttcgaaatcatgcaaaagcttggtattccatactactgtttccacgatgttgatcttgtttccgaaggtaactctattgaagaatacgaatccaaccttaaggctgtcgttgcttacctcaaggaaaagcaaaaggaaaccggtattaagcttctctggagtactgctaacgtcttcggtcacaagcgttacatgaacggtgcctccactaacccagactttgatgttgtcgcccgtgctattgttcaaattaagaacgccatagacgccggtattgaacttggtgctgaaaactacgtcttctggggtggtcgtgaaggttacatgagtctccttaacactgaccaaaagcgtgaaaaggaacacatggccactatgcttaccatggctcgtgactacgctcgttccaagggattcaagggtactttcctcattgaaccaaagccaatggaaccaaccaagcaccaatacgatgttgacactgaaaccgctattggtttccttaaggcccacaacttagacaaggacttcaaggtcaacattgaagttaaccacgctactcttgctggtcacactttcgaacacgaacttgcctgtgctgttgatgctggtatgctcggttccattgatgctaaccgtggtgactaccaaaacggttgggatactgatcaattcccaattgatcaatacgaactcgtccaagcttggatggaaatcatccgtggtggtggtttcgttactggtggtaccaacttcgatgccaagactcgtcgtaactctactgacctcgaagacatcatcattgcccacgtttctggtatggatgctatggctcgtgctcttgaaaacgctgccaagctcctccaagaatctccatacaccaagatgaagaaggaacgttacgcttccttcgacagtggtattggtaaggactttgaagatggtaagctcaccctcgaacaagtttacgaatacggtaagaagaacggtgaaccaaagcaaacttctggtaagcaagaactctacgaagctattgttgccatgtaccaataa

SEQ ID NO: 94 - DNA, 1314 bp, Artificial Sequence
  Pyromyces sp. xylose isomerase xylA codon optimized sequence
atggccaaggaatacttcccacaaatccaaaagattaaattcgaaggtaaagattccaagaacccattggcttttcactactacgatgctgagaaggaagttatgggtaagaagatgaaggattggttgagattcgctatggcttggtggcacactttgtgcgctgaaggtgctgaccaattcggtggtggtactaagtctttcccatggaacgaaggtactgatgctattgaaatcgctaagcaaaaagtcgatgctggttttgagattatgcaaaaattgggtatcccatactactgtttccacgacgtcgacttggtttctgaaggtaattctatcgaagaatacgaatctaatttgaaggctgttgtcgcttacttaaaagaaaagcaaaaggagactggtattaagttgttgtggtccaccgctaacgtctttggtcataaaagatacatgaacggtgcttccaccaacccagacttcgatgtcgtcgccagagctatcgttcaaattaaaaacgccatcgacgctggtattgaattgggtgctgaaaattacgtcttttggggtggtcgtgaaggttacatgtctttgttgaacactgaccaaaagagagaaaaagaacacatggccactatgttgaccatggccagagattacgccagatctaagggtttcaagggtaccttcttaattgaaccaaaacctatggaaccaactaagcaccaatacgacgttgacactgaaactgctatcggttttttgaaggctcacaacttggataaggattttaaagtcaacattgaagttaaccatgctactttggctggtcacacttttgaacatgaattggcctgtgctgttgatgctggtatgttgggttctatcgatgctaatagaggtgactatcaaaacggttgggacactgatcaattcccaatcgatcaatatgaattagttcaagcttggatggaaattatcagaggtggtggtttcgttactggtggtactaacttcgatgctaagaccagaagaaactctactgatttggaagatattatcattgcccacgtttccggtatggatgccatggccagagctttggaaaacgccgccaagttattgcaagagtccccatacaccaagatgaaaaaggaacgttacgcttctttcgactctggtatcggtaaagacttcgaagatggtaagttgaccttggaacaagtttacgaatacggtaagaagaacggtgaacctaaacaaacctctggtaaacaagaattgtatgaagctattgttgccatgtaccaataa

SEQ ID NO: 95 - protein, 437 aa, Pyromyces sp.
MAKEYFPQIQKIKFEGKDSKNPLAFHYYDAEKEVMGKKMKDWLRFAMAWWHTLCAEGADQFGGGTKSFPWNEGTDAIEIAKQKVDAGFEIMQKLGIPYYCFHDVDLVSEGNSIEEYESNLKAVVAYLKEKQKETGIKLLWSTANVFGHKRYMNGASTNPDFDVVARAIVQIKNAIDAGIELGAENYVFWGGREGYMSLLNTDQKREKEHMATMLTMARDYARSKGFKGTFLIEPKPMEPTKHQYDVDTETAIGFLKAHNLDKDFKVNIEVNHATLAGHTFEHELACAVDAGMLGSIDANRGDYQNGWDTDQFPIDQYELVQAWMEIIRGGGFVTGGTNFDAKTRRNSTDLEDIIIAHVSGMDAMARALENAAKLLQESPYTKMKKERYASFDSGIGKDFEDGKLTLEQVYEYGKKNGEPKQTSGKQELYEAIVAMYQ

SEQ ID NO: 96 - DNA, 657 bp, Clostridium acetobutylicum
atgaactctaaaataattagatttgaaaatttaaggtcattctttaaagatgggatgacaattatgattggaggttttttaaactgtggcactccaaccaaattaattgattttttagttaatttaaatataaagaatttaacgattataagtaatgatacatgttatcctaatacaggtattggtaagttaatatcaaataatcaagtaaaaaagcttattgcttcatatataggcagcaacccagatactggcaaaaaactttttaataatgaacttgaagtagagctctctccccaaggaactctagtggaaagaatacgtgcaggcggatctggcttaggtggtgtactaactaaaacaggtttaggaactttgattgaaaaaggaaagaaaaaaatatctataaatggaacggaatatttgttagagctacctcttacagccgatgtagcattaattaaaggtagtattgtagatgaggccggaaacaccttctataaaggtactactaaaaactttaatccctatatggcaatggcagctaaaaccgtaatagttgaagctgaaaatttagttagctgtgaaaaactagaaaaggaaaaagcaatgacccccggagttcttataaattatatagtaaaggagcctgcataa

SEQ ID NO: 97 - protein, 218 aa, Clostridium acetobutylicum
MNSKIIRFENLRSFFKDGMTIMIGGFLNCGTPTKLIDFLVNLNIKNLTIISNDTCYPNTGIGKLISNNQVKKLIASYIGSNPDTGKKLFNNELEVELSPQGTLVERIRAGGSGLGGVLTKTGLGTLIEKGKKKISINGTEYLLELPLTADVALIKGSIVDEAGNTFYKGTTKNFNPYMAMAAKTVIVEAENLVSCEKLEKEKAMTPGVLINYIVKEPA

SEQ ID NO: 98 - DNA, 666 bp, Clostridium acetobutylicum
atgattaatgataaaaacctagcgaaagaaataatagccaaaagagttgcaagagaattaaaaaatggtcaacttgtaaacttaggtgtaggtcttcctaccatggttgcagattatataccaaaaaatttcaaaattactttccaatcagaaaacggaatagttggaatgggcgctagtcctaaaataaatgaggcagataaagatgtagtaaatgcaggaggagactatacaacagtacttcctgacggcacatttttcgatagctcagtttcgttttcactaatccgtggtggtcacgtagatgttactgttttaggggctctccaggtagatgaaaagggtaatatagccaattggattgttcctggaaaaatgctctctggtatgggtggagctatggatttagtaaatggagctaagaaagtaataattgcaatgagacatacaaataaaggtcaacctaaaattttaaaaaaatgtacacttcccctcacggcaaagtctcaagcaaatctaattgtaacagaacttggagtaattgaggttattaatgatggtttacttctcactgaaattaataaaaacacaaccattgatgaaataaggtctttaactgctgcagatttactcatatccaatgaacttagacccatggctgtttag

SEQ ID NO: 99 - protein, 221 aa, Clostridium acetobutylicum
MINDKNLAKEIIAKRVARELKNGQLVNLGVGLPTMVADYIPKNFKITFQSENGIVGMGASPKINEADKDVVNAGGDYTTVLPDGTFFDSSVSFSLIRGGHVDVTVLGALQVDEKGNIANWIVPGKMLSGMGGAMDLVNGAKKVIIAMRHTNKGQPKILKKCTLPLTAKSQANLIVTELGVIEVINDGLLLTEINKNTTIDEIRSLTAADLLISNELRPMAV

SEQ ID NO: 100 - DNA, 651 bp, Escherichia coli; identical to SEQ ID NO: 41

SEQ ID NO: 101 - protein, 216 aa, Escherichia coli; identical to SEQ ID NO: 43

SEQ ID NO: 102 - DNA, 663 bp, Escherichia coli; identical to SEQ ID NO: 44

SEQ ID NO: 103 - protein, 220 aa, Escherichia coli; identical to SEQ ID NO: 46

SEQ ID NO: 104 - DNA, 1563 bp, Mus musculus
atgcctgggtcacttcctttgaatgcagaggcttgctggccaaaagatgtgggaatcgttgcccttgaaatctactttccttctcaatatgtcgatcaagctgagttggaaaaatacgatggtgtagatgctggaaagtataccatcggcctgggccaggccaggatgggcttctgcacggatcgtgaagacatcaactctctttgcctgactgtggttcagaaactgatggagagacatagcctttcctatgattgcattgggcggctagaagttggaacagagacaatcatcgacaaatcgaaatcagtgaagtctaatttgatgcagctgtttgaggagtctgggaatacagatatagaaggaatagatacaaccaatgcatgctatgggggcacagctgcagtcttcaatgccgtgaactgggtcgaatccagctcttgggatggacgatatgctctggtagttgcaggagacattgctatatatgccacaggaaatgccagacctacaggtggagttggagctgtggccctgctaattgggccaaacgctcctctaatttttgaccgagggctccgtgggacacacatgcagcatgcctatgacttttacaagcctgacatgctctccgagtaccctgtggtcgacggaaagctctccatacagtgctacctcagcgccctggaccgctgctattctgtctaccgcaaaaagatccgtgcccagtggcagaaagagggaaaggataaagattttaccctgaatgattttggcttcatgatctttcactcaccatattgtaaactggtgcagaaatctctagctcggatgttcctgaatgactttcttaatgatcaaaacagagataaaaacagtatttacagtggactggaagcctttggggacgttaaattagaagatacttactttgacagagatgtagaaaaggcatttatgaaggctagttctgagctattcaaccagaaaacaaaggcgtctttgcttgtgtctaatcagaatggaaatatgtacacatcctctgtctatggttccctggcttctgtcctggcacagtactcacctcagcagttggcagggaagagggttggagtgttctcttacggttctggcttggctgccacgctgtactcccttaaagtcacacaagacgccacaccaggatctgcccttgataaaataacagcaagtttatgtgaccttaaatcaaggcttgactcgagaacttgtgtggcaccggatgtctttgctgaaaacatgaagctcagagaggacacacatcacttagccaactatattccccagtgttcaatagactcactctttgaaggaacgtggtatctggtcagagtggatgaaaaacacagaaggacttacgcccggcgccccttcacaaatgaccacagtttggatgaaggaatggggctcgtgcatagtaacacagcaacagagcatattccaagccctgctaagaaagtgccaagactccctgcaacctcggccgaatctgaatcagctgtcatcagtaacggggagcactga

SEQ ID NO: 105 - protein, 520 aa, Mus musculus
MPGSLPLNAEACWPKDVGIVALEIYFPSQYVDQAELEKYDGVDAGKYTIGLGQARMGFCTDREDINSLCLTVVQKLMERHSLSYDCIGRLEVGTETIIDKSKSVKSKLMQLFEESGNTDIEGIDTTNACYGGTAAVFNAVNWVESSSWDGRYALVVAGDIAIYATGNARPTGGVGAVALLIGPNAPLIFDRGLRGTHMQHAYDFYKPDMLSEYPVVDGKLSIQCYLSALDRCYSVYRKKIRAQWQKEGKDKDFTLNDFGFMIFHSPYCKLVQKSLARMFLNDFLNDQNRDKNSIYSGLEAFGDVKLEDTYFDRDVEKAFMKASSELFNQKTKASLLVSNQNGNMYTSSVYGSLASVLAQYSPQQLAGKRVGVFSYGSGLAATLYSLKVTQDATPGSALDKITASLCDLKSRLDSRTCVAPDVFAENMKLREDTHHLANYIPQCSIDSLFEGTWYLVRVDEKHRRTYARRPFTNDHSLDEGMGLVHSNTATEHIPSPAKKVPRLPATSAESESAVISNGEH

SEQ ID NO: 106 - DNA, 1476 bp, Saccharomyces cerevisiae
atgaaactctcaactaaactttgttggtgtggtattaaaggaagacttaggccgcaaaagcaacaacaattacacaatacaaacttgcaaatgactgaactaaaaaaacaaaagaccgctgaacaaaaaaccagacctcaaaatgtcggtattaaaggtatccaaatttacatcccaactcaatgtgtcaaccaatctgagctagagaaatttgatggcgtttctcaaggtaaatacacaattggtctgggccaaaccaacatgtcttttgtcaatgacagagaagatatctactcgatgtccctaactgttttgtctaagttgatcaagagttacaacatcgacaccaacaaaattggtagattagaagtcggtactgaaactctgattgacaagtccaagtctgtcaagtctgtcttgatgcaattgtttggtgaaaacactgacgtcgaaggtattgacacgcttaatgcctgttacggtggtaccaacgcgttgttcaactctttgaactggattgaatctaacgcatgggatggtagagacgccattgtagtttgcggtgatattgccatctacgataagggtgccgcaagaccaaccggtggtgccggtactgttgctatgtggatcggtcctgatgctccaattgtatttgactctgtaagagcttcttacatggaacacgcctacgatttttacaagccagatttcaccagcgaatatccttacgtcgatggtcatttttcattaacttgttacgtcaaggctcttgatcaagtttacaagagttattccaagaaggctatttctaaagggttggttagcgatcccgctggttcggatgctttgaacgttttgaaatatttcgactacaacgttttccatgttccaacctgtaaattggtcacaaaatcatacggtagattactatataacgatttcagagccaatcctcaattgttcccagaagttgacgccgaattagctactcgcgattatgacgaatctttaaccgataagaacattgaaaaaacttttgttaatgttgctaagccattccacaaagagagagttgcccaatctttgattgttccaacaaacacaggtaacatgtacaccgcatctgtttatgccgcctttgcatctctattaaactatgttggatctgacgacttacaaggcaagcgtgttggtttattttcttacggttccggtttagctgcatctctatattcttgcaaaattgttggtgacgtccaacatattatcaaggaattagatattactaacaaattagccaagagaatcaccgaaactccaaaggattacgaagctgccatcgaattgagagaaaatgcccatttgaagaagaacttcaaacctcaaggttccattgagcatttgcaaagtggtgtttactacttgaccaacatcgatgacaaatttagaagatcttacgatgttaaaaaataa

SEQ ID NO: 107 - protein, 491 aa, Saccharomyces cerevisiae
MKLSTKLCWCGIKGRLRPQKQQQLHNTNLQMTELKKQKTAEQKTRPQNVGIKGIQIYIPTQCVNQSELEKFDGVSQGKYTIGLGQTNMSFVNDREDIYSMSLTVLSKLIKSYNIDTNKIGRLEVGTETLIDKSKSVKSVLMQLFGENTDVEGIDTLNACYGGTNALFNSLNWIESNAWDGRDAIVVCGDIAIYDKGAARPTGGAGTVAMWIGPDAPIVFDSVRASYMEHAYDFYKPDFTSEYPYVDGHFSLTCYVKALDQVYKSYSKKAISKGLVSDPAGSDALNVLKYFDYNVFHVPTCKLVTKSYGRLLYNDFRANPQLFPEVDAELATRDYDESLTDKNIEKTFVNVAKPFHKERVAQSLIVPTNTGNMYTASVYAAFASLLNYVGSDDLQGKRVGLFSYGSGLAASLYSCKIVGDVQHIIKELDITNKLAKRITETPKDYEAAIELRENAHLKKNFKPQGSIEHLQSGVYYLTNIDDKFRRSYDVKK

SEQ ID NO: 108 - DNA, 1167 bp, Lactobacillus crispatus
atgcaagtaggtattgatcaaattggactttttacgccagacaagtacgttgatatggtcgatttggcgaacgcaagaaagcaagatccaaataagtttttagttggcattggtcaaagagagatgagtgttgcggatattacgcaagatgcagtttcaatgggaattaatgcgactttaaagtttattgacaagattgataagaataaagtcggtctattaatttttgggactgaaagcagtattgatcaatcaaagtcagcttctttatttgtaaaaacagccttaaagttgaagccagaagtaagaacttttgaagtaaaggaagcttgctttggtttaacggcagccttaatgatggcacgtgattttgttcgggtacatcctgaacaaaccgcaattgtcatcggtagtgatattgctcgctatggtgttaatactgctggtgaggtaacacaaggagccggtagcgtggcaatgctcatcaaagctgacccaagcattttagccctaaataatggtcattcagcttacagcgaagatattaacgacttctggagaccaaattattctcgcaccgctttagttgacggcaagtattcgactcaagtttacttggatttcttcaagaagacctttactgattataagaagcaaaaaggattgaaaacaagcgactttgatgcgattacttaccatttgccatttactaagatggggatgaaggctaacaatattgctattgaggggcaagatcaagcaacaatgatcaagttggagcgtagttttgcggctgctaaacaattatcaagccgagtaggtaatatctacaccgcttcactttatatgagtttacttagtttgctagagaatggtgaattacccgctggctcattaattggcctgttttcatatggttcaggtgcaatggctgagttttatagtggcaagttggttgaaggatatgaaaaacaaattgatcctacagctgatcaagcaatgcttgatcggcggaagaagctgacagttaagcaatatgaagacgtcttcaatacggctttgttagatcctgaggatggcttagaattaactagcgacgatgaagtaggtacttggtattttgctggtactaaggatcatattcgtcaataccgcgtaaaagaataa

SEQ ID NO: 109 - protein, 388 aa, Lactobacillus crispatus
MQVGIDQIGLFTPDKYVDMVDLANARKQDPNKFLVGIGQREMSVADITQDAVSMGINATLKFIDKIDKNKVGLLIFGTESSIDQSKSASLFVKTALKLKPEVRTFEVKEACFGLTAALMMARDFVRVHPEQTAIVIGSDIARYGVNTAGEVTQGAGSVAMLIKADPSILALNNGHSAYSEDINDFWRPNYSRTALVDGKYSTQVYLDFFKKTFTDYKKQKGLKTSDFDAITYHLPFTKMGMKANNIAIEGQDQATMIKLERSFAAAKQLSSRVGNIYTASLYMSLLSLLENGELPAGSLIGLFSYGSGAMAEFYSGKLVEGYEKQIDPTADQAMLDRRKKLTVKQYEDVFNTALLDPEDGLELTSDDEVGTWYFAGTKDHIRQYRVKE

SEQ ID NO: 110 - DNA, 927 bp, Polaromonas naphthalenivorans
atgaccctgcatttaccttccagagtcaagctcgtcgatgtcggtccccgcgacggcctgcagaacgaaaaatcgcccgttcccgccgccgtgaaaatcgaactcgtgcatcgcctgcaggacgccggtctcacggaaatcgaggtcaccagttacgtttcgcccaaatgggtgccgcagatggctgacaacgccgaagtcatggccggcatccggcgcaagcccggcgtgcgctattcggtgctgacgcccaacatgcagggtctagaggcggcgatgaagccagcgcccgagttgtggcccgacgagatcgtggtgttcggcgcggccagcgaagccttcagccagcgcaacatcaactgctcgattgccgaaagcatcgagcgtttccggccggtggtggctgcggcccgggccaacaatatttatgttcgcggcgccatttcctgcaccgtcggctgcccctacgagggcgagattgcgccggagcgcgtcgaaatggtggcgcgcctgatgaaggacatcggcgtgcagcacatcggcgtggccgacacgattggcgtcggcacgccgctgaaggtgcagcgcgccctggaagcggcgctgaagcattacgacctcgatggcgtgtcgggccatttccatgacacctacggccaggcgctggccaacacgctggcgtcgttgcaaatgggcgtctggcagttcgacacctcggtcgcgggcctgggtggctgcccctacgccaagggcgcgaccggcaatgtggcgacggaagacgtggtctatctgctgcacggcatgggaatcgaaaccggcatcgacctggacaagctgattgacgcgggcaagttcatcagcgacttcctcggtcgccagcccaactcgcgcgccgcgacggccttactgaacaagaggatgaattga

SEQ ID NO: 111 - protein, 308 aa, Polaromonas naphthalenivorans
MTLHLPSRVKLVDVGPRDGLQNEKSPVPAAVKIELVHRLQDAGLTEIEVTSYVSPKWVPQMADNAEVMAGIRRKPGVRYSVLTPNMQGLEAAMKPAPELWPDEIVVFGAASEAFSQRNINCSIAESIERFRPVVAAARANNIYVRGAISCTVGCPYEGEIAPERVEMVARLMKDIGVQHIGVADTIGVGTPLKVQRALEAALKHYDLDGVSGHFHDTYGQALANTLASLQMGVWQFDTSVAGLGGCPYAKGATGNVATEDVVYLLHGMGIETGIDLDKLIDAGKFISDFLGRQPNSRAATALLNKRMN

SEQ ID NO: 112 - DNA, 957 bp, Thermoplasma acidophilum
atgacctacagatcaataggatcaactgcttatccaacgatcggcgttgttctcctgggcgggatagcaaatcccgtcacgaggacgccgctgcatacctcggcagggatagcatacagtgattcctgcggatcgataagatccgagaccaggatatacgccgatgaggctacgcacatatatttcaatggtacggaatcgacagatgacaatagatccgtgaggcgtgtgcttgatcgttattcaagcgtcttcgaggaggcgttcggtacgaagactgtttcatattcttcacagaacttcggcatactgagcggcagttccgatgctggagcggcatcgataggcgcagctatattgggcctcaaacccgatctggatccgcacgatgttgaaaatgacctcagggcggtctccgaaagcgcaggcaggagcctcttcggcggcttaacaataacttggagcgatggtttccatgcatatacggaaaagattcttgatcctgaggcattctccgggtattcgatcgttgccttcgcgtttgactatcagagaaatccgtccgatgtcatacaccagaatatagtgagaagcgatctctatccggccaggaagaagcatgccgatgaacatgcgcacatgatcaaggagtacgcaaagacaaacgacataaagggcatatttgatctggcccaggaagatacagaggaataccattccatacttcgtggcgtgggcgtgaacgtgataagggagaacatgcagaagctcatcagttacctgaagctgatcaggaaggattactggaatgcctacatcgtgaccggcggaagcaacgtctacgtggctgtagaaagcgagaatgcagataggctgttttcgatcgaaaataccttcggttcaaaaaagaagatgctcaggatagttggcggtgcctggcatagaagaccagagtga

SEQ ID NO: 113 - protein, 318 aa, Thermoplasma acidophilum
MTYRSIGSTAYPTIGVVLLGGIANPVTRTPLHTSAGIAYSDSCGSIRSETRIYADEATHIYFNGTESTDDNRSVRRVLDRYSSVFEEAFGTKTVSYSSQNFGILSGSSDAGAASIGAAILGLKPDLDPHDVENDLRAVSESAGRSLFGGLTITWSDGFHAYTEKILDPEAFSGYSIVAFAFDYQRNPSDVIHQNIVRSDLYPARKKHADEHAHMIKEYAKTNDIKGIFDLAQEDTEEYHSILRGVGVNVIRENMQKLISYLKLIRKDYWNAYIVTGGSNVYVAVESENADRLFSIENTFGSKKKMLRIVGGAWHRRPE

SEQ ID NO: 114 - DNA, 957 bp, Thermoplasma acidophilum
atgacctacagatcaataggatcaactgcttatccaacgatcggcgttgttctcctgggcgggatagcaaatcccgtcacgaggacgccgctgcatacctcggcagggatagcatacagtgattcctgcggatcgataagatccgagaccaggatatacgccgatgaggctacgcacatatatttcaatggtacggaatcgacagatgacaatagatccgtgaggcgtgtgcttgatcgttattcaagcgtcttcgaggaggcgttcggtacgaagactgtttcatattcttcacagaacttcggcatactgagcggcagttccgatgctggagcggcatcgataggcgcagctatattgggcctcaaacccgatctggatccgcacgatgttgaaaatgacctcagggcggtctccgaaagcgcaggcaggagcctcttcggcggcttaacaataacttggagcgatggtttccatgcatatacggaaaagattcttgatcctgaggcattctccgggtattcgatcgttgccttcgcgtttgactatcagagaaatccgtccgatgtcatacaccagaatatagtgagaagcgatgagtatccggccaggaagaagcatgccgatgaacatgcgcacatgatcaaggagtacgcaaagacaaacgacataaagggcatatttgatctggcccaggaagatacagaggaataccattccatacttcgtggcgtgggcgtgaacgtgataagggagaacatgcagaagctcatcagttacctgaagctgatcaggaaggattactggaatgcctacatcgtgaccggcggaagcaacgtctacgtggctgtagaaagcgagaatgcagataggctgttttcgatcgaaaataccttcggttcaaaaaagaagatgctcaggatagttggcggtgcctggcatagaagaccagagtga

SEQ ID NO: 115 - protein, 318 aa, Thermoplasma acidophilum
MTYRSIGSTAYPTIGVVLLGGIANPVTRTPLHTSAGIAYSDSCGSIRSETRIYADEATHIYFNGTESTDDNRSVRRVLDRYSSVFEEAFGTKTVSYSSQNFGILSGSSDAGAASIGAAILGLKPDLDPHDVENDLRAVSESAGRSLFGGLTITWSDGFHAYTEKILDPEAFSGYSIVAFAFDYQRNPSDVIHQNIVRSDEYPARKKHADEHAHMIKEYAKTNDIKGIFDLAQEDTEEYHSILRGVGVNVIRENMQKLISYLKLIRKDYWNAYIVTGGSNVYVAVESENADRLFSIENTFGSKKKMLRIVGGAWHRRPE

SEQ ID NO: 116 - DNA, 975 bp, Picrophilus torridus
atggaaaattacaatgttaagacaagggcgttcccaacaataggcataatactgcttggtgggatctcggataaaaagaacaggataccgctgcatacaacggcaggcatagcatatactggtataaacaatgatgtttacactgagacaaagctttatgtatcaaaagatgaaaaatgctatattgatggaaaggaaattgatttaaattcagatagatcaccatcgaaggttattgataaattcaagcatgaaatacttatgagagtaaatcttgatgatgaaaataacctttcaattgattcaaggaactttaatatattaagtggcagctcagattctggggccgctgcactgggagagtgcatagaatcaatttttgaatacaatataaatatatttacatttgaaaacgatcttcagaggatatcagaaagtgttggaagaagcctttacggtggtttaacagtaaactatgccaatggcagggaatcattaacagagccattacttgagcctgaggcatttaataactttacaataattggtgcacattttaacattgatagaaaaccatcaaatgagattcatgaaaatatcataaaacatgaaaattacagggaaagaataaaaagtgctgagagaaaggcgaaaaaacttgaggagctatcaaggaatgcaaacataaagggtatctttgaacttgcagaatccgatacagtggaataccataaaatgctccatgatgttggcgttgacataataaatgatagaatggagaacctcattgaaagggtaaaagaaatgaaaaataacttctggaattcatacatagttaccggcggcccgaacgtttttgtaataacagagaaaaaggacgttgataaggcaatggaaggattaaatgatctgtgcgatgatataagattattaaaagttgcaggaaagccacaggtcatttcaaaaaacttttaa

SEQ ID NO: 117 - protein, 324 aa, Picrophilus torridus
MENYNVKTRAFPTIGIILLGGISDKKNRIPLHTTAGIAYTGINNDVYTETKLYVSKDEKCYIDGKEIDLNSDRSPSKVIDKFKHEILMRVNLDDENNLSIDSRNFNILSGSSDSGAAALGECIESIFEYNINIFTFENDLQRISESVGRSLYGGLTVNYANGRESLTEPLLEPEAFNNFTIIGAHFNIDRKPSNEIHENIIKHENYRERIKSAERKAKKLEELSRNANIKGIFELAESDTVEYHKMLHDVGVDIINDRMENLIERVKEMKNNFWNSYIVTGGPNVFVITEKKDVDKAMEGLNDLCDDIRLLKVAGKPQVISKNF

SEQ ID NO: 118 - DNA, 954 bp, Streptococcus mitis
atggatagagagcctgtaacagtacgttcctacgcaaatattgccattatcaaatactggggaaagaaaaaagaaaaagagatggttcctgctactagcagtatttctctgactttggaaaatatgtacacggagacgaccttgtcatctctaccgacggatgcgacagctgatgcattttatatcaatggtcagttacaaaatgaggcagagcatgtcaagatgagtaagattattgaccgctaccgtccagatggtgatggctttgttcgtatcgatactcaaaacagtatgcctaccgcagcgggcttgtcatcaagttctagtggtttgtctgccttggtcaaggcttgtaatgcttatttcaagcttggtttgaatcggagtcagttggcacaggaggctaagtttgcctcaggctcttcctctcggagtttttatggaccactcggtgcctgggataaggatagcggagagatttacccggtagagacaggcctgaaactagctatgattatgttggtgctagaagacaagaaaaaaccaatctctagccgtgatgggatgaaactttgtgtggaaacttcgacgacctttgacgactgggtgcgtcagtctgagaaggattatcaggatatgctggtttacctcaaggcaaatgactttgccaaggttggggaattaacggagaaaaatgccctggctatgcacgctacgaccaaaacagcatcaccagccttttcttatctgacggatgcttcttatgaagccatggactttgtccgccagctccgtgagcaaggggaagcctgctactttactatggatgctggtcccaatgtcaaggtcctctgtcaggagaaagacttggagcatttatcagaaatcttcggtcaacgttatcgcttgattgtgtcaaaaacaaaggatttgagccaagatggttgctgttaa

SEQ ID NO: 119 - protein, 317 aa, Streptococcus mitis
MDREPVTVRSYANIAIIKYWGKKKEKEMVPATSSISLTLENMYTETTLSSLPTDATADAFYINGQLQNEAEHVKMSKIIDRYRPDGDGFVRIDTQNSMPTAAGLSSSSSGLSALVKACNAYFKLGLNRSQLAQEAKFASGSSSRSFYGPLGAWDKDSGEIYPVETGLKLAMIMLVLEDKKKPISSRDGMKLCVETSTTFDDWVRQSEKDYQDMLVYLKANDFAKVGELTEKNALAMHATTKTASPAFSYLTDASYEAMDFVRQLREQGEACYFTMDAGPNVKVLCQEKDLEHLSEIFGQRYRLIVSKTKDLSQDGCC

SEQ ID NO: 120 - DNA, 948 bp, Streptococcus gordonii
atggatcgaaagcctgtaagtgtcaaatcctatgccaatattgcaattgtcaaatattggggcaagaaagatgcagaaaagatgattccgtctacaagcagtatctcactgacactggaaaatatgtataccgagacgcagctgagtcctttgccggatacagcgactggtgatgagttttatattgacggtcagctgcaaagcccggcagaacatgccaaaatcagtaaaattattgaccgttttcgctctccagaagatggttttgtccgtgttgataccagcaataatatgccaacagcagcagggctgtcttccagctccagcggtctatctgctctggtcaaggcttgcaatgcttattttcagacaggctatcagacggaagaactggctcaactggccaagtttgcttcagggtcctctgctcggtctttctttggtccgctagcggcctgggataaggacagtggagccatctatccagtcaagacggatttgaagctagccatgattatgctggttctgcacgatgagaaaaagcccatttccagccgcgacggtatggagctctgtgctaaaacttccacaattttcccagattggattgcccaatctgccttggattatcaggccatgctaggctatttgcaggacaatgattttgccaaggttggtcaactgacggaagaaaatgcccttcggatgcatgctacaacagaaaaagcttatccgccgttttcttatctgacagaggagtcttatcaggctatggatgctgtcagaaagctacgggagcagggcgagcgttgctactttaccatggatgcggggccaaatgtcaaggtgctctgcttggaggaagatctagaccatttggctgccatatttgagaaggactatcgccttattgtctccaaaacaaaggacttgtcagatgaaagctag

SEQ ID NO: 121 - protein, 315 aa, Streptococcus gordonii
MDRKPVSVKSYANIAIVKYWGKKDAEKMIPSTSSISLTLENMYTETQLSPLPDTATGDEFYIDGQLQSPAEHAKISKIIDRFRSPEDGFVRVDTSNNMPTAAGLSSSSSGLSALVKACNAYFQTGYQTEELAQLAKFASGSSARSFFGPLAAWDKDSGAIYPVKTDLKLAMIMLVLHDEKKPISSRDGMELCAKTSTIFPDWIAQSALDYQAMLGYLQDNDFAKVGQLTEENALRMHATTEKAYPPFSYLTEESYQAMDAVRKLREQGERCYFTMDAGPNVKVLCLEEDLDHLAAIFEKDYRLIVSKTKDLSDES

SEQ ID NO: 122 - DNA, 1476 bp, Saccharomyces cerevisiae; identical to SEQ ID NO: 106

SEQ ID NO: 123 - protein, 491 aa, Saccharomyces cerevisiae; identical to SEQ ID NO: 107

SEQ ID NO: 124 - DNA, 816 bp, Pseudomonas putida
atgagcgatttcagcaccctcgaagtgatccgcgacccgcgcggcttcgccaccctgtggttaagccgtgaggacaagaacaacgccttcaatgcgcagatgatccgcgagctgatcgtggccatcgaccagttggccgaagacgccagcctgcgctttgtgctgctgcgcggccgtggccggcacttcagcgccggtgccgacctggcctggatgcagcagtcggcgcaactggacttcaacaccaacctggatgatgcccacgaactgggcgagctgatgtacgccctgcatcgcctcaaggccccgaccctggccgtggtgcaaggtgcagcctttggcggcgcgctgggcctgatcagctgctgcgacatggccatcggcgccgaagacgcccaactgtgcctgtcggaagtacgcatcggcctggccccggcggtcatcagcccgttcgtggtcaaggccattggcgaacgcgccgcgcgccgctacgccctcaccgccgagcgtttcactggcgtgcgcgcacgcgagctgggcctgctggccgaggtgtacccggccagcgaactggacgaccacgtcgaagcctgggtaagcaacctgctgcagaacagcccgcaagcattgcgcgccaccaaggacttgctgcgcgaagtggacgacggcgaactcagcccggccctgcgccgttactgcgaaaacaccatcgcccgtatccgcgtcagcgccgaaggccaggagggcctgcgcgctttcctggaaaaacgccgccctgcctggcaaaccgttgacaagaaggagccgcgcccatga

SEQ ID NO: 125 - protein, 271 aa, Pseudomonas putida
MSDFSTLEVIRDPRGFATLWLSREDKNNAFNAQMIRELIVAIDQLAEDASLRFVLLRGRGRHFSAGADLAWMQQSAQLDFNTNLDDAHELGELMYALHRLKAPTLAVVQGAAFGGALGLISCCDMAIGAEDAQLCLSEVRIGLAPAVISPFVVKAIGERAARRYALTAERFTGVRARELGLLAEVYPASELDDHVEAWVSNLLQNSPQALRATKDLLREVDDGELSPALRRYCENTIARIRVSAEGQEGLRAFLEKRRPAWQTVDKKEPRP

SEQ ID NO: 126 - DNA, 1608 bp, Pseudomonas aeruginosa
atggccatcctgcacacccagatcaatccacgctccgccgagttcgcggccaacgccgcgaccatgctggaacaggtcaacgccctgcgcaccctcctcggccgcatccacgaaggcggcggtagcgccgcccaggcccggcacagcgcgcgcggcaagctgctggtgcgcgaacgcatcaaccgcctgctcgaccccggctcgccgttcctcgagctttccgccctcgccgcccacgaagtctacggcgaagaggtcgccgccgccggcatcgtcgccgggatcggccgggtcgaaggggtggaatgcatgatcgtcggcaacgatgccacggtgaagggcggcacctactatccgctgaccgtgaagaagcacctgcgggcccaggccatcgccctggagaaccgcctgccatgcatctacctggtggactctggcggcgccaacctgccgcgccaggacgaggtgttccccgaccgcgagcacttcggccggatcttcttcaaccaggccaacatgagtgcccgcggcatcccgcagatcgccgtggtcatgggctcctgcaccgccggcggggcctacgtgccggcgatgtccgacgaaaccgtgatggtgcgcgaacaggccaccatcttcctcgccgggccgccgctggtgaaggccgctaccggcgaggtggtcagcgccgaggaacttggcggcgccgacgtgcactgcaaggtctcaggggtcgccgaccactatgccgaggacgatgaccatgccctggccatcgcccgccgctgcgtcgccaacctcaactggcgcaagcagggccagctgcaatgccgtgcaccgcgcgcgccgctgtaccccgccgaggagctgtacggggtgattcccgccgacagcaagcagccctacgacgtgcgcgaagtgatcgcacggctggtggacggctccgagttcgacgaattcaaggcgctgttcggcaccaccctggtctgcggcttcgcccacctgcacggctatccgatcgccatcctggccaacaacgggatcctcttcgccgaggccgcgcagaaaggcgcgcacttcatcgagttggcctgccagcgtggcatcccgctgctgttcctgcagaacatcaccggtttcatggtcggccagaagtacgaggctggcggcatcgccaagcacggcgccaagctggtgaccgcggtggcctgcgcgcgggtgccgaagttcaccgtgctgatcggcggcagcttcggcgccggcaactacggcatgtgcggacgcgcctacgacccgcgcttcctgtggatgtggccgaacgcgaggatcggcgtgatgggcggcgaacaggccgccggggtgctcgcccaggtcaagcgcgagcaggccgagcgcgccggccagcagctcggcgtcgaagaggaggcgaagatcaaggcgccgatcctcgagcagtacgagcaccagggccatccctactactccagcgcgcgactgtgggacgacggcgtgatcgatccggcgcagacccgcgaggtcctcgccctggcgctttccgccgccctcaacgcccccatcgagccgaccgccttcggcgtgttccgcatgtga

SEQ ID NO: 127 - protein, 535 aa, Pseudomonas aeruginosa
MAILHTQINPRSAEFAANAATMLEQVNALRTLLGRIHEGGGSAAQARHSARGKLLVRERINRLLDPGSPFLELSALAAHEVYGEEVAAAGIVAGIGRVEGVECMIVGNDATVKGGTYYPLTVKKHLRAQAIALENRLPCIYLVDSGGANLPRQDEVFPDREHFGRIFFNQANMSARGIPQIAVVMGSCTAGGAYVPAMSDETVMVREQATIFLAGPPLVKAATGEVVSAEELGGADVHCKVSGVADHYAEDDDHALAIARRCVANLNWRKQGQLQCRAPRAPLYPAEELYGVIPADSKQPYDVREVIARLVDGSEFDEFKALFGTTLVCGFAHLHGYPIAILANNGILFAEAAQKGAHFIELACQRGIPLLFLQNITGFMVGQKYEAGGIAKHGAKLVTAVACARVPKFTVLIGGSFGAGNYGMCGRAYDPRFLWMWPNARIGVMGGEQAAGVLAQVKREQAERAGQQLGVEEEAKIKAPILEQYEHQGHPYYSSARLWDDGVIDPAQTREVLALALSAALNAPIEPTAFGVFRM

SEQ ID NO: 128 - DNA, 1968 bp, Pseudomonas aeruginosa
atgaacccggactaccgcagcatccagcgcctcctggtcgccaaccgcggcgagatcgcctgccgggtgatgcgcagcgcgcgcgccctcggcatcggcagcgtcgccgtgcacagcgacatcgaccgccacgcccgccacgtcgccgaagccgacatcgccgtcgacctgggcggcgccaagcccgccgacagctacctgcgcggcgacaggatcatcgcagctgccctcgccagcggcgcccaggcgatccaccccggctacggcttcctctcggagaacgccgacttcgcccgcgcctgcgaggaagccggcctgctcttcctcggaccgccggcggcggccatcgacgccatgggcagcaagtcggccgccaaggcgctgatggaagaagccggcgtgccgctggtgcccggctatcacggcgaagcccaggacctcgagactttccgccgggaagccgggcgcatcggctacccggtgctgctcaaggccgccgctggcggcggcggcaagggcatgaaggtggtggagcgcgaggccgagctggccgaggccctttcctccgcccagcgcgaggccaaggccgccttcggcgacgcgcgtatgctggtggagaaatacctgctgaagccgcgccacgtggagatccaggtattcgccgaccgccatggccactgcctgtacctcaacgaacgcgactgttcgatccagcgccgccaccagaaggtcgtcgaggaagcccccgcccccggtctcggcgcggaactgcgccgggccatgggcgaggccgcggtacgcgcggcgcaggcgatcggctacgtcggcgccggtaccgtcgaattcctcctcgacgaacgcggccagttcttcttcatggaaatgaacacccgcctgcaggtggaacacccggtcaccgaggccatcaccggcctcgacctggtcgcctggcagatccgcgtcgctcgcggcgaggcgctaccgctgacgcaggaacaggtaccgctgaacggccacgccatcgaagtacggctctatgcggaagaccccgagggcgacttcctccccgccagcggccgcctgatgctataccgcgaagcggccgccggccccggccggcgggtcgacagcggggtacgcgaaggcgacgaggtctcgccgttctacgacccgatgctggccaagctgatcgcctggggcgaaacccgcgaggaagcccgccagcgcctgctggcgatgctggcggaaacctcggtcggcggcctgcgcaccaacctggcgttcctccgccgcatcctcggccatccggcgttcgccgccgccgaactggacaccgggttcatcgcgcgccaccaagacgacctgctgccggcgccgcaagcgctgccggagcatttctggcaagccgccgccgaagcctggctgcagagcgaaccgggccaccgtcgcgacgacgacccgcattcgccgtggagccgcaacgacggctggcgcagcgccctggcgcgggagtccgacctgatgctgcgctgccgcgacgaacgccgctgcgtccgcctgcgccacgccagccccagccagtatcgcctcgacggcgacgacctggtcagccgggtggacggtgtcacccgtcgctcggcggcgttgcgccgcggacgccaattgttcctcgaatgggagggcgaactgctggccatcgaagccgtcgacccgatcgccgaggccgaggccgcccatgcccaccagggcggtttgagcgcgccgatgaacggcagtatcgtgcgggtcctggtcgagcccggccagacggtcgaagccggcgccaccctggtggtgctggaggcgatgaagatggaacacagcatccgcgcgccccacgccggggtagtgaaggcgctgtattgcagcgagggcgaactggtcgaggaaggcacgccgctggtggagctggacgagaaccaggcctga

SEQ ID NO: 129 - protein, 655 aa, Pseudomonas aeruginosa
MNPDYRSIQRLLVANRGEIACRVMRSARALGIGSVAVHSDIDRHARHVAEADIAVDLGGAKPADSYLRGDRIIAAALASGAQAIHPGYGFLSENADFARACEEAGLLFLGPPAAAIDAMGSKSAAKALMEEAGVPLVPGYHGEAQDLETFRREAGRIGYPVLLKAAAGGGGKGMKVVEREAELAEALSSAQREAKAAFGDARMLVEKYLLKPRHVEIQVFADRHGHCLYLNERDCSIQRRHQKVVEEAPAPGLGAELRRAMGEAAVRAAQAIGYVGAGTVEFLLDERGQFFFMEMNTRLQVEHPVTEAITGLDLVAWQIRVARGEALPLTQEQVPLNGHAIEVRLYAEDPEGDFLPASGRLMLYREAAAGPGRRVDSGVREGDEVSPFYDPMLAKLIAWGETREEARQRLLAMLAETSVGGLRTNLAFLRRILGHPAFAAAELDTGFIARHQDDLLPAPQALPEHFWQAAAEAWLQSEPGHRRDDDPHSPWSRNDGWRSALARESDLMLRCRDERRCVRLRHASPSQYRLDGDDLVSRVDGVTRRSAALRRGRQLFLEWEGELLAIEAVDPIAEAEAAHAHQGGLSAPMNGSIVRVLVEPGQTVEAGATLVVLEAMKMEHSIRAPHAGVVKALYCSEGELVEEGTPLVELDENQA

SEQ ID NO: 130 - DNA, 1164 bp, Escherichia coli
atggaacaggttgtcattgtcgatgcaattcgcaccccgatgggccgttcgaagggcggtgcttttcgtaacgtgcgtgcagaagatctctccgctcatttaatgcgtagcctgctggcgcgtaacccggcgctggaagcggcggccctcgacgatatttactggggttgtgtgcagcagacgctggagcagggttttaatatcgcccgtaacgcggcgctgctggcagaagtaccacactctgtcccggcggttaccgttaatcgcttgtgtggttcatccatgcaggcactgcatgacgcagcacgaatgatcatgactggcgatgcgcaggcatgtctggttggcggcgtggagcatatgggccatgtgccgatgagtcacggcgtcgattttcaccccggcctgagccgcaatgtcgccaaagcggcgggcatgatgggcttaacggcagaaatgctggcgcgtatgcacggtatcagccgtgaaatgcaggatgcctttgccgcgcggtcacacgcccgcgcctgggccgccacgcagtcggccgcatttaaaaatgaaatcatcccgaccggtggtcacgatgccgacggcgtcctgaagcagtttaattacgacgaagtgattcgcccggaaaccaccgtggaagccctcgccacgctgcgtccggcgtttgatccagtaaacggtatggtaacggcgggcacatcttctgcactttccgatggcgcagctgccatgctggtgatgagtgaaagccgcgcccatgaattaggtcttaagccgcgcgctcgtgtgcgttcgatggcggtcgttggttgtgacccatcgattatgggttacggcccggttccggcctcgaaactggcgctgaaaaaagcggggctttctgccagcgatatcggcgtgtttgaaatgaacgaagcctttgccgcgcagatcctgccatgtattaaagatctgggactaattgagcagattgacgagaagatcaacctcaacggtggcgcgatcgcgctgggtcatccgctgggttgttccggtgcgcgtatcagcaccacgctgctgaatctgatggaacgcaaagacgttcagtttggtctggcgacgatgtgtatcggtctgggtcagggtattgcgacggtgtttgagcgggtttaa

SEQ ID NO: 131 - protein, 387 aa, Escherichia coli
MEQVVIVDAIRTPMGRSKGGAFRNVRAEDLSAHLMRSLLARNPALEAAALDDIYWGCVQQTLEQGFNIARNAALLAEVPHSVPAVTVNRLCGSSMQALHDAARMIMTGDAQACLVGGVEHMGHVPMSHGVDFHPGLSRNVAKAAGMMGLTAEMLARMHGISREMQDAFAARSHARAWAATQSAAFKNEIIPTGGHDADGVLKQFNYDEVIRPETTVEALATLRPAFDPVNGMVTAGTSSALSDGAAAMLVMSESRAHELGLKPRARVRSMAVVGCDPSIMGYGPVPASKLALKKAGLSASDIGVFEMNEAFAAQILPCIKDLGLIEQIDEKINLNGGAIALGHPLGCSGARISTTLLNLMERKDVQFGLATMCIGLGQGIATVFERV

SEQ ID NO: 132 - DNA, 2190 bp, Escherichia coli
atgctttacaaaggcgacaccctgtaccttgactggctggaagatggcattgccgaactggtatttgatgccccaggttcagttaataaactcgacactgcgaccgtcgccagcctcggcgaggccatcggcgtgctggaacagcaatcagatctaaaagggctgctgctgcgttcgaacaaagcagcctttatcgtcggtgctgatatcaccgaatttttgtccctgttcctcgttcctgaagaacagttaagtcagtggctgcactttgccaatagcgtgtttaatcgcctggaagatctgccggtgccgaccattgctgccgtcaatggctatgcgctgggcggtggctgcgaatgcgtgctggcgaccgattatcgtctggcgacgccggatctgcgcatcggtctgccggaaaccaaactgggcatcatgcctggctttggcggttctgtacgtatgccacgtatgctgggcgctgacagtgcgctggaaatcattgccgccggtaaagatgtcggcgcggatcaggcgctgaaaatcggtctggtggatggcgtagtcaaagcagaaaaactggttgaaggcgcaaaggcggttttacgccaggccattaacggcgacctcgactggaaagcaaaacgtcagccgaagctggaaccactaaaactgagcaagattgaagccaccatgagcttcaccatcgctaaagggatggtcgcacaaacagcggggaaacattatccggcccccatcaccgcagtaaaaaccattgaagctgcggcccgttttggtcgtgaagaagccttaaacctggaaaacaaaagttttgtcccgctggcgcataccaacgaagcccgcgcactggtcggcattttccttaacgatcaatatgtaaaaggcaaagcgaagaaactcaccaaagacgttgaaaccccgaaacaggccgcggtgctgggtgcaggcattatgggcggcggcatcgcttaccagtctgcgtggaaaggcgtgccggttgtcatgaaagatatcaacgacaagtcgttaaccctcggcatgaccgaagccgcgaaactgctgaacaagcagcttgagcgcggcaagatcgatggtctgaaactggctggcgtgatctccacaatccacccaacgctcgactacgccggatttgaccgcgtggatattgtggtagaagcggttgttgaaaacccgaaagtgaaaaaagccgtactggcagaaaccgaacaaaaagtacgccaggataccgtgctggcgtctaacacttcaaccattcctatcagcgaactggccaacgcgctggaacgcccggaaaacttctgcgggatgcacttctttaacccggtccaccgaatgccgttggtagaaattattcgcggcgagaaaagctccgacgaaaccatcgcgaaagttgtcgcctgggcgagcaagatgggcaagacgccgattgtggttaacgactgccccggcttctttgttaaccgcgtgctgttcccgtatttcgccggtttcagccagctgctgcgcgacggcgcggatttccgcaagatcgacaaagtgatggaaaaacagtttggctggccgatgggcccggcatatctgctggacgttgtgggcattgataccgcgcatcacgctcaggctgtcatggcagcaggcttcccgcagcggatgcagaaagattaccgcgatgccatcgacgcgctgtttgatgccaaccgctttggtcagaagaacggcctcggtttctggcgttataaagaagacagcaaaggtaagccgaagaaagaagaagacgccgccgttgaagacctgctggcagaagtgagccagccgaagcgcgatttcagcgaagaagagattatcgcccgcatgatgatcccgatggtcaacgaagtggtgcgctgtctggaggaaggcattatcgccactccggcggaagcggatatggcgctggtctacggcctgggcttccctccgttccacggcggcgcgttccgctggctggacaccctcggtagcgcaaaatacctcgatatggcacagcaatatcagcacctcggcccgctgtatgaagtgccggaaggtctgcgtaataaagcgcgtcataacgaaccgtactatcctccggttgagccagcccgtccggttggcgacctgaaaacggcttaa

SEQ ID NO: 133 - protein, 729 aa, Escherichia coli
MLYKGDTLYLDWLEDGIAELVFDAPGSVNKLDTATVASLGEAIGVLEQQSDLKGLLLRSNKAAFIVGADITEFLSLFLVPEEQLSQWLHFANSVFNRLEDLPVPTIAAVNGYALGGGCECVLATDYRLATPDLRIGLPETKLGIMPGFGGSVRMPRMLGADSALEIIAAGKDVGADQALKIGLVDGVVKAEKLVEGAKAVLRQAINGDLDWKAKRQPKLEPLKLSKIEATMSFTIAKGMVAQTAGKHYPAPITAVKTIEAAARFGREEALNLENKSFVPLAHTNEARALVGIFLNDQYVKGKAKKLTKDVETPKQAAVLGAGIMGGGIAYQSAWKGVPVVMKDINDKSLTLGMTEAAKLLNKQLERGKIDGLKLAGVISTIHPTLDYAGFDRVDIVVEAVVENPKVKKAVLAETEQKVRQDTVLASNTSTIPISELANALERPENFCGMHFFNPVHRMPLVEIIRGEKSSDETIAKVVAWASKMGKTPIVVNDCPGFFVNRVLFPYFAGFSQLLRDGADFRKIDKVMEKQFGWPMGPAYLLDVVGIDTAHHAQAVMAAGFPQRMQKDYRDAIDALFDANRFGQKNGLGFWRYKEDSKGKPKKEEDAAVEDLLAEVSQPKRDFSEEEIIARMMIPMVNEVVRCLEEGIIATPAEADMALVYGLGFPPFHGGAFRWLDTLGSAKYLDMAQQYQHLGPLYEVPEGLRNKARHNEPYYPPVEPARPVGDLKTA

SEQ ID NO: 134 - DNA, 861 bp, Escherichia coli
atgagtcaggcgctaaaaaatttactgacattgttaaatctggaaaaaattgaggaaggactctttcgcggccagagtgaagatttaggtttacgccaggtgtttggcggccaggtcgtgggtcaggccttgtatgctgcaaaagagaccgtccctgaagagcggctggtacattcgtttcacagctactttcttcgccctggcgatagtaagaagccgattatttatgatgtcgaaacgctgcgtgacggtaacagcttcagcgcccgccgggttgctgctattcaaaacggcaaaccgattttttatatgactgcctctttccaggcaccagaagcgggtttcgaacatcaaaaaacaatgccgtccgcgccagcgcctgatggcctcccttcggaaacgcaaatcgcccaatcgctggcgcacctgctgccgccagtgctgaaagataaattcatctgcgatcgtccgctggaagtccgtccggtggagtttcataacccactgaaaggtcacgtcgcagaaccacatcgtcaggtgtggatccgcgcaaatggtagcgtgccggatgacctgcgcgttcatcagtatctgctcggttacgcttctgatcttaacttcctgccggtagctctacagccgcacggcatcggttttctcgaaccggggattcagattgccaccattgaccattccatgtggttccatcgcccgtttaatttgaatgaatggctgctgtatagcgtggagagcacctcggcgtccagcgcacgtggctttgtgcgcggtgagttttatacccaagacggcgtactggttgcctcgaccgttcaggaaggggtgatgcgtaatcacaattaa

SEQ ID NO: 135 - protein, 286 aa, Escherichia coli
MSQALKNLLTLLNLEKIEEGLFRGQSEDLGLRQVFGGQVVGQALYAAKETVPEERLVHSFHSYFLRPGDSKKPIIYDVETLRDGNSFSARRVAAIQNGKPIFYMTASFQAPEAGFEHQKTMPSAPAPDGLPSETQIAQSLAHLLPPVLKDKFICDRPLEVRPVEFHNPLKGHVAEPHRQVWIRANGSVPDDLRVHQYLLGYASDLNFLPVALQPHGIGFLEPGIQIATIDHSMWFHRPFNLNEWLLYSVESTSASSARGFVRGEFYTQDGVLVASTVQEGVMRNHN

SEQ ID NO: 136 - protein, 520 aa, Mus musculus
MPGSLPLNAEACWPKDVGIVALEIYFPSQYVDQAELEKYDGVDAGKYTIGLGQARMGFCTDREDINSLCLTVVQKLMERHSLSYDCIGRLEVGTETIIDKSKSVKSKLMQLFEESGNTDIEGIDTTNACYGGTAAVFNAVNWVESSSWDGRYALVVAGDIAIYAPGNARPTGGVGAVALLIGPNAPLIFDRGLRGTHMQHAYDFYKPDMLSEYPVVDGKLSQQCYLSALDRCYSVYRKKIRAQWQKEGKDKDFTLNDFGFMIFHSPYCKLVQKSLARMFLNDFLNDQNRDKNSIYQGLEAFGDVKLEDTYFDRDVEKAFMKASSELFNQKTKASLLVSNQNGNMYTSSVYGSLASVLAQYSPQQLAGKRVGVFSYGSGLAATLYSLKVTQDATPGSALDKITASLCDLKSRLDSRTCVAPDVFAENMKLREDTHHLANYIPQCSIDSLFEGTWYLVRVDEKHRRTYARRPFTNDHSLDEGMGLVHSNTATEHIPSPAKKSPRLPATSAESESAVISNGEH

SEQ ID NO: 137 - protein, 317 aa, Streptococcus mitis
MDREPVTVRSYANIALIKYWGKKKEKEMVPATSSISLTLENMYTETTLSSLPTDATADAFYINGQLQNEAEHVKMSKIIDRYRPDGDGFVRIDTQNSMPTAAGLSSSSSGLSALVKALNARFKLGLNRSQLAQEAKFASGPSSRSFYGPLGAWDKDSGLIYPVETGLKLAMICLVLCDKPKPISSRDGMKLCVETSTTFDDWVRQSEKDYQDMLVYLKANDFAKVGELTEKNALAMHATTITASPAFTYLTDASYEAMDFVRQLREQGEACYFTMDAGPNVCVLCQEKDLDHLSEILGQRYRMIVSKSKDLSQDGCC

SEQ ID NO: 138 - protein, 329 aa, Unknown
  Streptomyces sp. acetoacetyl CoA synthase NphT7
MTDVRFRIIGTGAYVPERIVSNDEVGAPAGVDDDWITRKTGIRQRRWAADDQATSDLATAAGRAALKAAGITPEQLTVIAVATSTPDRPQPPTAAYVQHHLGATGTAAFDVNAVCSGTVFALSSVAGTLVYRGGYALVIGADLYSRILNPADRKTVVLFGDGAGAMVLGPTSTGTGPIVRRVALHTFGGLTDLIRVPAGGSRQPLDTDGLDAGLQYFAMDGREVRRFVTEHLPQLIKGFLHEAGVDAADISHFVPHQANGVMLDEVFGELHLPRATMHRTVETYGNTGAASIPITMDAAVRAGSFRPGELVLLAGFGGGMAASFALIEW

SEQ ID NO: 139 - DNA, 960 bp, Escherichia coli
atgagtctgaatttccttgattttgaacagccgattgcagagctggaagcgaaaatcgattctctgactgcggttagccgtcaggatgagaaactggatattaacatcgatgaagaagtgcatcgtctgcgtgaaaaaagcgtagaactgacacgtaaaatcttcgccgatctcggtgcatggcagattgcgcaactggcacgccatccacagcgtccttataccctggattacgttcgcctggcatttgatgaatttgacgaactggctggcgaccgcgcgtatgcagacgataaagctatcgtcggtggtatcgcccgtctcgatggtcgtccggtgatgatcattggtcatcaaaaaggtcgtgaaaccaaagaaaaaattcgccgtaactttggtatgccagcgccagaaggttaccgcaaagcactgcgtctgatgcaaatggctgaacgctttaagatgcctatcatcacctttatcgacaccccgggggcttatcctggcgtgggcgcagaagagcgtggtcagtctgaagccattgcacgcaacctgcgtgaaatgtctcgcctcggcgtaccggtagtttgtacggttatcggtgaaggtggttctggcggtgcgctggcgattggcgtgggcgataaagtgaatatgctgcaatacagcacctattccgttatctcgccggaaggttgtgcgtccattctgtggaagagcgccgacaaagcgccgctggcggctgaagcgatgggtatcattgctccgcgtctgaaagaactgaaactgatcgactccatcatcccggaaccactgggtggtgctcaccgtaacccggaagcgatggcggcatcgttgaaagcgcaactgctggcggatctggccgatctcgacgtgttaagcactgaagatttaaaaaatcgtcgttatcagcgcctgatgagctacggttacgcgtaa

SEQ ID NO: 140 - protein, 319 aa, Escherichia coli
MSLNFLDFEQPIAELEAKIDSLTAVSRQDEKLDINIDEEVHRLREKSVELTRKIFADLGAWQIAQLARHPQRPYTLDYVRLAFDEFDELAGDRAYADDKAIVGGIARLDGRPVMIIGHQKGRETKEKIRRNFGMPAPEGYRKALRLMQMAERFKMPIITFIDTPGAYPGVGAEERGQSEAIARNLREMSRLGVPVVCTVIGEGGSGGALAIGVGDKVNMLQYSTYSVISPEGCASILWKSADKAPLAAEAMGIIAPRLKELKLIDSIIPEPLGGAHRNPEAMAASLKAQLLADLADLDVLSTEDLKNRRYQRLMSYGYA

SEQ ID NO: 141 - DNA, 471 bp, Escherichia coli
atggatattcgtaagattaaaaaactgatcgagctggttgaagaatcaggcatctccgaactggaaatttctgaaggcgaagagtcagtacgcattagccgtgcagctcctgccgcaagtttccctgtgatgcaacaagcttacgctgcaccaatgatgcagcagccagctcaatctaacgcagccgctccggcgaccgttccttccatggaagcgccagcagcagcggaaatcagtggtcacatcgtacgttccccgatggttggtactttctaccgcaccccaagcccggacgcaaaagcgttcatcgaagtgggtcagaaagtcaacgtgggcgataccctgtgcatcgttgaagccatgaaaatgatgaaccagatcgaagcggacaaatccggtaccgtgaaagcaattctggtcgaaagtggacaaccggtagaatttgacgagccgctggtcgtcatcgagtaa

SEQ ID NO: 142 - protein, 156 aa, Escherichia coli
MDIRKIKKLIELVEESGISELEISEGEESVRISRAAPAASFPVMQQAYAAPMMQQPAQSNAAAPATVPSMEAPAAAEISGHIVRSPMVGTFYRTPSPDAKAFIEVGQKVNVGDTLCIVEAMKMMNQIEADKSGTVKAILVESGQPVEFDEPLVVIE

SEQ ID NO: 143 - DNA, 1350 bp, Escherichia coli
atgctggataaaattgttattgccaaccgcggcgagattgcattgcgtattcttcgtgcctgtaaagaactgggcatcaagactgtcgctgtgcactccagcgcggatcgcgatctaaaacacgtattactggcagatgaaacggtctgtattggccctgctccgtcagtaaaaagttatctgaacatcccggcaatcatcagcgccgctgaaatcaccggcgcagtagcaatccatccgggttacggcttcctctccgagaacgccaactttgccgagcaggttgaacgctccggctttatcttcattggcccgaaagcagaaaccattcgcctgatgggcgacaaagtatccgcaatcgcggcgatgaaaaaagcgggcgtcccttgcgtaccgggttctgacggcccgctgggcgacgatatggataaaaaccgtgccattgctaaacgcattggttatccggtgattatcaaagcctccggcggcggcggcggtcgcggtatgcgcgtagtgcgcggcgacgctgaactggcacaatccatctccatgacccgtgcggaagcgaaagctgctttcagcaacgatatggtttacatggagaaatacctggaaaatcctcgccacgtcgagattcaggtactggctgacggtcagggcaacgctatctatctggcggaacgtgactgctccatgcaacgccgccaccagaaagtggtcgaagaagcgccagcaccgggcattaccccggaactgcgtcgctacatcggcgaacgttgcgctaaagcgtgtgttgatatcggctatcgcggtgcaggtactttcgagttcctgttcgaaaacggcgagttctatttcatcgaaatgaacacccgtattcaggtagaacacccggttacagaaatgatcaccggcgttgacctgatcaaagaacagctgcgtatcgctgccggtcaaccgctgtcgatcaagcaagaagaagttcacgttcgcggccatgcggtggaatgtcgtatcaacgccgaagatccgaacaccttcctgccaagtccgggcaaaatcacccgtttccacgcacctggcggttttggcgtacgttgggagtctcatatctacgcgggctacaccgtaccgccgtactatgactcaatgatcggtaagctgatttgctacggtgaaaaccgtgacgtggcgattgcccgcatgaagaatgcgctgcaggagctgatcatcgacggtatcaaaaccaacgttgatctgcagatccgcatcatgaatgacgagaacttccagcatggtggcactaacatccactatctggagaaaaaactcggtcttcaggaaaaataa

SEQ ID NO: 144 - protein, 449 aa, Escherichia coli
MLDKIVIANRGEIALRILRACKELGIKTVAVHSSADRDLKHVLLADETVCIGPAPSVKSYLNIPAIISAAEITGAVAIHPGYGFLSENANFAEQVERSGFIFIGPKAETIRLMGDKVSAIAAMKKAGVPCVPGSDGPLGDDMDKNRAIAKRIGYPVIIKASGGGGGRGMRVVRGDAELAQSISMTRAEAKAAFSNDMVYMEKYLENPRHVEIQVLADGQGNAIYLAERDCSMQRRHQKVVEEAPAPGITPELRRYIGERCAKACVDIGYRGAGTFEFLFENGEFYFIEMNTRIQVEHPVTEMITGVDLIKEQLRIAAGQPLSIKQEEVHVRGHAVECRINAEDPNTFLPSPGKITRFHAPGGFGVRWESHIYAGYTVPPYYDSMIGKLICYGENRDVAIARMKNALQELIIDGIKTNVDLQIRIMNDENFQHGGTNIHYLEKKLGLQEK

SEQ ID NO: 145 - DNA, 960 bp, Escherichia coli; identical to SEQ ID NO: 139

SEQ ID NO: 146 - protein, 319 aa, Escherichia coli; identical to SEQ ID NO: 140

(19) United States
(12) Patent Application Publication
(10) Pub. No.: US 2020/0048662 A1
(43) Pub. Date: Feb. 13, 2020

(54) MICROORGANISMS AND METHODS FOR THE CO-PRODUCTION OF ETHYLENE GLYCOL AND ISOBUTENE

(71) Applicant: Braskem S.A., Sao Paulo (BR)

(72) Inventors: Felipe GALZERANI, Campinas (BR); Daniel Johannes KOCH, Campinas (BR); Mateus Schreiner LOPES, Campinas (BR)

(21) Appl. No.: 16/341,283

(22) PCT Filed: Oct. 11, 2017
(86) PCT No.: PCT/US2017/056174

§ 371 Date: Apr. 11, 2019

Related U.S. Application Data
(60) Provisional application No. 62/406,684, filed on Oct. 11, 2016.

(51) Int. Cl. C12P 7/18; C12P 5/02; C12N 15/70; C12N 9/90; C12N 9/12

(52) U.S. Cl. CPC C12P 7/18; C12P 5/026; C12Y 207/01047; C12N 9/1205; C12Y 207/01017; C12N 15/70; C12N 9/90; C12Y 501/03

(57) ABSTRACT

The present application relates to recombinant microorganisms useful in the biosynthesis of monoethylene glycol (MEG) and isobutene. The application further relates to recombinant microorganisms co-expressing a C2 branch pathway and a C3 branch pathway for the production of MEG and isobutene. Also provided are methods of producing MEG and isobutene using the recombinant microorganisms, as well as compositions comprising the recombinant microorganisms and/or optionally the products MEG and isobutene.

### CROSS-REFERENCE TO RELATED APPLICATIONS

This application claims the benefit of priority to U.S. Provisional Application No. 62/406,684, filed on Oct. 11, 2016, and it is herein incorporated by reference in its entirety.

### TECHNICAL FIELD

This application relates to recombinant microorganisms useful in the biosynthesis of monoethylene glycol and isobutene. The application further relates to methods of producing monoethylene glycol and isobutene using the recombinant microorganisms, as well as compositions comprising one or both of these compounds and/or the recombinant microorganisms.

### STATEMENT REGARDING SEQUENCE LISTING

The sequence listing associated with this application is provided in text format in lieu of a paper copy, and is hereby incorporated by reference into the specification. The name of the text file containing the sequence listing is BRSK_003_01WO_ST25.txt. The text file is 319 kb, was created on Oct. 11, 2017, and is being submitted electronically via EFS-Web.

### BACKGROUND

A large number of chemical compounds are currently derived from petrochemicals. Monoethylene glycol (MEG) is valuable as raw material in the production of products like polyethylene terephthalate (PET) resins and also finds use directly for industrial or household purposes. Alkenes (such as ethylene, propylene, different butenes, and pentenes, for example) are used in the plastics industry, fuels, and in other areas of the chemical industry. Isobutene is a small, highly reactive molecule, used extensively as a platform chemical to manufacture a wide variety of products including fuel additives, rubber and rubber additives, and specialty chemicals.

However, the compounds are currently produced from precursors that originate from fossil fuels, which contribute to climate change. To develop more environmentally friendly processes for the production of MEG and isobutene, researchers have engineered microorganisms with biosynthetic pathways to produce MEG or isobutene separately. However, these pathways are challenging to implement, with loss of product yield, redox balance and excess biomass formation being some major obstacles to overcome.

Thus there exists a need for improved biosynthesis pathways for the production of MEG and isobutene.

### SUMMARY OF THE DISCLOSURE

The present application relates to recombinant microorganisms having one or more biosynthesis pathways for the production of monoethylene glycol and isobutene.

The present disclosure provides a combination of an easy to implement, high yield C2 branch pathway for MEG production from xylose with an easy to implement C3 branch pathway for isobutene production from DHAP or pyruvate.

The presently disclosed process of co-producing MEG and isobutene is synergistic by utilizing the excess NADH produced in the C3 stream pathway to isobutene to feed the NADH requirement of the C2 stream to MEG.

In one aspect, the present application provides a recombinant microorganism co-producing monoethylene glycol (MEG) and isobutene. In one embodiment, the MEG and isobutene are co-produced from xylose. In another embodiment, the recombinant microorganism comprises a deletion, insertion, or loss of function mutation in a gene encoding a D-xylulose-5-kinase and/or in a gene encoding a glycoaldehyde dehydrogenase. In some embodiments, the gene encoding the D-xylulose-5-kinase is xylB. In some embodiments, the gene encoding the glycoaldehyde dehydrogenase is aldA. In a further embodiment, isobutene is synthesized via the intermediate 3-hydroxyisovalerate. In some embodiments, MEG is produced through the conversion of glycolaldehyde in a C-2 branch pathway and isobutene is produced through the conversion of DHAP or pyruvate in a C-3 branch pathway. In other embodiments, at least a portion of the excess NADH produced in the C-3 branch pathway is used as a source of reducing equivalents in the C-2 branch pathway. In further embodiments, at least a portion of the excess NADH produced in the C-3 branch pathway is used to produce ATP. In yet further embodiments, excess biomass formation is minimized and production of MEG and isobutene is maximized.

In one embodiment, MEG is produced from xylose via ribulose-1-phosphate. In another embodiment, MEG is produced from xylose via xylulose-1-phosphate. In a further embodiment, MEG is produced from xylose via xylonate.

In one embodiment, isobutene is produced from DHAP or pyruvate via acetone. In another embodiment, isobutene is produced from DHAP or pyruvate via HMG-CoA.

In one preferred embodiment, MEG and isobutene are produced from xylose using a ribulose-1-phosphate pathway for the conversion of xylose to MEG and dihydroxyacetone-phosphate (DHAP), and using an acetone based pathway for the conversion of DHAP to isobutene.

In another preferred embodiment, MEG and isobutene are produced from xylose using a ribulose-1-phosphate pathway for the conversion of xylose to MEG and dihydroxyacetone-phosphate (DHAP), and using an HMG-CoA based pathway for the conversion of DHAP to isobutene.

In another aspect, the present application relates to a recombinant microorganism capable of co-producing monoethylene glycol (MEG) and isobutene from exogenous D-xylose, wherein the recombinant microorganism expresses one or more of the following from (a) to (d):
(a) at least one endogenous or exogenous nucleic acid molecule encoding a D-tagatose 3-epimerase that catalyzes the conversion of D-xylulose to D-ribulose;
(b) at least one endogenous or exogenous nucleic acid molecule encoding a D-ribulokinase that catalyzes the conversion of D-ribulose from (a) to D-ribulose-1-phosphate,
(c) at least one endogenous or exogenous nucleic acid molecule encoding a D-ribulose-1-phosphate aldolase that catalyzes the conversion of D-ribulose-1-phosphate from (b) to glycolaldehyde and dihydroxyacetonephosphate (DHAP);
(d) at least one endogenous or exogenous nucleic acid molecule encoding a glycolaldehyde reductase that catalyzes the conversion of glycolaldehyde from (c) to MEG;
   wherein the recombinant microorganism further expresses one or more of the following from (e) to (h):
(e) at least one endogenous or exogenous nucleic acid molecule encoding a thiolase or acetyl coenzyme A acetyltransferase that catalyzes the conversion of acetyl-CoA to acetoacetyl-CoA;
(f) at least one endogenous or exogenous nucleic acid molecule encoding an acetyl-CoA:acetoacetate-CoA transferase or acetate:acetoacetyl-CoA hydrolase that catalyzes the conversion of acetoacetyl-CoA from (e) to acetoacetate;
(g) at least one endogenous or exogenous nucleic acid molecule encoding an acetoacetate decarboxylase that catalyzes the conversion of acetoacetate from (f) to acetone;
(h) at least one endogenous or exogenous nucleic acid molecule encoding a 3-hydroxyisovalerate synthase that catalyzes the conversion of acetone from (g) and acetyl-CoA to 3-hydroxyisovalerate (3HIV);
or
   wherein the recombinant microorganism expresses one or more of the nucleic acid molecule from (a) to (d) above and further expresses one or more of the following from (i) to (n):
(i) at least one endogenous or exogenous nucleic acid molecule encoding an acetyl coenzyme A acetyltransferase that catalyzes the conversion of acetyl-CoA to acetoacetyl-CoA;
(j) at least one endogenous or exogenous nucleic acid molecule encoding a hydroxymethylglutaryl-CoA synthase that catalyzes the conversion of acetoacetyl-CoA from (i) and acetyl-CoA to 3-hydroxy-3-methylglutaryl-CoA (HMG-CoA);
(k) at least one endogenous or exogenous nucleic acid molecule encoding a methylglutaconyl-CoA hydratase that catalyzes the conversion of HMG-CoA from (j) to 3-methylglutaconyl-CoA;
(l) at least one endogenous or exogenous nucleic acid molecule encoding a methylcrotonyl-CoA carboxylase that catalyzes the conversion of 3-methylglutaconyl-CoA from (k) to 3-methylcrotonyl-CoA;
(m) at least one endogenous or exogenous nucleic acid molecule encoding a methylcrotonyl-CoA hydratase that catalyzes the conversion of 3-methylcrotonyl-CoA from (l) to 3-hydroxyisovaleryl-CoA;
(n) at least one endogenous or exogenous nucleic acid molecule encoding a 3-hydroxyisovaleryl-CoA thioesterase that catalyzes the conversion of 3-hydroxyisovaleryl-CoA from (m) to 3HIV;
   wherein the recombinant microorganism further expresses (a1) and (a2), and/or (b1) selected from:
(a1) at least one endogenous or exogenous nucleic acid molecule encoding a 3HIV kinase that catalyzes the conversion of 3HIV from (h) or (n) to 3HIV-3-phosphate;
(a2) at least one endogenous or exogenous nucleic acid molecule encoding a 3HIV-3-phosphate decarboxylase that catalyzes the conversion of 3HIV-3-phosphate from (a1) to isobutene;
(b1) at least one endogenous or exogenous nucleic acid molecule encoding a 3HIV decarboxylase that catalyzes the conversion of 3HIV from (h) or (n) to isobutene;
   wherein the produced intermediate DHAP is converted to acetyl-CoA through the endogenous glycolysis pathway in the microorganism, and wherein MEG and isobutene are co-produced.

In one embodiment, the D-tagatose 3-epimerase is encoded by one or more nucleic acid molecules obtained from a microorganism selected from the group consisting of Pseudomonas sp., Mesorhizobium sp. and Rhodobacter sp. In some embodiments, the D-tagatose 3-epimerase is encoded by one or more nucleic acid molecules obtained from a microorganism selected from the group consisting of Pseudomonas cichorii, Pseudomonas sp. ST-24, Mesorhizobium loti and Rhodobacter sphaeroides. In some embodiments, the one or more nucleic acid molecules is dte and/or FJ851309.1, or homolog thereof. In a further embodiment, the D-tagatose 3-epimerase comprises an amino acid sequence selected from the group consisting of SEQ ID NOs: 3 and 5. In yet a further embodiment, the D-tagatose 3-epimerase is encoded by a nucleic acid sequence selected from the group consisting of SEQ ID NOs: 1, 2 and 4.

In one embodiment, the D-ribulokinase is encoded by one or more nucleic acid molecules obtained from E. coli. In some embodiments, the one or more nucleic acid molecules is fucK, or homolog thereof. In a further embodiment, the D-ribulokinase comprises an amino acid sequence set forth in SEQ ID NO: 8. In yet a further embodiment, the D-ribulokinase is encoded by a nucleic acid sequence selected from the group consisting of SEQ ID NOs: 6 and 7.

In one embodiment, the D-ribulose-1-phosphate aldolase is encoded by one or more nucleic acid molecules obtained from E. coli. In some embodiments, the one or more nucleic acid molecules is fucA, or homolog thereof. In a further embodiment, the D-ribulose-1-phosphate aldolase comprises an amino acid sequence set forth in SEQ ID NO: 11. In yet a further embodiment, the D-ribulose-1-phosphate aldolase is encoded by a nucleic acid sequence selected from the group consisting of SEQ ID NOs: 9 and 10.

In one preferred embodiment, MEG and isobutene are produced from xylose using a xylulose-1-phosphate pathway for the conversion of xylose to MEG and dihydroxyacetone-phosphate (DHAP), and using an acetone based pathway for the conversion of DHAP to isobutene.

In another preferred embodiment, MEG and isobutene are produced from xylose using a xylulose-1-phosphate pathway for the conversion of xylose to MEG and dihydroxyacetone-phosphate (DHAP), and using an HMG-CoA based pathway for the conversion of DHAP to isobutene.

In another aspect, the present application relates to a recombinant microorganism capable of co-producing monoethylene glycol (MEG) and isobutene from exogenous D-xylose, wherein the recombinant microorganism expresses one or more of the following from (a) to (c):
(a) at least one endogenous or exogenous nucleic acid molecule encoding a D-xylulose 1-kinase that catalyzes the conversion of D-xylulose to D-xylulose-1-phosphate;
(b) at least one endogenous or exogenous nucleic acid molecule encoding a D-xylulose-1-phosphate aldolase that catalyzes the conversion of D-xylulose-1-phosphate from (a) to glycolaldehyde and dihydroxyacetonephosphate (DHAP);
(c) at least one endogenous or exogenous nucleic acid molecule encoding a glycolaldehyde reductase that catalyzes the conversion of glycolaldehyde from (b) to MEG;
   wherein the recombinant microorganism further expresses one or more of the following from (d) to (g):
(d) at least one endogenous or exogenous nucleic acid molecule encoding a thiolase or acetyl coenzyme A acetyltransferase that catalyzes the conversion of acetyl-CoA to acetoacetyl-CoA;
(e) at least one endogenous or exogenous nucleic acid molecule encoding an acetyl-CoA:acetoacetate-CoA transferase or acetate:acetoacetyl-CoA hydrolase that catalyzes the conversion of acetoacetyl-CoA from (d) to acetoacetate;
(f) at least one endogenous or exogenous nucleic acid molecule encoding an acetoacetate decarboxylase that catalyzes the conversion of acetoacetate from (e) to acetone;
(g) at least one endogenous or exogenous nucleic acid molecule encoding a 3-hydroxyisovalerate synthase that catalyzes the conversion of acetone from (f) and acetyl-CoA to 3-hydroxyisovalerate (3HIV);
or
   wherein the recombinant microorganism expresses one or more of the nucleic acid molecule from (a) to (c) above and further expresses one or more of the following from (h) to (m):
(h) at least one endogenous or exogenous nucleic acid molecule encoding an acetyl coenzyme A acetyltransferase that catalyzes the conversion of acetyl-CoA to acetoacetyl-CoA;
(i) at least one endogenous or exogenous nucleic acid molecule encoding a hydroxymethylglutaryl-CoA synthase that catalyzes the conversion of acetoacetyl-CoA from (h) and acetyl-CoA to 3-hydroxy-3-methylglutaryl-CoA (HMG-CoA);
(j) at least one endogenous or exogenous nucleic acid molecule encoding a methylglutaconyl-CoA hydratase that catalyzes the conversion of HMG-CoA from (i) to 3-methylglutaconyl-CoA;
(k) at least one endogenous or exogenous nucleic acid molecule encoding a methylcrotonyl-CoA carboxylase that catalyzes the conversion of 3-methylglutaconyl-CoA from (j) to 3-methylcrotonyl-CoA;
(l) at least one endogenous or exogenous nucleic acid molecule encoding a methylcrotonyl-CoA hydratase that catalyzes the conversion of 3-methylcrotonyl-CoA from (k) to 3-hydroxyisovaleryl-CoA;
(m) at least one endogenous or exogenous nucleic acid molecule encoding a 3-hydroxyisovaleryl-CoA thioesterase that catalyzes the conversion of 3-hydroxyisovaleryl-CoA from (l) to 3HIV;
   wherein the recombinant microorganism further expresses (a1) and (a2), and/or (b1) selected from:
(a1) at least one endogenous or exogenous nucleic acid molecule encoding a 3HIV kinase that catalyzes the conversion of 3HIV from (g) or (m) to 3HIV-3-phosphate;
(a2) at least one endogenous or exogenous nucleic acid molecule encoding a 3HIV-3-phosphate decarboxylase that catalyzes the conversion of 3HIV-3-phosphate from (a1) to isobutene;
(b1) at least one endogenous or exogenous nucleic acid molecule encoding a 3HIV decarboxylase that catalyzes the conversion of 3HIV from (g) or (m) to isobutene;
   and wherein the produced intermediate DHAP is converted to acetyl-CoA through the endogenous glycolysis pathway in the microorganism, and wherein MEG and isobutene are co-produced.

In one embodiment, the D-xylulose 1-kinase is encoded by one or more nucleic acid molecules obtained from Homo sapiens. In some embodiments, the one or more nucleic acid molecules encoding the D-xylulose 1-kinase is ketohexokinase C (khk-C), or homolog thereof. In another embodiment, the one or more nucleic acid molecules encoding the D-xylulose 1-kinase comprises an amino acid sequence set forth in SEQ ID NO: 55. In a further embodiment, the one or more nucleic acid molecules encoding the D-xylulose 1-kinase is encoded by a nucleic acid sequence selected from the group consisting of SEQ ID NOs: 53 and 54.

In one embodiment, the D-xylulose-1-phosphate aldolase is encoded by one or more nucleic acid molecules obtained from Homo sapiens. In another embodiment, the one or more nucleic acid molecules encoding the D-xylulose-1-phosphate aldolase is aldolase B (ALDOB), or homolog thereof. In some embodiments, the one or more nucleic acid molecules encoding the D-xylulose-1-phosphate aldolase comprises an amino acid sequence set forth in SEQ ID NO: 58. In some embodiments, the one or more nucleic acid molecules encoding the D-xylulose-1-phosphate aldolase is encoded by a nucleic acid sequence selected from the group consisting of SEQ ID NOs: 56 and 57.

In one embodiment of any aspect disclosed above, the recombinant microorganism further comprises one or more modifications selected from the group consisting of:
(a) a deletion, insertion, or loss of function mutation in a gene encoding a D-xylulose-5-kinase that catalyzes the conversion of D-xylulose to D-xylulose-5-phosphate;
(b) a deletion, insertion, or loss of function mutation in a gene encoding a glycolaldehyde dehydrogenase that catalyzes the conversion of glycolaldehyde to glycolic acid; and
(c) a deletion, insertion, or loss of function mutation in a gene encoding a lactate dehydrogenase that catalyzes the conversion of pyruvate to lactate.

In some embodiments of any aspect disclosed above, a recombinant microorganism producing MEG and isobutene comprises a deletion, insertion, or loss of function mutation in a gene encoding a D-xylulose-5-kinase to prevent the conversion of D-xylulose to D-xylulose-5-phosphate and instead shunt the reaction toward conversion of D-xylulose to D-xylulose-1-phosphate. In some embodiments, the D-xylulose-5-kinase is from Escherichia coli. In some embodiments, the D-xylulose-5-kinase is encoded by the xylB gene, or homolog thereof.

In some embodiments of any aspect disclosed above, a recombinant microorganism producing MEG and isobutene comprises a deletion, insertion, or loss of function mutation in a gene encoding a glycolaldehyde dehydrogenase to prevent the production of glycolic acid from glycolaldehyde and instead shunt the reaction toward conversion of glycolaldehyde to MEG. In some embodiments, the glycolaldehyde dehydrogenase is from Escherichia coli. In some embodiments, the glycolaldehyde dehydrogenase is encoded by the aldA gene, or homolog thereof.

In some embodiments of any aspect disclosed above, a recombinant microorganism producing MEG and isobutene comprises a deletion, insertion, or loss of function mutation in a gene encoding a lactate dehydrogenase to prevent the production of lactate from pyruvate and instead shunt the reaction toward production of isobutene. In some embodiments, the lactate dehydrogenase is from Escherichia coli. In some embodiments, the lactate dehydrogenase is encoded by the IdhA gene, or homolog thereof.

In one embodiment of any aspect disclosed above, the recombinant microorganism further comprises an endogenous or exogenous xylose isomerase that catalyzes the conversion of D-xylose to D-xylulose. In one embodiment, the xylose isomerase is exogenous. In another embodiment, the xylose isomerase is encoded by one or more nucleic acid molecules obtained from Pyromyces sp. In another embodiment, the one or more nucleic acid molecules encoding the xylose isomerase is xylA, or homolog thereof. In yet another embodiment, the one or more nucleic acid molecules encoding the xylose isomerase comprises an amino acid sequence set forth in SEQ ID NO: 95. In a further embodiment, the one or more nucleic acid molecules encoding the xylose isomerase is encoded by a nucleic acid sequence selected from the group consisting of SEQ ID NOs: 93 and 94.

In one embodiment of any aspect disclosed above, the recombinant microorganism further expresses at least one exogenous nucleic acid molecule encoding a xylose reductase or aldose reductase that catalyzes the conversion of D-xylose to xylitol and at least one exogenous nucleic acid molecule encoding a xylitol dehydrogenase that catalyzes the conversion of xylitol to D-xylulose.

In some embodiments of any aspect disclosed above, the xylose reductase or aldose reductase is encoded by one or more nucleic acid molecules obtained from a microorganism selected from the group consisting of Hypocrea sp., Scheffersomyces sp., Saccharomyces sp., Pachysolen sp., Pichia sp., Candida sp., Aspergillus sp., Neurospora sp., and Cryptococcus sp. In some embodiments, the xylose reductase or aldose reductase is encoded by one or more nucleic acid molecules obtained from a microorganism selected from the group consisting of Hypocrea jecorina, Scheffersomyces stipitis, Saccharomyces cerevisiae, Pachysolen tannophilus, Pichia stipitis, Pichia quercuum, Candida shehatae, Candida tenuis, Candida tropicalis, Aspergillus niger, Neurospora crassa and Cryptococcus lactativorus. In another embodiment, the one or more nucleic acid molecules encoding the xylose reductase or aldose reductase is xyl1 and/or GRE3 or homolog thereof. In some embodiments, the one or more nucleic acid molecules encoding the xylose reductase or aldose reductase comprises an amino acid sequence selected from the group consisting of SEQ ID NOs: 84 and 87. In some embodiments, the one or more nucleic acid molecules encoding the xylose reductase or aldose reductase is encoded by a nucleic acid sequence selected from the group consisting of SEQ ID NOs: 82, 83, 85 and 86.

In one embodiment of any aspect disclosed above, the xylitol dehydrogenase is encoded by one or more nucleic acid molecules obtained from a microorganism selected from the group consisting of Scheffersomyces sp., Trichoderma sp., Pichia sp., Saccharomyces sp., Gluconobacter sp., Galactocandida sp., Neurospora sp., and Serratia sp. In another embodiment, the xylitol dehydrogenase is encoded by one or more nucleic acid molecules obtained from a microorganism selected from the group consisting of Scheffersomyces stipitis, Trichoderma reesei, Pichia stipitis, Saccharomyces cerevisiae, Gluconobacter oxydans, Galactocandida mastotermitis, Neurospora crassa and Serratia marcescens. In another embodiment, the one or more nucleic acid molecules encoding the xylitol dehydrogenase is xyl2 and/or xdh1, or homolog thereof. In some embodiments, the one or more nucleic acid molecules encoding the xylitol dehydrogenase comprises an amino acid sequence selected from the group consisting of SEQ ID NOs: 90 and 92. In some embodiments, the one or more nucleic acid molecules encoding the xylitol dehydrogenase is encoded by a nucleic acid sequence selected from the group consisting of SEQ ID NOs: 88, 89 and 91.

In one preferred embodiment, MEG and isobutene are produced from xylose using a xylonate pathway for the conversion of xylose to MEG and pyruvate, and using an acetone based pathway for the conversion of pyruvate to isobutene.

In another preferred embodiment, MEG and isobutene are produced from xylose using a xylonate pathway for the conversion of xylose to MEG and pyruvate, and using an HMG-CoA based pathway for the conversion of pyruvate to isobutene.

In another aspect, the present application relates to a recombinant microorganism capable of co-producing monoethylene glycol (MEG) and isobutene from exogenous D-xylose, wherein the recombinant microorganism expresses one or more of the following from (a) to (c):
(a) at least one endogenous or exogenous nucleic acid molecule encoding a xylose dehydrogenase that catalyzes the conversion of D-xylose to D-xylonolactone,
(b) at least one endogenous or exogenous nucleic acid molecule encoding a xylonolactonase that catalyzes the conversion of D-xylonolactone from (a) to D-xylonate,
(c) at least one endogenous or exogenous nucleic acid molecule encoding a xylose dehydrogenase that catalyzes the conversion of D-xylose to D-xylonate;
   wherein the recombinant microorganism further expresses one or more of the following from (d) to (f):
(d) at least one endogenous or exogenous nucleic acid molecule encoding a xylonate dehydratase that catalyzes the conversion of D-xylonate from (b) or (c) to 2-keto-3-deoxy-xylonate;
(e) at least one endogenous or exogenous nucleic acid molecule encoding a 2-keto-3-deoxy-D-pentonate aldolase that catalyzes the conversion of 2-keto-3-deoxy-xylonate from (d) to glycolaldehyde and pyruvate;
(f) at least one endogenous or exogenous nucleic acid molecule encoding a glycolaldehyde reductase that catalyzes the conversion of glycolaldehyde from (e) to MEG;
   wherein the recombinant microorganism further expresses one or more of the following from (g) to (j):
(g) at least one exogenous nucleic acid molecule encoding a thiolase or acetyl coenzyme A acetyltransferase that catalyzes the conversion of acetyl-CoA to acetoacetyl-CoA;
(h) at least one endogenous or exogenous nucleic acid molecule encoding an acetyl-CoA:acetoacetate-CoA transferase or acetate:acetoacetyl-CoA hydrolase that catalyzes the conversion of acetoacetyl-CoA from (g) to acetoacetate;
(i) at least one exogenous nucleic acid molecule encoding an acetoacetate decarboxylase that catalyzes the conversion of acetoacetate from (h) to acetone;
(j) at least one endogenous or exogenous nucleic acid molecule encoding a 3-hydroxyisovalerate synthase that catalyzes the conversion of acetone from (i) and acetyl-CoA to 3-hydroxy-isovalerate (3HIV);
or
   wherein the recombinant microorganism expresses one or more of the nucleic acid molecule from (a) to (c) above and one or more of the nucleic acid molecule from (d) to (f) above, and further expresses one or more of the following from (k) to (p):
(k) at least one endogenous or exogenous nucleic acid molecule encoding an acetyl coenzyme A acetyltransferase that catalyzes the conversion of acetyl-CoA to acetoacetyl-CoA;
(l) at least one endogenous or exogenous nucleic acid molecule encoding a hydroxymethylglutaryl-CoA synthase that catalyzes the conversion of acetoacetyl-CoA from (k) and acetyl-CoA to 3-hydroxy-3-methylglutaryl-CoA (HMG-CoA);
(m) at least one endogenous or exogenous nucleic acid molecule encoding a methylglutaconyl-CoA hydratase that catalyzes the conversion of HMG-CoA from (l) to 3-methylglutaconyl-CoA;
(n) at least one endogenous or exogenous nucleic acid molecule encoding a methylcrotonyl-CoA carboxylase that catalyzes the conversion of 3-methylglutaconyl-CoA from (m) to 3-methylcrotonyl-CoA;
(o) at least one endogenous or exogenous nucleic acid molecule encoding a methylcrotonyl-CoA hydratase that catalyzes the conversion of 3-methylcrotonyl-CoA from (n) to 3-hydroxyisovaleryl-CoA;
(p) at least one endogenous or exogenous nucleic acid molecule encoding a 3-hydroxyisovaleryl-CoA thioesterase that catalyzes the conversion of 3-hydroxyisovaleryl-CoA from (o) to 3HIV;
   wherein the recombinant microorganism further expresses (a1) and (a2), and/or (b1) selected from:
(a1) at least one endogenous or exogenous nucleic acid molecule encoding a 3HIV kinase that catalyzes the conversion of 3HIV from (j) or (p) to 3HIV-3-phosphate;
(a2) at least one endogenous or exogenous nucleic acid molecule encoding a 3HIV-3-phosphate decarboxylase that catalyzes the conversion of 3HIV-3-phosphate from (a1) to isobutene;
(b1) at least one endogenous or exogenous nucleic acid molecule encoding a 3HIV decarboxylase that catalyzes the conversion of 3HIV from (j) or (p) to isobutene;
   and wherein the produced intermediate pyruvate is converted to acetyl-CoA through the endogenous glycolysis pathway in the microorganism, and wherein MEG and isobutene are co-produced.

In one embodiment, the xylose dehydrogenase is encoded by one or more nucleic acid molecules obtained from a microorganism selected from the group consisting of Caulobacter sp., Haloarcula sp., Haloferax sp., Halorubrum sp. and Trichoderma sp. In another embodiment, the xylose dehydrogenase is encoded by one or more nucleic acid molecules obtained from a microorganism selected from the group consisting of Caulobacter crescentus, Haloarcula marismortui, Haloferax volcanii, Halorubrum lacusprofundi and Trichoderma reesei. In some embodiments, the one or more nucleic acid molecules encoding the xylose dehydrogenase is selected from xylB, xdh1 (HVO_B0028) and/or xyd1, or homolog thereof. In a further embodiment, the one or more nucleic acid molecules encoding the xylose dehydrogenase comprises an amino acid sequence selected from the group consisting of SEQ ID NOs: 61, 63 and 65. In yet another embodiment, the one or more nucleic acid molecules encoding the xylose dehydrogenase is encoded by a nucleic acid sequence selected from the group consisting of SEQ ID NOs: 59, 60, 62 and 64.

In one embodiment, the xylonolactonase is encoded by one or more nucleic acid molecules obtained from a microorganism selected from Caulobacter sp. and Haloferax sp. In another embodiment, the xylonolactonase is encoded by one or more nucleic acid molecules obtained from a microorganism selected from the group consisting of Caulobacter crescentus, Haloferax volcanii and Haloferax gibbonsii. In some embodiments, the one or more nucleic acid molecules encoding the xylonolactonase is xylC, or homolog thereof. In a further embodiment, the one or more nucleic acid molecules encoding the xylonolactonase comprises an amino acid sequence set forth in SEQ ID NO: 67. In yet another embodiment, the one or more nucleic acid molecules encoding the xylonolactonase is encoded by a nucleic acid sequence set forth in SEQ ID NO: 66.

In one embodiment, the xylonate dehydratase is encoded by one or more nucleic acid molecules obtained from a microorganism selected from the group consisting of Caulobacter sp., Sulfolobus sp. and E. coli. In another embodiment, the xylonate dehydratase is encoded by one or more nucleic acid molecules obtained from a microorganism selected from the group consisting of Caulobacter crescentus, Sulfolobus solfataricus and E. coli. In some embodiments, the one or more nucleic acid molecules encoding the xylonate dehydratase is selected from xylD, yjhG and/or yagF, or homolog thereof. In a further embodiment, the one or more nucleic acid molecules encoding the xylonate dehydratase comprises an amino acid sequence selected from the group consisting of SEQ ID NOs: 69, 72 and 75. In yet another embodiment, the one or more nucleic acid molecules encoding the xylonate dehydratase is encoded by a nucleic acid sequence selected from the group consisting of SEQ ID NOs: 68, 70, 71, 73 and 74.

In one embodiment, the 2-keto-3-deoxy-D-pentonate aldolase is encoded by one or more nucleic acid molecules obtained from a microorganism selected from Pseudomonas sp. and E. coli. In another embodiment, the 2-keto-3-deoxy-D-pentonate aldolase is encoded by one or more nucleic acid molecules obtained from E. coli. In some embodiments, the one or more nucleic acid molecules encoding the 2-keto-3-deoxy-D-pentonate aldolase is selected from yjhH and/or yagE, or homolog thereof. In a further embodiment, the one or more nucleic acid molecules encoding the 2-keto-3-deoxy-D-pentonate aldolase comprises an amino acid sequence selected from the group consisting of SEQ ID NOs: 78 and 81. In yet another embodiment, the one or more nucleic acid molecules encoding the 2-keto-3-deoxy-D-pentonate aldolase is encoded by a nucleic acid sequence selected from the group consisting of SEQ ID NOs: 76, 77, 79 and 80.

In one embodiment, the recombinant microorganism further comprises one or more modifications selected from the group consisting of:
(a) a deletion, insertion, or loss of function mutation in a gene encoding an enzyme that catalyzes the conversion of D-xylose to D-xylulose;
(b) a deletion, insertion, or loss of function mutation in a gene encoding an enzyme that catalyzes the conversion of glycolaldehyde to glycolic acid; and
(c) a deletion, insertion, or loss of function mutation in a gene encoding an enzyme that catalyzes the conversion of pyruvate to lactate.

In some embodiments, a recombinant microorganism producing MEG and isobutene comprises a deletion, insertion, or loss of function mutation in a gene encoding a D-xylose isomerase to prevent conversion of D-xylose to D-xylulose and instead shunt the reaction toward the conversion of D-xylose to D-xylonate. In one embodiment, the enzyme that catalyzes the conversion of D-xylose to D-xylulose is a D-xylose isomerase. In some embodiments, the D-xylose isomerase is from Escherichia coli. In some embodiments, the D-xylose isomerase is encoded by the xylA gene, or homolog thereof.

In some embodiments, a recombinant microorganism producing MEG and isobutene comprises a deletion, insertion, or loss of function mutation in a gene encoding a glycolaldehyde dehydrogenase to prevent the production of glycolic acid from glycolaldehyde and instead shunt the reaction toward conversion of glycolaldehyde to MEG. In one embodiment, the glycolaldehyde dehydrogenase is from Escherichia coli. In some embodiments, the glycolaldehyde dehydrogenase is encoded by the aldA gene, or homolog thereof.

In some embodiments, a recombinant microorganism producing MEG and isobutene comprises a deletion, insertion, or loss of function mutation in a gene encoding a lactate dehydrogenase to prevent the production of lactate from pyruvate and instead shunt the reaction toward production of isobutene. In one embodiment, the enzyme that catalyzes the conversion of pyruvate to lactate is a lactate dehydrogenase. In particular embodiments, the enzyme converts pyruvate to lactate. In some embodiments, the lactate dehydrogenase is from Escherichia coli. In some embodiments, the lactate dehydrogenase is encoded by the IdhA gene, or homolog thereof.

In one embodiment of any aspect disclosed above, the glycolaldehyde reductase is encoded by one or more nucleic acid molecules obtained from a microorganism selected from E. coli and S. cerevisiae. In another embodiment, the one or more nucleic acid molecules is selected from gldA, GRE2, GRE3, yqhD, ydjG, fucO, yafB (dkgB), and/or yqhE (dkgA), or homolog thereof. In another embodiment, the one or more nucleic acid molecules is yqhD. In some embodiments, the yqhD comprises a G149E mutation. In a further embodiment, the glycolaldehyde reductase comprises an amino acid sequence selected from the group consisting of SEQ ID NOs: 13, 15, 17, 20, 23, 25, 28, 30 and 32. In yet a further embodiment, the glycolaldehyde reductase is encoded by a nucleic acid sequence selected from the group consisting of SEQ ID NOs: 12, 14, 16, 18, 19, 21, 22, 24, 26, 27, 29 and 31.

In one embodiment of any aspect disclosed above, the thiolase or acetyl coenzyme A acetyltransferase is encoded by one or more nucleic acid molecules obtained from a microorganism selected from the group consisting of Clostridium sp., Bacillus sp., E. coli, Saccharomyces sp. and Marinobacter sp. In some embodiments, the thiolase or acetyl coenzyme A acetyltransferase is encoded by one or more nucleic acid molecules obtained from a microorganism selected from the group consisting of Clostridium acetobutylicum, Clostridium thermosaccharolyticum, Bacillus cereus, E. coli, Saccharomyces cerevisiae and Marinobacter hydrocarbonoclasticus. In some embodiments, the one or more nucleic acid molecules is thlA, atoB and/or ERG10, or homolog thereof. In a further embodiment, the thiolase or acetyl coenzyme A acetyltransferase comprises an amino acid sequence selected from the group consisting of SEQ ID NOs: 35, 37 and 40. In yet a further embodiment, the thiolase or acetyl coenzyme A acetyltransferase is encoded by a nucleic acid sequence selected from the group consisting of SEQ ID NOs: 33, 34, 36, 38 and 39.

In one embodiment of any aspect disclosed above, the acetyl-CoA:acetoacetate-CoA transferase or acetate:acetoacetyl-CoA hydrolase is encoded by one or more nucleic acid molecules obtained from a microorganism selected from Clostridium sp. and E. coli. In another embodiment, the acetyl-CoA:acetoacetate-CoA transferase or acetate:acetoacetyl-CoA hydrolase is encoded by one or more nucleic acid molecules obtained from E. coli. In some embodiments, the one or more nucleic acid molecules encoding the acetyl-CoA:acetoacetate-CoA transferase is atoA and/or atoD, or homolog thereof. In another embodiment, the acetyl-CoA:acetoacetate-CoA transferase or acetate:acetoacetyl-CoA hydrolase is encoded by one or more nucleic acid molecules obtained from Clostridium acetobutylicum. In some embodiments, the one or more nucleic acid molecules encoding the acetate:acetoacetyl-CoA hydrolase is ctfA and/or ctfB, or homolog thereof. In a further embodiment, the acetyl-CoA:acetoacetate-CoA transferase or acetate:acetoacetyl-CoA hydrolase comprises an amino acid sequence selected from the group consisting of SEQ ID NOs: 43, 46, 97, 99, 101 and 103. In yet a further embodiment, the acetyl-CoA:acetoacetate-CoA transferase or acetate:acetoacetyl-CoA hydrolase is encoded by a nucleic acid sequence selected from the group consisting of SEQ ID NOs: 41, 42, 44, 45, 96, 98, 100 and 102.

In one embodiment of any aspect disclosed above, the acetoacetate decarboxylase is encoded by one or more nucleic acid molecules obtained from a microorganism selected from the group consisting of Clostridium sp., Bacillus sp., Chromobacterium sp. and Pseudomonas sp. In another embodiment, the acetoacetate decarboxylase is encoded by one or more nucleic acid molecules obtained from a microorganism selected from the group consisting of Clostridium acetobutylicum, Clostridium beijerinckii, Clostridium cellulolyticum, Bacillus polymyxa, Chromobacterium violaceum and Pseudomonas putida. In some embodiments, the one or more nucleic acid molecules encoding the acetoacetate decarboxylase is adc, or homolog thereof. In a further embodiment, the acetoacetate decarboxylase comprises an amino acid sequence selected from the group consisting of SEQ ID NOs: 49 and 52. In yet another embodiment, the acetoacetate decarboxylase is encoded by a nucleic acid sequence selected from the group consisting of SEQ ID NOs: 47, 48, 50 and 51.

In one embodiment of any aspect disclosed above, the 3-hydroxyisovalerate synthase is encoded by one or more nucleic acid molecules obtained from a microorganism selected from Mus sp., Saccharomyces sp., Lactobacillus sp. and Polaromonas sp. In another embodiment, the 3-hydroxyisovalerate synthase is encoded by one or more nucleic acid molecules obtained from a microorganism selected from Mus musculus, Saccharomyces cerevisiae, Lactobacillus crispatus and Polaromonas naphthalenivorans. In some embodiments, the one or more nucleic acid molecules encoding the 3-hydroxyisovalerate synthase is selected from Hmgcs1, ERG13, PksG and/or Pnap_0477, or homolog thereof. In a further embodiment, the one or more nucleic acid molecules encoding the 3-hydroxyisovalerate synthase comprises an amino acid sequence selected from the group consisting of SEQ ID NOs: 105, 107, 109 and 111. In yet another embodiment, the one or more nucleic acid molecules encoding the 3-hydroxyisovalerate synthase is encoded by a nucleic acid sequence selected from the group consisting of SEQ ID NOs: 104, 106, 108 and 110.

In one embodiment of any aspect disclosed above, the hydroxymethylglutaryl-CoA synthase is encoded by one or more nucleic acid molecules obtained from Saccharomyces sp. In another embodiment, the hydroxymethylglutaryl-CoA synthase is encoded by one or more nucleic acid molecules obtained from Saccharomyces cerevisiae. In some embodiments, the one or more nucleic acid molecules encoding the hydroxymethylglutaryl-CoA synthase is HmgS, or homolog thereof. In a further embodiment, the one or more nucleic acid molecules encoding the hydroxymethylglutaryl-CoA synthase comprises an amino acid sequence set forth in SEQ ID NO: 123. In yet another embodiment, the one or more nucleic acid molecules encoding the hydroxymethylglutaryl-CoA synthase is encoded by a nucleic acid sequence set forth in SEQ ID NO: 122.

In one embodiment of any aspect disclosed above, the methylglutaconyl-CoA hydratase is encoded by one or more nucleic acid molecules obtained from Pseudomonas sp. In another embodiment, the methylglutaconyl-CoA hydratase is encoded by one or more nucleic acid molecules obtained from Pseudomonas putida. In some embodiments, the one or more nucleic acid molecules encoding the methylglutaconyl-CoA hydratase is liuC, or homolog thereof. In a further embodiment, the one or more nucleic acid molecules encoding the methylglutaconyl-CoA hydratase comprises an amino acid sequence set forth in SEQ ID NO: 125. In yet another embodiment, the one or more nucleic acid molecules encoding the methylglutaconyl-CoA hydratase is encoded by a nucleic acid sequence set forth in SEQ ID NO: 124.

In one embodiment of any aspect disclosed above, the methylcrotonyl-CoA carboxylase is encoded by one or more nucleic acid molecules obtained from Pseudomonas sp. In another embodiment, the methylcrotonyl-CoA carboxylase is encoded by one or more nucleic acid molecules obtained from Pseudomonas aeruginosa. In some embodiments, the one or more nucleic acid molecules encoding the methylcrotonyl-CoA carboxylase is liuB, and/or liuD, or homologs thereof. In a further embodiment, the one or more nucleic acid molecules encoding the methylcrotonyl-CoA carboxylase comprises an amino acid sequence selected from SEQ ID NOs: 127 and 129. In yet another embodiment, the one or more nucleic acid molecules encoding the methylcrotonyl-CoA carboxylase is encoded by a nucleic acid sequence selected from SEQ ID NOs: 126 and 128.

In one embodiment of any aspect disclosed above, the methylcrotonyl-CoA hydratase is a 3-ketoacyl-CoA thiolase. In another embodiment, the methylcrotonyl-CoA hydratase is an enoyl-CoA hydratase. In another embodiment, the methylcrotonyl-CoA hydratase is encoded by one or more nucleic acid molecules obtained from E. coli. In some embodiments, the one or more nucleic acid molecules encoding the methylcrotonyl-CoA hydratase is fadA, and/or fadB, or homologs thereof. In a further embodiment, the one or more nucleic acid molecules encoding the methylcrotonyl-CoA hydratase comprises an amino acid sequence selected from SEQ ID NOs: 131 and 133. In yet another embodiment, the one or more nucleic acid molecules encoding the methylcrotonyl-CoA hydratase is encoded by a nucleic acid sequence selected from SEQ ID NOs: 130 and 132.

In one embodiment of any aspect disclosed above, the 3-hydroxyisovaleryl-CoA thioesterase is encoded by one or more nucleic acid molecules obtained from E. coli. In some embodiments, the one or more nucleic acid molecules encoding the 3-hydroxyisovaleryl-CoA thioesterase is tesB, or homolog thereof. In a further embodiment, the one or more nucleic acid molecules encoding the 3-hydroxyisovaleryl-CoA thioesterase comprises an amino acid sequence set forth in SEQ ID NO: 135. In yet another embodiment, the one or more nucleic acid molecules encoding the 3-hydroxyisovaleryl-CoA thioesterase is encoded by a nucleic acid sequence set forth in SEQ ID NO: 134.

In one embodiment of any aspect disclosed above, the 3HIV kinase is encoded by one or more nucleic acid molecules obtained from a microorganism selected from the group consisting of Thermoplasma sp. and Picrophilus sp. In another embodiment, the 3HIV kinase is encoded by one or more nucleic acid molecules obtained from a microorganism selected from the group consisting of Thermoplasma acidophilum and Picrophilus torridus. In some embodiments, the one or more nucleic acid molecules encoding the 3HIV kinase is TA1305 and/or PTO1356, or homolog thereof. In some embodiments, the TA1305 comprises a L200E mutation. In a further embodiment, the one or more nucleic acid molecules encoding the 3HIV kinase comprises an amino acid sequence selected from the group consisting of SEQ ID NOs: 113, 115 and 117. In yet another embodiment, the one or more nucleic acid molecules encoding the 3HIV kinase is encoded by a nucleic acid sequence selected from the group consisting of SEQ ID NOs: 112, 114 and 116.

In one embodiment of any aspect disclosed above, the 3HIV-3-phosphate decarboxylase is encoded by one or more nucleic acid molecules obtained from Streptococcus sp. In another embodiment, the 3HIV-3-phosphate decarboxylase is encoded by one or more nucleic acid molecules obtained from a microorganism selected from Streptococcus mitis and Streptococcus gordonii. In some embodiments, the one or more nucleic acid molecules encoding the 3HIV-3-phosphate decarboxylase comprises smi_1746 and/or mvaD, or homolog thereof. In a further embodiment, the one or more nucleic acid molecules encoding the 3HIV-3-phosphate decarboxylase comprises an amino acid sequence selected from SEQ ID NOs: 119 and 121. In yet another embodiment, the one or more nucleic acid molecules encoding the 3HIV-3-phosphate decarboxylase is encoded by a nucleic acid sequence selected from SEQ ID NOs: 118 and 120.

In one embodiment of any aspect disclosed above, the 3HIV decarboxylase is encoded by one or more nucleic acid molecules obtained from a microorganism selected from the group consisting of Streptococcus sp., Thermoplasma sp. and Picrophilus sp. In another embodiment, the 3HIV decarboxylase is encoded by one or more nucleic acid molecules obtained from a microorganism selected from the group consisting of Streptococcus gordonii, Thermoplasma acidophilum and Picrophilus torridus. In some embodiments, the one or more nucleic acid molecules encoding the 3HIV decarboxylase comprises mvaD, TA1305 and/or PTO1356, or homolog thereof. In a further embodiment, the one or more nucleic acid molecules encoding the 3HIV decarboxylase comprises an amino acid sequence selected from the group consisting of SEQ ID NOs: 113, 117 and 121. In yet another embodiment, the one or more nucleic acid molecules encoding the 3HIV decarboxylase is encoded by a nucleic acid sequence selected from the group consisting of SEQ ID NOs: 112, 116 and 120.

In one embodiment, MEG is produced through the conversion of glycolaldehyde in a C2 branch pathway and isobutene is produced through the conversion of DHAP or pyruvate in a C3 branch pathway.

In one embodiment, at least a portion of the excess NADH produced in the C3 branch is used as a source of reducing equivalents in the C2 branch. In another embodiment, at least a portion of the excess NADH produced in the C3 branch is used to produce ATP.

In one embodiment, excess biomass formation is minimized and production of MEG and isobutene is maximized.

In yet another aspect, the present application provides a method of producing MEG and isobutene using a recombinant microorganism as described above, wherein the method comprises cultivating the recombinant microorganism in a culture medium containing a feedstock providing a carbon source until the MEG and isobutene are produced.

In yet another aspect, the present application provides methods of producing a recombinant microorganism that co-produces, produces or accumulates MEG and isobutene.

### BRIEF DESCRIPTION OF DRAWINGS

Illustrative embodiments of the disclosure are illustrated in the drawings, in which:

FIG. 1 illustrates MEG and isobutene co-production pathway via D-ribulose-1-phosphate and acetone.

FIG. 2 illustrates MEG and isobutene co-production pathway via D-xylulose-1-phosphate and acetone.

FIG. 3 illustrates MEG and isobutene co-production pathway via xylonate and acetone.

FIG. 4 illustrates possible pathways for fermentative isobutene production. Adapted from Van Leeuwen 2012 (Appl Microbiol Biotechnol 93:1377-1387).

FIG. 5 shows a representative chromatogram for volatile compounds showing acetone, which was co-produced with ethylene glycol and acetic acid, after 46 h of cultivation of a modified E. coli strain on xylose.

FIG. 6 shows a representative chromatogram for sugars, acids and alcohols showing ethylene glycol, which was co-produced with acetone and acetic acid, after 46 h of cultivation of a modified E. coli strain on xylose.

FIG. 7 depicts the in vivo production of isobutene from acetone supplemented in the minimal medium culture (17 mM, 250 mM, and 500 mM acetone, respectively).

FIG. 8 depicts the in vivo production of isobutene from 10 mM 3-hydroxyisovalerate (3-HIV) supplemented in the minimal culture medium.

### DETAILED DESCRIPTION

Definitions

The following definitions and abbreviations are to be used for the interpretation of the disclosure.

As used herein and in the appended claims, the singular forms “a,” “an,” and “the” include plural referents unless the context clearly dictates otherwise. Thus, for example, reference to “an enzyme” includes a plurality of such enzymes and reference to “the microorganism” includes reference to one or more microorganisms, and so forth.

As used herein, the terms “comprises,” “comprising,” “includes,” “including,” “has,” “having, “contains,” “containing,” or any other variation thereof, are intended to cover a non-exclusive inclusion. A composition, mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive “or” and not to an exclusive “or.”

The terms “about” and “around,” as used herein to modify a numerical value, indicate a close range surrounding that explicit value. If “X” were the value, “about X” or “around X” would indicate a value from 0.9× to 1.1×, or, in some embodiments, a value from 0.95× to 1.05×. Any reference to “about X” or “around X” specifically indicates at least the values X, 0.95×, 0.96×, 0.97×, 0.98×, 0.99×, 1.01×, 1.02×, 1.03×, 1.04×, and 1.05×. Thus, “about X” and “around X” are intended to teach and provide written description support for a claim limitation of, e.g., “0.98×.”

As used herein, the terms “microbial,” “microbial organism,” and “microorganism” include any organism that exists as a microscopic cell that is included within the domains of archaea, bacteria or eukarya, the latter including yeast and filamentous fungi, protozoa, algae, or higher Protista. Therefore, the term is intended to encompass prokaryotic or eukaryotic cells or organisms having a microscopic size and includes bacteria, archaea, and eubacteria of all species as well as eukaryotic microorganisms such as yeast and fungi. Also included are cell cultures of any species that can be cultured for the production of a chemical.

As described herein, in some embodiments, the recombinant microorganisms are prokaryotic microorganism. In some embodiments, the prokaryotic microorganisms are bacteria. “Bacteria”, or “eubacteria”, refers to a domain of prokaryotic organisms. Bacteria include at least eleven distinct groups as follows: (1) Gram-positive (gram+) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles.

“Gram-negative bacteria” include cocci, nonenteric rods, and enteric rods. The genera of Gram-negative bacteria include, for example, Neisseria, Spirillum, Pasteurella, Brucella, Yersinia, Francisella, Haemophilus, Bordetella, Escherichia, Salmonella, Shigella, Klebsiella, Proteus, Vibrio, Pseudomonas, Bacteroides, Acetobacter, Aerobacter, Agrobacterium, Azotobacter, Spirilla, Serratia, Vibrio, Rhizobium, Chlamydia, Rickettsia, Treponema, and Fusobacterium.

“Gram positive bacteria” include cocci, nonsporulating rods, and sporulating rods. The genera of gram positive bacteria include, for example, Actinomyces, Bacillus, Clostridium, Corynebacterium, Erysipelothrix, Lactobacillus, Listeria, Mycobacterium, Myxococcus, Nocardia, Staphylococcus, Streptococcus, and Streptomyces.

The term “recombinant microorganism” and “recombinant host cell” are used interchangeably herein and refer to microorganisms that have been genetically modified to express or to overexpress endogenous enzymes, to express heterologous enzymes, such as those included in a vector, in an integration construct, or which have an alteration in expression of an endogenous gene. By “alteration” it is meant that the expression of the gene, or level of a RNA molecule or equivalent RNA molecules encoding one or more polypeptides or polypeptide subunits, or activity of one or more polypeptides or polypeptide subunits is up regulated or down regulated, such that expression, level, or activity is greater than or less than that observed in the absence of the alteration. For example, the term “alter” can mean “inhibit,” but the use of the word “alter” is not limited to this definition. It is understood that the terms “recombinant microorganism” and “recombinant host cell” refer not only to the particular recombinant microorganism but to the progeny or potential progeny of such a microorganism. Because certain modifications may occur in succeeding generations due to either mutation or environmental influences, such progeny may not, in fact, be identical to the parent cell, but are still included within the scope of the term as used herein.

The term “expression” with respect to a gene sequence refers to transcription of the gene and, as appropriate, translation of the resulting mRNA transcript to a protein. Thus, as will be clear from the context, expression of a protein results from transcription and translation of the open reading frame sequence. The level of expression of a desired product in a host cell may be determined on the basis of either the amount of corresponding mRNA that is present in the cell, or the amount of the desired product encoded by the selected sequence. For example, mRNA transcribed from a selected sequence can be quantitated by qRT-PCR or by Northern hybridization (see Sambrook et al., Molecular Cloning: A Laboratory Manual, Cold Spring Harbor Laboratory Press (1989)). Protein encoded by a selected sequence can be quantitated by various methods, e.g., by ELISA, by assaying for the biological activity of the protein, or by employing assays that are independent of such activity, such as western blotting or radioimmunoassay, using antibodies that recognize and bind the protein. See Sambrook et al., 1989, supra.

The term “polynucleotide” is used herein interchangeably with the term “nucleic acid” and refers to an organic polymer composed of two or more monomers including nucleotides, nucleosides or analogs thereof, including but not limited to single stranded or double stranded, sense or antisense deoxyribonucleic acid (DNA) of any length and, where appropriate, single stranded or double stranded, sense or antisense ribonucleic acid (RNA) of any length, including siRNA. The term “nucleotide” refers to any of several compounds that consist of a ribose or deoxyribose sugar joined to a purine or a pyrimidine base and to a phosphate group, and that are the basic structural units of nucleic acids. The term “nucleoside” refers to a compound (as guanosine or adenosine) that consists of a purine or pyrimidine base combined with deoxyribose or ribose and is found especially in nucleic acids. The term “nucleotide analog” or “nucleoside analog” refers, respectively, to a nucleotide or nucleoside in which one or more individual atoms have been replaced with a different atom or with a different functional group. Accordingly, the term polynucleotide includes nucleic acids of any length, DNA, RNA, analogs and fragments thereof. A polynucleotide of three or more nucleotides is also called nucleotidic oligomer or oligonucleotide.

It is understood that the polynucleotides described herein include “genes” and that the nucleic acid molecules described herein include “vectors” or “plasmids.” Accordingly, the term “gene”, also called a “structural gene” refers to a polynucleotide that codes for a particular sequence of amino acids, which comprise all or part of one or more proteins or enzymes, and may include regulatory (non-transcribed) DNA sequences, such as promoter sequences, which determine for example the conditions under which the gene is expressed. The transcribed region of the gene may include untranslated regions, including introns, 5′-untranslated region (UTR), and 3′-UTR, as well as the coding sequence.

The term “enzyme” as used herein refers to any substance that catalyzes or promotes one or more chemical or biochemical reactions, which usually includes enzymes totally or partially composed of a polypeptide or polypeptides, but can include enzymes composed of a different molecule including polynucleotides.

As used herein, the term “non-naturally occurring,” when used in reference to a microorganism organism or enzyme activity of the disclosure, is intended to mean that the microorganism organism or enzyme has at least one genetic alteration not normally found in a naturally occurring strain of the referenced species, including wild-type strains of the referenced species. Genetic alterations include, for example, modifications introducing expressible nucleic acids encoding metabolic polypeptides, other nucleic acid additions, nucleic acid deletions and/or other functional disruption of the microorganism's genetic material. Such modifications include, for example, coding regions and functional fragments thereof, for heterologous, homologous, or both heterologous and homologous polypeptides for the referenced species. Additional modifications include, for example, non-coding regulatory regions in which the modifications alter expression of a gene or operon. Exemplary non-naturally occurring microorganism or enzyme activity includes the hydroxylation activity described above.

The term “exogenous” as used herein with reference to various molecules, e.g., polynucleotides, polypeptides, enzymes, etc., refers to molecules that are not normally or naturally found in and/or produced by a given yeast, bacterium, organism, microorganism, or cell in nature.

On the other hand, the term “endogenous” or “native” as used herein with reference to various molecules, e.g., polynucleotides, polypeptides, enzymes, etc., refers to molecules that are normally or naturally found in and/or produced by a given yeast, bacterium, organism, microorganism, or cell in nature.

The term “heterologous” as used herein in the context of a modified host cell refers to various molecules, e.g., polynucleotides, polypeptides, enzymes, etc., wherein at least one of the following is true: (a) the molecule(s) is/are foreign (“exogenous”) to (i.e., not naturally found in) the host cell; (b) the molecule(s) is/are naturally found in (e.g., is “endogenous to”) a given host microorganism or host cell but is either produced in an unnatural location or in an unnatural amount in the cell; and/or (c) the molecule(s) differ(s) in nucleotide or amino acid sequence from the endogenous nucleotide or amino acid sequence(s) such that the molecule differing in nucleotide or amino acid sequence from the endogenous nucleotide or amino acid as found endogenously is produced in an unnatural (e.g., greater than naturally found) amount in the cell.

The term “homolog,” as used herein with respect to an original enzyme or gene of a first family or species, refers to distinct enzymes or genes of a second family or species which are determined by functional, structural, or genomic analyses to be an enzyme or gene of the second family or species which corresponds to the original enzyme or gene of the first family or species. Homologs most often have functional, structural, or genomic similarities. Techniques are known by which homologs of an enzyme or gene can readily be cloned using genetic probes and PCR. Identity of cloned sequences as homologs can be confirmed using functional assays and/or by genomic mapping of the genes.

A protein has “homology” or is “homologous” to a second protein if the amino acid sequence encoded by a gene has a similar amino acid sequence to that of the second gene. Alternatively, a protein has homology to a second protein if the two proteins have “similar” amino acid sequences. Thus, the term “homologous proteins” is intended to mean that the two proteins have similar amino acid sequences. In certain instances, the homology between two proteins is indicative of its shared ancestry, related by evolution. The terms “homologous sequences” or “homologs” are thought, believed, or known to be functionally related. A functional relationship may be indicated in any one of a number of ways, including, but not limited to: (a) degree of sequence identity and/or (b) the same or similar biological function. Preferably, both (a) and (b) are indicated. The degree of sequence identity may vary, but in one embodiment, is at least 50% (when using standard sequence alignment programs known in the art), at least 60%, at least 65%, at least 70%, at least 75%, at least 80%, at least 85%, at least 90%, at least about 91%, at least about 92%, at least about 93%, at least about 94%, at least about 95%, at least about 96%, at least about 97%, at least about 98%, or at least 98.5%, or at least about 99%, or at least 99.5%, or at least 99.8%, or at least 99.9%. Homology can be determined using software programs readily available in the art, such as those discussed in Current Protocols in Molecular Biology (F. M. Ausubel et al., eds., 1987) Supplement 30, section 7.718, Table 7.71. Some alignment programs are MacVector (Oxford Molecular Ltd, Oxford, U.K.) and ALIGN Plus (Scientific and Educational Software, Pennsylvania). Other non-limiting alignment programs include Sequencher (Gene Codes, Ann Arbor, Mich.), AlignX, and Vector NTI (Invitrogen, Carlsbad, Calif.).

The term “variant” refers to any polypeptide or enzyme described herein. A variant also encompasses one or more components of a multimer, multimers comprising an individual component, multimers comprising multiples of an individual component (e.g., multimers of a reference molecule), a chemical breakdown product, and a biological breakdown product. In particular, non-limiting embodiments, a linalool dehydratase/isomerase enzyme may be a “variant” relative to a reference linalool dehydratase/isomerase enzyme by virtue of alteration(s) in any part of the polypeptide sequence encoding the reference linalool dehydratase/isomerase enzyme. A variant of a reference linalool dehydratase/isomerase enzyme can have enzyme activity of at least 10%, at least 30%, at least 50%, at least 80%, at least 90%, at least 100%, at least 105%, at least 110%, at least 120%, at least 130% or more in a standard assay used to measure enzyme activity of a preparation of the reference linalool dehydratase/isomerase enzyme. In some embodiments, a variant may also refer to polypeptides having at least 50%, at least 60%, at least 70%, at least 80%, at least 90%, at least 91%, at least 92%, at least 93%, at least 94%, at least 95%, at least 96%, at least 97%, at least 98%, or at least 99% sequence identity to the full-length, or unprocessed linalool dehydratase/isomerase enzymes of the present disclosure. In some embodiments, a variant may also refer to polypeptides having at least 50%, at least 60%, at least 70%, at least 80%, at least 90%, at least 91%, at least 92%, at least 93%, at least 94%, at least 95%, at least 96%, at least 97%, at least 98%, or at least 99% sequence identity to the mature, or processed linalool dehydratase/isomerase enzymes of the present disclosure.

The term “signal sequence” as used herein refers to an amino acid sequence that targets peptides and polypeptides to cellular locations or to the extracellular environment. Signal sequences are typically at the N-terminal portion of a polypeptide and are typically removed enzymatically. Polypeptides that have their signal sequences are referred to as being full-length and/or unprocessed. Polypeptides that have had their signal sequences removed are referred to as being mature and/or processed.

The term “yield potential” as used herein refers to a yield of a product from a biosynthetic pathway. In one embodiment, the yield potential may be expressed as a percent by weight of end product per weight of starting compound.

The term “thermodynamic maximum yield” as used herein refers to the maximum yield of a product obtained from fermentation of a given feedstock, such as glucose, based on the energetic value of the product compared to the feedstock. In a normal fermentation, without use of additional energy sources such as light, hydrogen gas or methane or electricity, for instance, the product cannot contain more energy than the feedstock. The thermodynamic maximum yield signifies a product yield at which all energy and mass from the feedstock is converted to the product. This yield can be calculated and is independent of a specific pathway. If a specific pathway towards a product has a lower yield than the thermodynamic maximum yield, then it loses mass and can most likely be improved upon or substituted with a more efficient pathway towards the product.

The term “redox balanced” refers to a set of reactions, which taken together produce as much redox cofactors as they consume. Designing metabolic pathways and engineering an organism such that the redox cofactors are balanced or close to being balanced usually results in a more efficient, higher yield production of the desired compounds. Redox reactions always occur together as two half-reactions happening simultaneously, one being an oxidation reaction and the other a reduction reaction. In redox processes, the reductant transfers electrons to the oxidant. Thus, in the reaction, the reductant or reducing agent loses electrons and is oxidized, and the oxidant or oxidizing agent gains electrons and is reduced. In one embodiment, the redox reactions take place in a biological system. Biological energy is frequently stored and released by means of redox reactions. Photosynthesis involves the reduction of carbon dioxide into sugars and the oxidation of water into molecular oxygen. The reverse reaction, respiration, oxidizes sugars to produce carbon dioxide and water. As intermediate steps, the reduced carbon compounds are used to reduce nicotinamide adenine dinucleotide (NAD+), which then contributes to the creation of a proton gradient, which drives the synthesis of adenosine triphosphate (ATP) and is maintained by the reduction of oxygen. The term redox state is often used to describe the balance of GSH/GSSG, NAD+/NADH and NADP+/NADPH in a biological system such as a cell or organ. The redox state is reflected in the balance of several sets of metabolites (e.g., lactate and pyruvate, beta-hydroxybutyrate, and acetoacetate), whose interconversion is dependent on these ratios. An abnormal redox state can develop in a variety of deleterious situations, such as hypoxia, shock, and sepsis.

The terms “C2 pathway”, “C2 branch pathway” or “C2 stream” as used herein refers to a biochemical pathway wherein MEG can be produced via glycolaldehyde.

The terms “C3 pathway”, “C3 branch pathway” or “C3 stream” as used herein refers to a biochemical pathway wherein isobutene can be produced via pyruvate or dihydroxyacetonephosphate (DHAP).

### Introduction

The present disclosure combines the production of monoethylene glycol (MEG) and isobutene in different hosts. The present disclosure avoids some of the biggest pathway engineering challenges for known MEG and isobutene pathways demonstrated so far. Surprisingly, the combination of a 2-carbon stream pathway and a 3-carbon stream pathway complements each other and is highly synergistic, avoiding or overcoming the biggest challenges and shortcomings of each complementing pathway, establishing a good redox balance but also delivering required ATP, without being in excess.

All pathways for direct isobutene production from a sugar actually demonstrated so far can only achieve 63% of the energetic maximum yield potential. However, if combined with any one of three known MEG from xylose pathways, overall yield potential increases to 88% of energetic yield potential due to the synergy of NADH producing and NADH requiring pathways.

The MEG+isobutene production pathways of the present disclosure avoid the biggest metabolic engineering and technical challenges of optimizing individual MEG or isobutene processes: difficult C3-stream MEG fermentation, the need for carbon fixation or phospho-transketolase/phospho-transacetylase (PK/PTA) pathway implementation or unfavorable/undescribed enzyme reactions for isobutene fermentations.

A demonstrated fermentative production of MEG from xylose (WO2013126721A1, which is herein referenced in its entirety), via ribulose-1-phosphate, has a high yield potential (82 wt %=0.82 g MEG/g xylose). MEG is produced via two different pathways which are active in parallel, a 2-carbon (C2) stream (via glycolaldehyde) and a 3-carbon (C3) stream (via dihydroxyacetonephosphate (DHAP)). The C2 stream is easy to implement at high efficiency, but the C3 stream is very difficult to implement at high efficiency via metabolic engineering. Several pathway options for DHAP→MEG exist, all of which are difficult to implement. Furthermore, the overall process is ATP neutral. Thus, some glucose and therefore yield will be lost in order to obtain some surplus ATP required for cell growth and maintenance.

A further demonstrated fermentative production of MEG from xylose (Alkim et al., Microb Cell Fact (2015) 14:127), via xylulose-1-phosphate, is very similar to the route described by WO2013126721A1. It has the same high yield potential (82 wt %), but the C3 stream for MEG production via DHAP is difficult to implement and there is an ATP shortage.

A further fermentative production of MEG was demonstrated from glucose (Chen et al., Met. Eng. (2016) 33:12-18). It uses exclusively a pathway identical to one of the C3 stream solutions of WO2013126721A1, going via DHAP and then ethanolamine to glyceraldehyde to MEG. Only in this case, DHAP is derived from glucose, not from xylose. Thus it suffers even more from the technical difficulty to implement a high productivity and high yield pathway from DHAP to MEG. It furthermore has a reduced total yield potential of 69 wt % versus the thermodynamic maximum yield for the product MEG derived from glucose (82 wt %). The pathway is furthermore ATP neutral, not generating any ATP that the cells need for growth and maintenance. This pathway is also not redox balanced and has a high excess of 2 mol NADH per mol of consumed glucose, all of which needs to be re-oxidized for the cell to be viable. In an aerobic fermentation, this NADH can be used to generate ATP, which however would be in high excess (2 NADH→6 ATP), leading to excess biomass formation during the production phase and therefore reduced product formation and yield. The only described solution for the loss of yield potential for MEG production from glucose is the production of MEG from xylose with a high yield potential. The only described solution for the excess NADH production in the MEG from glucose process is the production of MEG from xylose which can be redox neutral.

A review from Van Leeuwen et al. (Appl Microbiol Biotechnol (2012) 93:1377-1387) describes five known pathways for isobutene production.

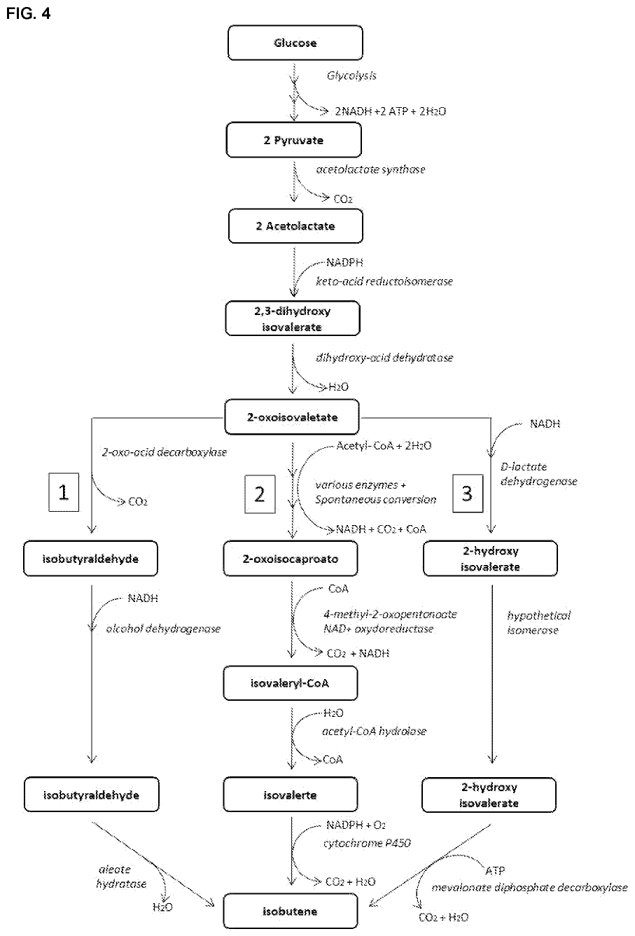

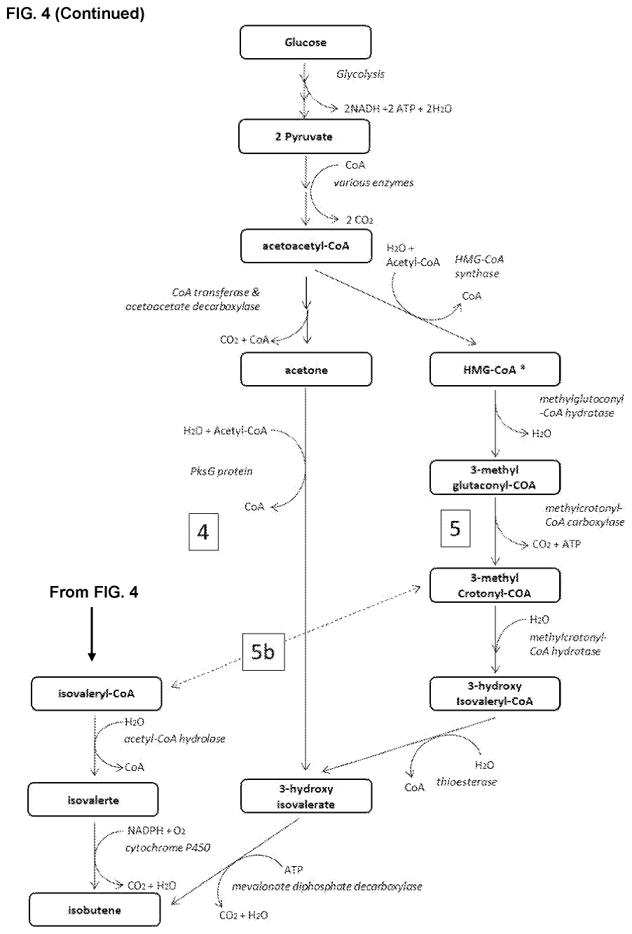

Pathway 1 (see FIG. 4) via isobutanol has the advantage of a well-established, efficient and productive isobutanol pathway (WO 2007050671, US2009081746). It would enable 0.31 g isobutene/g glucose. However, it requires a very challenging, energetically unfavorable last step, the dehydration of a non-activated simple alcohol to isobutene. While oleate hydratase reaction to dehydrate isobutanol supposedly has been achieved (WO2011076691), it is questionable if this reaction is or ever will be fast enough to enable an economic process. No data on reaction speed or efficiency with an oleate hydratase and no isobutene production from glucose using an oleate hydratase has been shown. A similar disclosure using isoflavonoid hydratase instead for general alcohol dehydrations (WO2011076689) remains prophetic as there was no experimental results or data.

Pathway 2 (see FIG. 4) produces isovalerate, followed by a P450 based oxidation to isobutene which has been described in academic articles (Fujii et al. 1988, Appl Environ Microbiol 54:583-584, Fukuda et al. 1994, Biochem Biophys Res Commun 201:516-522). This pathway has a limited yield of 0.21 g isobutene/g glucose plus a very challenging final reaction. Molecular oxygen based P450 enzymes typically require strong aeration of the culture to achieve sufficient productivity, which is very detrimental towards achieving an economic process for a bulk chemical.

Pathway 3 (see FIG. 4) via 2-hydroxyisovalerate has a good yield potential of 0.31 g isobutene/g glucose. However, the pathway is only prophetic, as it requires a hypothetical isomerase activity.

Pathway 4 (see FIG. 4, see also WO 2011032934) and pathway 5 are similar; both go through acetoacetyl-CoA to 3-hydroxyisovalerate to isobutene, and also share the key, final dehydration+decarboxylation step through a mevalonate-diphosphate decarboxylase (WO 2010001078, WO2012052427), which has been shown to work well and is very accessible to enzyme optimization (WO 2015004211). Pathway 4 via acetone represents a shortcut compared to pathway 5. Both have a less challenging final enzymatic step than pathways 1 or 2 and do not require a hypothetical enzyme activity like pathway 3. However, both share the same low isobutene yield potential of 0.21 g isobutene/g glucose.

Pathways 4 and 5 have a net equation of 1.5 C₆H₁₂O₆+H₂O→C₄H₈+5CO₂+2ATP+6NADH. Thus, each pathway produces excess NADH (6 mol per 1 mol of isobutene or per 1.5 mol of consumed glucose) and shows low yield potential (0.2076 g/g). This is just 63% of the calculated energetic maximum yield (0.3288 g/g). It would require efficient CO₂ carbon capture to reduce the loss of yield potential and re-oxidize NADH, which is very difficult to implement efficiently regarding the metabolic engineering as well as the fermentation process itself. If the NADH is not utilized via carbon fixation, it needs to be re-oxidized for the cell to stay viable, either via oxidized side-products and thus further loosing glucose in this process. Alternatively, NADH can be oxidized with oxygen for ATP production, which requires strong aeration of the fermenter and would lead to even more unwanted excess ATP and excess biomass formation (6 NADH ˜18 ATP).

Another pathway exists (FIG. 4, pathway 5b), reducing 3-methylcrotonyl-CoA to isovalerate by different possible reactions, followed by a P450 based oxidation to isobutene (WO2016042011). It basically represents a combination of pathways 4 and 5 and has the same limited yield, plus the difficulties of a P450 enzyme requiring high amounts of dissolved molecular oxygen.

Yet another pathway has been described in Gogerty and Bobik (Appl Env Microbiol, December 2010, p. 8004-8010) using L-leucine biosynthesis and part of a L-leucine biodegradation pathway. First, L-leucine or its direct pre-cursor 4-methyl-2-oxopentanoate is synthesized via the standard L-leucine biosynthesis pathway. L-leucine is then degraded by L-leucine:2-oxoglutarate aminotransferase to 4-methyl-2-oxopentanoate, by 4-methyl-2-oxopentanoate dehydrogenase to isovaleryl-CoA and further by isovaleryl-CoA dehydrogenase to 3-methylcrotonyl-CoA. Then enzymatic activities related to fatty acid degradation, namely methylcrotonyl-CoA hydratase and 3-hydroxyisovaleryl-CoA thioesterase, can be used to obtain 3-hydroxyisovalerate, which can then be transformed to isobutene as described for pathways 4 and 5 (FIG. 4). Since this L-leucine biosynthesis based pathway starts from pyruvate, it is also suitable for co-production with MEG. It has the exact same stoichiometry as pathways 4 and 5, i.e. 1.5 glucose→1 isobutene+5 CO₂+2ATP+6NAD(P)H, and thus the same excess NAD(P)H production and loss of yield potential. Therefore, it shares the same benefits if co-produced with MEG from xylose, namely significantly reduced surplus of reduction equivalents and greatly increased product yield potential of 88% of thermodynamic maximum yield (vs 63% for isobutene only). However, its large number of enzymatic steps and lack of demonstrated implementation makes it less preferable than pathways 4 and 5.

Thus, all pathways for a one-step direct production of isobutene, successfully demonstrated from a sugar so far, share the low yield potential of 0.21 g isobutene/g glucose and excess NADH production.

The present disclosure combines one of three easy to implement, high yield C2 streams for MEG production from xylose with easy to implement isobutene via DHAP or pyruvate pathways. Surprisingly, the problem of excess NADH production in previously described functional isobutene pathways complements the NADH requiring C2 part of MEG production. The combination of these pathways leads to a high total yield potential of 0.538 g/g from xylose (0.4134 g MEG/g xylose+0.1246 g isobutene/g xylose), which is 88% of the maximum calculated energetic yield of 0.614 g/g for degradation of xylose into MEG and isobutene, assuming these products are produced in a 3:1 ratio. This high yield potential stems from the synergies of coupling the isobutene pathway with the C2-branch of MEG production from xylose.

The proposed pathway in its basic form is not redox neutral, but has a small excess of 1 mol NADH per mol of consumed xylose. In an aerobic fermentation, oxidation of NADH can deliver just enough ATP to obtain sufficient, but not excessive ATP required for growth and maintenance during the production phase without having a significantly negative impact on product formation.

In one embodiment, the problem of a difficult to implement C3 pathway to produce MEG from xylose is solved by using the C3 stream to produce isobutene with an easier to implement pathway for MEG production. Several pathway options for DHAP→MEG exists, all of which are difficult to implement. In one embodiment, xylose is converted to MEG via glycoladehyde (C2 stream), while using the resulting DHAP or pyruvate (C3 stream) with a relatively easy to implement pathway for isobutene production.

In another embodiment, the problem of ATP shortage in production of MEG from xylose is solved. Even with the use of active, ATP utilizing xylose import, the proposed pathways of the present disclosure are ATP neutral or with a small negative ATP (0.33), but generate sufficient NADH to be used in an aerobic process to generate sufficient (˜3) ATP.

In some embodiments, the problem of ATP shortage in production of MEG from glucose is solved. The proposed pathways of the present disclosure are ATP neutral or with a small negative ATP (0.33), but generate sufficient NADH to be used in an aerobic process to generate sufficient (˜3) ATP. Most pathways that produce MEG from glucose lose yield potential due to excess CO₂ and NADH production. This NADH can be oxidized in an aerobic fermentation to generate ATP. However, this solution to the net ATP consumption of the pathway means loss of yield potential and can produce excess ATP (6 NADH˜18 ATP) and therefore excess biomass formation and further loss of yield.

In one embodiment, the proposed pathways of the present disclosure solve the problem of loss of yield potential in producing MEG from glucose. By producing MEG and isobutene from xylose, both compounds are produced at high yield with little overall loss. The only other described solution for the loss of yield potential for MEG production from glucose is the production of MEG (with or without co-products) from xylose.

In one embodiment, the proposed pathways of the present disclosure solve the problem of high excess NADH production in producing MEG from glucose. This not only reduces yield potential, but also requires potentially unwanted side-product formation, or strong aeration with excess ATP and biomass formation. The presently disclosed process is not redox neutral, but produces less NADH excess than the known glucose based pathway. This small excess NADH is used to produce extra ATP. The only other described solution for the excess NADH production in producing MEG from glucose is the production of MEG (with or without co-products) from xylose, which can be redox neutral.

In one embodiment, the proposed pathways of the present disclosure solve the problem of loss of yield potential of isobutene produced from glucose and solve the problem of excess NADH production when isobutene is produced from glucose. The presently disclosed process of co-producing MEG and isobutene is synergistic by utilizing the excess NADH produced in the C3 stream pathway to isobutene to feed the NADH requirement of the C2 stream to MEG. This way, excess NADH and CO₂ release is avoided and yield potential is significantly increased.

Other potential solutions exist for reducing NADH excess and therefore reduce excess CO₂ and increase isobutene yield potential: re-capturing CO₂ produced in excess during the fermentation and in doing so also re-oxidizing excess NADH (CO₂ fixation); avoiding excess CO₂ and NADH release altogether by diverting some flux from glycolysis to a phospho-transketolase/phospho-transacetylase (PK/PTA) pathway to generate more acetyl-CoA and less CO₂+NADH. However, so far none of these options have been technically demonstrated in the context of isobutene production and are generally known to be very challenging.

In a further embodiment, the inventive co-production pathway from xylose is implemented in an organism with natural or modified capability to fix CO₂ using excess reducing agents, thereby providing even higher yield potential. Various CO₂ fixation pathways are known and have been implemented in E. coli or other hosts. Acetogens, such as Clostridium ljungdahlii, can naturally utilize excess NADH generated in the presented xylose fermentation pathway especially efficient to re-capture released CO₂ in the Wood-Ljungdahl pathway to produce the intermediate acetyl-CoA, which can then be used to produce more acetone or related products. CO₂ is released for instance in the pyruvate+CoA+NAD+ acetyl-CoA+CO₂+2 NADH or acetoacetone→acetone+CO₂ reactions. Furthermore, adding a second feedstock, such as hydrogen gas (H₂) or syngas (a composition of H₂, CO, CO₂) or methanol, can provide more reducing agents and even allow re-capture of all CO₂ released in the xylose fermentation pathway or CO2 present in the second feedstock. Such a mixotrophic fermentation can thus further increase yield potential.

In the case of MEG+isobutene from xylose, CO₂ fixation can lead to an increase of 25% relative isobutene or 5.8% total MEG+isobutene product yield. With externally added reducing agents, calculated for full capture of all xylose carbon, the yield potential is +125% for isobutene which equals+30% total product yield.

Yield potentials without CO₂ fixation:

1 xylose→1 MEG+1/3 isobutene+5/3 CO₂+1 NADH

Yield potentials with CO₂ fixation:

1 xylose→1 MEG+5/12 isobutene+4/3 CO₂

Yield potentials with externally added reducing agents, calculated for fixation of CO₂ equivalent to all CO₂ released during xylose fermentation:

1 xylose→1 MEG+3/4 isobutene

Other potential solutions are higher yield isobutene pathways (see FIG. 4, pathways 1 and 3). However, none of these other isobutene pathways have been demonstrated in full, i.e. producing isobutene from a sugar, and have significant enzymatic challenges (unfavorable, slow dehydration of non-activated alcohol or unavailable isomerization reaction).

In one embodiment, co-producing MEG and isobutene from xylose is synergistic by strongly increasing yield potential of isobutene production with an easy to implement and technically less challenging pathway while using a high yield and easy to implement MEG pathway.

In one embodiment, MEG is produced through the conversion of glycolaldehyde in a C2 branch pathway and isobutene is produced through the conversion of DHAP or pyruvate in a C3 branch pathway.

In one embodiment, at least a portion of the excess NADH produced in the C3 branch pathway is used as a source of reducing equivalents in the C2 branch pathway. In another embodiment, at least a portion of the excess NADH produced in the C3 branch pathway is used to produce ATP.

In one embodiment, excess biomass formation is minimized and production of MEG and isobutene is maximized.

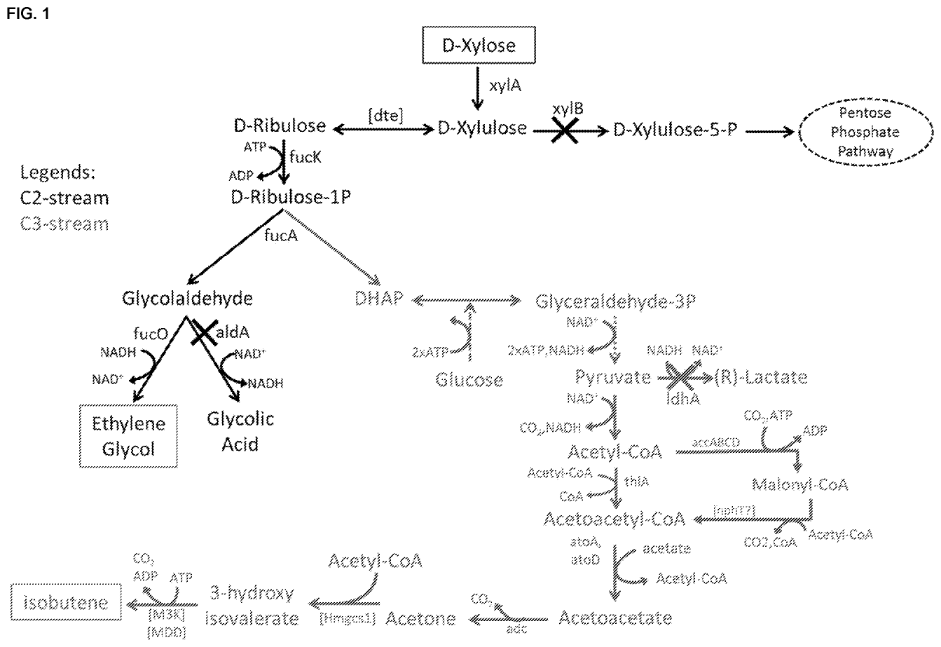

In some embodiments, MEG and isobutene co-production from xylose comprises the combination of any one of three pathways (via D-ribulose-1-phosphate, D-xylulose-1-phosphate or xylonate) for producing MEG via glycoladehyde (C2 stream) and DHAP or pyruvate (C3 stream) from xylose (see FIGS. 1-3), and any one of three compatible pathways for producing isobutene from pyruvate (via isovalerate, acetone or HMG-CoA; see FIG. 4, pathways 2, 4, 5).

In one preferred embodiment, MEG and isobutene are produced from xylose using a ribulose-1-phosphate pathway (see FIG. 1) for the conversion of xylose to MEG and dihydroxyacetone-phosphate (DHAP), and using an acetone based pathway for the conversion of DHAP to isobutene.

In another preferred embodiment, MEG and isobutene are produced from xylose using a ribulose-1-phosphate pathway (see FIG. 1) for the conversion of xylose to MEG and dihydroxyacetone-phosphate (DHAP), and using an HMG-CoA based pathway (see pathway 5 of FIG. 4) for the conversion of DHAP to isobutene.

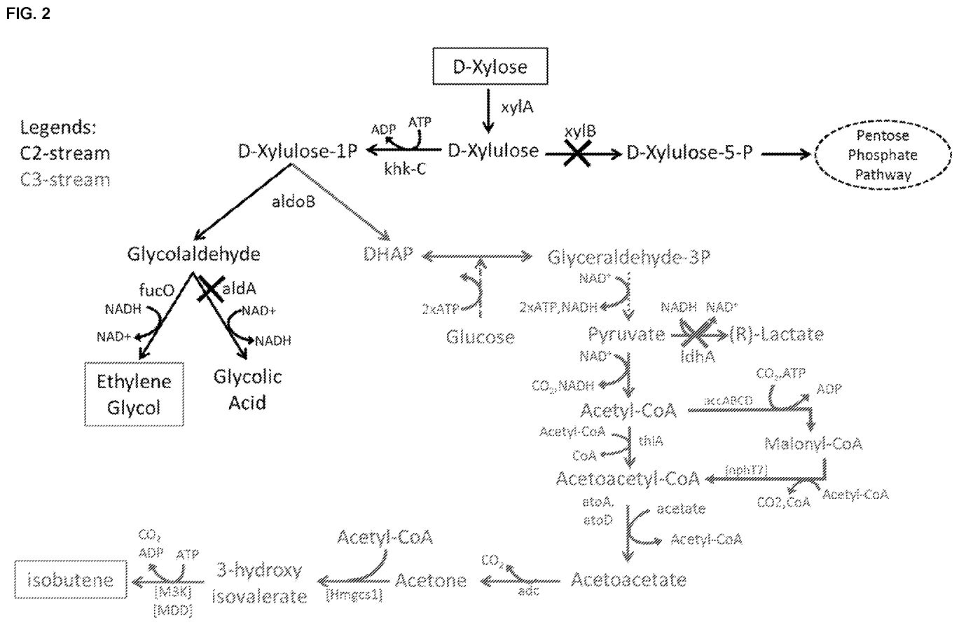

In one preferred embodiment, MEG and isobutene are produced from xylose using a xylulose-1-phosphate pathway (see FIG. 2) for the conversion of xylose to MEG and dihydroxyacetone-phosphate (DHAP), and using an acetone based pathway for the conversion of DHAP to isobutene.

In another preferred embodiment, MEG and isobutene are produced from xylose using a xylulose-1-phosphate pathway (see FIG. 1) for the conversion of xylose to MEG and dihydroxyacetone-phosphate (DHAP), and using an HMG-CoA based pathway (see pathway 5 of FIG. 4) for the conversion of DHAP to isobutene.

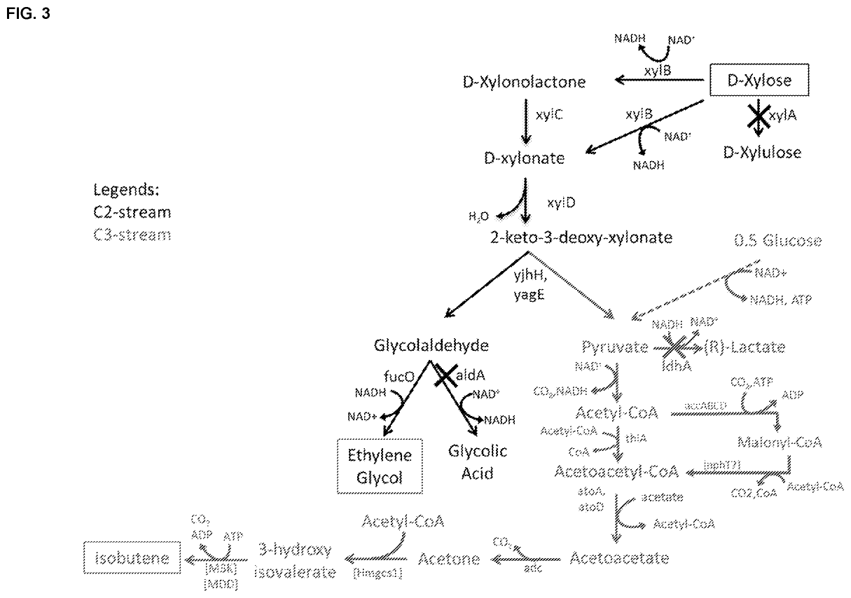

In one preferred embodiment, MEG and isobutene are produced from xylose using a xylonate pathway (see FIG. 3) for the conversion of xylose to MEG and pyruvate, and using an acetone based pathway for the conversion of pyruvate to isobutene.

In another preferred embodiment, MEG and isobutene are produced from xylose using a xylonate pathway (see FIG. 3) for the conversion of xylose to MEG and pyruvate, and using an HMG-CoA based pathway (see pathway 5 of FIG. 4) for the conversion of pyruvate to isobutene.

As xylose is used for the co-production of MEG and isobutene, the first step in a co-production pathway is the import of D-xylose and its conversion into D-xylulose, both natural functions in E. coli (transporter genes xylFGH or xylE, isomerase gene xylA). D-xylulose normally enters the pentose phosphate pathway for energy and biomass generation, which can be inhibited by the deletion of the xylB gene.

In some embodiments, xylose is converted to MEG via the ribulose-1-phosphate pathway (see FIG. 1). In some embodiments, all carbon can be re-directed to D-ribulose by the D-tagatose 3-epimerase enzyme in the engineered pathway. In another embodiment, D-ribulose can then be converted to D-ribulose-1-phosphate by the native E. coli enzyme D-ribulokinase (gene fucK). In another embodiment, D-ribulose-1-phosphate is cleaved into glycolaldehyde and dihydroxy acetone phosphate (DHAP) by the native E. coli L-fuculose-phosphate aldolase with D-ribulose-phosphate aldolase activity (gene fucA). Degradation of glycolaldehyde, termed the C2-branch, can lead to ethylene glycol or glycolate formation. Glycolate is the undesired by-product that can be produced by the native aldehyde dehydrogenase A (gene aldA), which therefore can be deleted. In one embodiment, ethylene glycol can be produced from glycolaldehyde using a native E. coli enzyme having glycolaldehyde reductase activity (for example, gene fucO, an L-1,2-propanediol oxidoreductase). The further degradation of DHAP is termed the C3 branch and can lead to isobutene production using either an acetone based pathway or an HMG-CoA based pathway as described below.

In some embodiments, xylose is converted to MEG via the xylulose-1-phosphate pathway (see FIG. 2).The first step of the pathway (FIG. 2) is the natural conversion of D-xylose into D-xylulose. In one embodiment, all carbon can be re-directed to D-xylulose-1-phosphate by the D-xylulose 1-kinase enzyme. D-xylulose-1-phosphate is then cleaved into glycolaldehyde and dihydroxy acetone phosphate (DHAP) by D-xylulose-1-phosphate aldolase. Production of MEG from glycolaldehyde proceeds as described for FIG. 1. Isobutene can be produced via further conversion of DHAP in the C3 branch using either an acetone based pathway or an HMG-CoA based pathway as described below.

In one embodiment, in order to increase carbon flux to either the ribulose-1-phosphate or the xylulose-1-phosphate pathway, three specific genes that could divert carbon flux were identified and deleted: xylB gene coding for a xylulokinase (this enzyme can divert carbon flux into the pentose phosphate pathway), the aldA gene coding for aldehyde dehydrogenase A (can divert carbon flux from glycolaldehyde to glycolate instead of to MEG) and the IdhA gene coding for lactate dehydrogenase (this enzyme can divert carbon flux from pyruvate to lactate instead of to acetyl-CoA).

In some embodiments, D-xylose can be converted to D-xylulose in the ribulose-1-phosphate pathway (FIG. 1) or in the D-xylulose-1-phosphate pathway (FIG. 2) by first converting D-xylose to xylitol, and then converting xylitol to D-xylulose. In one embodiment, D-xylose is converted to xylitol by a xylose reductase or aldose reductase. In another embodiment, xylitol is converted to D-xylulose by a xylitol dehydrogenase.

In some embodiments, xylose is converted to MEG via the xylonate pathway (see FIG. 3).The first step of the pathway (FIG. 3) is the conversion of D-xylose into D-xylonate, either by a two-step process using a xylose dehydrogenase to convert D-xylose to D-xylonolactone followed by conversion of D-xylonolactone to D-xylonate with a xylonolactonase enzyme, or by a one-step process using a xylose dehydrogenase to convert D-xylose directly to D-xylonate. The conversion of D-xylose to D-xylulose is inhibited by the deletion of the xylA gene. D-xylonate is then converted to 2-keto-3-deoxy-xylonate by a xylonate dehydratase. 2-keto-3-deoxy-xylonate is then cleaved into glycolaldehyde and pyruvate by 2-keto-3-deoxy-D-xylonate aldolase. Production of MEG from glycolaldehyde proceeds as described for FIG. 1. Isobutene can be produced via further conversion of pyruvate in the C3 branch using either an acetone based pathway or an HMG-CoA based pathway as described below.

In one embodiment, in order to increase carbon flux to the xylonate pathway, three specific genes that could divert carbon flux were identified and deleted: xylA gene coding for a D-xylose isomerase (this enzyme can divert carbon flux from D-xylose to D-xylulose instead of to D-xylonate or D-xylonolactone), the aldA gene coding for aldehyde dehydrogenase A (can divert carbon flux from glycolaldehyde to glycolate instead of to MEG) and the IdhA gene coding for lactate dehydrogenase (this enzyme can divert carbon flux from pyruvate to lactate instead of to acetyl-CoA).

In one embodiment, isobutene production in E. coli via an acetone based pathway comprises the following enzymes: thiolase or acetyl coenzyme A acetyltransferase; acetyl-CoA:acetoacetate-CoA transferase or acetate:acetoacetyl-CoA hydrolase; acetoacetate decarboxylase; 3-hydroxyisovalerate (3HIV) synthase; 3HIV kinase; 3HIV-3-phosphate decarboxylase and/or 3HIV decarboxylase.

The conversion of DHAP to acetyl-CoA (through glyceraldehyde-3-phosphate and pyruvate) is part of natural E. coli metabolism. One molecule of acetyl-CoA is condensed to another molecule of acetyl-CoA by the enzyme thiolase (acetyl-coenzyme A acetyltransferase, for example, gene thl from Clostridium acetobutylicum) to produce acetoacetyl-CoA. The CoA from acetoacetyl-CoA is recycled to a molecule of acetate by acetate:acetoacetyl-CoA transferase (for example, gene atoAD from E. coli) generating acetyl-CoA and acetoacetate. Acetoacetate is decarboxylated by acetoacetate decarboxylase (for example, gene adc from Clostridium acetobutylicum) to acetone which is further condensed with acetyl-CoA to 3-hydroxyisovalerate (3HIV, 3-hydroxy-3-methyl-butyrate) by 3HIV synthase (for example, HMG-CoA synthase gene Hmgcs1 from Mus musculus), which is finally phosphorylated by 3HIV kinase (for example, TA1305 from Thermoplasma acidophilum, preferably with a L200E mutation; it is also a 3HIV decarboxylase) and decarboxylated by a 3HIV-3-phosphate decarboxylase (for example, Streptococcus mitis strain B6 gene smi_1746). Alternatively, 3HIV can be dehydrated and decarboxylated by one 3HIV decarboxylase alone.

In one embodiment, isobutene production in E. coli via an HMG-CoA based pathway comprises the following enzymes: acetyl coenzyme A acetyltransferase; hydroxymethylglutaryl-CoA synthase; methylglutaconyl-CoA hydratase; methylcrotonyl-CoA carboxylase; methylcrotonyl-CoA hydratase; 3-hydroxyisovaleryl-CoA thioesterase; 3HIV kinase; 3HIV-3-phosphate decarboxylase and/or 3HIV decarboxylase.

Pyruvate is converted to acetyl-CoA by decarboxylation as part of the microorganism metabolism. One molecule of acetyl-CoA is condensed to another molecule of acetyl-CoA to form acetoacetyl-CoA by an acetyl coenzyme A acetyltransferase, such as thIA from Clostridium acetobutylicum. A hydroxymethylglutaryl-CoA synthase, such as HmgS from Saccharomyces cerevisiae, catalyzes the conversion of acetoacetyl-CoA and acetyl-CoA to 3-hydroxy-3-methylglutaryl-CoA (HMG-CoA). HMG-CoA is dehydrated by a methylglutaconyl-CoA hydratase, such as liuC from Pseudomonas putida, to 3-methylglutaconyl-CoA. 3-methylglutaconyl-CoA is decarboxylated by a methylcrotonyl-CoA carboxylase, such as liuB subunit beta/liuD subunit alpha from Pseudomonas aeruginosa, to 3-methylcrotonyl-CoA. 3-methylcrotonyl-CoA is hydrated by a methylcrotonyl-CoA hydratase, such as the 3-ketoacyl-CoA thiolase fadA or enoyl-CoA hydratase fadB from E. coli, to 3-hydroxyisovaleryl-CoA. 3-hydroxyisovaleryl-CoA is then hydrolyzed by a 3-hydroxyisovaleryl-CoA thioesterase, such as tesB from E. coli, to 3-hydroxyisovalerate (3HIV). 3HIV is then phosphorylated by a 3HIV kinase (such as TA1305 from Thermoplasma acidophilum with L200E mutation) and decarboxylated by a 3HIV-3-phosphate decarboxylase (such as gene smi_1746 from Streptococcus mitis) to isobutene. Alternatively, 3HIV can be dehydrated and decarboxylated by a 3HIV decarboxylase (for example, TA1305 from Thermoplasma acidophilum; PTO1356 from Picrophilus torridus; mvaD from Streptococcus gordonii) to isobutene.

In some embodiments, the present application provides a recombinant microorganism co-producing monoethylene glycol (MEG) and isobutene. In one embodiment, the MEG and isobutene are co-produced from xylose. In another embodiment, the recombinant microorganism comprises a deletion, insertion, or loss of function mutation in a gene encoding a D-xylulose-5-kinase and/or in a gene encoding a glycoaldehyde dehydrogenase. In some embodiments, the gene encoding the D-xylulose-5-kinase is xylB. In some embodiments, the gene encoding the glycoaldehyde dehydrogenase is aldA. In a further embodiment, isobutene is synthesized via the intermediate 3-hydroxyisovalerate. In some embodiments, MEG is produced through the conversion of glycolaldehyde in a C-2 branch pathway and isobutene is produced through the conversion of DHAP or pyruvate in a C-3 branch pathway. In other embodiments, at least a portion of the excess NADH produced in the C-3 branch pathway is used as a source of reducing equivalents in the C-2 branch pathway. In further embodiments, at least a portion of the excess NADH produced in the C-3 branch pathway is used to produce ATP. In yet further embodiments, excess biomass formation is minimized and production of MEG and isobutene is maximized.

### Monoethylene Glycol (MEG)

Monoethylene glycol (MEG) is an important raw material for industrial applications. A primary use of MEG is in the manufacture of polyethylene terephthalate (PET) resins, films and fibers. In addition, MEG is important in the production of antifreezes, coolants, aircraft anti-icer and deicers and solvents. MEG is also known as ethane-1,2-diol.

Ethylene glycol is also used as a medium for convective heat transfer in, for example, automobiles and liquid cooled computers.

Because of its high boiling point and affinity for water, ethylene glycol is a useful desiccant. Ethylene glycol is widely used to inhibit the formation of natural gas clathrates (hydrates) in long multiphase pipelines that convey natural gas from remote gas fields to a gas processing facility. Ethylene glycol can be recovered from the natural gas and reused as an inhibitor after purification treatment that removes water and inorganic salts.

Minor uses of ethylene glycol include in the manufacture of capacitors, as a chemical intermediate in the manufacture of 1,4-dioxane, and as an additive to prevent corrosion in liquid cooling systems for personal computers. Ethylene glycol is also used in the manufacture of some vaccines; as a minor ingredient in shoe polish, inks and dyes; as a rot and fungal treatment for wood; and as a preservative for biological specimens.

### Isobutene

Isobutene (also known as isobutylene or 2-methylpropene) is a hydrocarbon of industrial significance. It is a four-carbon branched alkene (olefin), one of the four isomers of butylene (butene). At standard temperature and pressure it is a colorless flammable gas.

Isobutene is used as an intermediate in the production of a variety of products. It is reacted with methanol and ethanol in the manufacture of the gasoline oxygenates methyl tert-butyl ether (MTBE) and ethyl tert-butyl ether (ETBE), respectively. Alkylation with butane produces isooctane, another fuel additive. Isobutene is also used in the production of methacrolein. Polymerization of isobutene produces butyl rubber (polyisobutene). Antioxidants such as butylated hydroxytoluene (BHT) and butylated hydroxyanisole (BHA) are produced by Friedel-Crafts alkylation of phenols using isobutene.

Polymer and chemical grade isobutene is typically obtained by dehydrating tertiary butyl alcohol or catalytic dehydrogenation of isobutane. Gasoline oxygenates MTBE and ETBE are generally produced by reacting methanol or ethanol with isobutene contained in butene streams from olefin steam crackers or refineries. Isobutene is not isolated before the reaction as separating the ethers from the remaining butenes is simpler.

The condensation of key intermediates in the isobutene pathway have been identified for optimal isobutene production.

Acetoacetyl-CoA thiolase enzyme condensates two molecules of acetyl-CoA into one molecule of acetoacetyl-CoA. Although acetoacetyl-CoA thiolase has been employed on synthetic metabolic pathways to favor acetoacetyl-CoA formation from the key intermediate acetyl-CoA, the enzyme prefers acetoacetyl-CoA thiolysis to acetoacetyl-CoA synthesis. An alternative route to overcome this reversibility issue would be the use of an acetoacetyl-CoA synthase enzyme, codified from the gene nphT7 from Streptomyces sp., which synthesizes acetoacetyl-CoA via the condensation of acetyl-CoA and malonyl-CoA, through an energy-favored reaction. Both acetyl-CoA and malonyl-CoA intracellular concentrations would have to be sufficiently available to favor their condensation via acetoacetyl-CoA synthase activity. While acetyl-CoA is a major intermediate of the E. coli central metabolism, malonyl-CoA intracellular concentration may be eventually increased by overexpressing a native acetyl-CoA carboxylase that catalysis the carboxylation of acetyl-CoA to malonyl-CoA. As previously described the acetyl-CoA carboxylase is an enzymatic complex composed by four subunits, E. coli genes named accABCD (carboxyltransferase, BCCP, biotin carboxylase, and carboxyltransferase, respectively). Through this alternative route, termed malonyl-CoA by-pass, increased intracellular concentration of acetoacetyl-CoA—key intermediate of isobutene pathways—can be achieved favoring the activity of the heterologous isobutene enzymes.

### Enzymes

Exemplary enzymes that may be used in the MEG and isobutene co-production pathways disclosed herein are listed in Table 1.

TABLE 1

| | | | | | | | | | |
|---|---|---|---|---|---|---|---|---|---|
| | | | Gene | | Natural/annotated | Gene Identifier | SEQ ID NO | | SEQ ID NO |
| Described Reaction | EC no. | Required enzyme activity | candidate | Source Organism | function | (nt) | (nt) | Uniprot ID | (AA) |

Isomerases that may be used in all xylulose dependent MEG pathways

| | | | | | | | | | |
|---|---|---|---|---|---|---|---|---|---|
| D-xylose + NAD(P)H <=> | 1.1.1.307 | xylose reductase | xyl1 | Scheffersomyces | D-xylose reductase | GeneID: | 82, 83 | P31867 | 84 |
| Xylitol + NAD(P)+ | | | | stipitis | | 4839234 | | | |
| D-xylose + NAD(P)H <=> | 1.1.1.307 | xylose reductase | GRE3 | Saccharomyces | aldose reductase | GeneID: | 85, 86 | P38715 | 87 |
| Xylitol + NAD(P)+ | | | | cerevisiae | | 856504 | | | |
| Xylitol + NAD + <=> D- | 1.1.1.9 | xylitol dehydrogenase | xyl2 | Scheffersomyces | D-xylulose reductase | GeneID: | 88, 89 | P22144 | 90 |
| xylulose + NADH | | | | stipitis | | 4852013 | | | |
| Xylitol + NAD + <=> D- | 1.1.1.9 | xylitol dehydrogenase | xdh1 | Trichoderma | Xylitol dehydrogenase | ENA Nr.: | 91 | Q876R2 | 92 |
| xylulose + NADH | | | | reesei | | AF428150.1 | | | |
| D-xylopyranose <=> D- | 5.3.1.5 | xylose isomerase | xylA | Pyromyces sp. | xylose isomerase | ENA Nr.: | 93, 94 | Q9P8C9 | 95 |
| xylulose | | | | | | CAB76571.1 | | | |

Glycolaldehyde reductases that may be used in all MEG pathways

| | | | | | | | | | |
|---|---|---|---|---|---|---|---|---|---|
| glycolaldehyde + | 1.1.1.— | glycolaldehyde reductase | gldA | Escherichia coli | glycerol dehydrogenase | GeneID: | 12 | P0A9S5 | 13 |
| NAD(P)H <=> | | | | | | 12933659 | | | |
| monoethylene glycol + | | | | | | | | | |
| NAD(P)+ | | | | | | | | | |
| glycolaldehyde + | 1.1.1.— | glycolaldehyde reductase | GRE2 | Saccharomyces | methylglyoxal reductase | GeneID: | 14 | Q12068 | 15 |
| NAD(P)H <=> | | | | cerevisiae | | 854014 | | | |
| monoethylene glycol + | | | | | | | | | |
| NAD(P)+ | | | | | | | | | |
| glycolaldehyde + | 1.1.1.— | glycolaldehyde reductase | GRE3 | Saccharomyces | aldose reductase | GeneID: | 16 | P38715 | 17 |
| NAD(P)H <=> | | | | cerevisiae | | 856504 | | | |
| monoethylene glycol + | | | | | | | | | |
| NAD(P)+ | | | | | | | | | |
| glycolaldehyde + | 1.1.1.— | glycolaldehyde reductase | yqhD* | Escherichia coli | Alcohol dehydrogenase | GeneID: | 18, 19 | Modified | 20 |
| NAD(P)H <=> | | | | | | 947493 | | version of | |
| monoethylene glycol + | | | | | | | | Q46856; | |
| NAD(P)+ | | | | | | | | G149E | |
| glycolaldehyde + | 1.1.1.— | glycolaldehyde reductase | yqhD | Escherichia coli | Alcohol dehydrogenase | GeneID: | 21, 22 | Q46856 | 23 |
| NAD(P)H <=> | | | | | | 947493 | | | |
| monoethylene glycol + | | | | | | | | | |
| NAD(P)+ | | | | | | | | | |
| glycolaldehyde + | 1.1.1.— | glycolaldehyde reductase | ydjg | Escherichia coli | methylglyoxal reductase | GeneID: | 24 | P77256 | 25 |
| NAD(P)H <=> | | | | | | 12930149 | | | |
| monoethylene glycol + | | | | | | | | | |
| NAD(P)+ | | | | | | | | | |
| glycolaldehyde + | 1.1.1.— | glycolaldehyde reductase | fucO | Escherichia coli | lactaldehyde reductase | GeneID: | 26, 27 | P0A9S1 | 28 |
| NAD(P)H <=> | | | | | | 947273 | | | |
| monoethylene glycol + | | | | | | | | | |
| NAD(P)+ | | | | | | | | | |
| glycolaldehyde + | 1.1.1.— | glycolaldehyde reductase | yafB | Escherichia coli | methylglyoxal reductase | 545778205 | 29 | P30863 | 30 |
| NAD(P)H <=> | | | (dkgB) | | [multifunctional] | | | | |
| monoethylene glycol + | | | | | | | | | |
| NAD(P)+ | | | | | | | | | |
| glycolaldehyde + | 1.1.1.— | glycolaldehyde reductase | yqhE | Escherichia coli | 2,5-diketo-D-gluconic | GeneID: | 31 | Q46857 | 32 |
| NAD(P)H <=> | | | (dkgA) | | acid reductase A | 947495 | | | |
| monoethylene glycol + | | | | | | | | | |
| NAD(P)+ | | | | | | | | | |

Enzymes that may be used in D-ribulose-1-phosphate pathway to MEG

| | | | | | | | | | |
|---|---|---|---|---|---|---|---|---|---|
| D-xylulose <=> D- | 5.1.3.— | D-ribulose-3-epimerase | DTE | Pseudomonas | D-tagatose 3-epimerase | ENA Nr.: | 1, 2 | O50580 | 3 |
| ribulose | | | | cichorii | | BAA24429.1 | | | |
| D-xylulose <=> D- | 5.1.3.— | D-ribulose-3-epimerase | C1KKR1 | Rhodobacter | D-tagatose 3-epimerase | ENA Nr.: | 4 | C1KKR1 | 5 |
| ribulose | | | | sphaeroides | | FJ851309.1 | | | |
| D-ribulose + ATP <=> D- | 2.7.1.— | D-ribulose-1-kinase | fucK | Escherichia coli | L-fuculokinase | GeneID: | 6, 7 | P11553 | 8 |
| ribulose-1-phosphate + ADP | | | | | | 946022 | | | |
| D-ribulose-1-phosphate <=> | 4.1.2.— | D-ribulose-1-phosphate | fucA | Escherichia coli | L-fuculose phosphate | GeneID: | 9, 10 | P0AB87 | 11 |
| glyceraldehyde + | | aldolase | | | aldolase | 947282 | | | |
| dihydroxyacetonephosphate | | | | | | | | | |

Enzymes that may be used in D-xylulose-1-phosphate pathway to MEG

| | | | | | | | | | |
|---|---|---|---|---|---|---|---|---|---|
| D-xylulose + ATP <=> D- | 2.7.1.— | D-xylulose 1-kinase | khk-C | Homo sapiens | ketohexokinase C | GenBank: | 53, 54 | P50053 | 55 |
| xylulose-1-phosphate + ADP | | | (cDNA) | | | CR456801.1 | | | |
| D-xylulose-1-phosphate <=> | 4.1.2.— | D-xylulose-1-phosphate | aldoB | Homo sapiens | Fructose-bisphosphate | CCDS6756.1 | 56, 57 | P05062 | 58 |
| glyceraldehyde + | | aldolase | (cDNA) | | aldolase B | | | | |
| dihydroxyacetonephosphate | | | | | | | | | |

Enzymes that may be used in xylonate pathway to MEG

| | | | | | | | | | |
|---|---|---|---|---|---|---|---|---|---|
| D-xylose + NAD + <=> D- | 1.1.1.175 | xylose dehydrogenase | xylB | Caulobacter | D-xylose 1-dehydrogenase | GeneID: | 59, 60 | B8H1Z0 | 61 |
| xylonolactone + NADH, | | | | crescentus | | 7329904 | | | |
| or D-xylose + NAD + <=> | | | | | | | | | |
| D-xylonate + NADH | | | | | | | | | |
| D-xylose + NADP + <=> | 1.1.1.179 | xylose dehydrogenase | xdh1, | Haloferax volcanii | D-xylose 1-dehydrogenase | GeneID: | 62 | D4GP29 | 63 |
| D-xylonolactone + | | | HVO_B0028 | | | 8919161 | | | |
| NADPH, or D-xylose + | | | | | | | | | |
| NADP + <=> D- | | | | | | | | | |
| xylonate + NADPH | | | | | | | | | |
| D-xylose + NADP + <=> | 1.1.1.179 | xylose dehydrogenase | xyd1 | Trichoderma | D-xylose 1-dehydrogenase | ENA Nr.: | 64 | A0A024SMV2 | 65 |
| D-xylonolactone + | | | | reesei | | EF136590.1 | | | |
| NADPH, or D-xylose + | | | | | | | | | |
| NADP + <=> D- | | | | | | | | | |
| xylonate + NADPH | | | | | | | | | |
| D-xylonolactone + H2O <=> | 3.1.1.68 | xylonolactonase | xylC | Caulobacter | Xylonolactonase | GeneID: | 66 | A0A0H3C6P8 | 67 |
| D-xylonate | | | | crescentus | | 7329903 | | | |
| D-xylonate <=> 2-keto- | 4.2.1.82 | xylonate dehydratase | xylD | Caulobacter | xylonate dehydratase | GeneID: | 68 | A0A0H3C6H6 | 69 |
| 3-deoxy-xylonate + H2O | | | | crescentus | | 7329902 | | | |
| D-xylonate <=> 2-keto- | 4.2.1.82 | xylonate dehydratase | yjhG | Escherichia coli | xylonate dehydratase | GeneID: | 70, 71 | P39358 | 72 |
| 3-deoxy-xylonate + H2O | | | | | | 946829 | | | |
| D-xylonate <=> 2-keto- | 4.2.1.82 | xylonate dehydratase | yagF | Escherichia coli | xylonate dehydratase | GeneID: | 73, 74 | P77596 | 75 |
| 3-deoxy-xylonate + H2O | | | | | | 944928 | | | |
| 2-keto-3-deoxy- | 4.1.2.— | 2-keto-3-deoxy-D-pentonate | yjhH | Escherichia coli | Uncharacterized lyase | GeneID: | 76, 77 | P39359 | 78 |
| xylonate <=> | | aldolase | | | | 948825 | | | |
| glycolaldehyde + | | | | | | | | | |
| pyruvate | | | | | | | | | |
| 2-keto-3-deoxy- | 4.1.2.— | 2-keto-3-deoxy-D-pentonate | yagE | Escherichia coli | Probable 2-keto-3-deoxy- | GeneID: | 79, 80 | P75682 | 81 |
| xylonate <=> | | aldolase | | | galactonate aldolase | 944925 | | | |
| glycolaldehyde + | | | | | | | | | |
| pyruvate | | | | | | | | | |

Enzymes that may be used in acetone pathway to isobutene (pathway 4 of FIG. 4)

| | | | | | | | | | |
|---|---|---|---|---|---|---|---|---|---|
| 2 acetyl-Coa −> | 2.3.1.9 | acetyl coenzyme A | thlA | Clostridium | acetyl coenzyme A | 3309200 | 33, 34 | P45359 | 35 |
| acetoacetyl-CoA + CoA | | acetyltransferase | | acetobutylicum | acetyltransferase | | | | |
| 2 acetyl-Coa −> | 2.3.1.9 | acetyl coenzyme A | atoB | Escherichia coli | acetyl coenzyme A | GeneID: | 36 | P76461 | 37 |
| acetoacetyl-CoA + CoA | | acetyltransferase | | | acetyltransferase | 946727 | | | |
| 2 acetyl-Coa −> | 2.3.1.9 | acetyl coenzyme A | ERG10 | Saccharomyces | acetyl coenzyme A | 856079 | 38 | P41338 | 39 |
| acetoacetyl-CoA + CoA | | acetyltransferase | | cerevisiae | acetyltransferase | | | | |
| acetoacetyl-CoA + | 2.8.3.8 | Acetyl-CoA:acetoacetate- | atoA | Escherichia coli | Acetyl-CoA:acetoacetate- | 48994873 | 41, 42 | P76459 | 43 |
| acetate −> acetoacetate + | | CoA transferase subunit | | | CoA transferase subunit | | | | |
| acetyl-CoA | | | | | | | | | |
| acetoacetyl-CoA + | 2.8.3.8 | Acetyl-CoA:acetoacetate- | atoD | Escherichia coli | Acetyl-CoA:acetoacetate- | 48994873 | 44, 45 | P76458 | 46 |
| acetate −> acetoacetate + | | CoA transferase subunit | | | CoA transferase subunit | | | | |
| acetyl-CoA | | | | | | | | | |
| acetoacetate −> | 4.1.1.4 | acetoacetate decarboxylase | adc | Clostridium | acetoacetate decarboxylase | 6466901 | 47, 48 | P23670 | 49 |
| acetone + CO2 | | | | acetobutylicum | | | | | |
| acetoacetate −> | 4.1.1.4 | acetoacetate decarboxylase | adc | Clostridium | acetoacetate decarboxylase | 149901357 | 50, 51 | A6M020 | 52 |
| acetone + CO2 | | | | beijerinckii | | | | | |
| acetone + acetyl-CoA + | 2.3.3.— | 3-hydroxy-isovalerate | Hmgcs1 | Mus musculus | hydroxymethylglutaryl- | CCDS56901.1; | 104 | Q3UWQ9 | 105 |
| H2O <−> 3-hydroxy- | | synthase | | | CoA synthase | GeneID: | | | |
| isovalerate | | | | | | 208715 | | | |
| acetone + acetyl-CoA + | 2.3.3.— | 3-hydroxy-isovalerate | ERG13 | Saccharomyces | hydroxymethylglutaryl- | GeneID: | 106 | P54839 | 107 |
| H2O <−> 3-hydroxy- | | synthase | | cerevisiae | CoA synthase | 854913 | | | |
| isovalerate | | | | | | | | | |
| acetone + acetyl-CoA + | 2.3.3.— | 3-hydroxy-isovalerate | PksG | Lactobacillus | hydroxymethylglutaryl- | GeneID: | 108 | AEL95_01455 | 109 |
| H2O <−> 3-hydroxy- | | synthase | | crispatus ST1 | CoA synthase/polyketide | 9107446 | | | |
| isovalerate | | | | | intermediate transferase | | | | |
| acetone + acetyl-CoA + | 2.3.3.— | 3-hydroxy-isovalerate | Pnap_0477 | Polaromonas | hydroxymethylglutaryl- | ABM35799.1 | 110 | A1VJH1 | 111 |
| H2O <−> 3-hydroxy- | | synthase | | naphthalenivorans | CoA lyase | | | | |
| isovalerate | | | | | | | | | |
| 3-hydroxy-isovalerate + | 2.7.1.— | hydroxyisovalerate kinase | TA1305 | Thermoplasma | mevalonate-diphosphate | GeneID: | 112 | Q9HIN1 | 113 |
| ATP <−> ADP + H(+) + 3- | | | | acidophilum | decarboxylase/mevalonate- | 1456782 | | | |
| phosphonoxyisovalerate | | | | | monophosphate decarboxylase | | | | |
| 3-hydroxy-isovalerate + | 2.7.1.— | hydroxyisovalerate kinase | TA1305* | Thermoplasma | mevalonate-diphosphate | GeneID: | 114 | Modified | 115 |
| ATP <−> ADP + H(+) + 3- | | | (L200E) | acidophilum | decarboxylase/mevalonate- | 1456782 | | version of | |
| phosphonoxyisovalerate | | | | | monophosphate decarboxylase | | | Q9HIN1; | |
| | | | | | | | | L200E | |
| 3-hydroxy-isovalerate + | 2.7.1.— | hydroxyisovalerate kinase | PTO1356 | Picrophilus | mevalonate-diphosphate | GeneID: | 116 | Q6KZB1 | 117 |
| ATP <−> ADP + H(+) + 3- | | | | torridus | decarboxylase | 2845209 | | | |
| phosphonoxyisovalerate | | | | | | | | | |
| 3- | 4.1.1.— | 3- | smi_1746 | Streptococcus | mevalonate-diphosphate | Genbank: | 118 | D3HAT7 | 119 |
| phosphonoxyisovalerate −> | | phosphonoxyisovalerate | | mitis | decarboxylase | CBJ22986.1 | | | |
| CO(2) + isobutene | | decarboxylase | | | | | | | |
| 3- | 4.1.1.— | 3- | mvaD | Streptococcus | mevalonate-diphosphate | GeneID: | 120 | A8AUU9 | 121 |
| phosphonoxyisovalerate −> | | phosphonoxyisovalerate | | gordonii | decarboxylase | 25051665 | | | |
| CO(2) + isobutene | | decarboxylase | | | | | | | |
| 3-hydroxy-isovalerate −> | 4.1.1.— | hydroxyisovalerate | TA1305 | Thermoplasma | mevalonate-diphosphate | GeneID: | 112 | Q9HIN1 | 113 |
| CO(2) + isobutene | | decarboxylase | | acidophilum | decarboxylase | 1456782 | | | |
| 3-hydroxy-isovalerate −> | 4.1.1.— | hydroxyisovalerate | PTO1356 | Picrophilus | mevalonate-diphosphate | GeneID: | 116 | Q6KZB1 | 117 |
| CO(2) + isobutene | | decarboxylase | | torridus | decarboxylase | 2845209 | | | |
| 3-hydroxy-isovalerate −> | 4.1.1.— | hydroxyisovalerate | mvaD | Streptococcus | mevalonate-diphosphate | GeneID: | 120 | A8AUU9 | 121 |
| CO(2) + isobutene | | decarboxylase | | gordonii | decarboxylase | 25051665 | | | |

Hydrolases that may be used in improved acetone pathway to isobutene

| | | | | | | | | | |
|---|---|---|---|---|---|---|---|---|---|
| Acetoacetyl-CoA + | 3.1.2.11 | acetate:acetoacetyl-CoA | ctfA | Clostridium | butyrate-acetoacetate CoA- | NCBI- | 96 | P33752 | 97 |
| H(2)O <=> CoA + | | hydrolase | | acetobutylicum | transferase, complex A | GeneID: | | | |
| acetoacetate | | | | | | 1116168 | | | |
| Acetoacetyl-CoA + | 3.1.2.11 | acetate:acetoacetyl-CoA | ctfB | Clostridium | butyrate-acetoacetate CoA- | NCBI- | 98 | P23673 | 99 |
| H(2)O <=> CoA + | | hydrolase | | acetobutylicum | transferase, subunit B | GeneID: | | | |
| acetoacetate | | | | | | 1116169 | | | |
| Acetoacetyl-CoA + | 3.1.2.11 | acetate:acetoacetyl-CoA | atoA | Escherichia coli | Acetyl-CoA:acetoacetate- | GeneID: | 100 | P76459 | 101 |
| H(2)O <=> CoA + | | hydrolase | | (strain K12) | CoA transferase subunit | 946719 | | | |
| acetoacetate | | | | | | | | | |
| Acetoacetyl-CoA + | 3.1.2.11 | acetate:acetoacetyl-CoA | atoD | Escherichia coli | Acetyl-CoA:acetoacetate- | GeneID: | 102 | P76458 | 103 |
| H(2)O <=> CoA + | | hydrolase | | (strain K12) | CoA transferase subunit | 947525 | | | |
| acetoacetate | | | | | | | | | |

Enzymes that may be used in HMG-CoA pathway to isobutene (pathway 5 of FIG. 4)

| | | | | | | | | | |
|---|---|---|---|---|---|---|---|---|---|
| acetyl-CoA + H2O + | 2.3.3.10 | HMG-CoA synthase | hmgS | Saccharomyces | HMG-CoA synthase | GeneID: | 122 | P54839 | 123 |
| acetoacetyl-CoA <=> | | | | cerevisiae | | 854913 | | | |
| (S)-3-hydroxy-3- | | | | | | | | | |
| methylglutaryl-CoA + | | | | | | | | | |
| CoA | | | | | | | | | |
| (S)-3-hydroxy-3- | 4.2.1.18 | methylglutaconyl-CoA | liuC | Pseudomonas | methylglutaconyl-CoA | GeneID: | 124 | Q88FM3 | 125 |
| methylglutaryl-CoA <=> | | hydratase | | putida | hydratase | 1041856 | | | |
| trans-3- | | | | | | | | | |
| methylglutaconyl-CoA + | | | | | | | | | |
| H(2)O | | | | | | | | | |
| ADP + phosphate + 3- | 6.4.1.4. | methylcrotonyl-CoA | liuB | Pseudomonas | methylcrotonyl-CoA | GeneID: | 126 | Q9I297 | 127 |
| methylglutaconyl-CoA <=> | | carboxylase | | aeruginosa | carboxylase subunit beta | 878244 | | | |
| ATP + 3- | | | | | | | | | |
| methylcrotonoyl-CoA + | | | | | | | | | |
| HCO(3)(−) | | | | | | | | | |
| ADP + phosphate + 3- | 6.4.1.4. | methylcrotonyl-CoA | liuD | Pseudomonas | methylcrotonyl-CoA | GeneID: | 128 | Q9I299 | 129 |
| methylglutaconyl-CoA <=> | | carboxylase | | aeruginosa | carboxylase subunit alpha | 879012 | | | |
| ATP + 3- | | | | | | | | | |
| methylcrotonoyl-CoA + | | | | | | | | | |
| HCO(3)(−) | | | | | | | | | |
| trans-2(or 3)-enoyl-CoA + | 4.2.1.17 | methylcrotonyl-CoA | fadA | E. coli | fatty acid oxidation | GeneID: | 130 | P21151 | 131 |
| H(2)O <=> (3S)-3- | | hydratase | | | complex, 3-ketoacyl-CoA | 948324 | | | |
| hydroxyacyl-CoA | | | | | thiolase | | | | |
| trans-2(or 3)-enoyl-CoA + | 4.2.1.17 | methylcrotonyl-CoA | fadB | E. coli | fatty acid oxidation | GeneID: | 132 | P21177 | 133 |
| H(2)O <=> (3S)-3- | | hydratase | | | complex, enoyl-CoA | 948336 | | | |
| hydroxyacyl-CoA | | | | | hydratase | | | | |
| 3-hydroxy-isovaleryl- | 3.1.2.— | 3-hydroxy-isovaleryl-CoA | tesB | E. coli | acyl-CoA thioesterase | GeneID: | 134 | P0AGG2 | 135 |
| CoA + H2O <=> 3- | | thioesterase | | | | 945074 | | | |
| hydroxy-isovalerate + | | | | | | | | | |
| CoA | | | | | | | | | |

### D-Tagatose 3-Epimerase (EC 5.1.3.31)

The present disclosure describes enzymes that can catalyze the epimerization of various ketoses at the C-3 position, interconverting D-fructose and D-psicose, D-tagatose and D-sorbose, D-ribulose and D-xylulose, and L-ribulose and L-xylulose. The specificity depends on the species. The enzymes from Pseudomonas cichorii and Rhodobacter sphaeroides require Mn²⁺. In one embodiment, the enzyme is D-tagatose 3-epimerase (dte). In another embodiment, the D-tagatose 3-epimerase catalyzes the conversion of D-xylulose to D-ribulose.

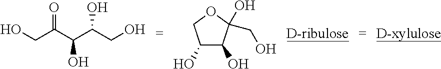 [SMILES: O=C(CO)[C@H](O)[C@H](O)CO ; OCC1(O)OC[C@@H](O)[C@@H]1O]

In one embodiment, the D-tagatose 3-epimerase is encoded by one or more nucleic acid molecules obtained from a microorganism selected from the group consisting of Pseudomonas sp., Mesorhizobium sp. and Rhodobacter sp. In some embodiments, the D-tagatose 3-epimerase is encoded by one or more nucleic acid molecules obtained from a microorganism selected from the group consisting of Pseudomonas cichorii, Pseudomonas sp. ST-24, Mesorhizobium loti and Rhodobacter sphaeroides. In some embodiments, the one or more nucleic acid molecules is dte and/or FJ851309.1, or homolog thereof. In a further embodiment, the D-tagatose 3-epimerase comprises an amino acid sequence selected from the group consisting of SEQ ID NOs: 3 and 5. In yet a further embodiment, the D-tagatose 3-epimerase is encoded by a nucleic acid sequence selected from the group consisting of SEQ ID NOs: 1, 2 and 4.

D-tagatose 3-epimerase may also be known as L-ribulose 3-epimerase or ketose 3-epimerase.

### D-Ribulokinase (EC 2.7.1.16)

The present disclosure describes enzymes that can catalyze the following reactions:

L-fuculose+ATP→L-fuculose 1-phosphate+ADP+H+

D-ribulose+ATP→D-ribulose 1-phosphate+ADP+H+

D-ribulokinase may also be known as L-fuculokinase, fuculokinase, ATP: L-fuculose 1-phosphotransferase or L-fuculose kinase.

Thus, in some embodiments, the disclosure provides for an enzyme that plays roles in the fucose degradation pathway, the super pathway of fucose and rhamnose degradation and/or the D-arabinose degradation I pathway.

In some embodiments, the enzyme can function as both an L-fucolokinase and a D-ribulokinase, the second enzyme of the L-fucose and D-arabinose degradation pathways, respectively.

In particular embodiments, the enzyme converts D-ribulose to D-ribulose-1-phosphate. In one embodiment, the D-ribulokinase is encoded by one or more nucleic acid molecules obtained from E. coli. In some embodiments, the one or more nucleic acid molecules is fucK, or homolog thereof. In a further embodiment, the D-ribulokinase comprises an amino acid sequence set forth in SEQ ID NO: 8. In yet a further embodiment, the D-ribulokinase is encoded by a nucleic acid sequence selected from the group consisting of SEQ ID NOs: 6 and 7.

### D-Ribulose-1-Phosphate Aldolase (EC 4.1.2.17)

The present disclosure describes enzymes that can catalyze the following reversible reactions:

L-fuculose 1-phosphate⇄(S)-lactaldehyde+dihydroxy acetone phosphate (DHAP)

D-ribulose 1-phosphate⇄glycolaldehyde+dihydroxy acetone phosphate (DHAP)

D-ribulose-1-phosphate aldolase may also be known as L-fuculose-phosphate aldolase, L-fuculose 1-phosphate aldolase or L-fuculose-1-phosphate (S)-lactaldehyde-lyase.

Thus, in some embodiments, the disclosure provides for an enzyme that plays roles in the fucose degradation pathway, the super pathway of fucose and rhamnose degradation and/or the D-arabinose degradation I pathway. In one embodiment, the enzyme may use Zn²⁺ as a cofactor. In another embodiment, an inhibitor of this enzyme may be phosphoglycolohydroxamate.

In some embodiments, the enzyme can function as both an L-fuculose-phosphate aldolase and a D-ribulose-phosphate aldolase, the third enzyme of the L-fucose and D-arabinose degradation pathways, respectively.

The substrate specificity of the enzyme has been tested with a partially purified preparation from an E. coli strain.

Crystal structures of the enzyme and a number of point mutants have been solved. The combination of structural data and enzymatic activity of mutants allowed modelling and refinement of the catalytic mechanism of the enzyme. The enantiomeric selectivity of the enzyme has been studied.

In particular embodiments, the enzyme converts D-ribulose-1-phosphate to glycolaldehyde and DHAP. In one embodiment, the D-ribulose-1-phosphate aldolase is encoded by one or more nucleic acid molecules obtained from E. coli. In some embodiments, the one or more nucleic acid molecules is fucA, or homolog thereof. In a further embodiment, the D-ribulose-1-phosphate aldolase comprises an amino acid sequence set forth in SEQ ID NO: 11. In yet a further embodiment, the D-ribulose-1-phosphate aldolase is encoded by a nucleic acid sequence selected from the group consisting of SEQ ID NOs: 9 and 10.

### Glycolaldehyde Reductase (EC 1.1.1.77)

The present disclosure describes enzymes that can catalyze the following reversible reactions:

ethylene glycol+NAD+⇄glycolaldehyde+NADH+H+

(S)-propane-1,2-diol+NAD+⇄(S)-lactaldehyde+NADH+H+

Glycolaldehyde reductase may also be known as lactaldehyde reductase, propanediol oxidoreductase, (R) [or(S)]-propane-1,2-diol:NAD+ oxidoreductase or L-1,2-propanediol oxidoreductase.

Thus, in some embodiments, the disclosure provides for an enzyme that plays roles in the ethylene glycol degradation pathway, the super pathway of glycol metabolism and degradation, the anaerobic L-lactaldehyde degradation pathway and/or the super pathway of fucose and rhamnose degradation. In one embodiment, the enzyme may use Fe²⁺ as a cofactor.

L-1,2-propanediol oxidoreductase is an iron-dependent group III dehydrogenase. It anaerobically reduces L-lactaldehyde, a product of both the L-fucose and L-rhamnose catabolic pathways, to L-1,2-propanediol, which is then excreted from the cell.

Crystal structures of the enzyme have been solved, showing a domain-swapped dimer in which the metal, cofactor and substrate binding sites could be located. An aspartate and three conserved histidine residues are required for Fe²⁺ binding and enzymatic activity.

In vitro, the enzyme can be reactivated by high concentrations of NAD+ and efficiently inactivated by a mixture of Fe³⁺ and ascorbate or Fe²⁺ and H₂O₂. Metal-catalyzed oxidation of the conserved His277 residue is proposed to be the cause of the inactivation.

Expression of FucO enables engineered one-turn reversal of the β-oxidation cycle. FucO activity contributes to the conversion of isobutyraldehyde to isobutanol in an engineered strain.

In particular embodiments, the enzyme converts glycolaldehyde to MEG. In some embodiments, the glycolaldehyde reductase is from Escherichia coli. In some embodiments, the glycolaldehyde reductase is encoded by the fucO gene.

In one embodiment, the glycolaldehyde reductase is encoded by one or more nucleic acid molecules obtained from a microorganism selected from E. coli and S. cerevisiae. In another embodiment, the one or more nucleic acid molecules is selected from gldA, GRE2, GRE3, yqhD, ydjG, fucO, yafB (dkgB), and/or yqhE (dkgA), or homolog thereof. In another embodiment, the one or more nucleic acid molecules is yqhD. In some embodiments, the yqhD comprises a G149E mutation. In a further embodiment, the glycolaldehyde reductase comprises an amino acid sequence selected from the group consisting of SEQ ID NOs: 13, 15, 17, 20, 23, 25, 28, 30 and 32. In yet a further embodiment, the glycolaldehyde reductase is encoded by a nucleic acid sequence selected from the group consisting of SEQ ID NOs: 12, 14, 16, 18, 19, 21, 22, 24, 26, 27, 29 and 31.

### Aldehyde Reductases

A number of aldehyde reductases may be used to convert glycolaldehyde to MEG.

An NADPH-dependent aldehyde reductase (YqhD) can catalyze the following reactions:

acetol+NADP+⇄methylglyoxal+NADPH+H+ (reversible, EC 1.1.1.-)

an alcohol+NADP+⇄an aldehyde+NADPH+H+ (reversibility unspecified, EC 1.1.1.2)

an aldehyde+NADP++H₂O→a carboxylate+NADPH+2H+ (EC 1.2.1.4)

1,3-propanediol+NADP+⇄3-hydroxypropionaldehyde+NADPH+H+ (reversibility unspecified, EC 1.1.1.-)

D-3,4-dihydroxybutanal+NADPH⇄1,3,4-butanetriol+NADP+ (reversibility unspecified)

YqhD is an NADPH-dependent aldehyde reductase that may be involved in glyoxal detoxification and/or be part of a glutathione-independent response to lipid peroxidation.

It has been reported that various alcohols, aldehydes, amino acids, sugars and α-hydroxy acids have been tested as substrates for YqhD. The purified protein only shows NADP-dependent alcohol dehydrogenase activity, with a preference for alcohols longer than C(3), but with Km values in the millimolar range, suggesting that they are not the physiological substrates. In contrast, YqhD does exhibit short-chain aldehyde reductase activity with substrates such as propanaldehyde, acetaldehyde, and butanaldehyde, as well as acrolein and malondialdehyde. In a metabolically engineered strain, phenylacetaldehyde and 4-hydroxyphenylacetaldehyde are reduced to 2-phenylethanol and 2-(4-hydroxyphenyl)ethanol by the endogenous aldehyde reductases YqhD, YjgB, and YahK.

Overexpression of YqhD increases 1,3-propanediol oxidoreductase activity of the cell. E. coli has been engineered to express YqhD for the industrial production of 1,3-propanediol. YqhD activity contributes to the production of isobutanol, 1,2-propanediol, 1,2,4-butanetriol and acetol as well. Mutation of yqhD enables production of butanol by an engineered one-turn reversal of the β-oxidation cycle.

YqhD has furfural reductase activity, which appears to cause growth inhibition due to depletion of NADPH in metabolically engineered strains that produce alcohol from lignocellulosic biomass.

The crystal structure of YqhD has been solved at 2 Å resolution. YqhD is an asymmetric dimer of dimers, and the active site contains a Zn²⁺ ion. The NADPH cofactor is modified by hydroxyl groups at positions 5 and 6 in the nicotinamide ring.

Overexpression of yqhD leads to increased resistance to reactive oxygen-generating compounds such as hydrogen peroxide, paraquat, chromate and potassium tellurite. A yqhD deletion mutant shows increased sensitivity to these compounds and to glyoxal, and contains increased levels of reactive aldehydes that are generated during lipid peroxidation. Conversely, yqhD deletion leads to increased furfural tolerance.

In particular embodiments, an NADPH-dependent aldehyde reductase converts glycolaldehyde to MEG. In some embodiments, the NADPH-dependent aldehyde reductase is from Escherichia coli. In some embodiments, the NADPH-dependent aldehyde reductase is encoded by the yqhD gene.

A multi-functional methylglyoxal reductase (DkgA) can catalyze the following reactions:

acetol+NADP+⇄methylglyoxal+NADPH+H+ (the reaction is physiologically favored in the opposite direction, EC 1.1.1.-)

isobutanol+NADP+⇄isobutanal+NADPH+H+ (reversibility unspecified, EC 1.1.1.-)

ethyl-(2R)-methyl-(3S)-hydroxybutanoate+NADP+⇄ethyl-2-methylacetoacetate+NADPH+H+ (reversibility unspecified, EC 1.1.1.-)

2-keto-L-gulonate+NADP+←2,5-didehydro-D-gluconate+NADPH+H+(the reaction is favored in the opposite direction, EC 1.1.1.346)

DkgA (YqhE) belongs to the aldo-keto reductase (AKR) family and has been shown to have methylglyoxal reductase and beta-keto ester reductase activity.

dkgA is reported to encode a 2,5-diketo-D-gluconate reductase (25DKGR) A, one of two 25DKG reductases in E. coli. The enzyme uses NADPH as the preferred electron donor and is thought to be involved in ketogluconate metabolism. The specific activity of the enzyme towards 2,5-diketo-D-gluconate is reported to be almost 1000-fold lower than its activity towards methylglyoxal.

Due to its low Km for NADPH, reduction of furans by DkgA may deplete NADPH pools and thereby limit cellular biosynthesis. A broad survey of aldehyde reductases showed that DkgA was one of several endogenous aldehyde reductases that contribute to the degradation of desired aldehyde end products of metabolic engineering.

A crystal structure of DkgA has been solved at 2.16 Å resolution.

In particular embodiments, a multi-functional methylglyoxal reductase converts glycolaldehyde to MEG. In some embodiments, the multi-functional methylglyoxal reductase is from Escherichia coli. In some embodiments, the multi-functional methylglyoxal reductase is encoded by the dkgA gene.

A multi-functional methylglyoxal reductase (DkgB) can catalyze the following reactions:

acetol+NADP+⇄methylglyoxal+NADPH+H+ (the reaction is physiologically favored in the opposite direction, EC 1.1.1.-)

4-nitrobenzyl alcohol+NADP+⇄4-nitrobenzaldehyde+NADPH+H+(reversibility unspecified, EC 1.1.1.91)

2-keto-L-gulonate+NADP+←2,5-didehydro-D-gluconate+NADPH+H+ (the reaction is favored in the opposite direction, EC 1.1.1.346)

DkgB (YafB) is a member of the aldo-keto reductase (AKR) subfamily 3F. DkgB was shown to have 2,5-diketo-D-gluconate reductase, methylglyoxal reductase and 4-nitrobenzaldehyde reductase activities.

dkgB is reported to encode 2,5-diketo-D-gluconate reductase (25DKGR) B, one of two 25DKG reductases in E. coli. The enzyme uses NADPH as the preferred electron donor and is thought to be involved in ketogluconate metabolism. However, the specific activity of the enzyme towards 2,5-diketo-D-gluconate is reported to be almost 1000-fold lower than its activity towards methylglyoxal.

In particular embodiments, a multi-functional methylglyoxal reductase converts glycolaldehyde to MEG. In some embodiments, the multi-functional methylglyoxal reductase is from Escherichia coli. In some embodiments, the multi-functional methylglyoxal reductase is encoded by the dkgB gene.

A methylglyoxal reductase (YeaE) can catalyze the following reaction:

acetol+NADP+⇄methylglyoxal+NADPH+H+ (the reaction is physiologically favored in the opposite direction, EC 1.1.1.-)

YeaE has been shown to have methylglyoxal reductase activity.

The subunit structure of YeaE has not been determined, but its amino acid sequence similarity to the aldo-keto reductases DkgA (YqhE) and DkgB (YafB) suggests that it may be monomeric.

In particular embodiments, a methylglyoxal reductase converts glycolaldehyde to MEG. In some embodiments, the methylglyoxal reductase is from Escherichia coli. In some embodiments, the methylglyoxal reductase is encoded by the yeaE gene.

A L-glyceraldehyde 3-phosphate reductase (yghZ) can catalyze the following reactions:

L-glyceraldehyde 3-phosphate+NADPH+H+→sn-glycerol 3-phosphate+NADP+ (EC 1.1.1.-)

acetol+NADP+⇄methylglyoxal+NADPH+H+(the reaction is physiologically favored in the opposite direction, EC 1.1.1.-)

YghZ is an L-glyceraldehyde 3-phosphate (L-GAP) reductase. The enzyme is also able to detoxify methylglyoxal at a low rate. YghZ defines the AKR14 (aldo-keto reductase 14) protein family.

L-GAP is not a natural metabolite and is toxic to E. coli. L-GAP is a substrate of both the glycerol-3-phosphate and hexose phosphate transport systems of E. coli K-12. It has been postulated that the physiological role of YghZ is the detoxification of L-GAP, which may be formed by non-enzymatic racemization of GAP or by an unknown cellular process.

The crystal structure of the E. coli enzyme has been determined and is suggested to be a tetramer. However, others have found that the protein forms an octamer based on gel filtration and electron microscopy studies.

In particular embodiments, a L-glyceraldehyde 3-phosphate reductase converts glycolaldehyde to MEG. In some embodiments, the L-glyceraldehyde 3-phosphate reductase is from Escherichia coli. In some embodiments, the L-glyceraldehyde 3-phosphate reductase is encoded by the yghZ gene.

An L-1,2-propanediol dehydrogenase/glycerol dehydrogenase (GldA) can catalyze the following reactions:

(S)-propane-1,2-diol+NAD+⇄acetol+NADH+H+ (reversible reaction)

aminoacetone+NADH+H+→(R)-1-aminopropan-2-ol+NAD+ (EC 1.1.1.75)

glycerol+NAD+⇄dihydroxyacetone+NADH+H+ (reversible reaction, EC 1.1.1.6)

The physiological function of the GldA enzyme has long been unclear. The enzyme was independently isolated as a glycerol dehydrogenase and a D-1-amino-2-propanol:NAD+ oxidoreductase. At that time, D-1-amino-2-propanol was thought to be an intermediate for the biosynthesis of vitamin B12, and although E. coli is unable to synthesize vitamin B12 de novo, enzymes catalyzing the synthesis of this compound were sought. It was later found that GldA was responsible for both activities.

The primary in vivo role of GldA was recently proposed to be the removal of dihydroxyacetone by converting it to glycerol. However, a dual role in the fermentation of glycerol has also recently been established. Glycerol dissimilation in E. coli can be accomplished by two different pathways. The glycerol and glycerophosphodiester degradation pathway requires the presence of a terminal electron acceptor and utilizes an ATP-dependent kinase of the Glp system, which phosphorylates glycerol to glycerol-3-phosphate. However, upon inactivation of the kinase and selection for growth on glycerol, it was found that an NAD+-linked dehydrogenase, GldA, was able to support glycerol fermentation. Recently, it was shown that GldA was involved in glycerol fermentation both as a glycerol dehydrogenase, producing dihydroxyacetone, and as a 1,2-propanediol dehydrogenase, regenerating NAD+ by producing 1,2-propanediol from acetol.

The enzyme is found in two catalytically active forms, a large form of eight subunits and a small form of two subunits. The large form appears to be the major species.

In particular embodiments, an L-1,2-propanediol dehydrogenase/glycerol dehydrogenase converts glycolaldehyde to MEG. In some embodiments, the L-1,2-propanediol dehydrogenase/glycerol dehydrogenase is from Escherichia coli. In some embodiments, the L-1,2-propanediol dehydrogenase/glycerol dehydrogenase is encoded by the gldA gene.

An NADPH-dependent methylglyoxal reductase (GRE2) from Saccharomyces cerevisiae can catalyze the following reactions:

(S)-lactaldehyde+NADP+⇄methylglyoxal+NADPH

3-methylbutanol+NAD(P)⁺⇄3-methylbutanal+NAD(P)H

Gre2 is a versatile enzyme that catalyzes the stereoselective reduction of a broad range of substrates including aliphatic and aromatic ketones, diketones, as well as aldehydes, using NADPH as the cofactor.

The crystal structures of Gre2 from S. cerevisiae in an apo-form at 2.00 Å and NADPH-complexed form at 2.40 Å resolution have been solved. Gre2 forms a homodimer, each subunit of which contains an N-terminal Rossmann-fold domain and a variable C-terminal domain, which participates in substrate recognition. The induced fit upon binding to the cofactor NADPH makes the two domains shift toward each other, producing an interdomain cleft that better fits the substrate. Computational simulation combined with site-directed mutagenesis and enzymatic activity analysis enabled characterization of a potential substrate-binding pocket that determines the stringent substrate stereoselectivity for catalysis.

Gre2 catalyzes the irreversible reduction of the cytotoxic compound methylglyoxal (MG) to (S)-lactaldehyde as an alternative to detoxification of MG by glyoxalase I GLO1. MG is synthesized via a bypath of glycolysis from dihydroxyacetone phosphate and is believed to play a role in cell cycle regulation and stress adaptation. GRE2 also catalyzes the reduction of isovaleraldehyde to isoamylalcohol. The enzyme serves to suppress isoamylalcohol-induced filamentation by modulating the levels of isovaleraldehyde, the signal to which cells respond by filamentation. GRE2 is also involved in ergosterol metabolism.

In particular embodiments, an NADPH-dependent methylglyoxal reductase converts glycolaldehyde to MEG. In some embodiments, the NADPH-dependent methylglyoxal reductase is from S. cerevisiae. In some embodiments, the NADPH-dependent methylglyoxal reductase is encoded by the GRE2 gene.

### Thiolase/Acetyl Coenzyme a Acetyltransferase (EC 2.3.1.9)

The present disclosure describes enzymes that can catalyze the following reaction:

2 acetyl-CoA⇄acetoacetyl-CoA+coenzyme A (reversible reaction)

Thiolase/Acetyl coenzyme A acetyltransferase may also be known as acetyl-CoA-C-acetyltransferase, acetoacetyl-CoA thiolase, acetyl-CoA:acetyl-CoA C-acetyltransferase or thiolase II.

Thus, in some embodiments, the disclosure provides for an enzyme that plays a role in acetoacetate degradation (to acetyl CoA). In one embodiment, an inhibitor of this enzyme may be acetoacetyl-CoA.

In particular embodiments, the enzyme converts acetyl-CoA to acetoacetyl-CoA. In one embodiment, the thiolase or acetyl coenzyme A acetyltransferase is encoded by one or more nucleic acid molecules obtained from a microorganism selected from the group consisting of Clostridium sp., Bacillus sp., E. coli, Saccharomyces sp. and Marinobacter sp. In some embodiments, the thiolase or acetyl coenzyme A acetyltransferase is encoded by one or more nucleic acid molecules obtained from a microorganism selected from the group consisting of Clostridium acetobutylicum, Clostridium thermosaccharolyticum, Bacillus cereus, E. coli, Saccharomyces cerevisiae and Marinobacter hydrocarbonoclasticus. In some embodiments, the one or more nucleic acid molecules is thIA, atoB and/or ERG10, or homolog thereof. In a further embodiment, the thiolase or acetyl coenzyme A acetyltransferase comprises an amino acid sequence selected from the group consisting of SEQ ID NOs: 35, 37 and 40. In yet a further embodiment, the thiolase or acetyl coenzyme A acetyltransferase is encoded by a nucleic acid sequence selected from the group consisting of SEQ ID NOs: 33, 34, 36, 38 and 39.

### Acetyl-CoA:Acetoacetate-CoA Transferase (EC 2.8.3.-)

The present disclosure describes enzymes that can catalyze the following reaction:

acetoacetate+acetyl-CoA acetoacetyl-CoA+acetate (reversible reaction, EC 2.8.3.-)

Acetyl-CoA:acetoacetate-CoA transferase may also be known as acetate:acetoacetyl-CoA transferase or acetoacetyl-CoA transferase.

Thus, in some embodiments, the disclosure provides for an enzyme that plays a role in acetoacetate degradation (to acetyl CoA). In one embodiment, inhibitors of this enzyme may include acetyl-CoA and coenzyme A.

The growth of E. coli on short-chain fatty acids (C3-C6) requires the activation of the acids to their respective thioesters. This activation is catalyzed by acetoacetyl-CoA transferase. The reaction takes place in two half-reactions which involves a covalent enzyme-CoA. The enzyme undergoes two detectable conformational changes during the reaction. It is thought likely that the reaction proceeds by a ping-pong mechanism. The enzyme can utilize a variety of short-chain acyl-CoA and carboxylic acid substrates but exhibits maximal activity with normal and 3-keto substrates.

In particular embodiments, the enzyme converts acetoacetyl-CoA to acetoacetate. In some embodiments, the acetyl-CoA:acetoacetate-CoA transferase is from Clostridium spp. In some embodiments, the acetyl-CoA:acetoacetate-CoA transferase is from Clostridium acetobutylicum. In some embodiments, the acetyl-CoA:acetoacetate-CoA transferase is from Escherichia coli. In some embodiments, the acetyl-CoA:acetoacetate-CoA transferase is encoded by the atoA and atoD genes. In another embodiment, the subunit composition of acetoacetyl-CoA transferase is [(AtoA)₂][(AtoD)₂], with (AtoA)₂ being the β complex and (AtoD)₂ being the α complex. In one embodiment, the acetyl-CoA:acetoacetate-CoA transferase is a fused acetyl-CoA:acetoacetate-CoA transferase: a subunit/6 subunit. In another embodiment, the acetyl-CoA:acetoacetate-CoA transferase is encoded by the ydiF gene.

Acetate:Acetoacetyl-CoA hydrolase (EC 3.1.2.11)

The present disclosure describes enzymes that can catalyze the following reaction:

acetoacetyl-CoA+H₂O [symbol] CoA+acetoacetate

Acetoacetyl-CoA hydrolase may also be known as acetoacetyl coenzyme A hydrolase, acetoacetyl CoA deacylase or acetoacetyl coenzyme A deacylase.

This enzyme belongs to the family of hydrolases, specifically those acting on thioester bonds.

In particular embodiments, the enzyme converts acetoacetyl-CoA to acetoacetate. In some embodiments, the acetate:acetoacetyl-CoA hydrolase is from Clostridium spp. In some embodiments, the acetate:acetoacetyl-CoA hydrolase is from Clostridium acetobutylicum. In another embodiment, the Acetoacetyl-CoA hydrolase is encoded by the ctfA (subunit A) and/or ctfB (subunit B) genes.

In a further embodiment, the acetyl-CoA:acetoacetate-CoA transferase or acetate:acetoacetyl-CoA hydrolase comprises an amino acid sequence selected from the group consisting of SEQ ID NOs: 43, 46, 97, 99, 101 and 103. In yet a further embodiment, the acetyl-CoA:acetoacetate-CoA transferase or acetate:acetoacetyl-CoA hydrolase is encoded by a nucleic acid sequence selected from the group consisting of SEQ ID NOs: 41, 42, 44, 45, 96, 98, 100 and 102.

Acetoacetate decarboxylase (EC 4.1.1.4)

The present disclosure describes enzymes that can catalyze the following reaction:

acetoacetate+H+→acetone+CO₂

Acetoacetate decarboxylase may also be known as ADC, AADC or acetoacetate carboxy-lyase.

Thus, in some embodiments, the disclosure provides for an enzyme that plays roles in isopropanol biosynthesis, pyruvate fermentation to acetone, the super pathway of Clostridium acetobutylicum acidogenic and solventogenic fermentation and/or the super pathway of Clostridium acetobutylicum solventogenic fermentation.

Acetoacetate decarboxylase (ADC) plays a key role in solvent production in Clostridium acetobutylicum. During the acidogenic phase of growth, acids accumulate causing a metabolic shift to solvent production. In this phase acids are re-assimilated and metabolized to produce acetone, butanol and ethanol.

Preliminary purification and crystallization of the enzyme has revealed that a lysine residue is implicated in the active site. The enzyme is a large complex composed of 12 copies of a single type of subunit.

The enzyme of Clostridium acetobutylicum ATCC 824 has been purified and the adc gene encoding it cloned. The enzyme has also been purified from the related strain Clostridium acetobutylicum DSM 792 and the gene cloned and sequenced. The decarboxylation reaction proceeds by the formation of a Schiff base intermediate.

ADC is a key enzyme in acid uptake, effectively pulling the CoA-transferase reaction in the direction of acetoacetate formation.

In particular embodiments, the enzyme converts acetoacetate to acetone. In one embodiment, the acetoacetate decarboxylase is encoded by one or more nucleic acid molecules obtained from a microorganism selected from the group consisting of Clostridium sp., Bacillus sp., Chromobacterium sp. and Pseudomonas sp. In another embodiment, the acetoacetate decarboxylase is encoded by one or more nucleic acid molecules obtained from a microorganism selected from the group consisting of Clostridium acetobutylicum, Clostridium beijerinckii, Clostridium cellulolyticum, Bacillus polymyxa, Chromobacterium violaceum and Pseudomonas putida. In some embodiments, the one or more nucleic acid molecules encoding the acetoacetate decarboxylase is adc, or homolog thereof. In a further embodiment, the acetoacetate decarboxylase comprises an amino acid sequence selected from the group consisting of SEQ ID NOs: 49 and 52. In yet another embodiment, the acetoacetate decarboxylase is encoded by a nucleic acid sequence selected from the group consisting of SEQ ID NOs: 47, 48, 50 and 51.

D-xylulose 1-kinase (EC 2.7.1.-)

The present disclosure describes enzymes that can catalyze the conversion of D-xylulose to D-xylulose-1-phosphate. In some embodiments, the conversion can be catalyzed by a human ketohexokinase C (khk-C), also known as fructokinase.

Ketohexokinase, or fructokinase, phosphorylates fructose to fructose-1-phosphate. The enzyme is involved in fructose metabolism, which is part of carbohydrate metabolism. It is found in the liver, intestine and kidney cortex.

In human liver, purified fructokinase, when coupled with aldolase, has been discovered to contribute to an alternative mechanism to produce oxalate from xylitol. In coupled sequence, fructokinase and aldolase produce glycolaldehyde, a precursor to oxalate, from D-xylulose via D-xylulose 1-phosphate.

In particular embodiments, the enzyme converts D-xylulose to D-xylulose-1-phosphate. In one embodiment, the D-xylulose 1-kinase is encoded by one or more nucleic acid molecules obtained from Homo sapiens. In some embodiments, the one or more nucleic acid molecules encoding the D-xylulose 1-kinase is ketohexokinase C (khk-C), or homolog thereof. In another embodiment, the one or more nucleic acid molecules encoding the D-xylulose 1-kinase comprises an amino acid sequence set forth in SEQ ID NO: 55. In a further embodiment, the one or more nucleic acid molecules encoding the D-xylulose 1-kinase is encoded by a nucleic acid sequence selected from the group consisting of SEQ ID NOs: 53 and 54.

D-xylulose-1-phosphate aldolase (EC 4.1.2.-)

The present disclosure describes enzymes that can catalyze the conversion of D-xylulose-1-phosphate to glycolaldehyde and DHAP. In some embodiments, the conversion can be catalyzed by a human aldolase B, which is also known as fructose-bisphosphate aldolase B or liver-type aldolase.

Aldolase B is one of three isoenzymes (A, B, and C) of the class I fructose 1,6-bisphosphate aldolase enzyme (EC 4.1.2.13), and plays a key role in both glycolysis and gluconeogenesis. The generic fructose 1,6-bisphosphate aldolase enzyme catalyzes the reversible cleavage of fructose 1,6-bisphosphate (FBP) into glyceraldehyde 3-phosphate and dihydroxyacetone phosphate (DHAP) as well as the reversible cleavage of fructose 1-phosphate (F1P) into glyceraldehyde and dihydroxyacetone phosphate. In mammals, aldolase B is preferentially expressed in the liver, while aldolase A is expressed in muscle and erythrocytes and aldolase C is expressed in the brain. Slight differences in isozyme structure result in different activities for the two substrate molecules: FBP and fructose 1-phosphate. Aldolase B exhibits no preference and thus catalyzes both reactions, while aldolases A and C prefer FBP.

Aldolase B is a homotetrameric enzyme, composed of four subunits. Each subunit has a molecular weight of 36 kDa and contains an eight-stranded a/1 barrel, which encloses lysine 229 (the Schiff-base forming amino acid that is key for catalysis).

In particular embodiments, the enzyme converts D-xylulose-1-phosphate to glycolaldehyde and DHAP. In one embodiment, the D-xylulose-1-phosphate aldolase is encoded by one or more nucleic acid molecules obtained from Homo sapiens. In another embodiment, the one or more nucleic acid molecules encoding the D-xylulose-1-phosphate aldolase is aldolase B (ALDOB), or homolog thereof. In some embodiments, the one or more nucleic acid molecules encoding the D-xylulose-1-phosphate aldolase comprises an amino acid sequence set forth in SEQ ID NO: 58. In some embodiments, the one or more nucleic acid molecules encoding the D-xylulose-1-phosphate aldolase is encoded by a nucleic acid sequence selected from the group consisting of SEQ ID NOs: 56 and 57.

D-xylose isomerase (EC 5.3.1.5)

The present disclosure describes enzymes that can catalyze the following reversible reaction:

D-xylopyranose⇄D-xylulose

D-xylose isomerase may also be known as xylose isomerase or D-xylose ketol-isomerase.

Thus, in some embodiments, the disclosure provides for an enzyme that plays a role in xylose degradation.

Xylose isomerase catalyzes the first reaction in the catabolism of D-xylose.

Two conserved histidine residues, H101 and H271, were shown to be essential for catalytic activity. The fluorescence of two conserved tryptophan residues, W49 and W188, is quenched during binding of xylose, and W49 was shown to be essential for catalytic activity. The presence of Mg²⁺, Mn²⁺ or Co²⁺ protects the enzyme from thermal denaturation.

The subunit composition has not been established experimentally.

In particular embodiments, the enzyme converts D-xylose to D-xylulose. In one embodiment, the recombinant microorganism further comprises an endogenous or exogenous xylose isomerase that catalyzes the conversion of D-xylose to D-xylulose. In one embodiment, the xylose isomerase is exogenous. In another embodiment, the xylose isomerase is encoded by one or more nucleic acid molecules obtained from Pyromyces sp. In another embodiment, the one or more nucleic acid molecules encoding the xylose isomerase is xylA, or homolog thereof. In yet another embodiment, the one or more nucleic acid molecules encoding the xylose isomerase comprises an amino acid sequence set forth in SEQ ID NO: 95. In a further embodiment, the one or more nucleic acid molecules encoding the xylose isomerase is encoded by a nucleic acid sequence selected from the group consisting of SEQ ID NOs: 93 and 94.

In some embodiments, a recombinant microorganism producing MEG and isobutene comprises a deletion, insertion, or loss of function mutation in a gene encoding a D-xylose isomerase to prevent conversion of D-xylose to D-xylulose and instead shunt the reaction toward the conversion of D-xylose to D-xylonate.

### D-Xylulose-5-Kinase/Xylulokinase

The present disclosure describes enzymes that can catalyze the following reactions:

D-xylulose+ATP→D-xylulose 5-phosphate+ADP+H+(EC 2.7.1.17)

ATP+1-deoxy-D-xylulose→1-deoxy-D-xylulose 5-phosphate+ADP+H+(EC 2.7.1.-)

D-xylulose-5-kinase may also be known as xylulose kinase or xylulokinase.

Xylulokinase catalyzes the phosphorylation of D-xylulose, the second step in the xylose degradation pathway, producing D-xylulose-5-phosphate, an intermediate of the pentose phosphate pathway.

In the absence of substrate, xylulokinase has weak ATPase activity. Xylulokinase can also catalyze the phosphorylation of 1-deoxy-D-xylulose. This would allow a potential salvage pathway for generating 1-deoxy-D-xylulose 5-phosphate for use in the biosynthesis of terpenoids, thiamine and pyridoxal. The rate of phosphorylation of 1-deoxy-D-xylulose is 32-fold lower than the rate of phosphorylation of D-xylulose.

The kinetic mechanism of the bacterial enzyme has been studied, suggesting a predominantly ordered reaction mechanism. The enzyme undergoes significant conformational changes upon binding of the substrate and of ATP. Two conserved aspartate residues, D6 and D233, were found to be essential for catalytic activity, and a catalytic mechanism has been proposed.

Crystal structures of bacterial xylulokinase in the apo form and bound to D-xylulose have been determined at 2.7 and 2.1 Å resolution, respectively.

In particular embodiments, the enzyme converts D-xylulose to D-xylulose-5-phosphate. In some embodiments, the D-xylulose-5-kinase is from Escherichia coli. In some embodiments, the D-xylulose-5-kinase is encoded by the xylB gene. In some embodiments, the D-xylulose-5-kinase is from Saccharomyces cerevisiae. In some embodiments the D-xylulose-5-kinase is encoded by the XKS1 gene. In some embodiments, the D-xylulose-5-kinase is from Pichia stipitis. In some embodiments the D-xylulose-5-kinase is encoded by the XYL3 gene.

In some embodiments, a recombinant microorganism producing MEG and isobutene comprises a deletion, insertion, or loss of function mutation in a gene encoding a D-xylulose-5-kinase to prevent the conversion of D-xylulose to D-xylulose-5-phosphate and instead shunt the reaction toward conversion of D-xylulose to D-xylulose-1-phosphate.

### Xylose Dehydrogenase (EC 1.1.1.175 or EC 1.1.1.179)

The present disclosure describes enzymes that can catalyze the following reactions:

aldehydo-D-xylose+NAD++H₂O→D-xylonate+NADH+2H+

α-D-xylopyranose+NAD+⇄D-xylonolactone+NADH+H+ (reversibility unspecified, EC 1.1.1.175)

Xylose dehydrogenase may also be known as D-xylose dehydrogenase, D-xylose 1-dehydrogenase, (NAD+)-linked D-xylose dehydrogenase, NAD+-D-xylose dehydrogenase, D-xylose: NAD+ 1-oxidoreductase

D-xylose dehydrogenase catalyzes the NAD+-dependent oxidation of D-xylose to D-xylonolactone. This is the first reaction in the oxidative, non-phosphorylative pathway for the degradation of D-xylose in Caulobacter crescentus. This pathway is similar to the pathway for L-arabinose degradation in Azospirillum brasilense. The amino acid sequence of the C. crescentus enzyme is unrelated to that of xylose dehydrogenase from the archaeon Haloarcula marismortui, or the L-arabinose 1-dehydrogenase of Azospirillum brasilense.

D-xylose is the preferred substrate for recombinant D-xylose dehydrogenase from Caulobacter crescentus. The enzyme can use L-arabinose, but it is a poorer substrate. The Km for L-arabinose is 166 mM. Other substrates such as D-arabinose, L-xylose, D-ribose, D-galactose, D-glucose and D-glucose-6-phosphate showed little or no activity in the assay, as measured by NADH production. C. crescentus D-xylose dehydrogenase can convert D-xylose to D-xylonate directly.

Partially purified, native D-xylose dehydrogenase from C. crescentus had a Km of 70 μM for D-xylose. This value was lower than the Km of 760 μM for the recombinant, His-tagged enzyme.

In some embodiments, the D-xylose dehydrogenase is from the halophilic archaeon Haloferax volcanii. The Haloferax volcanii D-xylose dehydrogenase catalyzes the first reaction in the oxidative xylose degradation pathway of the halophilic archaeon Haloferax volcanii. The H. volcanii D-xylose dehydrogenase shows 59% amino acid sequence identity to a functionally characterized xylose dehydrogenase from Haloarcula marismortui and 56% identity to an ortholog in Halorubrum lacusprofundi, but is only 11% identical to the bacterial NAD+-dependent xylose dehydrogenase from Caulobacter crescentus CB15.

In particular embodiments, the enzyme converts D-xylose to D-xylonolactone. In one embodiment, the xylose dehydrogenase is encoded by one or more nucleic acid molecules obtained from a microorganism selected from the group consisting of Caulobacter sp., Haloarcula sp., Haloferax sp., Halorubrum sp. and Trichoderma sp. In another embodiment, the xylose dehydrogenase is encoded by one or more nucleic acid molecules obtained from a microorganism selected from the group consisting of Caulobacter crescentus, Haloarcula marismortui, Haloferax volcanii, Halorubrum lacusprofundi and Trichoderma reesei. In some embodiments, the one or more nucleic acid molecules encoding the xylose dehydrogenase is selected from xylB, xdh1 (HVO_B0028) and/or xyd1, or homolog thereof. In a further embodiment, the one or more nucleic acid molecules encoding the xylose dehydrogenase comprises an amino acid sequence selected from the group consisting of SEQ ID NOs: 61, 63 and 65. In yet another embodiment, the one or more nucleic acid molecules encoding the xylose dehydrogenase is encoded by a nucleic acid sequence selected from the group consisting of SEQ ID NOs: 59, 60, 62 and 64.

### Xylonolactonase (3.1.1.68)

The present disclosure describes enzymes that can catalyze the following reaction:

D-xylono-1,4-lactone+H₂O [symbol] D-xylonate

This enzyme belongs to the family of hydrolases, specifically those acting on carboxylic ester bonds. This enzyme participates in pentose and glucuronate interconversions.

Xylonolactonase may also be known as D-xylonolactonase, xylono-1,4-lactonase, xylono-gamma-lactonase or D-xylono-1,4-lactone lactonohydrolase.

In particular embodiments, the enzyme converts D-xylonolactone to D-xylonate. In one embodiment, the xylonolactonase is encoded by one or more nucleic acid molecules obtained from a microorganism selected from Caulobacter sp. and Haloferax sp. In another embodiment, the xylonolactonase is encoded by one or more nucleic acid molecules obtained from a microorganism selected from the group consisting of Caulobacter crescentus, Haloferax volcanii and Haloferax gibbonsii. In some embodiments, the one or more nucleic acid molecules encoding the xylonolactonase is xylC, or homolog thereof. In a further embodiment, the one or more nucleic acid molecules encoding the xylonolactonase comprises an amino acid sequence set forth in SEQ ID NO: 67. In yet another embodiment, the one or more nucleic acid molecules encoding the xylonolactonase is encoded by a nucleic acid sequence set forth in SEQ ID NO: 66.

### Xylonate Dehydratase (EC 4.2.1.82)

The present disclosure describes enzymes that can catalyze the following reaction:

D-xylonate [symbol] 2-keto-3-deoxy-D-xylonate+H₂O

This enzyme belongs to the family of lyases, specifically the hydro-lyases, which cleave carbon-oxygen bonds. This enzyme participates in pentose and glucuronate interconversions.

Xylonate dehydratase may also be known as D-xylonate hydro-lyase, D-xylo-aldonate dehydratase or D-xylonate dehydratase.

In particular embodiments, the enzyme converts D-xylonate to 2-keto-3-deoxy-D-xylonate. In one embodiment, the xylonate dehydratase is encoded by one or more nucleic acid molecules obtained from a microorganism selected from the group consisting of Caulobacter sp., Sulfolobus sp. and E. coli. In another embodiment, the xylonate dehydratase is encoded by one or more nucleic acid molecules obtained from a microorganism selected from the group consisting of Caulobacter crescentus, Suffolobus soffataricus and E. coli. In some embodiments, the one or more nucleic acid molecules encoding the xylonate dehydratase is selected from xylD, yjhG and/or yagF, or homolog thereof. In a further embodiment, the one or more nucleic acid molecules encoding the xylonate dehydratase comprises an amino acid sequence selected from the group consisting of SEQ ID NOs: 69, 72 and 75. In yet another embodiment, the one or more nucleic acid molecules encoding the xylonate dehydratase is encoded by a nucleic acid sequence selected from the group consisting of SEQ ID NOs: 68, 70, 71, 73 and 74.

2-keto-3-deoxy-D-pentonate Aldolase (4.1.2.28)

The present disclosure describes enzymes that can catalyze the following reaction:

2-dehydro-3-deoxy-D-pentonate glycolaldehyde+pyruvate (reversibility unspecified)

This enzyme belongs to the family of lyases, specifically the aldehyde-lyases, which cleave carbon-carbon bonds. This enzyme participates in pentose and glucuronate interconversions.

2-keto-3-deoxy-D-pentonate aldolase may also be known as 2-dehydro-3-deoxy-D-pentonate glycolaldehyde-lyase (pyruvate-forming), 2-dehydro-3-deoxy-D-pentonate aldolase, 3-deoxy-D-pentulosonic acid aldolase, and 2-dehydro-3-deoxy-D-pentonate glycolaldehyde-lyase.

YjhH appears to be a 2-dehydro-3-deoxy-D-pentonate aldolase. Genetic evidence suggests that YagE may also function as a 2-dehydro-3-deoxy-D-pentonate aldolase. yagE is part of the prophage CP4-6.

A yjhH yagE double mutant cannot use D-xylonate as the sole source of carbon, and crude cell extracts do not contain 2-dehydro-3-deoxy-D-pentonate aldolase activity. Both phenotypes are complemented by providing yjhH on a plasmid.

ArcA appears to activate yjhH gene expression under anaerobiosis. Two putative ArcA binding sites were identified 211 and 597 bp upstream of this gene, but no promoter upstream of it has been identified.

The crystal structure of YagE suggests that the protein is a homotetramer. Co-crystal structures of YagE in the presence of pyruvate and 2-keto-3-deoxygalactonate have been solved.

In particular embodiments, the enzyme converts 2-keto-3-deoxy-xylonate to glycolaldehyde and pyruvate. In one embodiment, the 2-keto-3-deoxy-D-pentonate aldolase is encoded by one or more nucleic acid molecules obtained from a microorganism selected from Pseudomonas sp. and E. coli. In another embodiment, the 2-keto-3-deoxy-D-pentonate aldolase is encoded by one or more nucleic acid molecules obtained from E. coli. In some embodiments, the one or more nucleic acid molecules encoding the 2-keto-3-deoxy-D-pentonate aldolase is selected from yjhH and/or yagE, or homolog thereof. In a further embodiment, the one or more nucleic acid molecules encoding the 2-keto-3-deoxy-D-pentonate aldolase comprises an amino acid sequence selected from the group consisting of SEQ ID NOs: 78 and 81. In yet another embodiment, the one or more nucleic acid molecules encoding the 2-keto-3-deoxy-D-pentonate aldolase is encoded by a nucleic acid sequence selected from the group consisting of SEQ ID NOs: 76, 77, 79 and 80.

### Glycolaldehyde Dehydrogenase (1.2.1.21)

The present disclosure describes enzymes that can catalyze the following reaction:

glycolaldehyde+NAD⁺+H₂O [symbol] glycolate+NADH+2H⁺

This enzyme belongs to the family of oxidoreductases, specifically those acting on the aldehyde or oxo group of donor with NAD+ or NADP+ as acceptor. This enzyme participates in glyoxylate and dicarboxylate metabolism.

Glycolaldehyde dehydrogenase may also be known as glycolaldehyde:NAD+ oxidoreductase or glycol aldehyde dehydrogenase.

In E. coli aldehyde dehydrogenase A (AldA) is an enzyme of relatively broad substrate specificity for small α-hydroxyaldehyde substrates. It is thus utilized in several metabolic pathways.

L-fucose and L-rhamnose are metabolized through parallel pathways which converge after their corresponding aldolase reactions yielding the same products: dihydoxy-acetone phosphate and L-lactaldehyde. Aerobically, aldehyde dehydrogenase A oxidizes L-lactaldehyde to L-lactate.

In parallel pathways utilizing the same enzymes, D-arabinose and L-xylose can be metabolized to dihydoxy-acetone phosphate and glycolaldehyde, which is oxidized to glycolate by aldehyde dehydrogenase A.

Crystal structures of the enzyme alone and in ternary and binary complexes have been solved.

Aldehyde dehydrogenase A is only present under aerobic conditions and is most highly induced by the presence of fucose, rhamnose or glutamate. The enzyme is inhibited by NADH, which may act as a switch to shift from oxidation of lactaldehyde to its reduction by propanediol oxidoreductase. AldA is upregulated during short-term adaptation to glucose limitation.

Based on sequence similarity, AldA was predicted to be a succinate-semialdehyde dehydrogenase.

Regulation of aldA expression has been investigated. The gene is regulated by catabolite repression, repression under anaerobic conditions via ArcA, and induction by the carbon source.

In particular embodiments, the enzyme converts glycolaldehyde to glycolate. In some embodiments, the glycolaldehyde dehydrogenase is from Escherichia coli. In some embodiments, the glycolaldehyde dehydrogenase is encoded by the aldA gene.

In some embodiments, a recombinant microorganism producing MEG and isobutene comprises a deletion, insertion, or loss of function mutation in a gene encoding a glycolaldehyde dehydrogenase to prevent the production of glycolic acid from glycolaldehyde and instead shunt the reaction toward conversion of glycolaldehyde to MEG.

### Lactate Dehydrogenase (1.1.1.28)

The present disclosure describes enzymes that can catalyze the following reaction:

(R)-lactate+NAD+←pyruvate+NADH+H+

Lactate dehydrogenase (LDH) is an enzyme found in nearly all living cells such as in animals, plants and prokaryotes. LDH catalyzes the conversion of lactate to pyruvic acid and back, as it converts NADH to NAD+ and back. A dehydrogenase is an enzyme that transfers a hydride from one molecule to another.

LDH exist in four distinct enzyme classes. The most common one is NAD(P)-dependent L-lactate dehydrogenase. Other LDHs act on D-lactate and/or are dependent on cytochrome c: D-lactate dehydrogenase (cytochrome) and L-lactate dehydrogenase (cytochrome).

LDH has been of medical significance because it is found extensively in body tissues, such as blood cells and heart muscle. Because it is released during tissue damage, it is a marker of common injuries and disease such as heart failure.

Lactate dehydrogenase may also be known as lactic acid dehydrogenase, (R)-lactate:NAD+ oxidoreductase or D-lactate dehydrogenase-fermentative.

In E. coli, lactate dehydrogenase (LdhA) is a soluble NAD-linked lactate dehydrogenase (LDH) that is specific for the production of D-lactate. LdhA is a homotetramer and shows positive homotropic cooperativity under higher pH conditions.

E. coli contains two other lactate dehydrogenases: D-lactate dehydrogenase and L-lactate dehydrogenase. Both are membrane-associated flavoproteins required for aerobic growth on lactate.

LdhA is present under aerobic conditions but is induced when E. coli is grown on a variety of sugars under anaerobic conditions at acidic pH. Unlike most of the genes involved in anaerobic respiration, IdhA is not activated by Fnr; rather the ArcAB system and several genes involved in the control of carbohydrate metabolism (csrAB and mlc) appear to regulate expression. The expression of IdhA is negatively affected by the transcriptional regulator ArcA. IdhA belongs to the σ32 regulon.

The IdhA gene is a frequent target for mutations in metabolic engineering, most often to eliminate production of undesirable fermentation side products, but also to specifically produce D-lactate.

In particular embodiments, the enzyme converts pyruvate to lactate. In some embodiments, the lactate dehydrogenase is from Escherichia coli. In some embodiments, the lactate dehydrogenase is encoded by the IdhA gene.

In some embodiments, a recombinant microorganism producing MEG and isobutene comprises a deletion, insertion, or loss of function mutation in a gene encoding a lactate dehydrogenase to prevent the production of lactate from pyruvate and instead shunt the reaction toward production of isobutene.

### Xylose Reductase or Aldose Reductase (EC 1.1.1.21)

The present disclosure describes enzymes that can catalyze the following reactions:

α-D-xylose+NADPH+H+ [symbol] xylitol+NADP

an alditol+NAD(P)+ [symbol] NAD(P)H+aldose

Aldose reductase may also be known as alditol:NAD(P)+1-oxidoreductase, polyol dehydrogenase or aldehyde reductase.

Aldose reductase is a cytosolic oxidoreductase that catalyzes the reduction of a variety of aldehydes and carbonyls, including monosaccharides.

Aldose reductase may be considered a prototypical enzyme of the aldo-keto reductase enzyme superfamily. The enzyme comprises 315 amino acid residues and folds into a β/α-barrel structural motif composed of eight parallel 13 strands. Adjacent strands are connected by eight peripheral α-helical segments running anti-parallel to the β sheet. The catalytic active site is situated in the barrel core. The NADPH cofactor is situated at the top of the β/α barrel, with the nicotinamide ring projecting down in the center of the barrel and pyrophosphate straddling the barrel lip.

The reaction mechanism of aldose reductase in the direction of aldehyde reduction follows a sequential ordered path where NADPH binds, followed by the substrate. Binding of NADPH induces a conformational change (Enzyme⋅NADPH→Enzyme*⋅NADPH) that involves hinge-like movement of a surface loop (residues 213-217) so as to cover a portion of the NADPH in a manner similar to that of a safety belt. The alcohol product is formed via a transfer of the pro-R hydride of NADPH to the face of the substrate's carbonyl carbon. Following release of the alcohol product, another conformational change occurs (E*⋅NAD(P)+→E⋅NAD(P)+) in order to release NADP+. Kinetic studies have shown that reorientation of this loop to permit release of NADP+ appears to represent the rate-limiting step in the direction of aldehyde reduction. As the rate of coenzyme release limits the catalytic rate, it can be seen that perturbation of interactions that stabilize coenzyme binding can have dramatic effects on the maximum velocity (Vmax).

D-xylose-fermenting Pichia stipitis and Candida shehatae were shown to produce one single aldose reductase (ALR) that is active both with NADPH and NADH. Other yeasts such as Pachysolen tannophilus and C. tropicalis synthesize multiple forms of ALR with different coenzyme specificities. The significant dual coenzyme specificity distinguishes the P. stipitis and the C. shehatae enzymes from most other ALRs so far isolated from mammalian or microbial sources. The yeast Candida tenuis CBS 4435 produces comparable NADH- and NADPH-linked aldehyde-reducing activities during growth on D-xylose.

In particular embodiments, the enzyme converts D-xylose to xylitol. In some embodiments, the xylose reductase or aldose reductase is encoded by one or more nucleic acid molecules obtained from a microorganism selected from the group consisting of Hypocrea sp., Scheffersomyces sp., Saccharomyces sp., Pachysolen sp., Pichia sp., Candida sp., Aspergillus sp., Neurospora sp., and Cryptococcus sp. In some embodiments, the xylose reductase or aldose reductase is encoded by one or more nucleic acid molecules obtained from a microorganism selected from the group consisting of Hypocrea jecorina, Scheffersomyces stipitis, Saccharomyces cerevisiae, Pachysolen tannophilus, Pichia stipitis, Pichia quercuum, Candida shehatae, Candida tenuis, Candida tropicalis, Aspergillus niger, Neurospora crassa and Cryptococcus lactativorus. In another embodiment, the one or more nucleic acid molecules encoding the xylose reductase or aldose reductase is xyl1 and/or GRE3 or homolog thereof. In some embodiments, the one or more nucleic acid molecules encoding the xylose reductase or aldose reductase comprises an amino acid sequence selected from the group consisting of SEQ ID NOs: 84 and 87. In some embodiments, the one or more nucleic acid molecules encoding the xylose reductase or aldose reductase is encoded by a nucleic acid sequence selected from the group consisting of SEQ ID NOs: 82, 83, 85 and 86.

### Xylitol Dehydrogenase (1.1.1.9)

The present disclosure describes enzymes that can catalyze the following reaction:

xylitol+NAD+ [symbol] D-xylulose+NADH+H+

Xylitol dehydrogenase may also be known as D-xylulose reductase, NAD+-dependent xylitol dehydrogenase, erythritol dehydrogenase, 2,3-cis-polyol(DPN) dehydrogenase (C3-5), pentitol-DPN dehydrogenase, xylitol-2-dehydrogenase or xylitol: NAD+ 2-oxidoreductase (D-xylulose-forming).

Xylitol dehydrogenase (XDH) is one of several enzymes responsible for assimilating xylose into eukaryotic metabolism and is useful for fermentation of xylose contained in agricultural byproducts to produce ethanol. For efficient xylose utilization at high flux rates, cosubstrates should be recycled between the NAD+-specific XDH and the NADPH-preferring xylose reductase, another enzyme in the pathway.

In particular embodiments, the enzyme converts xylitol to D-xylulose. In one embodiment of any aspect disclosed above, the xylitol dehydrogenase is encoded by one or more nucleic acid molecules obtained from a microorganism selected from the group consisting of Scheffersomyces sp., Trichoderma sp., Pichia sp., Saccharomyces sp., Gluconobacter sp., Galactocandida sp., Neurospora sp., and Serratia sp. In another embodiment, the xylitol dehydrogenase is encoded by one or more nucleic acid molecules obtained from a microorganism selected from the group consisting of Scheffersomyces stipitis, Trichoderma reesei, Pichia stipitis, Saccharomyces cerevisiae, Gluconobacter oxydans, Galactocandida mastotermitis, Neurospora crassa and Serratia marcescens. In another embodiment, the one or more nucleic acid molecules encoding the xylitol dehydrogenase is xyl2 and/or xdh1, or homolog thereof. In some embodiments, the one or more nucleic acid molecules encoding the xylitol dehydrogenase comprises an amino acid sequence selected from the group consisting of SEQ ID NOs: 90 and 92. In some embodiments, the one or more nucleic acid molecules encoding the xylitol dehydrogenase is encoded by a nucleic acid sequence selected from the group consisting of SEQ ID NOs: 88, 89 and 91.

### Hydroxymethylglutaryl-CoA Synthase (EC 2.3.3.-)

The present disclosure describes enzymes that can catalyze the following reaction:

acetoacetyl-CoA+acetyl-CoA+H₂O↔(S)-3-hydroxy-3-methylglutaryl-CoA+coenzyme A+H⁺

Hydroxymethylglutaryl-CoA synthase may also be known as (S)-3-hydroxy-3-methylglutaryl-CoA acetoacetyl-CoA-lyase (CoA-acetylating), 3-hydroxy-3-methylglutaryl CoA synthetase, 3-hydroxy-3-methylglutaryl coenzyme A synthase, 3-hydroxy-3-methylglutaryl coenzyme A synthetase, 3-hydroxy-3-methylglutaryl-CoA synthase, 3-hydroxy-3-methylglutaryl-coenzyme A synthase, β-hydroxy-β-methylglutaryl-CoA synthase, HMG-CoA synthase, acetoacetyl coenzyme A transacetase, hydroxymethylglutaryl coenzyme A synthase, and hydroxymethylglutaryl coenzyme A-condensing enzyme.

Hydroxymethylglutaryl-CoA synthase catalyzes the condensation of acetyl-CoA with acetoacetyl-CoA to form (S)-3-hydroxy-3-methylglutaryl-CoA, an early stage in the synthesis of (R)-mevalonate, a precursor of cholesterol.

The enzyme catalyzes a complex reaction that can be divided into four steps. The first step involves the formation of an enzyme acetyl-CoA binary complex, followed by the transfer of the acetyl group from the CoA thioester to a cysteine residue on the enzyme, forming a thioester acyl-enzyme intermediate. In the next step the now reduced CoA dissociates, and the second substrate, acetoacetyl-CoA, binds the enzyme. The third step involves the formation of a carbanion by removal of a proton from the methyl of the acetylcysteine. The activated acetylcysteine then undergoes a Claisen-like condensation with the γ-carbon of the acetoacetyl-CoA ligand, which forms the HMG-CoA while retaining the thioester bond to the enzyme. The last step comprises the hydrolysis of this bond, resulting in free HMG-CoA.

The HMGCS1 gene from Homo sapiens has been cloned and sequenced (Russ A P et al. (1992) Amplification and direct sequencing of a cDNA encoding human cytosolic 3-hydroxy-3-methylglutaryl-coenzyme A synthase. Biochim Biophys Acta 1132(3): 329-31). The gene was expressed in Escherichia coli, and the recombinant protein was purified and characterized (Rokosz L L et al. (1994) Human cytoplasmic 3-hydroxy-3-methylglutaryl coenzyme A synthase: expression, purification, and characterization of recombinant wild-type and Cys129 mutant enzymes. Arch Biochem Biophys 312(1): 1-13). The enzyme is a homodimer of 120 kDa. Catalysis proceeds by formation of a covalent acetyl-enzyme intermediate. Kinetic data suggest that the two substrates (acetyl-CoA and acetoacetyl-CoA) compete for binding to the same site.

A variant of Hmgcs1 has been identified (Protein Q3UWQ9: hydroxymethylglutaryl-CoA synthase from Mus musculus), which comprises the four mutations T165P I222Q S296Q V500S.

In one embodiment, the hydroxymethylglutaryl-CoA synthase can have a 3-hydroxyisovalerate (3HIV) synthase activity and can catalyze the following reaction:

acetone+acetyl-CoA+H2O↔3-hydroxyisovalerate

In one embodiment, the 3HIV synthase is encoded by one or more nucleic acid molecules obtained from a microorganism selected from Mus sp., Saccharomyces sp., Lactobacillus sp. and Polaromonas sp. In another embodiment, the 3HIV synthase is encoded by one or more nucleic acid molecules obtained from a microorganism selected from Mus musculus, Saccharomyces cerevisiae, Lactobacillus crispatus and Polaromonas naphthalenivorans. In some embodiments, the one or more nucleic acid molecules encoding the 3HIV synthase is selected from Hmgcs1, ERG13, PksG and/or Pnap_0477, or homolog thereof. In a further embodiment, the one or more nucleic acid molecules encoding the 3HIV synthase comprises an amino acid sequence selected from the group consisting of SEQ ID NOs: 105, 107, 109 and 111. In yet another embodiment, the one or more nucleic acid molecules encoding the 3HIV synthase is encoded by a nucleic acid sequence selected from the group consisting of SEQ ID NOs: 104, 106, 108 and 110. In some embodiments, the one or more nucleic acid molecules encoding the hydroxymethylglutaryl-CoA synthase is hmgS, or homolog thereof. In a further embodiment, the one or more nucleic acid molecules encoding the hydroxymethylglutaryl-CoA synthase comprises an amino acid sequence set forth in SEQ ID NO: 123. In yet another embodiment, the one or more nucleic acid molecules encoding the hydroxymethylglutaryl-CoA synthase is encoded by a nucleic acid sequence set forth in SEQ ID NO: 122.

### Methylglutaconyl-CoA Hydratase (EC 4.2.1.18)

The present disclosure describes enzymes that can catalyze the following reaction:

(S)-3-hydroxy-3-methylglutaryl-CoA↔trans-3-methylglutaconyl-CoA+H₂O

This enzyme catalyzes the syn-hydration of 3-methylglutaconyl-CoA to (S)-3-hydroxy-3-methylglutaryl-CoA in the leucine degradation pathway. The bacterial enzyme has been characterized in Pseudomonas putida. It differs from the mammalian enzyme in having only one glutamyl residue in its active site rather than two, resulting in a different reaction mechanism. These enzymes are members of the crotonase superfamily (Wong B J and Gerlt J A (2004) Evolution of function in the crotonase superfamily: (3S)-methylglutaconyl-CoA hydratase from Pseudomonas putida. Biochemistry 43(16): 4646-4654) and reviewed in (Hamed R B et al. (2008) Mechanisms and structures of crotonase superfamily enzymes—how nature controls enolate and oxyanion reactivity. Cell Mol Life Sci 65(16): 2507-2527).

Recombinant enzyme was expressed in Escherichia coli, purified and characterized. The apparent molecular mass of the 10-His-tagged polypeptide was determined to be 32.251 kDa by ESI-MS. The 10-His-tag was subsequently removed before characterization of the enzyme (Wong and Gerlt 2004).

In one embodiment, the methylglutaconyl-CoA hydratase is encoded by one or more nucleic acid molecules obtained from Pseudomonas sp. In another embodiment, the methylglutaconyl-CoA hydratase is encoded by one or more nucleic acid molecules obtained from Pseudomonas putida. In some embodiments, the one or more nucleic acid molecules encoding the methylglutaconyl-CoA hydratase is liuC, or homolog thereof. In a further embodiment, the one or more nucleic acid molecules encoding the methylglutaconyl-CoA hydratase comprises an amino acid sequence set forth in SEQ ID NO: 125. In yet another embodiment, the one or more nucleic acid molecules encoding the methylglutaconyl-CoA hydratase is encoded by a nucleic acid sequence set forth in SEQ ID NO: 124.

### Methylcrotonyl-CoA Carboxylase (EC 6.4.1.4)

The present disclosure describes enzymes that can catalyze the following reaction:

ADP+phosphate+H⁺+3-methylglutaconyl-CoA↔ATP+3-methylcrotonoyl-CoA+HCO₃⁻

The enzyme activity is associated with the 3-methylcrotonyl-CoA carboxylase complex. This enzyme is a biotin-containing, biotin-dependent carboxylase involved in the L-leucine (and isovalerate) degradation pathway of Pseudomonas aeruginosa PAO1. This pathway is also the last phase of the acyclic terpene utilization pathway (citronellol degradation and cis-genanyl-CoA degradation pathways). The enzyme is not expressed in citronellol or citronellate grown cells, but is expressed in isovalerate grown cells. Genes liuB and liuD encode the two subunits of 3-methylcrotonyl-CoA carboxylase. The subunits are encoded in the liuRABCDE gene cluster of this organism (Hoschle B et al. (2005) Methylcrotonyl-CoA and geranyl-CoA carboxylases are involved in leucine/isovalerate utilization (Liu) and acyclic terpene utilization (Atu), and are encoded by liuB/liuD and atuC/atuF, in Pseudomonas aeruginosa. Microbiology 151(Pt 11): 3649-3656; Forster-Fromme K and Jendrossek D (2010). Catabolism of citronellol and related acyclic terpenoids in pseudomonads. Appl Microbiol Biotechnol 87(3): 859-869).

The enzyme was purified from cell extracts by avidin-affinity chromatography and the SDS-gel-isolated subunits were subjected to trypsin fingerprint analysis and ESI-MS which allowed identification of their corresponding genes (Hoschle et al. 2005).

The 3-methylcrotonyl-CoA carboxylase of Pseudomonas citronellolis was characterized in earlier work (Hector M L and Fall R R (1976) Multiple acyl-coenzyme A carboxylases in Pseudomonas citronellolis. Biochemistry 15(16): 3465-3472; Fall R R and Hector M L (1977) Acyl-coenzyme A carboxylases. Homologous 3-methylcrotonyl-CoA and geranyl-CoA carboxylases from Pseudomonas citronellolis. Biochemistry 16(18): 4000-4005; Fall R R (1981) 3-Methylcrotonyl-CoA and geranyl-CoA carboxylases from Pseudomonas citronellolis. Methods Enzymol 71 Pt C: 791-799).

In one embodiment, the methylcrotonyl-CoA carboxylase is encoded by one or more nucleic acid molecules obtained from Pseudomonas sp. In another embodiment, the methylcrotonyl-CoA carboxylase is encoded by one or more nucleic acid molecules obtained from Pseudomonas aeruginosa. In some embodiments, the one or more nucleic acid molecules encoding the methylcrotonyl-CoA carboxylase is selected from liuB and/or liuD, or homologs thereof. In a further embodiment, the one or more nucleic acid molecules encoding the methylcrotonyl-CoA carboxylase comprises an amino acid sequence selected from SEQ ID NOs: 127 and 129. In yet another embodiment, the one or more nucleic acid molecules encoding the methylcrotonyl-CoA carboxylase is encoded by a nucleic acid sequence selected from SEQ ID NOs: 126 and 128.

### Methylcrotonyl-CoA Hydratase (EC 4.2.1.17)

The present disclosure describes enzymes that can catalyze the following reaction:

trans-2(or 3)-enoyl-CoA+H₂O↔(3S)-3-hydroxyacyl-CoA

An exemplary enzyme is a 3-ketoacyl-CoA thiolase. It is involved in the degradation of fatty acids via the β-oxidation cycle. It has broad chain-length specificity for substrates although it exhibits its highest activity with medium-chain substrates. It is part of a multienzyme complex and is coded for by the fadA gene (Yang S Y et al (1990) Nucleotide sequence of the fadA gene. Primary structure of 3-ketoacyl-coenzyme A thiolase from Escherichia coli and the structural organization of the fadAB operon. J Biol Chem 265(18): 10424-10429; Binstock J F and Schulz H (1981) Fatty acid oxidation complex from Escherichia coli. Methods Enzymol 71 Pt C:403-11).

3-ketoacyl-CoA thiolase may also be known as acetyl-CoA C-acyltransferase, β-ketothiolase, acetyl-CoA acyltransferase and acyl-CoA:acetyl-CoA C-acyltransferase.

Another exemplary enzyme is an enoyl-CoA hydratase. The alpha subunit has four enzymatic activities associated with it. It is part of a multienzyme complex. Two of the activities, enoyl-CoA hydratase (EC 4.2.1.17) and 3-OH acyl-CoA epimerase (EC 5.1.2.3) are carried out by the same N terminal active site (Yang S Y and Elzinga M (1993) Association of both enoyl coenzyme A hydratase and 3-hydroxyacyl coenzyme A epimerase with an active site in the amino-terminal domain of the multifunctional fatty acid oxidation protein from Escherichia coli. J Biol Chem 268(9): 6588-6592).

In one embodiment, the methylcrotonyl-CoA hydratase is a 3-ketoacyl-CoA thiolase. In another embodiment, the methylcrotonyl-CoA hydratase is encoded by one or more nucleic acid molecules obtained from Escherichia coli. In some embodiments, the one or more nucleic acid molecules encoding the methylcrotonyl-CoA hydratase is fadA, or homolog thereof. In a further embodiment, the one or more nucleic acid molecules encoding the methylcrotonyl-CoA hydratase comprises an amino acid sequence set forth in SEQ ID NO: 131. In yet another embodiment, the one or more nucleic acid molecules encoding the methylcrotonyl-CoA hydratase is encoded by a nucleic acid sequence set forth in SEQ ID NO: 130.

In one embodiment, the methylcrotonyl-CoA hydratase is an enoyl-CoA hydratase. In another embodiment, the methylcrotonyl-CoA hydratase is encoded by one or more nucleic acid molecules obtained from Escherichia coli. In some embodiments, the one or more nucleic acid molecules encoding the methylcrotonyl-CoA hydratase is fadB, or homolog thereof. In a further embodiment, the one or more nucleic acid molecules encoding the methylcrotonyl-CoA hydratase comprises an amino acid sequence set forth in SEQ ID NO: 133. In yet another embodiment, the one or more nucleic acid molecules encoding the methylcrotonyl-CoA hydratase is encoded by a nucleic acid sequence set forth in SEQ ID NO: 132.

### 3-Hydroxy-Isovaleryl-CoA Thioesterase (EC 3.1.2.-)

The present disclosure describes enzymes that can catalyze the following reactions:

3-hydroxyisovaleryl-CoA+H₂O↔3-hydroxyisovalerate+CoA

an acyl-CoA+H₂O→a carboxylate+coenzyme A+H⁺

An exemplary acyl-CoA thioesterase is TesB. Thioesterase II (TesB) is one of a number of thioesterases present in E. coli. The enzyme has relatively broad substrate specificity, cleaving medium- and long-chain acyl-CoA substrates; the best tested substrate was 3,5-tetradecadienoyl-CoA (Nie L et al. (2008) A novel paradigm of fatty acid beta-oxidation exemplified by the thioesterase-dependent partial degradation of conjugated linoleic acid that fully supports growth of Escherichia coli. Biochemistry 47(36): 9618-9626). Thioesterase II is one of the thioesterases supporting growth on oleate or conjugated linoleic acid as the sole source of carbon (Nie et al. 2008).

A crystal structure of the enzyme has been solved at 1.9 Å resolution. The D204 residue was predicted to be in the active site; its importance was confirmed by kinetic analysis of mutants (Li J et al. (2000) Crystal structure of the Escherichia coli thioesterase II, a homolog of the human Nef binding enzyme. Nat Struct Biol 7(7): 555-559).

Strains either lacking or overproducing tesB have no obvious defect (Narasimhan M L et al. (1986) Genetic and biochemical characterization of an Escherichia coli K-12 mutant deficient in acyl-coenzyme A thioesterase II. J Bacteriol 165(3): 911-917; Naggert J et al. (1991) Cloning, sequencing, and characterization of Escherichia coli thioesterase II. J Biol Chem 266(17): 11044-11050). Overproduction of TesB relieves inhibition of fatty acid synthesis by long-chain acyl-ACP molecules that accumulate upon glycerol starvation (Jiang P and Cronan J E (1994) Inhibition of fatty acid synthesis in Escherichia coli in the absence of phospholipid synthesis and release of inhibition by thioesterase action. J Bacteriol 176(10): 2814-2821).

In one embodiment, the 3-hydroxy-isovaleryl-CoA thioesterase is encoded by one or more nucleic acid molecules obtained from E. coli. In some embodiments, the one or more nucleic acid molecules encoding the 3-hydroxy-isovaleryl-CoA thioesterase is tesB, or homolog thereof. In a further embodiment, the one or more nucleic acid molecules encoding the 3-hydroxy-isovaleryl-CoA thioesterase comprises an amino acid sequence set forth in SEQ ID NO: 135. In yet another embodiment, the one or more nucleic acid molecules encoding the 3-hydroxy-isovaleryl-CoA thioesterase is encoded by a nucleic acid sequence set forth in SEQ ID NO: 134.

Mevalonate-3-Kinase (EC 2.7.1.-)

The present disclosure describes enzymes that can catalyze the following reaction:

3-hydroxyisovalerate+ATP [symbol] ADP+H(+)+3-phosphonoxyisovalerate

(R)-mevalonate+ATP→(R)-mevalonate 3-phosphate+ADP+H+

Mevalonate-3-kinase may also be known as (R)-MVA 3-phosphotransferase or 3-hydroxyisovalerate (3HIV) kinase.

The subunit structure of this enzyme from Thermoplasma acidophilum has not been reported.

The mevalonate-3-kinase from the thermophilic archaeon Thermoplasma acidophilum is thought to participate in a variant of the mevalonate pathway found in archaea Azami Y et al. (2014) (R)-Mevalonate 3-Phosphate Is an Intermediate of the Mevalonate Pathway in Thermoplasma acidophilum. J Biol Chem 289(23): 15957-15967; Vinokur J M et al. (2014) Evidence of a Novel Mevalonate Pathway in Archaea. Biochemistry 53(25): 4161-4168).

Recombinant His-tagged enzyme was expressed in Escherichia coli, purified and characterized. Despite its homology with diphosphomevalonate decarboxylase, it showed no decarboxylase activity (Azami et al. 2014; Vinokur et al. 2014). The enzyme showed weak phosphomevalonate kinase activity, producing small amounts of (R)-mevalonate diphosphate (Azami et al. 2014). It had no mevalonate-5-kinase activity (Vinokur et al. 2014).

In one embodiment, the 3HIV kinase is encoded by one or more nucleic acid molecules obtained from a microorganism selected from the group consisting of Thermoplasma sp. and Picrophilus sp. In another embodiment, the 3HIV kinase is encoded by one or more nucleic acid molecules obtained from a microorganism selected from the group consisting of Thermoplasma acidophilum and Picrophilus torridus. In some embodiments, the one or more nucleic acid molecules encoding the 3HIV kinase is TA1305 and/or PTO1356, or homolog thereof. In some embodiments, the TA1305 comprises a L200E mutation. In a further embodiment, the one or more nucleic acid molecules encoding the 3HIV-kinase comprises an amino acid sequence selected from the group consisting of SEQ ID NOs: 113, 115 and 117. In yet another embodiment, the one or more nucleic acid molecules encoding the 3HIV kinase is encoded by a nucleic acid sequence selected from the group consisting of SEQ ID NOs: 112, 114 and 116.

### Mevalonate Diphosphate Decarboxylase (EC 4.1.1.-)

The present disclosure describes enzymes that can catalyze the following reactions:

(R)-mevalonate diphosphate+ATP→isopentenyl diphosphate+CO₂+ADP+phosphate

3-phosphonoxyisovalerate→CO₂+isobutene

3-hydroxyisovalerate→CO₂+isobutene

Mevalonate diphosphate decarboxylase may also be known as pyrophosphomevalonate decarboxylase, mevalonate-5-pyrophosphate decarboxylase, pyrophosphomevalonic acid decarboxylase, 5-pyrophosphomevalonate decarboxylase, mevalonate 5-diphosphate decarboxylase, and ATP:(R)-5-diphosphomevalonate carboxy-lyase (dehydrating), 3-phosphonoxyisovalerate decarboxylase, 3-hydroxyisovalerate-3-phosphate decarboxylase, 3HIV-3-phosphate decarboxylase, 3-hydroxyisovalerate decarboxylase and 3HIV decarboxylase.

This enzyme converts mevalonate 5-diphosphate (MVAPP) to isopentenyl diphosphate (IPP) through ATP dependent decarboxylation. The two substrates of this enzyme are ATP and mevalonate 5-diphosphate, whereas its four products are ADP, phosphate, isopentenyl diphosphate, and CO₂.

Mevalonate diphosphate decarboxylase catalyzes the final step in the mevalonate pathway. The mevalonate pathway is responsible for the biosynthesis of isoprenoids from acetate. This pathway plays a key role in multiple cellular processes by synthesizing sterol isoprenoids, such as cholesterol, and non-sterol isoprenoids, such as dolichol, heme A, tRNA isopentenyltransferase, and ubiquinone. This enzyme belongs to the family of lyases, specifically the carboxy-lyases, which cleave carbon-carbon bonds.

Mevalonate diphosphate decarboxylase recognizes and binds two substrates: ATP and mevalonate 5-diphosphate. After binding, the enzyme performs three types of reactions that can be separated into two main stages. First, phosphorylation occurs. This creates a reactive intermediate, which in the second stage undergoes concerted dephosphorylation and decarboxylation.

In one embodiment, the enzyme that catalyzes the reaction 3-phosphonoxyisovalerate→CO₂+isobutene is a 3HIV-3-phosphate decarboxylase. In another embodiment, the 3HIV-3-phosphate decarboxylase is encoded by one or more nucleic acid molecules obtained from Streptococcus sp. In some embodiments, the microorganism is selected from Streptococcus mitis and/or Streptococcus gordonii. In some embodiments, the one or more nucleic acid molecules encoding the 3HIV-3-phosphate decarboxylase comprises an amino acid sequence selected from SEQ ID NOs: 119 and 121. In further embodiments, the one or more nucleic acid molecules encoding the 3HIV-3-phosphate decarboxylase is encoded by a nucleic acid sequence selected from SEQ ID NOs: 118 and 120.

In one embodiment, the enzyme that catalyzes the reaction 3-hydroxyisovalerate→CO₂+isobutene is a 3HIVdecarboxylase. In another embodiment, the 3HIV decarboxylase is encoded by one or more nucleic acid molecules obtained from a microorganism selected from the group consisting of Streptococcus sp., Thermoplasma sp. and Picrophilus sp. In another embodiment, the 3HIV decarboxylase is encoded by one or more nucleic acid molecules obtained from a microorganism selected from the group consisting of Streptococcus gordonii, Thermoplasma acidophilum and Picrophilus torridus. In some embodiments, the one or more nucleic acid molecules encoding the 3HIV decarboxylase comprises mvaD, TA1305 and/or PTO1356, or homolog thereof. In a further embodiment, the one or more nucleic acid molecules encoding the 3HIV decarboxylase comprises an amino acid sequence selected from the group consisting of SEQ ID NOs: 113, 117 and 121. In yet another embodiment, the one or more nucleic acid molecules encoding the 3HIV decarboxylase is encoded by a nucleic acid sequence selected from the group consisting of SEQ ID NOs: 112, 116 and 120.

### Biosynthesis of MEG and Isobutene Using a Recombinant Microorganism

As discussed above, in one aspect, the present application provides a recombinant microorganism co-producing monoethylene glycol (MEG) and isobutene. In one embodiment, the MEG and isobutene are co-produced from xylose. In another embodiment, the recombinant microorganism comprises a deletion, insertion, or loss of function mutation in a gene encoding a D-xylulose-5-kinase and/or in a gene encoding a glycoaldehyde dehydrogenase. In some embodiments, the gene encoding the D-xylulose-5-kinase is xylB. In some embodiments, the gene encoding the glycoaldehyde dehydrogenase is aldA. In a further embodiment, isobutene is synthesized via the intermediate 3-hydroxyisovalerate.

In one embodiment, MEG is produced from xylose via ribulose-1-phosphate. In another embodiment, MEG is produced from xylose via xylulose-1-phosphate. In a further embodiment, MEG is produced from xylose via xylonate.

In one embodiment, isobutene is produced from DHAP or pyruvate via acetone. In another embodiment, isobutene is produced from DHAP or pyruvate via HMG-CoA.

In one preferred embodiment, MEG and isobutene are produced from xylose using a ribulose-1-phosphate pathway for the conversion of xylose to MEG and dihydroxyacetone-phosphate (DHAP), and using an acetone based pathway for the conversion of DHAP to isobutene.

In another preferred embodiment, MEG and isobutene are produced from xylose using a ribulose-1-phosphate pathway for the conversion of xylose to MEG and dihydroxyacetone-phosphate (DHAP), and using an HMG-CoA based pathway for the conversion of DHAP to isobutene.

As discussed above, in a second aspect, the present application relates to a recombinant microorganism capable of co-producing monoethylene glycol (MEG) and isobutene from exogenous D-xylose, wherein the recombinant microorganism expresses one or more of the following from (a) to (d):
(a) at least one endogenous or exogenous nucleic acid molecule encoding a D-tagatose 3-epimerase that catalyzes the conversion of D-xylulose to D-ribulose;
(b) at least one endogenous or exogenous nucleic acid molecule encoding a D-ribulokinase that catalyzes the conversion of D-ribulose from (a) to D-ribulose-1-phosphate,
(c) at least one endogenous or exogenous nucleic acid molecule encoding a D-ribulose-1-phosphate aldolase that catalyzes the conversion of D-ribulose-1-phosphate from (b) to glycolaldehyde and dihydroxyacetonephosphate (DHAP);
(d) at least one endogenous or exogenous nucleic acid molecule encoding a glycolaldehyde reductase that catalyzes the conversion of glycolaldehyde from (c) to MEG;
   wherein the recombinant microorganism further expresses one or more of the following from (e) to (h):
(e) at least one endogenous or exogenous nucleic acid molecule encoding a thiolase or acetyl coenzyme A acetyltransferase that catalyzes the conversion of acetyl-CoA to acetoacetyl-CoA;
(f) at least one endogenous or exogenous nucleic acid molecule encoding an acetyl-CoA:acetoacetate-CoA transferase or acetate:acetoacetyl-CoA hydrolase that catalyzes the conversion of acetoacetyl-CoA from (e) to acetoacetate;
(g) at least one endogenous or exogenous nucleic acid molecule encoding an acetoacetate decarboxylase that catalyzes the conversion of acetoacetate from (f) to acetone;
(h) at least one endogenous or exogenous nucleic acid molecule encoding a 3-hydroxyisovalerate synthase that catalyzes the conversion of acetone from (g) and acetyl-CoA to 3-hydroxyisovalerate (3HIV);
or
   wherein the recombinant microorganism expresses one or more of the nucleic acid molecule from (a) to (d) above and further expresses one or more of the following from (i) to (n):
(i) at least one endogenous or exogenous nucleic acid molecule encoding an acetyl coenzyme A acetyltransferase that catalyzes the conversion of acetyl-CoA to acetoacetyl-CoA;
(j) at least one endogenous or exogenous nucleic acid molecule encoding a hydroxymethylglutaryl-CoA synthase that catalyzes the conversion of acetoacetyl-CoA from (i) and acetyl-CoA to 3-hydroxy-3-methylglutaryl-CoA (HMG-CoA);
(k) at least one endogenous or exogenous nucleic acid molecule encoding a methylglutaconyl-CoA hydratase that catalyzes the conversion of HMG-CoA from (j) to 3-methylglutaconyl-CoA;
(l) at least one endogenous or exogenous nucleic acid molecule encoding a methylcrotonyl-CoA carboxylase that catalyzes the conversion of 3-methylglutaconyl-CoA from (k) to 3-methylcrotonyl-CoA;
(m) at least one endogenous or exogenous nucleic acid molecule encoding a methylcrotonyl-CoA hydratase that catalyzes the conversion of 3-methylcrotonyl-CoA from (l) to 3-hydroxyisovaleryl-CoA;
(n) at least one endogenous or exogenous nucleic acid molecule encoding a 3-hydroxyisovaleryl-CoA thioesterase that catalyzes the conversion of 3-hydroxyisovaleryl-CoA from (m) to 3HIV;
   wherein the recombinant microorganism further expresses (a1) and (a2), and/or (b1) selected from:
(a1) at least one endogenous or exogenous nucleic acid molecule encoding a 3HIV kinase that catalyzes the conversion of 3HIV from (h) or (n) to 3HIV-3-phosphate;
(a2) at least one endogenous or exogenous nucleic acid molecule encoding a 3HIV-3-phosphate decarboxylase that catalyzes the conversion of 3HIV-3-phosphate from (a1) to isobutene;
(b1) at least one endogenous or exogenous nucleic acid molecule encoding a 3HIV decarboxylase that catalyzes the conversion of 3HIV from (h) or (n) to isobutene;
   wherein the produced intermediate DHAP is converted to acetyl-CoA through the endogenous glycolysis pathway in the microorganism, and wherein MEG and isobutene are co-produced.

In one embodiment, the D-tagatose 3-epimerase is encoded by one or more nucleic acid molecules obtained from a microorganism selected from the group consisting of Pseudomonas sp., Mesorhizobium sp. and Rhodobacter sp. In some embodiments, the D-tagatose 3-epimerase is encoded by one or more nucleic acid molecules obtained from a microorganism selected from the group consisting of Pseudomonas cichorii, Pseudomonas sp. ST-24, Mesorhizobium loti and Rhodobacter sphaeroides. In some embodiments, the one or more nucleic acid molecules is dte and/or FJ851309.1, or homolog thereof. In a further embodiment, the D-tagatose 3-epimerase comprises an amino acid sequence selected from the group consisting of SEQ ID NOs: 3 and 5. In yet a further embodiment, the D-tagatose 3-epimerase is encoded by a nucleic acid sequence selected from the group consisting of SEQ ID NOs: 1, 2 and 4.

In one embodiment, the D-ribulokinase is encoded by one or more nucleic acid molecules obtained from E. coli. In some embodiments, the one or more nucleic acid molecules is fucK, or homolog thereof. In a further embodiment, the D-ribulokinase comprises an amino acid sequence set forth in SEQ ID NO: 8. In yet a further embodiment, the D-ribulokinase is encoded by a nucleic acid sequence selected from the group consisting of SEQ ID NOs: 6 and 7.

In one embodiment, the D-ribulose-1-phosphate aldolase is encoded by one or more nucleic acid molecules obtained from E. coli. In some embodiments, the one or more nucleic acid molecules is fucA, or homolog thereof. In a further embodiment, the D-ribulose-1-phosphate aldolase comprises an amino acid sequence set forth in SEQ ID NO: 11. In yet a further embodiment, the D-ribulose-1-phosphate aldolase is encoded by a nucleic acid sequence selected from the group consisting of SEQ ID NOs: 9 and 10.

In one preferred embodiment, MEG and isobutene are produced from xylose using a xylulose-1-phosphate pathway for the conversion of xylose to MEG and dihydroxyacetone-phosphate (DHAP), and using an acetone based pathway for the conversion of DHAP to isobutene.

In another preferred embodiment, MEG and isobutene are produced from xylose using a xylulose-1-phosphate pathway for the conversion of xylose to MEG and dihydroxyacetone-phosphate (DHAP), and using an HMG-CoA based pathway for the conversion of DHAP to isobutene.

As discussed above, in a third aspect, the present application relates to a recombinant microorganism capable of co-producing monoethylene glycol (MEG) and isobutene from exogenous D-xylose, wherein the recombinant microorganism expresses one or more of the following from (a) to (c):
(a) at least one endogenous or exogenous nucleic acid molecule encoding a D-xylulose 1-kinase that catalyzes the conversion of D-xylulose to D-xylulose-1-phosphate;
(b) at least one endogenous or exogenous nucleic acid molecule encoding a D-xylulose-1-phosphate aldolase that catalyzes the conversion of D-xylulose-1-phosphate from (a) to glycolaldehyde and dihydroxyacetonephosphate (DHAP);
(c) at least one endogenous or exogenous nucleic acid molecule encoding a glycolaldehyde reductase that catalyzes the conversion of glycolaldehyde from (b) to MEG;
   wherein the recombinant microorganism further expresses one or more of the following from (d) to (g):
(d) at least one endogenous or exogenous nucleic acid molecule encoding a thiolase or acetyl coenzyme A acetyltransferase that catalyzes the conversion of acetyl-CoA to acetoacetyl-CoA;
(e) at least one endogenous or exogenous nucleic acid molecule encoding an acetyl-CoA:acetoacetate-CoA transferase or acetate:acetoacetyl-CoA hydrolase that catalyzes the conversion of acetoacetyl-CoA from (d) to acetoacetate;
(f) at least one endogenous or exogenous nucleic acid molecule encoding an acetoacetate decarboxylase that catalyzes the conversion of acetoacetate from (e) to acetone;
(g) at least one endogenous or exogenous nucleic acid molecule encoding a 3-hydroxyisovalerate synthase that catalyzes the conversion of acetone from (f) and acetyl-CoA to 3-hydroxyisovalerate (3HIV);
or
   wherein the recombinant microorganism expresses one or more of the nucleic acid molecule from (a) to (c) above and further expresses one or more of the following from (h) to (m):
(h) at least one endogenous or exogenous nucleic acid molecule encoding an acetyl coenzyme A acetyltransferase that catalyzes the conversion of acetyl-CoA to acetoacetyl-CoA;
(i) at least one endogenous or exogenous nucleic acid molecule encoding a hydroxymethylglutaryl-CoA synthase that catalyzes the conversion of acetoacetyl-CoA from (h) and acetyl-CoA to 3-hydroxy-3-methylglutaryl-CoA (HMG-CoA);
(j) at least one endogenous or exogenous nucleic acid molecule encoding a methylglutaconyl-CoA hydratase that catalyzes the conversion of HMG-CoA from (i) to 3-methylglutaconyl-CoA;
(k) at least one endogenous or exogenous nucleic acid molecule encoding a methylcrotonyl-CoA carboxylase that catalyzes the conversion of 3-methylglutaconyl-CoA from (j) to 3-methylcrotonyl-CoA;
(l) at least one endogenous or exogenous nucleic acid molecule encoding a methylcrotonyl-CoA hydratase that catalyzes the conversion of 3-methylcrotonyl-CoA from (k) to 3-hydroxyisovaleryl-CoA;
(m) at least one endogenous or exogenous nucleic acid molecule encoding a 3-hydroxyisovaleryl-CoA thioesterase that catalyzes the conversion of 3-hydroxyisovaleryl-CoA from (l) to 3HIV; wherein the recombinant microorganism further expresses (a1) and (a2), and/or (b1) selected from:
(a1) at least one endogenous or exogenous nucleic acid molecule encoding a 3HIV kinase that catalyzes the conversion of 3HIV from (g) or (m) to 3HIV-3-phosphate;
(a2) at least one endogenous or exogenous nucleic acid molecule encoding a 3HIV-3-phosphate decarboxylase that catalyzes the conversion of 3HIV-3-phosphate from (a1) to isobutene;
(b1) at least one endogenous or exogenous nucleic acid molecule encoding a 3HIV decarboxylase that catalyzes the conversion of 3HIV from (g) or (m) to isobutene;
   and wherein the produced intermediate DHAP is converted to acetyl-CoA through the endogenous glycolysis pathway in the microorganism, and wherein MEG and isobutene are co-produced.

In one embodiment, the D-xylulose 1-kinase is encoded by one or more nucleic acid molecules obtained from Homo sapiens. In some embodiments, the one or more nucleic acid molecules encoding the D-xylulose 1-kinase is ketohexokinase C (khk-C), or homolog thereof. In another embodiment, the one or more nucleic acid molecules encoding the D-xylulose 1-kinase comprises an amino acid sequence set forth in SEQ ID NO: 55. In a further embodiment, the one or more nucleic acid molecules encoding the D-xylulose 1-kinase is encoded by a nucleic acid sequence selected from the group consisting of SEQ ID NOs: 53 and 54.

In one embodiment, the D-xylulose-1-phosphate aldolase is encoded by one or more nucleic acid molecules obtained from Homo sapiens. In another embodiment, the one or more nucleic acid molecules encoding the D-xylulose-1-phosphate aldolase is aldolase B (ALDOB), or homolog thereof. In some embodiments, the one or more nucleic acid molecules encoding the D-xylulose-1-phosphate aldolase comprises an amino acid sequence set forth in SEQ ID NO: 58. In some embodiments, the one or more nucleic acid molecules encoding the D-xylulose-1-phosphate aldolase is encoded by a nucleic acid sequence selected from the group consisting of SEQ ID NOs: 56 and 57.

In one embodiment of any aspect disclosed above, the recombinant microorganism further comprises one or more modifications selected from the group consisting of:
(a) a deletion, insertion, or loss of function mutation in a gene encoding a D-xylulose-5-kinase that catalyzes the conversion of D-xylulose to D-xylulose-5-phosphate;
(b) a deletion, insertion, or loss of function mutation in a gene encoding a glycolaldehyde dehydrogenase that catalyzes the conversion of glycolaldehyde to glycolic acid; and
(c) a deletion, insertion, or loss of function mutation in a gene encoding a lactate dehydrogenase that catalyzes the conversion of pyruvate to lactate.

In some embodiments of any aspect disclosed above, a recombinant microorganism producing MEG and isobutene comprises a deletion, insertion, or loss of function mutation in a gene encoding a D-xylulose-5-kinase to prevent the conversion of D-xylulose to D-xylulose-5-phosphate and instead shunt the reaction toward conversion of D-xylulose to D-xylulose-1-phosphate. In some embodiments, the D-xylulose-5-kinase is from Escherichia coli. In some embodiments, the D-xylulose-5-kinase is encoded by the xylB gene, or homolog thereof.

In some embodiments of any aspect disclosed above, a recombinant microorganism producing MEG and isobutene comprises a deletion, insertion, or loss of function mutation in a gene encoding a glycolaldehyde dehydrogenase to prevent the production of glycolic acid from glycolaldehyde and instead shunt the reaction toward conversion of glycolaldehyde to MEG. In some embodiments, the glycolaldehyde dehydrogenase is from Escherichia coli. In some embodiments, the glycolaldehyde dehydrogenase is encoded by the aldA gene, or homolog thereof.

In some embodiments of any aspect disclosed above, a recombinant microorganism producing MEG and isobutene comprises a deletion, insertion, or loss of function mutation in a gene encoding a lactate dehydrogenase to prevent the production of lactate from pyruvate and instead shunt the reaction toward production of isobutene. In some embodiments, the lactate dehydrogenase is from Escherichia coli. In some embodiments, the lactate dehydrogenase is encoded by the IdhA gene, or homolog thereof.

In one embodiment of any aspect disclosed above, the recombinant microorganism further comprises an endogenous or exogenous xylose isomerase that catalyzes the conversion of D-xylose to D-xylulose. In one embodiment, the xylose isomerase is exogenous. In another embodiment, the xylose isomerase is encoded by one or more nucleic acid molecules obtained from Pyromyces sp. In another embodiment, the one or more nucleic acid molecules encoding the xylose isomerase is xylA, or homolog thereof. In yet another embodiment, the one or more nucleic acid molecules encoding the xylose isomerase comprises an amino acid sequence set forth in SEQ ID NO: 95. In a further embodiment, the one or more nucleic acid molecules encoding the xylose isomerase is encoded by a nucleic acid sequence selected from the group consisting of SEQ ID NOs: 93 and 94.

In one embodiment of any aspect disclosed above, the recombinant microorganism further expresses at least one exogenous nucleic acid molecule encoding a xylose reductase or aldose reductase that catalyzes the conversion of D-xylose to xylitol and at least one exogenous nucleic acid molecule encoding a xylitol dehydrogenase that catalyzes the conversion of xylitol to D-xylulose.

In some embodiments of any aspect disclosed above, the xylose reductase or aldose reductase is encoded by one or more nucleic acid molecules obtained from a microorganism selected from the group consisting of Hypocrea sp., Scheffersomyces sp., Saccharomyces sp., Pachysolen sp., Pichia sp., Candida sp., Aspergillus sp., Neurospora sp., and Cryptococcus sp. In some embodiments, the xylose reductase or aldose reductase is encoded by one or more nucleic acid molecules obtained from a microorganism selected from the group consisting of Hypocrea jecorina, Scheffersomyces stipitis, Saccharomyces cerevisiae, Pachysolen tannophilus, Pichia stipitis, Pichia quercuum, Candida shehatae, Candida tenuis, Candida tropicalis, Aspergillus niger, Neurospora crassa and Cryptococcus lactativorus. In another embodiment, the one or more nucleic acid molecules encoding the xylose reductase or aldose reductase is xyl1 and/or GRE3 or homolog thereof. In some embodiments, the one or more nucleic acid molecules encoding the xylose reductase or aldose reductase comprises an amino acid sequence selected from the group consisting of SEQ ID NOs: 84 and 87. In some embodiments, the one or more nucleic acid molecules encoding the xylose reductase or aldose reductase is encoded by a nucleic acid sequence selected from the group consisting of SEQ ID NOs: 82, 83, 85 and 86.

In one embodiment of any aspect disclosed above, the xylitol dehydrogenase is encoded by one or more nucleic acid molecules obtained from a microorganism selected from the group consisting of Scheffersomyces sp., Trichoderma sp., Pichia sp., Saccharomyces sp., Gluconobacter sp., Galactocandida sp., Neurospora sp., and Serratia sp. In another embodiment, the xylitol dehydrogenase is encoded by one or more nucleic acid molecules obtained from a microorganism selected from the group consisting of Scheffersomyces stipitis, Trichoderma reesei, Pichia stipitis, Saccharomyces cerevisiae, Gluconobacter oxydans, Galactocandida mastotermitis, Neurospora crassa and Serratia marcescens. In another embodiment, the one or more nucleic acid molecules encoding the xylitol dehydrogenase is xyl2 and/or xdh1, or homolog thereof. In some embodiments, the one or more nucleic acid molecules encoding the xylitol dehydrogenase comprises an amino acid sequence selected from the group consisting of SEQ ID NOs: 90 and 92. In some embodiments, the one or more nucleic acid molecules encoding the xylitol dehydrogenase is encoded by a nucleic acid sequence selected from the group consisting of SEQ ID NOs: 88, 89 and 91.

In one preferred embodiment, MEG and isobutene are produced from xylose using a xylonate pathway for the conversion of xylose to MEG and pyruvate, and using an acetone based pathway for the conversion of pyruvate to isobutene.

In another preferred embodiment, MEG and isobutene are produced from xylose using a xylonate pathway for the conversion of xylose to MEG and pyruvate, and using an HMG-CoA based pathway for the conversion of pyruvate to isobutene.

As discussed above, in a fourth aspect, the present application relates to a recombinant microorganism capable of co-producing monoethylene glycol (MEG) and isobutene from exogenous D-xylose, wherein the recombinant microorganism expresses one or more of the following from (a) to (c):
(a) at least one endogenous or exogenous nucleic acid molecule encoding a xylose dehydrogenase that catalyzes the conversion of D-xylose to D-xylonolactone;
(b) at least one endogenous or exogenous nucleic acid molecule encoding a xylonolactonase that catalyzes the conversion of D-xylonolactone from (a) to D-xylonate;
(c) at least one endogenous or exogenous nucleic acid molecule encoding a xylose dehydrogenase that catalyzes the conversion of D-xylose to D-xylonate;
   wherein the recombinant microorganism further expresses one or more of the following from (d) to (f):
(d) at least one endogenous or exogenous nucleic acid molecule encoding a xylonate dehydratase that catalyzes the conversion of D-xylonate from (b) or (c) to 2-keto-3-deoxy-xylonate;
(e) at least one endogenous or exogenous nucleic acid molecule encoding a 2-keto-3-deoxy-D-pentonate aldolase that catalyzes the conversion of 2-keto-3-deoxy-xylonate from (d) to glycolaldehyde and pyruvate;
(f) at least one endogenous or exogenous nucleic acid molecule encoding a glycolaldehyde reductase that catalyzes the conversion of glycolaldehyde from (e) to MEG;
   wherein the recombinant microorganism further expresses one or more of the following from (g) to (j):
(g) at least one exogenous nucleic acid molecule encoding a thiolase or acetyl coenzyme A acetyltransferase that catalyzes the conversion of acetyl-CoA to acetoacetyl-CoA;
(h) at least one endogenous or exogenous nucleic acid molecule encoding an acetyl-CoA:acetoacetate-CoA transferase or acetate:acetoacetyl-CoA hydrolase that catalyzes the conversion of acetoacetyl-CoA from (g) to acetoacetate;
(i) at least one exogenous nucleic acid molecule encoding an acetoacetate decarboxylase that catalyzes the conversion of acetoacetate from (h) to acetone;
(j) at least one endogenous or exogenous nucleic acid molecule encoding a 3-hydroxyisovalerate synthase that catalyzes the conversion of acetone from (i) and acetyl-CoA to 3-hydroxy-isovalerate (3HIV);
or
   wherein the recombinant microorganism expresses one or more of the nucleic acid molecule from (a) to (c) above and one or more of the nucleic acid molecule from (d) to (f) above, and further expresses one or more of the following from (k) to (p):
(k) at least one endogenous or exogenous nucleic acid molecule encoding an acetyl coenzyme A acetyltransferase that catalyzes the conversion of acetyl-CoA to acetoacetyl-CoA;
(l) at least one endogenous or exogenous nucleic acid molecule encoding a hydroxymethylglutaryl-CoA synthase that catalyzes the conversion of acetoacetyl-CoA from (k) and acetyl-CoA to 3-hydroxy-3-methylglutaryl-CoA (HMG-CoA);
(m) at least one endogenous or exogenous nucleic acid molecule encoding a methylglutaconyl-CoA hydratase that catalyzes the conversion of HMG-CoA from (l) to 3-methylglutaconyl-CoA;
(n) at least one endogenous or exogenous nucleic acid molecule encoding a methylcrotonyl-CoA carboxylase that catalyzes the conversion of 3-methylglutaconyl-CoA from (m) to 3-methylcrotonyl-CoA;
(o) at least one endogenous or exogenous nucleic acid molecule encoding a methylcrotonyl-CoA hydratase that catalyzes the conversion of 3-methylcrotonyl-CoA from (n) to 3-hydroxyisovaleryl-CoA;
(p) at least one endogenous or exogenous nucleic acid molecule encoding a 3-hydroxyisovaleryl-CoA thioesterase that catalyzes the conversion of 3-hydroxyisovaleryl-CoA from (o) to 3HIV;
   wherein the recombinant microorganism further expresses (a1) and (a2), and/or (b1) selected from:
(a1) at least one endogenous or exogenous nucleic acid molecule encoding a 3HIV kinase that catalyzes the conversion of 3HIV from (j) or (p) to 3HIV-3-phosphate;
(a2) at least one endogenous or exogenous nucleic acid molecule encoding a 3HIV-3-phosphate decarboxylase that catalyzes the conversion of 3HIV-3-phosphate from (a1) to isobutene;
(b1) at least one endogenous or exogenous nucleic acid molecule encoding a 3HIV decarboxylase that catalyzes the conversion of 3HIV from (j) or (p) to isobutene;
   and wherein the produced intermediate pyruvate is converted to acetyl-CoA through the endogenous glycolysis pathway in the microorganism, and wherein MEG and isobutene are co-produced.

In one embodiment, the xylose dehydrogenase is encoded by one or more nucleic acid molecules obtained from a microorganism selected from the group consisting of Caulobacter sp., Haloarcula sp., Haloferax sp., Halorubrum sp. and Trichoderma sp. In another embodiment, the xylose dehydrogenase is encoded by one or more nucleic acid molecules obtained from a microorganism selected from the group consisting of Caulobacter crescentus, Haloarcula marismortui, Haloferax volcanii, Halorubrum lacusprofundi and Trichoderma reesei. In some embodiments, the one or more nucleic acid molecules encoding the xylose dehydrogenase is selected from xylB, xdh1 (HVO_B0028) and/or xyd1, or homolog thereof. In a further embodiment, the one or more nucleic acid molecules encoding the xylose dehydrogenase comprises an amino acid sequence selected from the group consisting of SEQ ID NOs: 61, 63 and 65. In yet another embodiment, the one or more nucleic acid molecules encoding the xylose dehydrogenase is encoded by a nucleic acid sequence selected from the group consisting of SEQ ID NOs: 59, 60, 62 and 64.

In one embodiment, the xylonolactonase is encoded by one or more nucleic acid molecules obtained from a microorganism selected from Caulobacter sp. and Haloferax sp. In another embodiment, the xylonolactonase is encoded by one or more nucleic acid molecules obtained from a microorganism selected from the group consisting of Caulobacter crescentus, Haloferax volcanii and Haloferax gibbonsii. In some embodiments, the one or more nucleic acid molecules encoding the xylonolactonase is xylC, or homolog thereof. In a further embodiment, the one or more nucleic acid molecules encoding the xylonolactonase comprises an amino acid sequence set forth in SEQ ID NO: 67. In yet another embodiment, the one or more nucleic acid molecules encoding the xylonolactonase is encoded by a nucleic acid sequence set forth in SEQ ID NO: 66.

In one embodiment, the xylonate dehydratase is encoded by one or more nucleic acid molecules obtained from a microorganism selected from the group consisting of Caulobacter sp., Sulfolobus sp. and E. coli. In another embodiment, the xylonate dehydratase is encoded by one or more nucleic acid molecules obtained from a microorganism selected from the group consisting of Caulobacter crescentus, Sulfolobus solfataricus and E. coli. In some embodiments, the one or more nucleic acid molecules encoding the xylonate dehydratase is selected from xylD, yjhG and/or yagF, or homolog thereof. In a further embodiment, the one or more nucleic acid molecules encoding the xylonate dehydratase comprises an amino acid sequence selected from the group consisting of SEQ ID NOs: 69, 72 and 75. In yet another embodiment, the one or more nucleic acid molecules encoding the xylonate dehydratase is encoded by a nucleic acid sequence selected from the group consisting of SEQ ID NOs: 68, 70, 71, 73 and 74.

In one embodiment, the 2-keto-3-deoxy-D-pentonate aldolase is encoded by one or more nucleic acid molecules obtained from a microorganism selected from Pseudomonas sp. and E. coli. In another embodiment, the 2-keto-3-deoxy-D-pentonate aldolase is encoded by one or more nucleic acid molecules obtained from E. coli. In some embodiments, the one or more nucleic acid molecules encoding the 2-keto-3-deoxy-D-pentonate aldolase is selected from yjhH and/or yagE, or homolog thereof. In a further embodiment, the one or more nucleic acid molecules encoding the 2-keto-3-deoxy-D-pentonate aldolase comprises an amino acid sequence selected from the group consisting of SEQ ID NOs: 78 and 81. In yet another embodiment, the one or more nucleic acid molecules encoding the 2-keto-3-deoxy-D-pentonate aldolase is encoded by a nucleic acid sequence selected from the group consisting of SEQ ID NOs: 76, 77, 79 and 80.

In one embodiment, the recombinant microorganism further comprises one or more modifications selected from the group consisting of:
(a) a deletion, insertion, or loss of function mutation in a gene encoding an enzyme that catalyzes the conversion of D-xylose to D-xylulose;
(b) a deletion, insertion, or loss of function mutation in a gene encoding an enzyme that catalyzes the conversion of glycolaldehyde to glycolic acid; and
(c) a deletion, insertion, or loss of function mutation in a gene encoding an enzyme that catalyzes the conversion of pyruvate to lactate.

In some embodiments, a recombinant microorganism producing MEG and isobutene comprises a deletion, insertion, or loss of function mutation in a gene encoding a D-xylose isomerase to prevent conversion of D-xylose to D-xylulose and instead shunt the reaction toward the conversion of D-xylose to D-xylonate. In one embodiment, the enzyme that catalyzes the conversion of D-xylose to D-xylulose is a D-xylose isomerase. In some embodiments, the D-xylose isomerase is from Escherichia coli. In some embodiments, the D-xylose isomerase is encoded by the xylA gene, or homolog thereof.

In some embodiments, a recombinant microorganism producing MEG and isobutene comprises a deletion, insertion, or loss of function mutation in a gene encoding a glycolaldehyde dehydrogenase to prevent the production of glycolic acid from glycolaldehyde and instead shunt the reaction toward conversion of glycolaldehyde to MEG. In one embodiment, the glycolaldehyde dehydrogenase is from Escherichia coli. In some embodiments, the glycolaldehyde dehydrogenase is encoded by the aldA gene, or homolog thereof.

In some embodiments, a recombinant microorganism producing MEG and isobutene comprises a deletion, insertion, or loss of function mutation in a gene encoding a lactate dehydrogenase to prevent the production of lactate from pyruvate and instead shunt the reaction toward production of isobutene. In one embodiment, the enzyme that catalyzes the conversion of pyruvate to lactate is a lactate dehydrogenase. In particular embodiments, the enzyme converts pyruvate to lactate. In some embodiments, the lactate dehydrogenase is from Escherichia coli. In some embodiments, the lactate dehydrogenase is encoded by the IdhA gene, or homolog thereof.

In one embodiment of any aspect disclosed above, the glycolaldehyde reductase is encoded by one or more nucleic acid molecules obtained from a microorganism selected from E. coli and S. cerevisiae. In another embodiment, the one or more nucleic acid molecules is selected from gldA, GRE2, GRE3, yqhD, ydjG, fucO, yafB (dkgB), and/or yqhE (dkgA), or homolog thereof. In another embodiment, the one or more nucleic acid molecules is yqhD. In some embodiments, the yqhD comprises a G149E mutation. In a further embodiment, the glycolaldehyde reductase comprises an amino acid sequence selected from the group consisting of SEQ ID NOs: 13, 15, 17, 20, 23, 25, 28, 30 and 32. In yet a further embodiment, the glycolaldehyde reductase is encoded by a nucleic acid sequence selected from the group consisting of SEQ ID NOs: 12, 14, 16, 18, 19, 21, 22, 24, 26, 27, 29 and 31.

In one embodiment of any aspect disclosed above, the thiolase or acetyl coenzyme A acetyltransferase is encoded by one or more nucleic acid molecules obtained from a microorganism selected from the group consisting of Clostridium sp., Bacillus sp., E. coli, Saccharomyces sp. and Marinobacter sp. In some embodiments, the thiolase or acetyl coenzyme A acetyltransferase is encoded by one or more nucleic acid molecules obtained from a microorganism selected from the group consisting of Clostridium acetobutylicum, Clostridium thermosaccharolyticum, Bacillus cereus, E. coli, Saccharomyces cerevisiae and Marinobacter hydrocarbonoclasticus. In some embodiments, the one or more nucleic acid molecules is thlA, atoB and/or ERG10, or homolog thereof. In a further embodiment, the thiolase or acetyl coenzyme A acetyltransferase comprises an amino acid sequence selected from the group consisting of SEQ ID NOs: 35, 37 and 40. In yet a further embodiment, the thiolase or acetyl coenzyme A acetyltransferase is encoded by a nucleic acid sequence selected from the group consisting of SEQ ID NOs: 33, 34, 36, 38 and 39.

In one embodiment of any aspect disclosed above, the acetyl-CoA:acetoacetate-CoA transferase or acetate:acetoacetyl-CoA hydrolase is encoded by one or more nucleic acid molecules obtained from a microorganism selected from Clostridium sp. and E. coli. In another embodiment, the acetyl-CoA:acetoacetate-CoA transferase or acetate:acetoacetyl-CoA hydrolase is encoded by one or more nucleic acid molecules obtained from E. coli. In some embodiments, the one or more nucleic acid molecules encoding the acetyl-CoA:acetoacetate-CoA transferase is atoA and/or atoD, or homolog thereof. In another embodiment, the acetyl-CoA:acetoacetate-CoA transferase or acetate:acetoacetyl-CoA hydrolase is encoded by one or more nucleic acid molecules obtained from Clostridium acetobutylicum. In some embodiments, the one or more nucleic acid molecules encoding the acetate:acetoacetyl-CoA hydrolase is ctfA and/or ctfB, or homolog thereof. In a further embodiment, the acetyl-CoA:acetoacetate-CoA transferase or acetate:acetoacetyl-CoA hydrolase comprises an amino acid sequence selected from the group consisting of SEQ ID NOs: 43, 46, 97, 99, 101 and 103. In yet a further embodiment, the acetyl-CoA:acetoacetate-CoA transferase or acetate:acetoacetyl-CoA hydrolase is encoded by a nucleic acid sequence selected from the group consisting of SEQ ID NOs: 41, 42, 44, 45, 96, 98, 100 and 102.

In one embodiment of any aspect disclosed above, the acetoacetate decarboxylase is encoded by one or more nucleic acid molecules obtained from a microorganism selected from the group consisting of Clostridium sp., Bacillus sp., Chromobacterium sp. and Pseudomonas sp. In another embodiment, the acetoacetate decarboxylase is encoded by one or more nucleic acid molecules obtained from a microorganism selected from the group consisting of Clostridium acetobutylicum, Clostridium beijerinckii, Clostridium cellulolyticum, Bacillus polymyxa, Chromobacterium violaceum and Pseudomonas putida. In some embodiments, the one or more nucleic acid molecules encoding the acetoacetate decarboxylase is adc, or homolog thereof. In a further embodiment, the acetoacetate decarboxylase comprises an amino acid sequence selected from the group consisting of SEQ ID NOs: 49 and 52. In yet another embodiment, the acetoacetate decarboxylase is encoded by a nucleic acid sequence selected from the group consisting of SEQ ID NOs: 47, 48, 50 and 51.

In one embodiment of any aspect disclosed above, the 3-hydroxyisovalerate synthase is encoded by one or more nucleic acid molecules obtained from a microorganism selected from Mus sp., Saccharomyces sp., Lactobacillus sp. and Polaromonas sp. In another embodiment, the 3-hydroxyisovalerate synthase is encoded by one or more nucleic acid molecules obtained from a microorganism selected from Mus musculus, Saccharomyces cerevisiae, Lactobacillus crispatus and Polaromonas naphthalenivorans. In some embodiments, the one or more nucleic acid molecules encoding the 3-hydroxyisovalerate synthase is selected from Hmgcs1, ERG13, PksG and/or Pnap_0477, or homolog thereof. In a further embodiment, the one or more nucleic acid molecules encoding the 3-hydroxyisovalerate synthase comprises an amino acid sequence selected from the group consisting of SEQ ID NOs: 105, 107, 109 and 111. In yet another embodiment, the one or more nucleic acid molecules encoding the 3-hydroxyisovalerate synthase is encoded by a nucleic acid sequence selected from the group consisting of SEQ ID NOs: 104, 106, 108 and 110.

In one embodiment of any aspect disclosed above, the hydroxymethylglutaryl-CoA synthase is encoded by one or more nucleic acid molecules obtained from Saccharomyces sp. In another embodiment, the hydroxymethylglutaryl-CoA synthase is encoded by one or more nucleic acid molecules obtained from Saccharomyces cerevisiae. In some embodiments, the one or more nucleic acid molecules encoding the hydroxymethylglutaryl-CoA synthase is HmgS, or homolog thereof. In a further embodiment, the one or more nucleic acid molecules encoding the hydroxymethylglutaryl-CoA synthase comprises an amino acid sequence set forth in SEQ ID NO: 123. In yet another embodiment, the one or more nucleic acid molecules encoding the hydroxymethylglutaryl-CoA synthase is encoded by a nucleic acid sequence set forth in SEQ ID NO: 122.

In one embodiment of any aspect disclosed above, the methylglutaconyl-CoA hydratase is encoded by one or more nucleic acid molecules obtained from Pseudomonas sp. In another embodiment, the methylglutaconyl-CoA hydratase is encoded by one or more nucleic acid molecules obtained from Pseudomonas putida. In some embodiments, the one or more nucleic acid molecules encoding the methylglutaconyl-CoA hydratase is liuC, or homolog thereof. In a further embodiment, the one or more nucleic acid molecules encoding the methylglutaconyl-CoA hydratase comprises an amino acid sequence set forth in SEQ ID NO: 125. In yet another embodiment, the one or more nucleic acid molecules encoding the methylglutaconyl-CoA hydratase is encoded by a nucleic acid sequence set forth in SEQ ID NO: 124.

In one embodiment of any aspect disclosed above, the methylcrotonyl-CoA carboxylase is encoded by one or more nucleic acid molecules obtained from Pseudomonas sp. In another embodiment, the methylcrotonyl-CoA carboxylase is encoded by one or more nucleic acid molecules obtained from Pseudomonas aeruginosa. In some embodiments, the one or more nucleic acid molecules encoding the methylcrotonyl-CoA carboxylase is liuB, and/or liuD, or homologs thereof. In a further embodiment, the one or more nucleic acid molecules encoding the methylcrotonyl-CoA carboxylase comprises an amino acid sequence selected from SEQ ID NOs: 127 and 129. In yet another embodiment, the one or more nucleic acid molecules encoding the methylcrotonyl-CoA carboxylase is encoded by a nucleic acid sequence selected from SEQ ID NOs: 126 and 128.

In one embodiment of any aspect disclosed above, the methylcrotonyl-CoA hydratase is a 3-ketoacyl-CoA thiolase. In another embodiment, the methylcrotonyl-CoA hydratase is an enoyl-CoA hydratase. In another embodiment, the methylcrotonyl-CoA hydratase is encoded by one or more nucleic acid molecules obtained from E. coli. In some embodiments, the one or more nucleic acid molecules encoding the methylcrotonyl-CoA hydratase is fadA, and/or fadB, or homologs thereof. In a further embodiment, the one or more nucleic acid molecules encoding the methylcrotonyl-CoA hydratase comprises an amino acid sequence selected from SEQ ID NOs: 131 and 133. In yet another embodiment, the one or more nucleic acid molecules encoding the methylcrotonyl-CoA hydratase is encoded by a nucleic acid sequence selected from SEQ ID NOs: 130 and 132.

In one embodiment of any aspect disclosed above, the 3-hydroxyisovaleryl-CoA thioesterase is encoded by one or more nucleic acid molecules obtained from E. coli. In some embodiments, the one or more nucleic acid molecules encoding the 3-hydroxyisovaleryl-CoA thioesterase is tesB, or homolog thereof. In a further embodiment, the one or more nucleic acid molecules encoding the 3-hydroxyisovaleryl-CoA thioesterase comprises an amino acid sequence set forth in SEQ ID NO: 135. In yet another embodiment, the one or more nucleic acid molecules encoding the 3-hydroxyisovaleryl-CoA thioesterase is encoded by a nucleic acid sequence set forth in SEQ ID NO: 134.

In one embodiment of any aspect disclosed above, the 3HIV kinase is encoded by one or more nucleic acid molecules obtained from a microorganism selected from the group consisting of Thermoplasma sp. and Picrophilus sp. In another embodiment, the 3HIV kinase is encoded by one or more nucleic acid molecules obtained from a microorganism selected from the group consisting of Thermoplasma acidophilum and Picrophilus torridus. In some embodiments, the one or more nucleic acid molecules encoding the 3HIV kinase is TA1305 and/or PTO1356, or homolog thereof. In some embodiments, the TA1305 comprises a L200E mutation. In a further embodiment, the one or more nucleic acid molecules encoding the 3HIV kinase comprises an amino acid sequence selected from the group consisting of SEQ ID NOs: 113, 115 and 117. In yet another embodiment, the one or more nucleic acid molecules encoding the 3HIV kinase is encoded by a nucleic acid sequence selected from the group consisting of SEQ ID NOs: 112, 114 and 116.

In one embodiment of any aspect disclosed above, the 3HIV-3-phosphate decarboxylase is encoded by one or more nucleic acid molecules obtained from Streptococcus sp. In another embodiment, the 3HIV-3-phosphate decarboxylase is encoded by one or more nucleic acid molecules obtained from a microorganism selected from Streptococcus mitis and Streptococcus gordonii. In some embodiments, the one or more nucleic acid molecules encoding the 3HIV-3-phosphate decarboxylase comprises smi_1746 and/or mvaD, or homolog thereof. In a further embodiment, the one or more nucleic acid molecules encoding the 3HIV-3-phosphate decarboxylase comprises an amino acid sequence selected from SEQ ID NOs: 119 and 121. In yet another embodiment, the one or more nucleic acid molecules encoding the 3HIV-3-phosphate decarboxylase is encoded by a nucleic acid sequence selected from SEQ ID NOs: 118 and 120.

A new variang of smi_1746 has been identified (Protein D3HAT7: mevalonate-diphosphate decarboxylase from Streptococcus mitis), which comprises the following sixteen mutations I16L R24K C118L Y121R S141P E159L M173C E177C K180P K241I S248T K282C E291D F297L L303M T308S.

In one embodiment of any aspect disclosed above, the 3HIV decarboxylase is encoded by one or more nucleic acid molecules obtained from a microorganism selected from the group consisting of Streptococcus sp., Thermoplasma sp. and Picrophilus sp. In another embodiment, the 3HIV decarboxylase is encoded by one or more nucleic acid molecules obtained from a microorganism selected from the group consisting of Streptococcus gordonii, Thermoplasma acidophilum and Picrophilus torridus. In some embodiments, the one or more nucleic acid molecules encoding the 3HIV decarboxylase comprises mvaD, TA1305 and/or PTO1356, or homolog thereof. In a further embodiment, the one or more nucleic acid molecules encoding the 3HIV decarboxylase comprises an amino acid sequence selected from the group consisting of SEQ ID NOs: 113, 117 and 121. In yet another embodiment, the one or more nucleic acid molecules encoding the 3HIV decarboxylase is encoded by a nucleic acid sequence selected from the group consisting of SEQ ID NOs: 112, 116 and 120.

### Recombinant Microorganism

The disclosure provides microorganisms that can be engineered to express various endogenous or exogenous enzymes.

In various embodiments described herein, the recombinant microorganism is a eukaryotic microorganism. In some embodiments, the eukaryotic microorganism is a yeast. In exemplary embodiments, the yeast is a member of a genus selected from the group consisting of Yarrowia, Candida, Saccharomyces, Pichia, Hansenula, Kluyveromyces, Issatchenkia, Zygosaccharomyces, Debaryomyces, Schizosaccharomyces, Pachysolen, Cryptococcus, Trichosporon, Rhodotorula, and Myxozyma.

In some embodiments, the recombinant microorganism is a prokaryotic microorganism. In exemplary embodiments, the prokaryotic microorganism is a member of a genus selected from the group consisting of Escherichia, Clostridium, Zymomonas, Salmonella, Rhodococcus, Pseudomonas, Bacillus, Lactobacillus, Enterococcus, Alcaligenes, Klebsiella, Paenibacillus, Arthrobacter, Corynebacterium, and Brevibacterium.

In some embodiments, the recombinant microorganism is used to produce monoethylene glycol (MEG) disclosed herein.

Accordingly, in another aspect, the present inventions provide a method of producing MEG and isobutene using a recombinant microorganism described herein. In one embodiment, the method comprises cultivating the recombinant microorganism in a culture medium containing a feedstock providing a carbon source until MEG and isobutene is produced. In a further embodiment, the MEG and isobutene is recovered. Recovery can be by methods known in the art, such as distillation, membrane-based separation gas stripping, solvent extraction, and expanded bed adsorption.

In some embodiments, the feedstock comprises a carbon source. In various embodiments described herein, the carbon source may be selected from sugars, glycerol, alcohols, organic acids, alkanes, fatty acids, lignocellulose, proteins, carbon dioxide, and carbon monoxide. In an exemplary embodiment, the carbon source is a sugar. In a further exemplary embodiment, the sugar is D-xylose. In alternative embodiments, the sugar is selected from the group consisting of glucose, fructose, and sucrose.

Methods of Producing a Recombinant Microorganism that Produces or Accumulates MEG and Isobutene

As discussed above, in another aspect, the present disclosure relates to a method of producing a recombinant microorganism that produces or accumulates MEG and isobutene from exogenous D-xylose, comprising introducing into the recombinant microorganism and/or overexpressing one or more of the following from (a) to (d):
(a) at least one endogenous or exogenous nucleic acid molecule encoding a D-tagatose 3-epimerase that catalyzes the conversion of D-xylulose to D-ribulose;
(b) at least one endogenous or exogenous nucleic acid molecule encoding a D-ribulokinase that catalyzes the conversion of D-ribulose from (a) to D-ribulose-1-phosphate,
(c) at least one endogenous or exogenous nucleic acid molecule encoding a D-ribulose-1-phosphate aldolase that catalyzes the conversion of D-ribulose-1-phosphate from (b) to glycolaldehyde and dihydroxyacetonephosphate (DHAP);
(d) at least one endogenous or exogenous nucleic acid molecule encoding a glycolaldehyde reductase that catalyzes the conversion of glycolaldehyde from (c) to MEG;
   wherein the method further comprises introducing into and/or overexpressing in the recombinant microorganism one or more of the following from (e) to (h):
(e) at least one endogenous or exogenous nucleic acid molecule encoding a thiolase or acetyl coenzyme A acetyltransferase that catalyzes the conversion of acetyl-CoA to acetoacetyl-CoA;
(f) at least one endogenous or exogenous nucleic acid molecule encoding an acetyl-CoA:acetoacetate-CoA transferase or acetate:acetoacetyl-CoA hydrolase that catalyzes the conversion of acetoacetyl-CoA from (e) to acetoacetate;
(g) at least one endogenous or exogenous nucleic acid molecule encoding an acetoacetate decarboxylase that catalyzes the conversion of acetoacetate from (f) to acetone;
(h) at least one endogenous or exogenous nucleic acid molecule encoding a hydroxymethylglutaryl-CoA synthase that catalyzes the conversion of acetone from (g) and acetyl-CoA to 3-hydroxyisovalerate (3HIV);
or
   wherein the method comprises introducing into and/or overexpressing in the recombinant microorganism one or more of the nucleic acid molecule from (a) to (d) above and further comprises introducing into and/or overexpressing in the recombinant microorganism one or more of the following from (i) to (n):
(i) at least one endogenous or exogenous nucleic acid molecule encoding an acetyl coenzyme A acetyltransferase that catalyzes the conversion of acetyl-CoA to acetoacetyl-CoA;
(j) at least one endogenous or exogenous nucleic acid molecule encoding a hydroxymethylglutaryl-CoA synthase that catalyzes the conversion of acetoacetyl-CoA from (i) and acetyl-CoA to 3-hydroxy-3-methylglutaryl-CoA (HMG-CoA);
(k) at least one endogenous or exogenous nucleic acid molecule encoding a methylglutaconyl-CoA hydratase that catalyzes the conversion of HMG-CoA from (j) to 3-methylglutaconyl-CoA,
(l) at least one endogenous or exogenous nucleic acid molecule encoding a methylcrotonyl-CoA carboxylase that catalyzes the conversion of 3-methylglutaconyl-CoA from (k) to 3-methylcrotonyl-CoA;
(m) at least one endogenous or exogenous nucleic acid molecule encoding a methylcrotonyl-CoA hydratase that catalyzes the conversion of 3-methylcrotonyl-CoA from (l) to 3-hydroxyisovaleryl-CoA;
(n) at least one endogenous or exogenous nucleic acid molecule encoding a 3-hydroxyisovaleryl-CoA thioesterase that catalyzes the conversion of 3-hydroxyisovaleryl-CoA from (m) to 3HIV;
   wherein the method further comprises introducing into and/or overexpressing in the recombinant microorganism (a1) and (a2), and/or (b1) selected from:
(a1) at least one endogenous or exogenous nucleic acid molecule encoding a 3HIV kinase that catalyzes the conversion of 3HIV from (h) or (n) to 3HIV-3-phosphate;
(a2) at least one endogenous or exogenous nucleic acid molecule encoding a 3HIV-3-phosphate decarboxylase that catalyzes the conversion of 3HIV-3phosphate from (a1) to isobutene;
(b1) at least one endogenous or exogenous nucleic acid molecule encoding a 3HIV decarboxylase that catalyzes the conversion of 3HIV from (h) or (n) to isobutene;
   and wherein the produced intermediate DHAP is converted to acetyl-CoA through the endogenous glycolysis pathway in the microorganism, and wherein MEG and isobutene are co-produced.

In one embodiment, the D-tagatose 3-epimerase is encoded by one or more nucleic acid molecules obtained from a microorganism selected from the group consisting of Pseudomonas sp., Mesorhizobium sp. and Rhodobacter sp. In some embodiments, the D-tagatose 3-epimerase is encoded by one or more nucleic acid molecules obtained from a microorganism selected from the group consisting of Pseudomonas cichorii, Pseudomonas sp. ST-24, Mesorhizobium loti and Rhodobacter sphaeroides. In some embodiments, the one or more nucleic acid molecules is dte and/or FJ851309.1, or homolog thereof. In a further embodiment, the D-tagatose 3-epimerase comprises an amino acid sequence selected from the group consisting of SEQ ID NOs: 3 and 5. In yet a further embodiment, the D-tagatose 3-epimerase is encoded by a nucleic acid sequence selected from the group consisting of SEQ ID NOs: 1, 2 and 4.

In one embodiment, the D-ribulokinase is encoded by one or more nucleic acid molecules obtained from E. coli. In some embodiments, the one or more nucleic acid molecules is fucK, or homolog thereof. In a further embodiment, the D-ribulokinase comprises an amino acid sequence set forth in SEQ ID NO: 8. In yet a further embodiment, the D-ribulokinase is encoded by a nucleic acid sequence selected from the group consisting of SEQ ID NOs: 6 and 7.

In one embodiment, the D-ribulose-1-phosphate aldolase is encoded by one or more nucleic acid molecules obtained from E. coli. In some embodiments, the one or more nucleic acid molecules is fucA, or homolog thereof. In a further embodiment, the D-ribulose-1-phosphate aldolase comprises an amino acid sequence set forth in SEQ ID NO: 11. In yet a further embodiment, the D-ribulose-1-phosphate aldolase is encoded by a nucleic acid sequence selected from the group consisting of SEQ ID NOs: 9 and 10.

As discussed above, in another aspect, the present disclosure relates to a method of producing a recombinant microorganism that produces or accumulates MEG and isobutene from exogenous D-xylose, comprising introducing into and/or overexpressing in the recombinant microorganism one or more of the following from (a) to (c):
(a) at least one endogenous or exogenous nucleic acid molecule encoding a D-xylulose 1-kinase that catalyzes the conversion of D-xylulose to D-xylulose-1-phosphate;
(b) at least one endogenous or exogenous nucleic acid molecule encoding a D-xylulose-1-phosphate aldolase that catalyzes the conversion of D-xylulose-1-phosphate from (a) to glycolaldehyde and dihydroxyacetonephosphate (DHAP);
(c) at least one endogenous or exogenous nucleic acid molecule encoding a glycolaldehyde reductase that catalyzes the conversion of glycolaldehyde from (b) to MEG;
   wherein the method further comprises introducing into and/or overexpressing in the recombinant microorganism one or more of the following from (d) to (g):
(d) at least one endogenous or exogenous nucleic acid molecule encoding a thiolase or acetyl coenzyme A acetyltransferase that catalyzes the conversion of acetyl-CoA to acetoacetyl-CoA;
(e) at least one endogenous or exogenous nucleic acid molecule encoding an acetyl-CoA:acetoacetate-CoA transferase or acetate:acetoacetyl-CoA hydrolase that catalyzes the conversion of acetoacetyl-CoA from (d) to acetoacetate;
(f) at least one endogenous or exogenous nucleic acid molecule encoding an acetoacetate decarboxylase that catalyzes the conversion of acetoacetate from (e) to acetone;
(g) at least one endogenous or exogenous nucleic acid molecule encoding a 3-hydroxyisovalerate synthase that catalyzes the conversion of acetone from (f) and acetyl-CoA to 3-hydroxyisovalerate (3HIV);
or
   wherein the method comprises introducing into and/or overexpressing in the recombinant microorganism one or more of the nucleic acid molecule from (a) to (c) above and further comprises introducing into and/or overexpressing in the recombinant microorganism one or more of the following from (h) to (m):
(h) at least one endogenous or exogenous nucleic acid molecule encoding an acetyl coenzyme A acetyltransferase that catalyzes the conversion of acetyl-CoA to acetoacetyl-CoA;
(i) at least one endogenous or exogenous nucleic acid molecule encoding a hydroxymethylglutaryl-CoA synthase that catalyzes the conversion of acetoacetyl-CoA from (h) and acetyl-CoA to 3-hydroxy-3-methylglutaryl-CoA (HMG-CoA);
(j) at least one endogenous or exogenous nucleic acid molecule encoding a methylglutaconyl-CoA hydratase that catalyzes the conversion of HMG-CoA from (i) to 3-methylglutaconyl-CoA;
(k) at least one endogenous or exogenous nucleic acid molecule encoding a methylcrotonyl-CoA carboxylase that catalyzes the conversion of 3-methylglutaconyl-CoA from (j) to 3-methylcrotonyl-CoA;
(l) at least one endogenous or exogenous nucleic acid molecule encoding a methylcrotonyl-CoA hydratase that catalyzes the conversion of 3-methylcrotonyl-CoA from (k) to 3-hydroxyisovaleryl-CoA;
(m) at least one endogenous or exogenous nucleic acid molecule encoding a 3-hydroxyisovaleryl-CoA thioesterase that catalyzes the conversion of 3-hydroxyisovaleryl-CoA from (l) to 3HIV; wherein the method further comprises introducing into and/or overexpressing in the recombinant microorganism (a1) and (a2), and/or (b1) selected from:
(a1) at least one endogenous or exogenous nucleic acid molecule encoding a 3HIV kinase that catalyzes the conversion of 3HIV from (g) or (m) to 3HIV-3-phosphate;
(a2) at least one endogenous or exogenous nucleic acid molecule encoding a 3HIV-3-phosphate decarboxylase that catalyzes the conversion of 3HIV-3phosphate from (a1) to isobutene;
(b1) at least one endogenous or exogenous nucleic acid molecule encoding a 3HIV decarboxylase that catalyzes the conversion of 3HIV from (g) or (m) to isobutene;
   and wherein the produced intermediate DHAP is converted to acetyl-CoA through the endogenous glycolysis pathway in the microorganism, and wherein MEG and isobutene are co-produced.

In one embodiment, the D-xylulose 1-kinase is encoded by one or more nucleic acid molecules obtained from Homo sapiens. In some embodiments, the one or more nucleic acid molecules encoding the D-xylulose 1-kinase is ketohexokinase C (khk-C), or homolog thereof. In another embodiment, the one or more nucleic acid molecules encoding the D-xylulose 1-kinase comprises an amino acid sequence set forth in SEQ ID NO: 55. In a further embodiment, the one or more nucleic acid molecules encoding the D-xylulose 1-kinase is encoded by a nucleic acid sequence selected from the group consisting of SEQ ID NOs: 53 and 54.

In one embodiment, the D-xylulose-1-phosphate aldolase is encoded by one or more nucleic acid molecules obtained from Homo sapiens. In another embodiment, the one or more nucleic acid molecules encoding the D-xylulose-1-phosphate aldolase is aldolase B (ALDOB), or homolog thereof. In some embodiments, the one or more nucleic acid molecules encoding the D-xylulose-1-phosphate aldolase comprises an amino acid sequence set forth in SEQ ID NO: 58. In some embodiments, the one or more nucleic acid molecules encoding the D-xylulose-1-phosphate aldolase is encoded by a nucleic acid sequence selected from the group consisting of SEQ ID NOs: 56 and 57.

In one embodiment of any aspect disclosed above, the method further comprises introducing into the recombinant microorganism one or more modifications selected from the group consisting of:
(a) a deletion, insertion, or loss of function mutation in a gene encoding a D-xylulose-5-kinase that catalyzes the conversion of D-xylulose to D-xylulose-5-phosphate;
(b) a deletion, insertion, or loss of function mutation in a gene encoding a glycolaldehyde dehydrogenase that catalyzes the conversion of glycolaldehyde to glycolic acid; and
(c) a deletion, insertion, or loss of function mutation in a gene encoding a lactate dehydrogenase that catalyzes the conversion of pyruvate to lactate.

In some embodiments of any aspect disclosed above, a method of producing a recombinant microorganism that produces or accumulates MEG and isobutene from exogenous D-xylose comprises introducing into the recombinant microorganism a deletion, insertion, or loss of function mutation in a gene encoding a D-xylulose-5-kinase to prevent the conversion of D-xylulose to D-xylulose-5-phosphate and instead shunt the reaction toward conversion of D-xylulose to D-xylulose-1-phosphate. In some embodiments, the D-xylulose-5-kinase is from Escherichia coli. In some embodiments, the D-xylulose-5-kinase is encoded by the xylB gene, or homolog thereof.

In some embodiments of any aspect disclosed above, a method of producing a recombinant microorganism that produces or accumulates MEG and isobutene from exogenous D-xylose comprises introducing into the recombinant microorganism a deletion, insertion, or loss of function mutation in a gene encoding a glycolaldehyde dehydrogenase to prevent the production of glycolic acid from glycolaldehyde and instead shunt the reaction toward conversion of glycolaldehyde to MEG. In some embodiments, the glycolaldehyde dehydrogenase is from Escherichia coli. In some embodiments, the glycolaldehyde dehydrogenase is encoded by the aldA gene, or homolog thereof.

In some embodiments of any aspect disclosed above, a method of producing a recombinant microorganism that produces or accumulates MEG and isobutene from exogenous D-xylose comprises introducing into the recombinant microorganism a deletion, insertion, or loss of function mutation in a gene encoding a lactate dehydrogenase to prevent the production of lactate from pyruvate and instead shunt the reaction toward production of isobutene. In some embodiments, the lactate dehydrogenase is from Escherichia coli. In some embodiments, the lactate dehydrogenase is encoded by the IdhA gene, or homolog thereof.

In one embodiment of any aspect disclosed above, the method further comprises introducing into the recombinant microorganism and/or overexpressing an endogenous or exogenous xylose isomerase that catalyzes the conversion of D-xylose to D-xylulose. In one embodiment, the xylose isomerase is exogenous. In another embodiment, the xylose isomerase is encoded by one or more nucleic acid molecules obtained from Pyromyces sp. In another embodiment, the one or more nucleic acid molecules encoding the xylose isomerase is xylA, or homolog thereof. In yet another embodiment, the one or more nucleic acid molecules encoding the xylose isomerase comprises an amino acid sequence set forth in SEQ ID NO: 95. In a further embodiment, the one or more nucleic acid molecules encoding the xylose isomerase is encoded by a nucleic acid sequence selected from the group consisting of SEQ ID NOs: 93 and 94.

In one embodiment of any aspect disclosed above, the method further comprises introducing into the recombinant microorganism and/or overexpressing at least one exogenous nucleic acid molecule encoding a xylose reductase or aldose reductase that catalyzes the conversion of D-xylose to xylitol and at least one exogenous nucleic acid molecule encoding a xylitol dehydrogenase that catalyzes the conversion of xylitol to D-xylulose.

In some embodiments of any aspect disclosed above, the xylose reductase or aldose reductase is encoded by one or more nucleic acid molecules obtained from a microorganism selected from the group consisting of Hypocrea sp., Scheffersomyces sp., Saccharomyces sp., Pachysolen sp., Pichia sp., Candida sp., Aspergillus sp., Neurospora sp., and Cryptococcus sp. In some embodiments, the xylose reductase or aldose reductase is encoded by one or more nucleic acid molecules obtained from a microorganism selected from the group consisting of Hypocrea jecorina, Scheffersomyces stipitis, Saccharomyces cerevisiae, Pachysolen tannophilus, Pichia stipitis, Pichia quercuum, Candida shehatae, Candida tenuis, Candida tropicalis, Aspergillus niger, Neurospora crassa and Cryptococcus lactativorus. In another embodiment, the one or more nucleic acid molecules encoding the xylose reductase or aldose reductase is xyl1 and/or GRE3 or homolog thereof. In some embodiments, the one or more nucleic acid molecules encoding the xylose reductase or aldose reductase comprises an amino acid sequence selected from the group consisting of SEQ ID NOs: 84 and 87. In some embodiments, the one or more nucleic acid molecules encoding the xylose reductase or aldose reductase is encoded by a nucleic acid sequence selected from the group consisting of SEQ ID NOs: 82, 83, 85 and 86.

In one embodiment of any aspect disclosed above, the xylitol dehydrogenase is encoded by one or more nucleic acid molecules obtained from a microorganism selected from the group consisting of Scheffersomyces sp., Trichoderma sp., Pichia sp., Saccharomyces sp., Gluconobacter sp., Galactocandida sp., Neurospora sp., and Serratia sp. In another embodiment, the xylitol dehydrogenase is encoded by one or more nucleic acid molecules obtained from a microorganism selected from the group consisting of Scheffersomyces stipitis, Trichoderma reesei, Pichia stipitis, Saccharomyces cerevisiae, Gluconobacter oxydans, Galactocandida mastotermitis, Neurospora crassa and Serratia marcescens. In another embodiment, the one or more nucleic acid molecules encoding the xylitol dehydrogenase is xyl2 and/or xdh1, or homolog thereof. In some embodiments, the one or more nucleic acid molecules encoding the xylitol dehydrogenase comprises an amino acid sequence selected from the group consisting of SEQ ID NOs: 90 and 92. In some embodiments, the one or more nucleic acid molecules encoding the xylitol dehydrogenase is encoded by a nucleic acid sequence selected from the group consisting of SEQ ID NOs: 88, 89 and 91.

As discussed above, in another aspect, the present disclosure relates to a method of producing a recombinant microorganism that produces or accumulates MEG and isobutene from exogenous D-xylose, comprising introducing into and/or overexpressing in the recombinant microorganism one or more of the following from (a) to (c):
(a) at least one endogenous or exogenous nucleic acid molecule encoding a xylose dehydrogenase that catalyzes the conversion of D-xylose to D-xylonolactone; and
(b) at least one endogenous or exogenous nucleic acid molecule encoding a xylonolactonase that catalyzes the conversion of D-xylonolactone from (a) to D-xylonate;
(c) at least one endogenous or exogenous nucleic acid molecule encoding a xylose dehydrogenase that catalyzes the conversion of D-xylose to D-xylonate;
   wherein the method further comprises introducing into and/or overexpressing in the recombinant microorganism one or more of the following from (d) to (f):
(d) at least one endogenous or exogenous nucleic acid molecule encoding a xylonate dehydratase that catalyzes the conversion of D-xylonate from (b) or (c) to 2-keto-3-deoxy-xylonate;
(e) at least one endogenous or exogenous nucleic acid molecule encoding a 2-keto-3-deoxy-D-pentonate aldolase that catalyzes the conversion of 2-keto-3-deoxy-xylonate from (d) to glycolaldehyde and pyruvate;
(f) at least one endogenous or exogenous nucleic acid molecule encoding a glycolaldehyde reductase that catalyzes the conversion of glycolaldehyde from (e) to MEG;
   wherein the method further comprises introducing into and/or overexpressing in the recombinant microorganism one or more of the following from (g) to (j):
(g) at least one exogenous nucleic acid molecule encoding a thiolase or acetyl coenzyme A acetyltransferase that catalyzes the conversion of acetyl-CoA to acetoacetyl-CoA;
(h) at least one endogenous or exogenous nucleic acid molecule encoding an acetyl-CoA:acetoacetate-CoA transferase or acetate:acetoacetyl-CoA hydrolase that catalyzes the conversion of acetoacetyl-CoA from (g) to acetoacetate;
(i) at least one exogenous nucleic acid molecule encoding an acetoacetate decarboxylase that catalyzes the conversion of acetoacetate from (h) to acetone;
(j) at least one endogenous or exogenous nucleic acid molecule encoding a 3-hydroxyisovalerate synthase that catalyzes the conversion of acetone from (i) and acetyl-CoA to 3-hydroxy-isovalerate (3HIV);
or
   wherein the method comprises introducing into and/or overexpressing in the recombinant microorganism one or more of the nucleic acid molecule from (a) to (c) above and one or more of the nucleic acid molecule from (d) to (f) above, and further comprises introducing into and/or overexpressing in the recombinant microorganism one or more of the following from (k) to (p):
(k) at least one endogenous or exogenous nucleic acid molecule encoding an acetyl coenzyme A acetyltransferase that catalyzes the conversion of acetyl-CoA to acetoacetyl-CoA;
(l) at least one endogenous or exogenous nucleic acid molecule encoding a hydroxymethylglutaryl-CoA synthase that catalyzes the conversion of acetoacetyl-CoA from (k) and acetyl-CoA to 3-hydroxy-3-methylglutaryl-CoA (HMG-CoA);
(m) at least one endogenous or exogenous nucleic acid molecule encoding a methylglutaconyl-CoA hydratase that catalyzes the conversion of HMG-CoA from (l) to 3-methylglutaconyl-CoA;
(n) at least one endogenous or exogenous nucleic acid molecule encoding a methylcrotonyl-CoA carboxylase that catalyzes the conversion of 3-methylglutaconyl-CoA from (m) to 3-methylcrotonyl-CoA;
(o) at least one endogenous or exogenous nucleic acid molecule encoding a methylcrotonyl-CoA hydratase that catalyzes the conversion of 3-methylcrotonyl-CoA from (n) to 3-hydroxyisovaleryl-CoA;
(p) at least one endogenous or exogenous nucleic acid molecule encoding a 3-hydroxyisovaleryl-CoA thioesterase that catalyzes the conversion of 3-hydroxyisovaleryl-CoA from (o) to 3HIV;
   wherein the method further comprises introducing into and/or overexpressing in the recombinant microorganism (a1) and (a2), and/or (b1) selected from:
(a1) at least one endogenous or exogenous nucleic acid molecule encoding a 3HIV kinase that catalyzes the conversion of 3HIV from (j) or (p) to 3HIV-3-phosphate;
(a2) at least one endogenous or exogenous nucleic acid molecule encoding a 3HIV-3-phosphate decarboxylase that catalyzes the conversion of 3HIV-3phosphate from (a1) to isobutene;
(b1) at least one endogenous or exogenous nucleic acid molecule encoding a 3HIV decarboxylase that catalyzes the conversion of 3HIV from (j) or (p) to isobutene;
   and wherein the produced intermediate pyruvate is converted to acetyl-CoA through the endogenous glycolysis pathway in the microorganism, and wherein MEG and isobutene are co-produced.

In one embodiment, the xylose dehydrogenase is encoded by one or more nucleic acid molecules obtained from a microorganism selected from the group consisting of Caulobacter sp., Haloarcula sp., Haloferax sp., Halorubrum sp. and Trichoderma sp. In another embodiment, the xylose dehydrogenase is encoded by one or more nucleic acid molecules obtained from a microorganism selected from the group consisting of Caulobacter crescentus, Haloarcula marismortui, Haloferax volcanii, Halorubrum lacusprofundi and Trichoderma reesei. In some embodiments, the one or more nucleic acid molecules encoding the xylose dehydrogenase is selected from xylB, xdh1 (HVO_B0028) and/or xyd1, or homolog thereof. In a further embodiment, the one or more nucleic acid molecules encoding the xylose dehydrogenase comprises an amino acid sequence selected from the group consisting of SEQ ID NOs: 61, 63 and 65. In yet another embodiment, the one or more nucleic acid molecules encoding the xylose dehydrogenase is encoded by a nucleic acid sequence selected from the group consisting of SEQ ID NOs: 59, 60, 62 and 64.

In one embodiment, the xylonolactonase is encoded by one or more nucleic acid molecules obtained from a microorganism selected from Caulobacter sp. and Haloferax sp. In another embodiment, the xylonolactonase is encoded by one or more nucleic acid molecules obtained from a microorganism selected from the group consisting of Caulobacter crescentus, Haloferax volcanii and Haloferax gibbonsii. In some embodiments, the one or more nucleic acid molecules encoding the xylonolactonase is xylC, or homolog thereof. In a further embodiment, the one or more nucleic acid molecules encoding the xylonolactonase comprises an amino acid sequence set forth in SEQ ID NO: 67. In yet another embodiment, the one or more nucleic acid molecules encoding the xylonolactonase is encoded by a nucleic acid sequence set forth in SEQ ID NO: 66.

In one embodiment, the xylonate dehydratase is encoded by one or more nucleic acid molecules obtained from a microorganism selected from the group consisting of Caulobacter sp., Sulfolobus sp. and E. coli. In another embodiment, the xylonate dehydratase is encoded by one or more nucleic acid molecules obtained from a microorganism selected from the group consisting of Caulobacter crescentus, Sulfolobus solfataricus and E. coli. In some embodiments, the one or more nucleic acid molecules encoding the xylonate dehydratase is selected from xylD, yjhG and/or yagF, or homolog thereof. In a further embodiment, the one or more nucleic acid molecules encoding the xylonate dehydratase comprises an amino acid sequence selected from the group consisting of SEQ ID NOs: 69, 72 and 75. In yet another embodiment, the one or more nucleic acid molecules encoding the xylonate dehydratase is encoded by a nucleic acid sequence selected from the group consisting of SEQ ID NOs: 68, 70, 71, 73 and 74.

In one embodiment, the 2-keto-3-deoxy-D-pentonate aldolase is encoded by one or more nucleic acid molecules obtained from a microorganism selected from Pseudomonas sp. and E. coli. In another embodiment, the 2-keto-3-deoxy-D-pentonate aldolase is encoded by one or more nucleic acid molecules obtained from E. coli. In some embodiments, the one or more nucleic acid molecules encoding the 2-keto-3-deoxy-D-pentonate aldolase is selected from yjhH and/or yagE, or homolog thereof. In a further embodiment, the one or more nucleic acid molecules encoding the 2-keto-3-deoxy-D-pentonate aldolase comprises an amino acid sequence selected from the group consisting of SEQ ID NOs: 78 and 81. In yet another embodiment, the one or more nucleic acid molecules encoding the 2-keto-3-deoxy-D-pentonate aldolase is encoded by a nucleic acid sequence selected from the group consisting of SEQ ID NOs: 76, 77, 79 and 80.

In one embodiment, the method further comprises introducing into the recombinant microorganism one or more modifications selected from the group consisting of:
(a) a deletion, insertion, or loss of function mutation in a gene encoding an enzyme that catalyzes the conversion of D-xylose to D-xylulose;
(b) a deletion, insertion, or loss of function mutation in a gene encoding an enzyme that catalyzes the conversion of glycolaldehyde to glycolic acid; and
(c) a deletion, insertion, or loss of function mutation in a gene encoding an enzyme that catalyzes the conversion of pyruvate to lactate.

In some embodiments, a method of producing a recombinant microorganism that produces or accumulates MEG and isobutene from exogenous D-xylose comprises introducing into the recombinant microorganism a deletion, insertion, or loss of function mutation in a gene encoding a D-xylose isomerase to prevent conversion of D-xylose to D-xylulose and instead shunt the reaction toward the conversion of D-xylose to D-xylonate. In one embodiment, the enzyme that catalyzes the conversion of D-xylose to D-xylulose is a D-xylose isomerase. In some embodiments, the D-xylose isomerase is from Escherichia coli. In some embodiments, the D-xylose isomerase is encoded by the xylA gene, or homolog thereof.

In some embodiments, a method of producing a recombinant microorganism that produces or accumulates MEG and isobutene from exogenous D-xylose comprises introducing into the recombinant microorganism a deletion, insertion, or loss of function mutation in a gene encoding a glycolaldehyde dehydrogenase to prevent the production of glycolic acid from glycolaldehyde and instead shunt the reaction toward conversion of glycolaldehyde to MEG. In one embodiment, the glycolaldehyde dehydrogenase is from Escherichia coli. In some embodiments, the glycolaldehyde dehydrogenase is encoded by the aldA gene, or homolog thereof.

In some embodiments, a method of producing a recombinant microorganism that produces or accumulates MEG and isobutene from exogenous D-xylose comprises introducing into the recombinant microorganism a deletion, insertion, or loss of function mutation in a gene encoding a lactate dehydrogenase to prevent the production of lactate from pyruvate and instead shunt the reaction toward production of isobutene. In one embodiment, the enzyme that catalyzes the conversion of pyruvate to lactate is a lactate dehydrogenase. In particular embodiments, the enzyme converts pyruvate to lactate. In some embodiments, the lactate dehydrogenase is from Escherichia coli. In some embodiments, the lactate dehydrogenase is encoded by the IdhA gene, or homolog thereof.

In one embodiment of any aspect disclosed above, the glycolaldehyde reductase is encoded by one or more nucleic acid molecules obtained from a microorganism selected from E. coli and S. cerevisiae. In another embodiment, the one or more nucleic acid molecules is selected from gldA, GRE2, GRE3, yqhD, ydjG, fucO, yafB (dkgB), and/or yqhE (dkgA), or homolog thereof. In another embodiment, the one or more nucleic acid molecules is yqhD. In some embodiments, the yqhD comprises a G149E mutation. In a further embodiment, the glycolaldehyde reductase comprises an amino acid sequence selected from the group consisting of SEQ ID NOs: 13, 15, 17, 20, 23, 25, 28, 30 and 32. In yet a further embodiment, the glycolaldehyde reductase is encoded by a nucleic acid sequence selected from the group consisting of SEQ ID NOs: 12, 14, 16, 18, 19, 21, 22, 24, 26, 27, 29 and 31.

In one embodiment of any aspect disclosed above, the thiolase or acetyl coenzyme A acetyltransferase is encoded by one or more nucleic acid molecules obtained from a microorganism selected from the group consisting of Clostridium sp., Bacillus sp., E. coli, Saccharomyces sp. and Marinobacter sp. In some embodiments, the thiolase or acetyl coenzyme A acetyltransferase is encoded by one or more nucleic acid molecules obtained from a microorganism selected from the group consisting of Clostridium acetobutylicum, Clostridium thermosaccharolyticum, Bacillus cereus, E. coli, Saccharomyces cerevisiae and Marinobacter hydrocarbonoclasticus. In some embodiments, the one or more nucleic acid molecules is thIA, atoB and/or ERG10, or homolog thereof. In a further embodiment, the thiolase or acetyl coenzyme A acetyltransferase comprises an amino acid sequence selected from the group consisting of SEQ ID NOs: 35, 37 and 40. In yet a further embodiment, the thiolase or acetyl coenzyme A acetyltransferase is encoded by a nucleic acid sequence selected from the group consisting of SEQ ID NOs: 33, 34, 36, 38 and 39.

In one embodiment of any aspect disclosed above, the acetyl-CoA:acetoacetate-CoA transferase or acetate:acetoacetyl-CoA hydrolase is encoded by one or more nucleic acid molecules obtained from a microorganism selected from Clostridium sp. and E. coli. In another embodiment, the acetyl-CoA:acetoacetate-CoA transferase or acetate:acetoacetyl-CoA hydrolase is encoded by one or more nucleic acid molecules obtained from E. coli. In some embodiments, the one or more nucleic acid molecules encoding the acetyl-CoA:acetoacetate-CoA transferase is atoA and/or atoD, or homolog thereof. In another embodiment, the acetyl-CoA:acetoacetate-CoA transferase or acetate:acetoacetyl-CoA hydrolase is encoded by one or more nucleic acid molecules obtained from Clostridium acetobutylicum. In some embodiments, the one or more nucleic acid molecules encoding the acetate:acetoacetyl-CoA hydrolase is ctfA and/or ctfB, or homolog thereof. In a further embodiment, the acetyl-CoA:acetoacetate-CoA transferase or acetate:acetoacetyl-CoA hydrolase comprises an amino acid sequence selected from the group consisting of SEQ ID NOs: 43, 46, 97, 99, 101 and 103. In yet a further embodiment, the acetyl-CoA:acetoacetate-CoA transferase or acetate:acetoacetyl-CoA hydrolase is encoded by a nucleic acid sequence selected from the group consisting of SEQ ID NOs: 41, 42, 44, 45, 96, 98, 100 and 102.

In one embodiment of any aspect disclosed above, the acetoacetate decarboxylase is encoded by one or more nucleic acid molecules obtained from a microorganism selected from the group consisting of Clostridium sp., Bacillus sp., Chromobacterium sp. and Pseudomonas sp. In another embodiment, the acetoacetate decarboxylase is encoded by one or more nucleic acid molecules obtained from a microorganism selected from the group consisting of Clostridium acetobutylicum, Clostridium beijerinckii, Clostridium cellulolyticum, Bacillus polymyxa, Chromobacterium violaceum and Pseudomonas putida. In some embodiments, the one or more nucleic acid molecules encoding the acetoacetate decarboxylase is adc, or homolog thereof. In a further embodiment, the acetoacetate decarboxylase comprises an amino acid sequence selected from the group consisting of SEQ ID NOs: 49 and 52. In yet another embodiment, the acetoacetate decarboxylase is encoded by a nucleic acid sequence selected from the group consisting of SEQ ID NOs: 47, 48, 50 and 51.

In one embodiment of any aspect disclosed above, the 3-hydroxyisovalerate synthase is encoded by one or more nucleic acid molecules obtained from a microorganism selected from Mus sp., Saccharomyces sp., Lactobacillus sp. and Polaromonas sp. In another embodiment, the 3-hydroxyisovalerate synthase is encoded by one or more nucleic acid molecules obtained from a microorganism selected from Mus musculus, Saccharomyces cerevisiae, Lactobacillus crispatus and Polaromonas naphthalenivorans. In some embodiments, the one or more nucleic acid molecules encoding the 3-hydroxyisovalerate synthase is selected from Hmgcs1, ERG13, PksG and/or Pnap_0477, or homolog thereof. In a further embodiment, the one or more nucleic acid molecules encoding the 3-hydroxyisovalerate synthase comprises an amino acid sequence selected from the group consisting of SEQ ID NOs: 105, 107, 109 and 111. In yet another embodiment, the one or more nucleic acid molecules encoding the 3-hydroxyisovalerate synthase is encoded by a nucleic acid sequence selected from the group consisting of SEQ ID NOs: 104, 106, 108 and 110.

In one embodiment of any aspect disclosed above, the hydroxymethylglutaryl-CoA synthase is encoded by one or more nucleic acid molecules obtained from Saccharomyces sp. In another embodiment, the hydroxymethylglutaryl-CoA synthase is encoded by one or more nucleic acid molecules obtained from Saccharomyces cerevisiae. In some embodiments, the one or more nucleic acid molecules encoding the hydroxymethylglutaryl-CoA synthase is HmgS, or homolog thereof. In a further embodiment, the one or more nucleic acid molecules encoding the hydroxymethylglutaryl-CoA synthase comprises an amino acid sequence set forth in SEQ ID NO: 123. In yet another embodiment, the one or more nucleic acid molecules encoding the hydroxymethylglutaryl-CoA synthase is encoded by a nucleic acid sequence set forth in SEQ ID NO: 122.

In one embodiment of any aspect disclosed above, the methylglutaconyl-CoA hydratase is encoded by one or more nucleic acid molecules obtained from Pseudomonas sp. In another embodiment, the methylglutaconyl-CoA hydratase is encoded by one or more nucleic acid molecules obtained from Pseudomonas putida. In some embodiments, the one or more nucleic acid molecules encoding the methylglutaconyl-CoA hydratase is liuC, or homolog thereof. In a further embodiment, the one or more nucleic acid molecules encoding the methylglutaconyl-CoA hydratase comprises an amino acid sequence set forth in SEQ ID NO: 125. In yet another embodiment, the one or more nucleic acid molecules encoding the methylglutaconyl-CoA hydratase is encoded by a nucleic acid sequence set forth in SEQ ID NO: 124.

In one embodiment of any aspect disclosed above, the methylcrotonyl-CoA carboxylase is encoded by one or more nucleic acid molecules obtained from Pseudomonas sp. In another embodiment, the methylcrotonyl-CoA carboxylase is encoded by one or more nucleic acid molecules obtained from Pseudomonas aeruginosa. In some embodiments, the one or more nucleic acid molecules encoding the methylcrotonyl-CoA carboxylase is liuB, and/or liuD, or homologs thereof. In a further embodiment, the one or more nucleic acid molecules encoding the methylcrotonyl-CoA carboxylase comprises an amino acid sequence selected from SEQ ID NOs: 127 and 129. In yet another embodiment, the one or more nucleic acid molecules encoding the methylcrotonyl-CoA carboxylase is encoded by a nucleic acid sequence selected from SEQ ID NOs: 126 and 128.

In one embodiment of any aspect disclosed above, the methylcrotonyl-CoA hydratase is a 3-ketoacyl-CoA thiolase. In another embodiment, the methylcrotonyl-CoA hydratase is an enoyl-CoA hydratase. In another embodiment, the methylcrotonyl-CoA hydratase is encoded by one or more nucleic acid molecules obtained from E. coli. In some embodiments, the one or more nucleic acid molecules encoding the methylcrotonyl-CoA hydratase is fadA, and/or fadB, or homologs thereof. In a further embodiment, the one or more nucleic acid molecules encoding the methylcrotonyl-CoA hydratase comprises an amino acid sequence selected from SEQ ID NOs: 131 and 133. In yet another embodiment, the one or more nucleic acid molecules encoding the methylcrotonyl-CoA hydratase is encoded by a nucleic acid sequence selected from SEQ ID NOs: 130 and 132.

In one embodiment of any aspect disclosed above, the 3-hydroxyisovaleryl-CoA thioesterase is encoded by one or more nucleic acid molecules obtained from E. coli. In some embodiments, the one or more nucleic acid molecules encoding the 3-hydroxyisovaleryl-CoA thioesterase is tesB, or homolog thereof. In a further embodiment, the one or more nucleic acid molecules encoding the 3-hydroxyisovaleryl-CoA thioesterase comprises an amino acid sequence set forth in SEQ ID NO: 135. In yet another embodiment, the one or more nucleic acid molecules encoding the 3-hydroxyisovaleryl-CoA thioesterase is encoded by a nucleic acid sequence set forth in SEQ ID NO: 134.

In one embodiment of any aspect disclosed above, the 3HIV kinase is encoded by one or more nucleic acid molecules obtained from a microorganism selected from the group consisting of Thermoplasma sp. and Picrophilus sp. In another embodiment, the 3HIV kinase is encoded by one or more nucleic acid molecules obtained from a microorganism selected from the group consisting of Thermoplasma acidophilum and Picrophilus torridus. In some embodiments, the one or more nucleic acid molecules encoding the 3HIV kinase is TA1305 and/or PTO1356, or homolog thereof. In some embodiments, the TA1305 comprises a L200E mutation. In a further embodiment, the one or more nucleic acid molecules encoding the 3HIV kinase comprises an amino acid sequence selected from the group consisting of SEQ ID NOs: 113, 115 and 117. In yet another embodiment, the one or more nucleic acid molecules encoding the 3HIV kinase is encoded by a nucleic acid sequence selected from the group consisting of SEQ ID NOs: 112, 114 and 116.

In one embodiment of any aspect disclosed above, the 3HIV-3-phosphate decarboxylase is encoded by one or more nucleic acid molecules obtained from Streptococcus sp. In another embodiment, the 3HIV-3-phosphate decarboxylase is encoded by one or more nucleic acid molecules obtained from a microorganism selected from Streptococcus mitis and Streptococcus gordonii. In some embodiments, the one or more nucleic acid molecules encoding the 3HIV-3-phosphate decarboxylase comprises smi_1746 and/or mvaD, or homolog thereof. In a further embodiment, the one or more nucleic acid molecules encoding the 3HIV-3-phosphate decarboxylase comprises an amino acid sequence selected from SEQ ID NOs: 119 and 121. In yet another embodiment, the one or more nucleic acid molecules encoding the 3HIV-3-phosphate decarboxylase is encoded by a nucleic acid sequence selected from SEQ ID NOs: 118 and 120.

In one embodiment of any aspect disclosed above, the 3HIV decarboxylase is encoded by one or more nucleic acid molecules obtained from a microorganism selected from the group consisting of Streptococcus sp., Thermoplasma sp. and Picrophilus sp. In another embodiment, the 3HIV decarboxylase is encoded by one or more nucleic acid molecules obtained from a microorganism selected from the group consisting of Streptococcus gordonii, Thermoplasma acidophilum and Picrophilus torridus. In some embodiments, the one or more nucleic acid molecules encoding the 3HIV decarboxylase comprises mvaD, TA1305 and/or PTO1356, or homolog thereof. In a further embodiment, the one or more nucleic acid molecules encoding the 3HIV decarboxylase comprises an amino acid sequence selected from the group consisting of SEQ ID NOs: 113, 117 and 121. In yet another embodiment, the one or more nucleic acid molecules encoding the 3HIV decarboxylase is encoded by a nucleic acid sequence selected from the group consisting of SEQ ID NOs: 112, 116 and 120.

### Enzyme Engineering

The enzymes in the recombinant microorganism can be engineered to improve one or more aspects of the substrate to product conversion. Non-limiting examples of enzymes that can be further engineered for use in methods of the disclosure include an aldolase, an aldehyde reductase, an acetoacetyl coenzyme A hydrolase, a xylose isomerase, a xylitol dehydrogenase, a mevalonate diphosphate decarboxylase, and combinations thereof. These enzymes can be engineered for improved catalytic activity, improved selectivity, improved stability, improved tolerance to various fermentation conditions (temperature, pH, etc.), or improved tolerance to various metabolic substrates, products, by-products, intermediates, etc. The term “improved catalytic activity” as used herein with respect to a particular enzymatic activity refers to a higher level of enzymatic activity than that measured relative to a comparable non-engineered enzyme.

For example, engineering methods have been used to alter the stability, substrate specificity and stereospecificity of aldolases to produce excellent enzymes for biocatalytic processes. The thermostability and solvent tolerance of fructose-1,6-bisphosphate aldolase (FBP-aldolase) was increased using family DNA shuffling of the fda genes from Escherichia coli and Edwardsiella ictaluri. A fourth generation variant was identified which displayed an average 280-fold higher half-life at 53° C. than either parent. The same variant also displayed enhanced activity in various polar and non-polar organic solvents (Hao and Berry 2004 Protein Eng Des Sel 17:689-697).

As another example, acetoacetyl coenzyme A hydrolase can convert acetoacetyl-CoA to acetoacetate. However, the hydrolase is unspecific in that it also reacts with the same magnitude of order with acetyl-CoA, which is the substrate required for acetoacetyl-CoA formation by the enzyme thiolase. Thus, to create more efficient acetoacetyl-CoA hydrolases, these enzymes have been engineered to have at least 10× higher activity for the acetoacetyl-CoA substrate than for acetyl-CoA substrate by replacing several glutamic acid residues in the enzyme beta subunit that is important for catalysis (WO 2015/042588).

As another example, the E. coli YqhD enzyme is a broad substrate aldehyde reductase with NADPH-dependent reductase activity for more than 10 aldehyde substrates and is a useful enzyme to produce biorenewable fuels and chemicals (Jarboe 2010 Applied Microbiology and Biotechnology 89:249). Though YqhD enzyme activity is beneficial through its scavenging of toxic aldehydes, the enzyme is also NADPH-dependent and contributes to NADPH depletion and growth inhibition of organisms. Error-prone PCR of YqhD was performed in order to improve 1,3-propanediol production from 3-hydroxypropionaldehyde (3-HPA). This directed engineering yielded two mutants, D99QN147H and Q202A, with decreased Km and increased kcat for certain aldehydes, particularly 3-HPA (Li et al. 2008 Prog. Nat. Sci. 18 (12):1519-1524). The improved catalytic activity of the D99QN147H mutant is consistent with what is known about the structure of YqhD (Sulzenbacher et al. 2004 J. Mol. Biol. 342 (2):489-502), as residues Asp99 and Asn147 both interact with NADPH. Use of the D99QN147H mutant increased 1,3-propanediol production from 3-HPA 2-fold. Mutant YqhD enzymes with increased catalytic efficiency (increased Kcat/Km) toward NADPH have also been described in WO 2011012697 A2, which is herein incorporated in its entirety.

As another example, xylose isomerase is a metal-dependent enzyme that catalyzes the interconversion of aldose and ketose sugars, primarily between xylose to xylulose and glucose to fructose. It has lower affinity for lyxose, arabinose and mannose sugars. The hydroxyl groups of sugars may define the substrate preference of sugar isomerases. The aspartate at residue 256 of Thermus thermophilus xylose isomerase was replaced with arginine (Patel et al. 2012 Protein Engineering, Design & Selection vol. 25 no. 7 pp. 331-336). This mutant xylose isomerase exhibited an increase in specificity for D-lyxose, L-arabinose and D-mannose. The catalytic efficiency of the D256R xylose isomerase mutant was also higher for these 3 substrates compared to the wild type enzyme. It was hypothesized that the arginine at residue 256 in the mutant enzyme may play a role in the catalytic reaction or influence changes in substrate orientation.

As another example, the enzyme xylitol dehydrogenase plays a role in the utilization of xylose along with xylose reductase. Xylose reductase (XR) reduces xylose to xylitol and then xylitol dehydrogenase (XDH) reoxidizes xylitol to form xylulose. However, since XR prefers NADPH as cosubstrate, while XDH exclusively uses NAD+ as cosubstrate, a cosubstrate recycling problem is encountered. One solution is to engineer XDH such that its cosubstrate specificity is altered from NAD+ to NADP+ (Ehrensberger et al. 2006 Structure 14: 567-575). A crystal structure of the Gluconobacter oxydans holoenzyme revealed that Asp38 is largely responsible for the NAD+ specificity of XDH. Asp38 interacts with the hydroxyls of the adenosine ribose, and Met39 stacks under the purine ring and is also located near the 2′ hydroxyl. A double mutant (D38S/M39R) XDH was constructed that exclusively used NADP+ without loss of enzyme activity.

As another example, the enzyme mevalonate diphosphate decarboxylase (MVD) is an ATP-dependent enzyme which catalyzes the phosphorylation/decarboxylation of (R)-mevalonate-5-diphosphate to isopentenyl pyrophosphate (IPP) in the mevalonate (MVA) pathway. In the classical MVA pathway, MVD catalyzes the final step, where it produces IPP from (R)-mevalonate-5-diphosphate (MVAPP) in an irreversible reaction dependent upon ATP. MVAPP is phosphorylated first, and consequent decarboxylation occurs with the concomitant release of inorganic phosphate. With the same mechanism, classical MVDs also catalyze the conversion of the nonphosphorylated 3-hydroxyisovalerate (3-HIV) to isobutene. Mevalonate diphosphate (MDP) decarboxylase variants having improved activity in converting 3-phosphonoxyisovalerate into isobutene are disclosed in, for example, WO 2012052427 and WO 2015004211, each of which is herein incorporated in its entirety.

### Metabolic Engineering—Enzyme Overexpression or Enzyme Downregulation/Deletion for Increased Pathway Flux

In various embodiments described herein, the exogenous and endogenous enzymes in the recombinant microorganism participating in the biosynthesis pathways described herein may be overexpressed.

The terms “overexpressed” or “overexpression” refers to an elevated level (e.g., aberrant level) of mRNAs encoding for a protein(s), and/or to elevated levels of protein(s) in cells as compared to similar corresponding unmodified cells expressing basal levels of mRNAs or having basal levels of proteins. In particular embodiments, mRNA(s) or protein(s) may be overexpressed by at least 2-fold, 3-fold, 4-fold, 5-fold, 6-fold, 8-fold, 10-fold, 12-fold, 15-fold or more in microorganisms engineered to exhibit increased gene mRNA, protein, and/or activity.

In some embodiments, a recombinant microorganism of the disclosure is generated from a host that contains the enzymatic capability to synthesize substrates such as D-xylulose, D-ribulose, D-ribulose-1-phosphate, D-xylulose-1-phosphate, D-xylonolactone, D-xylonate, 2-keto-3-deoxy-xylonate, glycolaldehyde, DHAP, pyruvate, acetoacetyl-CoA, acetoacetate or 3-hydroxyisovalerate. In some embodiments, it can be useful to increase the synthesis or accumulation of, for example, D-xylulose, D-ribulose, D-ribulose-1-phosphate, D-xylulose-1-phosphate, D-xylonolactone, D-xylonate, 2-keto-3-deoxy-xylonate, glycolaldehyde, DHAP, pyruvate, acetoacetyl-CoA, acetoacetate or 3-hydroxyisovalerate, to increase the production of MEG and isobutene.

In some embodiments, it may be useful to increase the expression of endogenous or exogenous enzymes involved in the MEG and isobutene biosynthesis pathways to increase flux from, for example, D-xylulose, D-ribulose, D-ribulose-1-phosphate, D-xylulose-1-phosphate, D-xylonolactone, D-xylonate, 2-keto-3-deoxy-xylonate, glycolaldehyde, DHAP, pyruvate, acetoacetyl-CoA, acetoacetate or 3-hydroxyisovalerate, thereby resulting in increased synthesis or accumulation of MEG and isobutene.

Increased synthesis or accumulation can be accomplished by, for example, overexpression of nucleic acids encoding one or more of the above-described MEG and isobutene biosynthesis pathway enzymes. Overexpression of a MEG and isobutene biosynthesis pathway enzyme or enzymes can occur, for example, through increased expression of an endogenous gene or genes, or through the expression, or increased expression, of an exogenous gene or genes. Therefore, naturally occurring organisms can be readily modified to generate non-natural, MEG and isobutene producing microorganisms through overexpression of one or more nucleic acid molecules encoding a MEG and isobutene compound biosynthesis pathway enzyme. In addition, a non-naturally occurring organism can be generated by mutagenesis of an endogenous gene that results in an increase in activity of an enzyme in the MEG and isobutene biosynthesis pathways.

Equipped with the present disclosure, the skilled artisan will be able to readily construct the recombinant microorganisms described herein, as the recombinant microorganisms of the disclosure can be constructed using methods well known in the art as exemplified above to exogenously express at least one nucleic acid encoding a MEG and isobutene biosynthesis pathway enzyme in sufficient amounts to produce MEG and isobutene.

Methods for constructing and testing the expression levels of a non-naturally occurring MEG and isobutene-producing host can be performed, for example, by recombinant and detection methods well known in the art. Such methods can be found described in, for example, Sambrook et al., Molecular Cloning: A Laboratory Manual, Third Ed., Cold Spring Harbor Laboratory, New York (2001); Ausubo et al., Current Protocols in Molecular Biology, John Wiley and Sons, Baltimore, Md. (1999).

A variety of mechanisms known in the art can be used to express, or overexpress, exogenous or endogenous genes. For example, an expression vector or vectors can be constructed to harbor one or more MEG and isobutene biosynthesis pathway enzymes encoding nucleic acids as exemplified herein operably linked to expression control sequences functional in the host organism. Expression vectors applicable for use in the microbial host organisms of the invention include, for example, plasmids, phage vectors, viral vectors, episomes and artificial chromosomes, including vectors and selection sequences or markers operable for stable integration into a host chromosome. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of exogenous nucleic acid sequences involved in a metabolic or synthetic pathway can be confirmed using methods well known in the art.

As will be understood by those of skill in the art, it can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host, a process sometimes called “codon optimization” or “controlling for species codon bias.”

Optimized coding sequences containing codons preferred by a particular prokaryotic or eukaryotic host (see also, Murray et al. (1989) Nucl. Acids Res. 17:477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon (Dalphin et al. (1996) Nucl. Acids Res. 24: 216-218).

Those of skill in the art will recognize that, due to the degenerate nature of the genetic code, a variety of nucleic acid sequences can be used to encode a given enzyme of the disclosure. The nucleic acid sequences encoding the biosynthetic enzymes are referenced herein merely to illustrate an embodiment of the disclosure, and the disclosure includes any nucleic acid sequences that encode the amino acid sequences of the polypeptides and proteins of the enzymes of the present disclosure. In similar fashion, a polypeptide can typically tolerate one or more amino acid substitutions, deletions, and insertions in its amino acid sequence without loss or significant loss of a desired activity. The disclosure includes such polypeptides with different amino acid sequences than the specific proteins described herein so long as the modified or variant polypeptides have the enzymatic anabolic or catabolic activity of the reference polypeptide. Furthermore, the amino acid sequences encoded by the nucleic acid sequences shown herein merely illustrate embodiments of the disclosure.

Expression control sequences are known in the art and include, for example, promoters, enhancers, polyadenylation signals, transcription terminators, internal ribosome entry sites (IRES), and the like, that provide for the expression of the polynucleotide sequence in a host cell. Expression control sequences interact specifically with cellular proteins involved in transcription (Maniatis et al., Science, 236: 1237-1245 (1987)). Exemplary expression control sequences are described in, for example, Goeddel, Gene Expression Technology: Methods in Enzymology, Vol. 185, Academic Press, San Diego, Calif. (1990).

In various embodiments, an expression control sequence may be operably linked to a polynucleotide sequence. By “operably linked” is meant that a polynucleotide sequence and an expression control sequence(s) are connected in such a way as to permit gene expression when the appropriate molecules (e.g., transcriptional activator proteins) are bound to the expression control sequence(s). Operably linked promoters are located upstream of the selected polynucleotide sequence in terms of the direction of transcription and translation. Operably linked enhancers can be located upstream, within, or downstream of the selected polynucleotide.

In some embodiments, the recombinant microorganism is manipulated to delete, disrupt, mutate, and/or reduce the activity of one or more endogenous enzymes that catalyzes a reaction in a pathway that competes with the biosynthesis pathway for the production of MEG and isobutene.

In some embodiments, the recombinant microorganism is manipulated to delete, disrupt, mutate, and/or reduce the activity of one or more endogenous enzymes that catalyzes the conversion of D-xylulose to D-xylulose-5-phosphate. In some such embodiments, the enzyme that catalyzes the conversion of D-xylulose to D-xylulose-5-phosphate is a D-xylulose-5-kinase. In some embodiments, the D-xylulose-5-kinase is from Escherichia coli. In some embodiments, the D-xylulose-5-kinase is encoded by the xylB gene or homologs thereof. In some embodiments, the manipulation prevents the conversion of D-xylulose to D-xylulose-5-phosphate and instead shunts the reaction toward conversion of D-xylulose to D-xylulose-1-phosphate.

In some embodiments, the recombinant microorganism is manipulated to delete, disrupt, mutate, and/or reduce the activity of one or more endogenous enzymes that catalyzes the conversion of glycolaldehyde to glycolic acid. In some such embodiments, the enzyme that catalyzes the conversion of glycolaldehyde to glycolic acid is a glycolaldehyde dehydrogenase. In some embodiments, the glycolaldehyde dehydrogenase is from Escherichia coli. In some embodiments, the glycolaldehyde dehydrogenase is encoded by the aldA gene or homologs thereof. In some embodiments, the manipulation prevents the production of glycolic acid from glycolaldehyde and instead shunts the reaction toward conversion of glycolaldehyde to MEG.

In some embodiments, the recombinant microorganism is manipulated to delete, disrupt, mutate, and/or reduce the activity of one or more endogenous enzymes that catalyzes the conversion of pyruvate to lactate. In some such embodiments, the enzyme that catalyzes the conversion of pyruvate to lactate is a lactate dehydrogenase. In some embodiments, the lactate dehydrogenase is from Escherichia coli. In some embodiments, the lactate dehydrogenase is encoded by the IdhA gene or homologs thereof. In some embodiments, the manipulation prevents the production of lactate from pyruvate and instead shunts the reaction toward production of isobutene.

In some embodiments, the recombinant microorganism is manipulated to delete, disrupt, mutate, and/or reduce the activity of one or more endogenous enzymes that catalyzes the conversion of D-xylose to D-xylulose. In some such embodiments, the enzyme that catalyzes the conversion of D-xylose to D-xylulose is a D-xylose isomerase. In some embodiments, the D-xylose isomerase is from E. coli. In some embodiments, the D-xylose isomerase is encoded by the xylA gene or homologs thereof. In some embodiments, the manipulation prevents conversion of D-xylose to D-xylulose and instead shunts the reaction toward the conversion of D-xylose to D-xylonate.

### Examples

### Example 1: Production of ethylene glycol in E. coli via ribulose-1-phosphate pathway

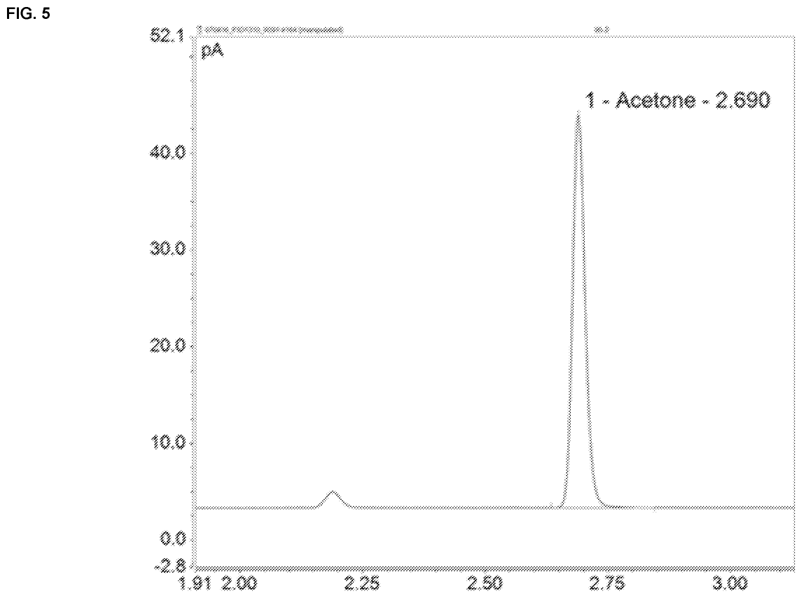

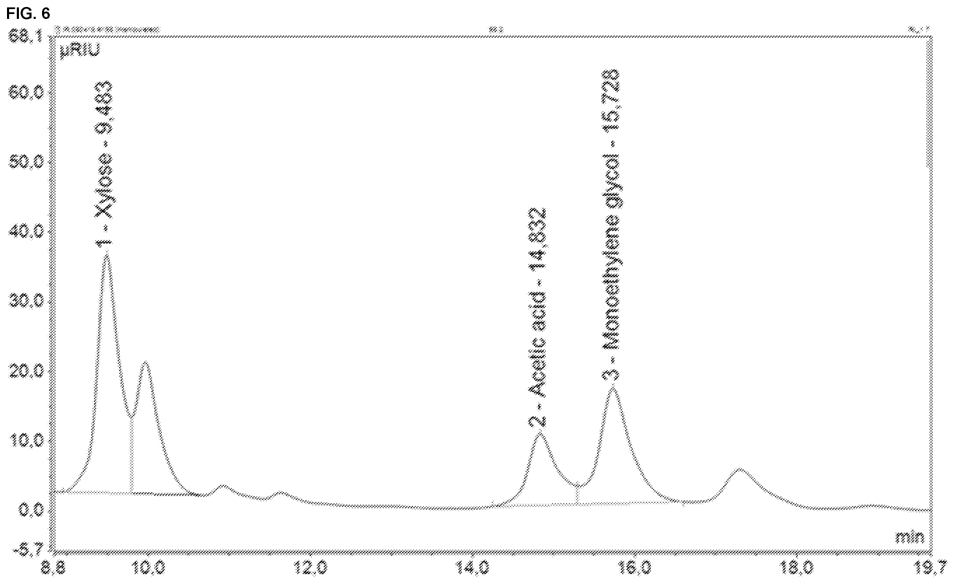

E. coli K12 strain MG1655 was used as host for the deletion of two genes that could divert the carbon flux from MEG+acetone pathway: aldA and xylB. The genes were successfully deleted and deletion confirmed by sequencing. The E. coli K12 strain MG1655 with aldA and xylB genes deleted was used as host for the implementation of both MEG and acetone pathway. An operon containing dte (D-tagatose 3-epimerase enzyme, SEQ ID NO: 3, encoded by nucleic acid sequence SEQ ID NO: 2), fucA (D-ribulose-1-phosphate aldolase enzyme, SEQ ID NO: 11, encoded by nucleic acid sequence SEQ ID NO: 10), fucO (aldehyde reductase enzyme, SEQ ID NO: 28, encoded by nucleic acid sequence SEQ ID NO: 27) and fucK (D-ribulokinase enzyme, SEQ ID NO: 8, encoded by nucleic acid sequence SEQ ID NO: 7) genes under the control of the proD promoter was constructed in a pET28a backbone. The plasmid was constructed using In-fusion commercial kit and confirmed by sequencing. A second operon, for acetone production, containing thl (thiolase enzyme, SEQ ID NO: 35, encoded by nucleic acid sequence SEQ ID NO: 34), atoA/atoD (acetate:acetoacetyl-CoA transferase, SEQ ID NOs: 43 and 46, encoded by nucleic acid sequences SEQ ID NOs: 42 and 45, respectively) and adc (acetoacetate decarboxylase, SEQ ID NO: 49, encoded by nucleic acid sequence SEQ ID NO: 48) genes under the control of the proD promoter was constructed in a pZA31 backbone. The acetone pathway was also constructed using In-fusion commercial kit. Both plasmids were co-transformed into an MG1655 strain with aldA and xylB genes deleted. Colonies from the transformation were inoculated in 3 mL of LB media for pre-culture. After 16 hours of cultivation the pre-culture was transferred to 50 mL of TB media containing 15 g/L of xylose to an initial OD of 0.2. The flasks were incubated at 37° C., 250 rpm until complete consumption of xylose. After 46 hours of cultivation 2.8 g/L of MEG and 1.5 g/L of acetone were co-produced from 11 g/L of xylose (see FIGS. 5 and 6). Maximum yield and productivity were, respectively, 0.39 g/g and 0.09 g/L·h.

### Example 2: Production of Ethylene Glycol in E. coli Via Xylulose-1-Phosphate Pathway

An operon containing genes for D-xylulose 1-kinase (ketohexokinase C, SEQ ID NO:55, encoded by nucleic acid sequence SEQ ID NO: 54), D-xylulose-1-phosphate aldolase (Fructose-bisphosphate aldolase B, SEQ ID NO: 58, encoded by nucleic acid sequence SEQ ID NO: 57), and fucO (aldehyde reductase enzyme, SEQ ID NO: 28, encoded by nucleic acid sequence SEQ ID NO: 27) under the control of the proD promoter was constructed in a pET28a backbone using In-fusion commercial kit and confirmed by sequencing. Subsequently, the operon, which constitutes the MEG via xylulose-1-phosphate pathway, was integrated into the E. coli K12 MG1655 ΔaldA ΔxylB strain. The operon for acetone production, containing thl, atoA/atoD and adc genes under the control of the proD promoter (see Example 1) was re-cloned from pZA31 into a pZS13* backbone. Another operon with the genes for the enzymes 3HIV synthase (HMG-CoA synthase, SEQ ID NO: 105, encoded by nucleic acid sequence SEQ ID NO: 104), HIV kinase (mevalonate 3-kinase, SEQ ID NO: 115, encoded by nucleic acid sequence SEQ ID NO: 114) and 3-phosphonoxyisovalerate decarboxylase (mevalonate-diphosphate decarboxylase, SEQ ID NO: 119, encoded by nucleic acid sequence SEQ ID NO: 118), under the control of the proD promotor, was cloned into pET28 using In-fusion commercial kit. The two plasmids, which together compose the complete isobutene pathway, were co-transformed into the strain carrying the integrated MEG via xylulose-1-phosphate pathway. Colonies from the transformation were inoculated in 3 mL of LB media for pre-culture. After 16 hours of cultivation the pre-culture was transferred to 50 mL of TB media containing 15 g/L of xylose to an initial OD of 0.2. The flasks were incubated at 37° C., 250 rpm until complete consumption of xylose. After 46 hours of cultivation, MEG and isobutene are co-produced.

### Example 3: In Vivo Isobutene Production from Acetone and 3-Hydroxyisovalerate (3HIV) Supplemented in the Minimal Medium

All plasmids harboring the isobutene route genes optimized for expression in Escherichia coli were constructed using standard molecular cloning techniques. The full-length of the wild type or mutated coding sequence of 3-HIV synthase, mevalonate-3-kinase (M3K) and mevalonate diphosphate decarboxylase (MDD) were cloned into pET28a under the control of an inducible promoter T5 and terminator T1. Constructs 01 and 03 harbored only the mutated sequences of 3-HIV synthase, MDD and M3K (SEQ ID NOs: 136, 115 and 137, respectively). The difference between them was the order of the three genes in the operon: while construct 01 had the inducible promoter T5 followed by the coding sequences of MDD+3HIV synthase+M3K, the construct 03 had 3HIV synthase and MDD sequence order switched. Instead, construct 02 harbored the inducible promoter T5 followed by the wild type sequences of 3-HIV synthase (SEQ ID NO: 105) and MDD (SEQ ID NO: 119), respectively, being the mutated coding sequence of M3K (SEQ ID NO: 115) placed at last, upstream the terminator T1. DNA sequencing was carried out on randomly selected clones to make sure there was no undesired nucleotide or amino acid mutation.

The isobutene production plasmids were transformed into BL21 (DE3) competent cells and plated out onto LB agar plates supplemented with the appropriate antibiotic. Cells were grown overnight at 30° C. Single colonies were picked and transferred to 20 ml of liquid TB medium supplemented with the appropriate antibiotic. Cell growth was carried out with agitation for 16-20 hours at 30° C. Cell cultures were then used to inoculate 100 ml of liquid TB medium supplemented with the appropriate antibiotic. Cultures were grown at 30° C. for 3-4 hours in shaking incubator. 1 mM IPTG final concentration was added once culture OD₆₀₀ reached 0.6-0.7 in order to favor the overexpression of the isobutene route recombinant enzymes. After 4-5 hours of IPTG induction, cells were pelleted by centrifugation and clarified medium discarded.

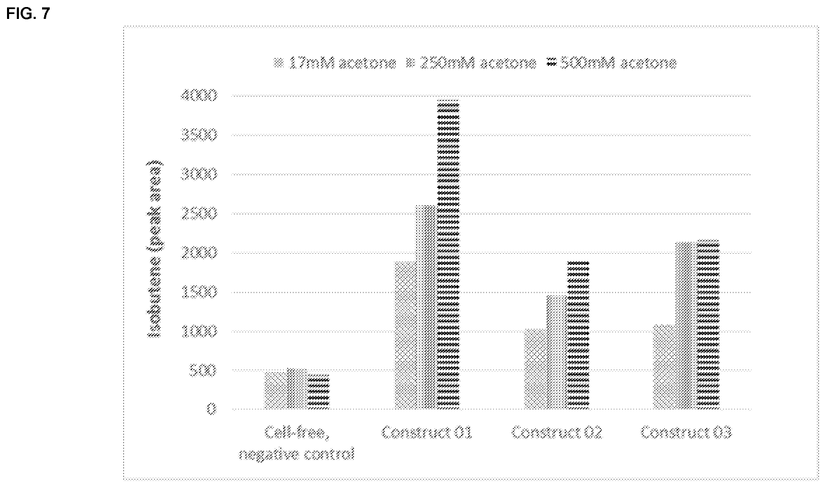

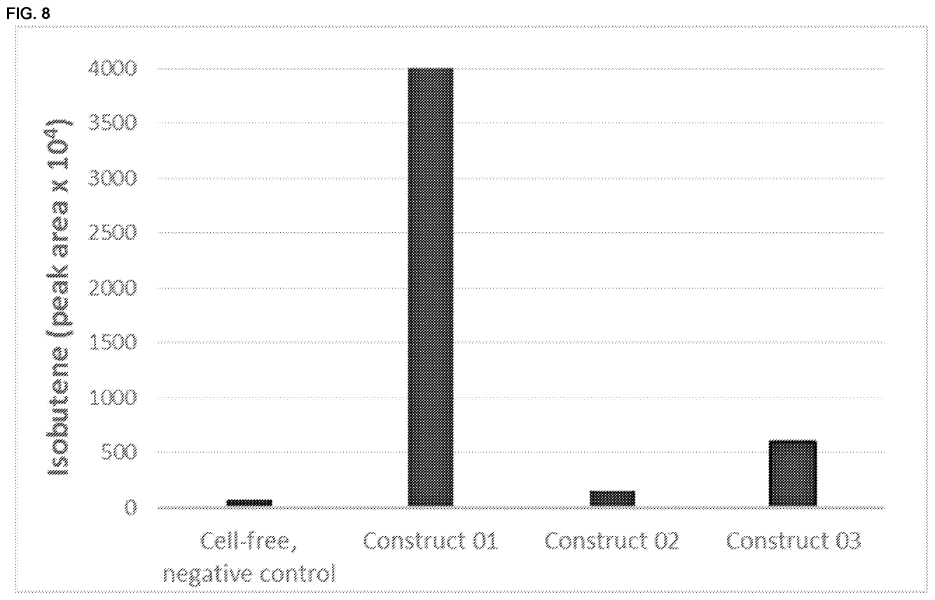

Bacterial pellets harboring the three overexpressed isobutene route enzymes were ressuspended in a minimal medium (200 mM K2HPO4, 20 mM NH4Cl, 4 mM Citric Acid, 3 mM CaCl2), 0.3 mM Cl2Co, 1 mM Cl2Mn, 0.3 mM Cl2Cu, 0.3 mM Na203Se, 0.3 mM NiSO4.6H2O, 0.3 mM ZnCl2, 0.3 mM C6H5FeO7, 0.4 mM H3BO3) supplemented with the appropriate antibiotic and 40 g/L glucose to reach a final culture OD₆₀₀=40. The in-vivo isobutene production assay was then set up transferring 500 ul of the concentrated cultures to 2 ml vials followed by 500 ul addition of minimal medium supplemented with the appropriate antibiotic, 40 g/L glucose and desired substrate in order to have a 1 ml final volume culture at OD₆₀₀=20. The final substrate concentration in the in-vivo assay was 17 mM, 250 mM and 500 mM for acetone and 10 mM for 3-hydroxyisovalarate (3-HIV). The glass vials were tightly closed and incubated at 37° C. for 72 hs in a shaking incubator. Bacterial cultures were deactivated by 5 min incubation at 80° C. before submitting samples for isobutene analytical detection via gas chromatography. Results showing enzymatic conversion of acetone or 3-HIV to isobutene are described in FIG. 7 and FIG. 8.

### DESCRIPTION OF SEQUENCES

| | |
|---|---|
| SEQ ID NO: 1 | Pseudomonas cichorii D-tagatose 3-epimerase DTE wild type NT |
| | sequence |
| SEQ ID NO: 2 | Pseudomonas cichorii D-tagatose 3-epimerase DTE codon |
| | optimized NT sequence |
| SEQ ID NO: 3 | Pseudomonas cichorii D-tagatose 3-epimerase DTE AA sequence |
| SEQ ID NO: 4 | Rhodobacter sphaeroides D-tagatose 3-epimerase FJ851309.1 wild |
| | type NT sequence |
| SEQ ID NO: 5 | Rhodobacter sphaeroides D-tagatose 3-epimerase FJ851309.1 AA |
| | sequence |
| SEQ ID NO: 6 | Escherichia coli L-fuculokinase FucK wild type NT sequence |
| SEQ ID NO: 7 | Escherichia coli L-fuculokinase FucK codon optimized NT sequence |
| SEQ ID NO: 8 | Escherichia coli L-fuculokinase fucK AA sequence |
| SEQ ID NO: 9 | Escherichia coli L-fuculose phosphate aldolase fucA wild type NT |
| | sequence |
| SEQ ID NO: 10 | Escherichia coli L-fuculose phosphate aldolase fucA |
| SEQ ID NO: 11 | Escherichia coli L-fuculose phosphate aldolase fucA AA sequence |
| SEQ ID NO: 12 | Escherichia coli glycerol dehydrogenase gldA wild type NT |
| | sequence |
| SEQ ID NO: 13 | Escherichia coli glycerol dehydrogenase gldA AA sequence |
| SEQ ID NO: 14 | Saccharomyces cerevisiae methylglyoxal reductase GRE2 wild type |
| | NT sequence |
| SEQ ID NO: 15 | Saccharomyces cerevisiae methylglyoxal reductase GRE2 AA |
| | sequence |
| SEQ ID NO: 16 | Saccharomyces cerevisiae aldose reductase GRE3 wild type NT |
| | sequence |
| SEQ ID NO: 17 | Saccharomyces cerevisiae aldose reductase GRE3 AA sequence |
| SEQ ID NO: 18 | Escherichia coli alcohol dehydrogenase yqhD* wild type NT |
| | sequence |
| SEQ ID NO: 19 | Escherichia coli alcohol dehydrogenase yqhD* codon optimized NT |
| | sequence |
| SEQ ID NO: 20 | Escherichia coli alcohol dehydrogenase yqhD* AA sequence |
| SEQ ID NO: 21 | Escherichia coli alcohol dehydrogenase yqhD wild type NT |
| | sequence |
| SEQ ID NO: 22 | Escherichia coli alcohol dehydrogenase yqhD codon optimized NT |
| | sequence |
| SEQ ID NO: 23 | Escherichia coli alcohol dehydrogenase yqhD AA sequence |
| SEQ ID NO: 24 | Escherichia coli methylglyoxal reductase ydjG wild type NT |
| | sequence |
| SEQ ID NO: 25 | Escherichia coli methylglyoxal reductase ydjG AA sequence |
| SEQ ID NO: 26 | Escherichia coli lactaldehyde reductase fucO wild type NT sequence |
| SEQ ID NO: 27 | Escherichia coli lactaldehyde reductase fucO codon optimized NT |
| | sequence |
| SEQ ID NO: 28 | Escherichia coli lactaldehyde reductase fucO AA sequence |
| SEQ ID NO: 29 | Escherichia coli methylglyoxal reductase yafB (dkgB) |
| | [multifunctional] wild type NT sequence |
| SEQ ID NO: 30 | Escherichia coli methylglyoxal reductase yafB (dkgB) |
| | [multifunctional] AA sequence |
| SEQ ID NO: 31 | Escherichia coli 2,5-diketo-D-gluconic acid reductase A yqhE (dkgA) |
| | wild type NT sequence |
| SEQ ID NO: 32 | Escherichia coli 2,5-diketo-D-gluconic acid reductase A yqhE (dkgA) |
| | AA sequence |
| SEQ ID NO: 33 | Clostridium acetobutylicum acetyl coenzyme A acetyltransferase |
| | thlA wild type NT sequence |
| SEQ ID NO: 34 | Clostridium acetobutylicum acetyl coenzyme A acetyltransferase |
| | thlA codon optimized NT sequence |
| SEQ ID NO: 35 | Clostridium acetobutylicum acetyl coenzyme A acetyltransferase |
| | thlA AA sequence |
| SEQ ID NO: 36 | Escherichia coli acetyl coenzyme A acetyltransferase atoB wild type |
| | NT sequence |
| SEQ ID NO: 37 | Escherichia coli acetyl coenzyme A acetyltransferase atoB AA |
| | sequence |
| SEQ ID NO: 38 | Saccharomyces cerevisiae acetyl coenzyme A acetyltransferase |
| | ERG10 wild type NT sequence |
| SEQ ID NO: 39 | Saccharomyces cerevisiae acetyl coenzyme A acetyltransferase |
| | ERG10 codon optimized NT sequence |
| SEQ ID NO: 40 | Saccharomyces cerevisiae acetyl coenzyme A acetyltransferase |
| | ERG10 AA sequence |
| SEQ ID NO: 41 | Escherichia coli Acetyl-CoA:acetoacetate-CoA transferase subunit |
| | atoA wild type NT sequence |
| SEQ ID NO: 42 | Escherichia coli Acetyl-CoA:acetoacetate-CoA transferase subunit |
| | atoA codon optimized NT sequence |
| SEQ ID NO: 43 | Escherichia coli Acetyl-CoA:acetoacetate-CoA transferase subunit |
| | atoA AA sequence |
| SEQ ID NO: 44 | Escherichia coli Acetyl-CoA:acetoacetate-CoA transferase subunit |
| | atoD wild type NT sequence |
| SEQ ID NO: 45 | Escherichia coli Acetyl-CoA:acetoacetate-CoA transferase subunit |
| | atoD codon optimized NT sequence |
| SEQ ID NO: 46 | Escherichia coli Acetyl-CoA:acetoacetate-CoA transferase subunit |
| | atoD AA sequence |
| SEQ ID NO: 47 | Clostridium acetobutylicum acetoacetate decarboxylase adc wild |
| | type NT sequence |
| SEQ ID NO: 48 | Clostridium acetobutylicum acetoacetate decarboxylase adc codon |
| | optimized NT sequence |
| SEQ ID NO: 49 | Clostridium acetobutylicum acetoacetate decarboxylase adc AA |
| | sequence |
| SEQ ID NO: 50 | Clostridium beijerinckii acetoacetate decarboxylase adc wild type |
| | NT sequence |
| SEQ ID NO: 51 | Clostridium beijerinckii acetoacetate decarboxylase adc codon |
| | optimized NT sequence |
| SEQ ID NO: 52 | Clostridium beijerinckii acetoacetate decarboxylase adc AA |
| | sequence |
| SEQ ID NO: 53 | Homo sapiens ketohexokinase C khk-C wild type cDNA sequence |
| SEQ ID NO: 54 | Homo sapiens ketohexokinase C khk-C codon optimized cDNA |
| | sequence |
| SEQ ID NO: 55 | Homo sapiens ketohexokinase C khk-C AA sequence |
| SEQ ID NO: 56 | Homo sapiens Fructose-bisphosphate aldolase B aldoB wild type |
| | cDNA sequence |
| SEQ ID NO: 57 | Homo sapiens Fructose-bisphosphate aldolase B aldoB codon |
| | optimized cDNA sequence |
| SEQ ID NO: 58 | Homo sapiens Fructose-bisphosphate aldolase B aldoB AA |
| | sequence |
| SEQ ID NO: 59 | Caulobacter crescentus D-xylose 1-dehydrogenase xylB wild type |
| | NT sequence |
| SEQ ID NO: 60 | Caulobacter crescentus D-xylose 1-dehydrogenase xylB codon |
| | optimized NT sequence |
| SEQ ID NO: 61 | Caulobacter crescentus D-xylose 1-dehydrogenase xylB AA |
| | sequence |
| SEQ ID NO: 62 | Haloferax volcanii D-xylose 1-dehydrogenase xdh1, HVO_B0028 |
| | wild type NT sequence |
| SEQ ID NO: 63 | Haloferax volcanii D-xylose 1-dehydrogenase xdh1, HVO_B0028 |
| SEQ ID NO: 64 | Trichoderma reesei D-xylose 1-dehydrogenase xyd1 wild type NT |
| | sequence |
| SEQ ID NO: 65 | Trichoderma reesei D-xylose 1-dehydrogenase xyd1 AA sequence |
| SEQ ID NO: 66 | Caulobacter crescentus Xylonolactonase xylC wild type NT |
| | sequence |
| SEQ ID NO: 67 | Caulobacter crescentus Xylonolactonase xylC AA sequence |
| SEQ ID NO: 68 | Caulobacter crescentus xylonate dehydratase xylD wild type NT |
| | sequence |
| SEQ ID NO: 69 | Caulobacter crescentus xylonate dehydratase xylD AA sequence |
| SEQ ID NO: 70 | Escherichia coli xylonate dehydratase yjhG wild type NT sequence |
| SEQ ID NO: 71 | Escherichia coli xylonate dehydratase yjhG codon optimized NT |
| | sequence |
| SEQ ID NO: 72 | Escherichia coli xylonate dehydratase yjhG AA sequence |
| SEQ ID NO: 73 | Escherichia coli xylonate dehydratase yagF wild type NT sequence |
| SEQ ID NO: 74 | Escherichia coli xylonate dehydratase yagF codon optimized NT |
| | sequence |
| SEQ ID NO: 75 | Escherichia coli xylonate dehydratase yagF AA sequence |
| SEQ ID NO: 76 | Escherichia coli Uncharacterized lyase yjhH wild type NT sequence |
| SEQ ID NO: 77 | Escherichia coli Uncharacterized lyase yjhH codon optimized NT |
| | sequence |
| SEQ ID NO: 78 | Escherichia coli Uncharacterized lyase yjhH AA sequence |
| SEQ ID NO: 79 | Escherichia coli Probable 2-keto-3-deoxy-galactonate aldolase |
| | yagE wild type NT sequence |
| SEQ ID NO: 80 | Escherichia coli Probable 2-keto-3-deoxy-galactonate aldolase yagE |
| | codon optimized NT sequence |
| SEQ ID NO: 81 | Escherichia coli Probable 2-keto-3-deoxy-galactonate aldolase yagE |
| | AA sequence |
| SEQ ID NO: 82 | Scheffersomyces stipitis D-xylose reductase xyl1 wild type NT |
| | sequence |
| SEQ ID NO: 83 | Scheffersomyces stipitis D-xylose reductase xyl1 codon optimized |
| | NT sequence |
| SEQ ID NO: 84 | Scheffersomyces stipitis D-xylose reductase xyl1 AA sequence |
| SEQ ID NO: 85 | Saccharomyces cerevisiae aldose reductase GRE3 wild type NT |
| | sequence |
| SEQ ID NO: 86 | Saccharomyces cerevisiae aldose reductase GRE3 codon |
| | optimized NT sequence |
| SEQ ID NO: 87 | Saccharomyces cerevisiae aldose reductase GRE3 AA sequence |
| SEQ ID NO: 88 | Scheffersomyces stipitis D-xylulose reductase xyl2 wild type NT |
| | sequence |
| SEQ ID NO: 89 | Scheffersomyces stipitis D-xylulose reductase xyl2 codon optimized |
| | NT sequence |
| SEQ ID NO: 90 | Scheffersomyces stipitis D-xylulose reductase xyl2 AA sequence |
| SEQ ID NO: 91 | Trichoderma reesei Xylitol dehydrogenase xdh1 wild type NT |
| | sequence |
| SEQ ID NO: 92 | Trichoderma reesei Xylitol dehydrogenase xdh1 AA sequence |
| SEQ ID NO: 93 | Pyromyces sp. xylose isomerase xylA wild type NT sequence |
| SEQ ID NO: 94 | Pyromyces sp. xylose isomerase xylA codon optimized NT |
| | sequence |
| SEQ ID NO: 95 | Pyromyces sp. xylose isomerase xylA AA sequence |
| SEQ ID NO: 96 | Clostridium acetobutylicum butyrate-acetoacetate CoA-transferase, |
| | complex A ctfA wild type NT sequence |
| SEQ ID NO: 97 | Clostridium acetobutylicum butyrate-acetoacetate CoA-transferase, |
| | complex A ctfA AA sequence |
| SEQ ID NO: 98 | Clostridium acetobutylicum butyrate-acetoacetate CoA-transferase, |
| | subunit B ctfB wild type NT sequence |
| SEQ ID NO: 99 | Clostridium acetobutylicum butyrate-acetoacetate CoA-transferase, |
| | subunit B ctfB AA sequence |
| SEQ ID NO: 100 | Escherichia coli (strain K12) Acetyl-CoA:acetoacetate-CoA |
| | transferase subunit atoA wild type NT sequence |
| SEQ ID NO: 101 | Escherichia coli (strain K12) Acetyl-CoA:acetoacetate-CoA |
| | transferase subunit atoA AA sequence |
| SEQ ID NO: 102 | Escherichia coli (strain K12) Acetyl-CoA:acetoacetate-CoA |
| | transferase subunit atoD wild type NT sequence |
| SEQ ID NO: 103 | Escherichia coli (strain K12) Acetyl-CoA:acetoacetate-CoA |
| | transferase subunit atoD AA sequence |
| SEQ ID NO: 104 | Mus musculus hydroxymethylglutaryl-CoA synthase Hmgcs1 wild |
| | type NT sequence |
| SEQ ID NO: 105 | Mus musculus hydroxymethylglutaryl-CoA synthase Hmgcs1 AA |
| | sequence |
| SEQ ID NO: 106 | Saccharomyces cerevisiae hydroxymethylglutaryl-CoA synthase |
| | ERG13 wild type NT sequence |
| SEQ ID NO: 107 | Saccharomyces cerevisiae hydroxymethylglutaryl-CoA synthase |
| | ERG13 AA sequence |
| SEQ ID NO: 108 | Lactobacillus crispatus ST1 hydroxymethylglutaryl-CoA synthase/ |
| | polyketide intermediate transferase PksG wild type NT sequence |
| SEQ ID NO: 109 | Lactobacillus crispatus ST1 hydroxymethylglutaryl-CoA synthase/ |
| | polyketide intermediate transferase PksG AA sequence |
| SEQ ID NO: 110 | Polaromonas naphthalenivorans hydroxymethylglutaryl-CoA lyase |
| | Pnap_0477 wild type NT sequence |
| SEQ ID NO: 111 | Polaromonas naphthalenivorans hydroxymethylglutaryl-CoA lyase |
| | Pnap_0477 AA sequence |
| SEQ ID NO: 112 | Thermoplasma acidophilum mevalonate-diphosphate |
| | decarboxylase/mevalonate-monophosphate decarboxylase TA1305 |
| | wild type NT sequence |
| SEQ ID NO: 113 | Thermoplasma acidophilum mevalonate-diphosphate |
| | decarboxylase/mevalonate-monophosphate decarboxylase TA1305 |
| | AA sequence |
| SEQ ID NO: 114 | Thermoplasma acidophilum mevalonate-diphosphate |
| | decarboxylase/mevalonate-monophosphate decarboxylase |
| | TA1305* wild type NT sequence |
| SEQ ID NO: 115 | Thermoplasma acidophilum mevalonate-diphosphate |
| | decarboxylase/mevalonate-monophosphate decarboxylase |
| | TA1305* AA sequence |
| SEQ ID NO: 116 | Picrophilus torridus mevalonate-diphosphate decarboxylase |
| | PTO1356 wild type NT sequence |
| SEQ ID NO: 117 | Picrophilus torridus mevalonate-diphosphate decarboxylase |
| | PTO1356 AA sequence |
| SEQ ID NO: 118 | Streptococcus mitis mevalonate-diphosphate decarboxylase |
| | smi_1746 wild type NT sequence |
| SEQ ID NO: 119 | Streptococcus mitis mevalonate-diphosphate decarboxylase |
| | smi_1746 AA sequence |
| SEQ ID NO: 120 | Streptococcus gordonii mevalonate-diphosphate decarboxylase |
| | mvaD wild type NT sequence |
| SEQ ID NO: 121 | Streptococcus gordonii mevalonate-diphosphate decarboxylase |
| | mvaD AA sequence |
| SEQ ID NO: 122 | Saccharomyces cerevisiae HMG-CoA synthase HmgS NT |
| | sequence |
| SEQ ID NO: 123 | Saccharomyces cerevisiae HMG-CoA synthase HmgS AA |
| | sequence |
| SEQ ID NO: 124 | Pseudomonas putida methylglutaconyl-CoA hydratase liuC NT |
| | sequence |
| SEQ ID NO: 125 | Pseudomonas putida methylglutaconyl-CoA hydratase liuC AA |
| | sequence |
| SEQ ID NO: 126 | Pseudomonas aeruginosa methylcrotonyl-CoA carboxylase subunit |
| | beta liuB NT sequence |
| SEQ ID NO: 127 | Pseudomonas aeruginosa methylcrotonyl-CoA carboxylase subunit |
| | beta liuB AA sequence |
| SEQ ID NO: 128 | Pseudomonas aeruginosa methylcrotonyl-CoA carboxylase subunit |
| | alpha liuD NT sequence |
| SEQ ID NO: 129 | Pseudomonas aeruginosa methylcrotonyl-CoA carboxylase subunit |
| | alpha liuD AA sequence |
| SEQ ID NO: 130 | Escherichia coli fatty acid oxidation complex, 3-ketoacyl-CoA |
| | thiolase fadA NT sequence |
| SEQ ID NO: 131 | Escherichia coli fatty acid oxidation complex, 3-ketoacyl-CoA |
| | thiolase fadA AA sequence |
| SEQ ID NO: 132 | Escherichia coli fatty acid oxidation complex, enoyl-CoA hydratase |
| | fadB NT sequence |
| SEQ ID NO: 133 | Escherichia coli fatty acid oxidation complex, enoyl-CoA hydratase |
| | fadB AA sequence |
| SEQ ID NO: 134 | Escherichia coli acyl-CoA thioesterase tesB NT sequence |
| SEQ ID NO: 135 | Escherichia coli acyl-CoA thioesterase tesB AA sequence |
| SEQ ID NO: 136 | Mus musculus hydroxymethylglutaryl-CoA synthase Hmgcs1*, |
| | mutated amino acid sequence (Q3UWQ9 T165P I222Q S296Q |
| | V500S) |
| SEQ ID NO: 137 | Streptococcus mitis mevalonate-diphosphate decarboxylase |
| | smi_1746*, mutated amino acid sequence (D3HAT7_I16L R24K |
| | C118L Y121R S141P E159L M173C E177C K180P K241I S248T |
| | K282C E291D F297L L303M T308S) |
| SEQ ID NO: 138 | Streptomyces sp. acetoacetyl CoA synthase NphT7, amino acid |
| | sequence |
| SEQ ID NO: 139 | Escherichia coli acetyl-CoA carboxylase accA, nucleotide sequence |
| SEQ ID NO: 140 | Escherichia coli acetyl-CoA carboxylase accA, amino acid sequence |
| SEQ ID NO: 141 | Escherichia coli acetyl-CoA carboxylase accB, nucleotide sequence |
| SEQ ID NO: 142 | Escherichia coli acetyl-CoA carboxylase accB, amino acid sequence |
| SEQ ID NO: 143 | Escherichia coli acetyl-CoA carboxylase accC, nucleotide sequence |
| SEQ ID NO: 144 | Escherichia coli acetyl-CoA carboxylase accC, amino acid sequence |
| SEQ ID NO: 145 | Escherichia coli acetyl-CoA carboxylase accD, nucleotide sequence |
| SEQ ID NO: 146 | Escherichia coli acetyl-CoA carboxylase accD, amino acid sequence |

The foregoing detailed description has been given for clearness of understanding only and no unnecessary limitations should be understood there from as modifications will be obvious to those skilled in the art.

While the disclosure has been described in connection with specific embodiments thereof, it will be understood that it is capable of further modifications and this application is intended to cover any variations, uses, or adaptations of the disclosure following, in general, the principles of the disclosure and including such departures from the present disclosure as come within known or customary practice within the art to which the disclosure pertains and as may be applied to the essential features hereinbefore set forth and as follows in the scope of the appended claims.

### INCORPORATION BY REFERENCE

All references, articles, publications, patents, patent publications, and patent applications cited herein are incorporated by reference in their entireties for all purposes.

However, mention of any reference, article, publication, patent, patent publication, and patent application cited herein is not, and should not be taken as, an acknowledgment or any form of suggestion that they constitute valid prior art or form part of the common general knowledge in any country in the world.

What is claimed is:

1. A recombinant microorganism capable of co-producing monoethylene glycol (MEG) and isobutene from exogenous D-xylose, wherein the recombinant microorganism expresses one or more of the following from (a) to (d):
(a) at least one endogenous or exogenous nucleic acid molecule encoding a D-tagatose 3-epimerase that catalyzes the conversion of D-xylulose to D-ribulose;
(b) at least one endogenous or exogenous nucleic acid molecule encoding a D-ribulokinase that catalyzes the conversion of D-ribulose from (a) to D-ribulose-1-phosphate,
(c) at least one endogenous or exogenous nucleic acid molecule encoding a D-ribulose-1-phosphate aldolase that catalyzes the conversion of D-ribulose-1-phosphate from (b) to glycolaldehyde and dihydroxyacetonephosphate (DHAP);
(d) at least one endogenous or exogenous nucleic acid molecule encoding a glycolaldehyde reductase that catalyzes the conversion of glycolaldehyde from (c) to MEG;
wherein the recombinant microorganism further expresses one or more of the following from (e) to (h):
(e) at least one endogenous or exogenous nucleic acid molecule encoding a thiolase or acetyl coenzyme A acetyltransferase that catalyzes the conversion of acetyl-CoA to acetoacetyl-CoA;
(f) at least one endogenous or exogenous nucleic acid molecule encoding an acetyl-CoA:acetoacetate-CoA transferase or acetate:acetoacetyl-CoA hydrolase that catalyzes the conversion of acetoacetyl-CoA from (e) to acetoacetate;
(g) at least one endogenous or exogenous nucleic acid molecule encoding an acetoacetate decarboxylase that catalyzes the conversion of acetoacetate from (f) to acetone;
(h) at least one endogenous or exogenous nucleic acid molecule encoding a 3-hydroxyisovalerate synthase that catalyzes the conversion of acetone from (g) and acetyl-CoA to 3-hydroxyisovalerate (3HIV);
or
wherein the recombinant microorganism expresses one or more of the nucleic acid molecule from (a) to (d) above and further expresses one or more of the following from (i) to (n):
(i) at least one endogenous or exogenous nucleic acid molecule encoding an acetyl coenzyme A acetyltransferase that catalyzes the conversion of acetyl-CoA to acetoacetyl-CoA;
(j) at least one endogenous or exogenous nucleic acid molecule encoding a hydroxymethylglutaryl-CoA synthase that catalyzes the conversion of acetoacetyl-CoA from (i) and acetyl-CoA to 3-hydroxy-3-methylglutaryl-CoA (HMG-CoA);
(k) at least one endogenous or exogenous nucleic acid molecule encoding a methylglutaconyl-CoA hydratase that catalyzes the conversion of HMG-CoA from (j) to 3-methylglutaconyl-CoA;
(l) at least one endogenous or exogenous nucleic acid molecule encoding a methylcrotonyl-CoA carboxylase that catalyzes the conversion of 3-methylglutaconyl-CoA from (k) to 3-methylcrotonyl-CoA;
(m) at least one endogenous or exogenous nucleic acid molecule encoding a methylcrotonyl-CoA hydratase that catalyzes the conversion of 3-methylcrotonyl-CoA from (l) to 3-hydroxyisovaleryl-CoA;
(n) at least one endogenous or exogenous nucleic acid molecule encoding a 3-hydroxyisovaleryl-CoA thioesterase that catalyzes the conversion of 3-hydroxyisovaleryl-CoA from (m) to 3HIV;
wherein the recombinant microorganism further expresses (a1) and (a2), and/or (b1) selected from:
(a1) at least one endogenous or exogenous nucleic acid molecule encoding a 3HIV kinase that catalyzes the conversion of 3HIV from (h) or (n) to 3HIV-3-phosphate;
(a2) at least one endogenous or exogenous nucleic acid molecule encoding a 3HIV-3-phosphate decarboxylase that catalyzes the conversion of 3HIV-3-phosphate from (a1) to isobutene;
(b1) at least one endogenous or exogenous nucleic acid molecule encoding a 3HIV decarboxylase that catalyzes the conversion of 3HIV from (h) or (n) to isobutene;
wherein the produced intermediate DHAP is converted to acetyl-CoA through the endogenous glycolysis pathway in the microorganism, and wherein MEG and isobutene are co-produced.

2. A recombinant microorganism capable of co-producing monoethylene glycol (MEG) and isobutene from exogenous D-xylose, wherein the recombinant microorganism expresses one or more of the following from (a) to (c):
(a) at least one endogenous or exogenous nucleic acid molecule encoding a D-xylulose 1-kinase that catalyzes the conversion of D-xylulose to D-xylulose-1-phosphate;
(b) at least one endogenous or exogenous nucleic acid molecule encoding a D-xylulose-1-phosphate aldolase that catalyzes the conversion of D-xylulose-1-phosphate from (a) to glycolaldehyde and dihydroxyacetonephosphate (DHAP);
(c) at least one endogenous or exogenous nucleic acid molecule encoding a glycolaldehyde reductase that catalyzes the conversion of glycolaldehyde from (b) to MEG;
wherein the recombinant microorganism further expresses one or more of the following from (d) to (g):
(d) at least one endogenous or exogenous nucleic acid molecule encoding a thiolase or acetyl coenzyme A acetyltransferase that catalyzes the conversion of acetyl-CoA to acetoacetyl-CoA;
(e) at least one endogenous or exogenous nucleic acid molecule encoding an acetyl-CoA:acetoacetate-CoA transferase or acetate:acetoacetyl-CoA hydrolase that catalyzes the conversion of acetoacetyl-CoA from (d) to acetoacetate;
(f) at least one endogenous or exogenous nucleic acid molecule encoding an acetoacetate decarboxylase that catalyzes the conversion of acetoacetate from (e) to acetone;
(g) at least one endogenous or exogenous nucleic acid molecule encoding a 3-hydroxyisovalerate synthase that catalyzes the conversion of acetone from (f) and acetyl-CoA to 3-hydroxyisovalerate (3HIV);
or
wherein the recombinant microorganism expresses one or more of the nucleic acid molecule from (a) to (c) above and further expresses one or more of the following from (h) to (m):
(h) at least one endogenous or exogenous nucleic acid molecule encoding an acetyl coenzyme A acetyltransferase that catalyzes the conversion of acetyl-CoA to acetoacetyl-CoA;
(i) at least one endogenous or exogenous nucleic acid molecule encoding a hydroxymethylglutaryl-CoA synthase that catalyzes the conversion of acetoacetyl-CoA from (h) and acetyl-CoA to 3-hydroxy-3-methylglutaryl-CoA (HMG-CoA);
(j) at least one endogenous or exogenous nucleic acid molecule encoding a methylglutaconyl-CoA hydratase that catalyzes the conversion of HMG-CoA from (i) to 3-methylglutaconyl-CoA;
(k) at least one endogenous or exogenous nucleic acid molecule encoding a methylcrotonyl-CoA carboxylase that catalyzes the conversion of 3-methylglutaconyl-CoA from (j) to 3-methylcrotonyl-CoA;
(l) at least one endogenous or exogenous nucleic acid molecule encoding a methylcrotonyl-CoA hydratase that catalyzes the conversion of 3-methylcrotonyl-CoA from (k) to 3-hydroxyisovaleryl-CoA;
(m) at least one endogenous or exogenous nucleic acid molecule encoding a 3-hydroxyisovaleryl-CoA thioesterase that catalyzes the conversion of 3-hydroxyisovaleryl-CoA from (l) to 3HIV;
wherein the recombinant microorganism further expresses (a1) and (a2), and/or (b1) selected from:
(a1) at least one endogenous or exogenous nucleic acid molecule encoding a 3HIV kinase that catalyzes the conversion of 3HIV from (g) or (m) to 3HIV-3-phosphate;
(a2) at least one endogenous or exogenous nucleic acid molecule encoding a 3HIV-3-phosphate decarboxylase that catalyzes the conversion of 3HIV-3-phosphate from (a1) to isobutene;
(b1) at least one endogenous or exogenous nucleic acid molecule encoding a 3HIV decarboxylase that catalyzes the conversion of 3HIV from (g) or (m) to isobutene;
and wherein the produced intermediate DHAP is converted to acetyl-CoA through the endogenous glycolysis pathway in the microorganism, and wherein MEG and isobutene are co-produced.

3. The recombinant microorganism of claim 1 or claim 2, wherein the recombinant microorganism further comprises one or more modifications selected from the group consisting of:
(a) a deletion, insertion, or loss of function mutation in a gene encoding a D-xylulose-5-kinase that catalyzes the conversion of D-xylulose to D-xylulose-5-phosphate;
(b) a deletion, insertion, or loss of function mutation in a gene encoding a glycolaldehyde dehydrogenase that catalyzes the conversion of glycolaldehyde to glycolic acid; and
(c) a deletion, insertion, or loss of function mutation in a gene encoding a lactate dehydrogenase that catalyzes the conversion of pyruvate to lactate.

4. The recombinant microorganism of any one of claims 1-3, wherein an endogenous or exogenous xylose isomerase catalyzes the conversion of D-xylose to D-xylulose.

5. The recombinant microorganism of any one of claims 1-3, wherein the recombinant microorganism further expresses at least one exogenous nucleic acid molecule encoding a xylose reductase or aldose reductase that catalyzes the conversion of D-xylose to xylitol and at least one exogenous nucleic acid molecule encoding a xylitol dehydrogenase that catalyzes the conversion of xylitol to D-xylulose.

6. A recombinant microorganism capable of co-producing monoethylene glycol (MEG) and isobutene from exogenous D-xylose, wherein the recombinant microorganism expresses one or more of the following from (a) to (c):
(a) at least one endogenous or exogenous nucleic acid molecule encoding a xylose dehydrogenase that catalyzes the conversion of D-xylose to D-xylonolactone,
(b) at least one endogenous or exogenous nucleic acid molecule encoding a xylonolactonase that catalyzes the conversion of D-xylonolactone from (a) to D-xylonate,
(c) at least one endogenous or exogenous nucleic acid molecule encoding a xylose dehydrogenase that catalyzes the conversion of D-xylose to D-xylonate;
wherein the recombinant microorganism further expresses one or more of the following from (d) to (f):
(d) at least one endogenous or exogenous nucleic acid molecule encoding a xylonate dehydratase that catalyzes the conversion of D-xylonate from (b) or (c) to 2-keto-3-deoxy-xylonate;
(e) at least one endogenous or exogenous nucleic acid molecule encoding a 2-keto-3-deoxy-D-pentonate aldolase that catalyzes the conversion of 2-keto-3-deoxy-xylonate from (d) to glycolaldehyde and pyruvate;
(f) at least one endogenous or exogenous nucleic acid molecule encoding a glycolaldehyde reductase that catalyzes the conversion of glycolaldehyde from (e) to MEG;
wherein the recombinant microorganism further expresses one or more of the following from (g) to (j):
(g) at least one exogenous nucleic acid molecule encoding a thiolase or acetyl coenzyme A acetyltransferase that catalyzes the conversion of acetyl-CoA to acetoacetyl-CoA;
(h) at least one endogenous or exogenous nucleic acid molecule encoding an acetyl-CoA:acetoacetate-CoA transferase or acetate:acetoacetyl-CoA hydrolase that catalyzes the conversion of acetoacetyl-CoA from (g) to acetoacetate;
(i) at least one exogenous nucleic acid molecule encoding an acetoacetate decarboxylase that catalyzes the conversion of acetoacetate from (h) to acetone;
(j) at least one endogenous or exogenous nucleic acid molecule encoding a 3-hydroxyisovalerate synthase that catalyzes the conversion of acetone from (i) and acetyl-CoA to 3-hydroxy-isovalerate (3HIV);
or
wherein the recombinant microorganism expresses one or more of the nucleic acid molecule from (a) to (c) above and one or more of the nucleic acid molecule from (d) to (f) above, and further expresses one or more of the following from (k) to (p):
(k) at least one endogenous or exogenous nucleic acid molecule encoding an acetyl coenzyme A acetyltransferase that catalyzes the conversion of acetyl-CoA to acetoacetyl-CoA;
(l) at least one endogenous or exogenous nucleic acid molecule encoding a hydroxymethylglutaryl-CoA synthase that catalyzes the conversion of acetoacetyl-CoA from (k) and acetyl-CoA to 3-hydroxy-3-methylglutaryl-CoA (HMG-CoA);
(m) at least one endogenous or exogenous nucleic acid molecule encoding a methylglutaconyl-CoA hydratase that catalyzes the conversion of HMG-CoA from (l) to 3-methylglutaconyl-CoA;
(n) at least one endogenous or exogenous nucleic acid molecule encoding a methylcrotonyl-CoA carboxylase that catalyzes the conversion of 3-methylglutaconyl-CoA from (m) to 3-methylcrotonyl-CoA;
(o) at least one endogenous or exogenous nucleic acid molecule encoding a methylcrotonyl-CoA hydratase that catalyzes the conversion of 3-methylcrotonyl-CoA from (n) to 3-hydroxyisovaleryl-CoA;
(p) at least one endogenous or exogenous nucleic acid molecule encoding a 3-hydroxyisovaleryl-CoA thioesterase that catalyzes the conversion of 3-hydroxyisovaleryl-CoA from (o) to 3HIV;
wherein the recombinant microorganism further expresses (a1) and (a2), and/or (b1) selected from:
(a1) at least one endogenous or exogenous nucleic acid molecule encoding a 3HIV kinase that catalyzes the conversion of 3HIV from (j) or (p) to 3HIV-3-phosphate;
(a2) at least one endogenous or exogenous nucleic acid molecule encoding a 3HIV-3-phosphate decarboxylase that catalyzes the conversion of 3HIV-3-phosphate from (a1) to isobutene;
(b1) at least one endogenous or exogenous nucleic acid molecule encoding a 3HIV decarboxylase that catalyzes the conversion of 3HIV from (j) or (p) to isobutene;
and wherein the produced intermediate pyruvate is converted to acetyl-CoA through the endogenous glycolysis pathway in the microorganism, and wherein MEG and isobutene are co-produced.

7. The recombinant microorganism of claim 6, wherein the recombinant microorganism further comprises one or more modifications selected from the group consisting of:
(a) a deletion, insertion, or loss of function mutation in a gene encoding a D-xylose isomerase that catalyzes the conversion of D-xylose to D-xylulose;
(b) a deletion, insertion, or loss of function mutation in a gene encoding a glycolaldehyde dehydrogenase that catalyzes the conversion of glycolaldehyde to glycolic acid; and
(c) a deletion, insertion, or loss of function mutation in a gene encoding a lactate dehydrogenase that catalyzes the conversion of pyruvate to lactate.

8. The recombinant microorganism of claim 1, wherein the D-tagatose 3-epimerase is encoded by one or more nucleic acid molecules obtained from a microorganism selected from the group consisting of Pseudomonas sp., Mesorhizobium sp. and Rhodobacter sp.

9. The recombinant microorganism of claim 8, wherein the microorganism is selected from the group consisting of Pseudomonas cichorii, Pseudomonas sp. ST-24, Mesorhizobium loti and Rhodobacter sphaeroides.

10. The recombinant microorganism of claim 8, wherein the one or more nucleic acid molecules is dte and/or FJ851309.1.

11. The recombinant microorganism of claim 1, wherein the D-tagatose 3-epimerase comprises an amino acid sequence selected from the group consisting of SEQ ID NOs: 3 and 5.

12. The recombinant microorganism of claim 1, wherein the D-tagatose 3-epimerase is encoded by a nucleic acid sequence selected from the group consisting of SEQ ID NOs: 1, 2 and 4.

13. The recombinant microorganism of claim 1, wherein the D-ribulokinase is encoded by one or more nucleic acid molecules obtained from E. coli.

14. The recombinant microorganism of claim 13, wherein the one or more nucleic acid molecules is fucK.

15. The recombinant microorganism of claim 1, wherein the D-ribulokinase comprises an amino acid sequence set forth in SEQ ID NO: 8.

16. The recombinant microorganism of claim 1, wherein the D-ribulokinase is encoded by a nucleic acid sequence selected from the group consisting of SEQ ID NOs: 6 and 7.

17. The recombinant microorganism of claim 1, wherein the D-ribulose-1-phosphate aldolase is encoded by one or more nucleic acid molecules obtained from E. coli.

18. The recombinant microorganism of claim 17, wherein the one or more nucleic acid molecules is fucA.

19. The recombinant microorganism of claim 1, wherein the D-ribulose-1-phosphate aldolase comprises an amino acid sequence set forth in SEQ ID NO: 11.

20. The recombinant microorganism of claim 1, wherein the D-ribulose-1-phosphate aldolase is encoded by a nucleic acid sequence selected from the group consisting of SEQ ID NOs: 9 and 10.

21. The recombinant microorganism of any one of claims 1-7, wherein the glycolaldehyde reductase is encoded by one or more nucleic acid molecules obtained from a microorganism selected from E. coli and S. cerevisiae.

22. The recombinant microorganism of claim 21, wherein the one or more nucleic acid molecules is selected from gldA, GRE2, GRE3, yqhD, ydjG, fucO, yafB (dkgB), and/or yqhE (dkgA).

23. The recombinant microorganism of claim 22, wherein the one or more nucleic acid molecules is yqhD.

24. The recombinant microorganism of claim 23, wherein the yqhD comprises a G149E mutation.

25. The recombinant microorganism of any one of claims 1-7, wherein the glycolaldehyde reductase comprises an amino acid sequence selected from the group consisting of SEQ ID NOs: 13, 15, 17, 20, 23, 25, 28, 30 and 32.

26. The recombinant microorganism of any one of claims 1-7, wherein the glycolaldehyde reductase is encoded by a nucleic acid sequence selected from the group consisting of SEQ ID NOs: 12, 14, 16, 18, 19, 21, 22, 24, 26, 27, 29 and 31.

27. The recombinant microorganism of any one of claims 1-7, wherein the thiolase or acetyl coenzyme A acetyltransferase is encoded by one or more nucleic acid molecules obtained from a microorganism selected from the group consisting of Clostridium sp., Bacillus sp., E. coli, Saccharomyces sp. and Marinobacter sp.

28. The recombinant microorganism of claim 27, wherein the microorganism is selected from the group consisting of Clostridium acetobutylicum, Clostridium thermosaccharolyticum, Bacillus cereus, E. coli, Saccharomyces cerevisiae and Marinobacter hydrocarbonoclasticus.

29. The recombinant microorganism of claim 27, wherein the one or more nucleic acid molecules is thIA, atoB and/or ERG10.

30. The recombinant microorganism of any one of claims 1-7, wherein the thiolase or acetyl coenzyme A acetyltransferase comprises an amino acid sequence selected from the group consisting of SEQ ID NOs: 35, 37 and 40.

31. The recombinant microorganism of any one of claims 1-7, wherein the thiolase or acetyl coenzyme A acetyltransferase is encoded by a nucleic acid sequence selected from the group consisting of SEQ ID NOs: 33, 34, 36, 38 and 39.

32. The recombinant microorganism of any one of claims 1-7, wherein the acetyl-CoA:acetoacetate-CoA transferase or acetate:acetoacetyl-CoA hydrolase is encoded by one or more nucleic acid molecules obtained from a microorganism selected from Clostridium sp. and E. coli.

33. The recombinant microorganism of claim 32, wherein the microorganism is E. coll.

34. The recombinant microorganism of claim 32, wherein the one or more nucleic acid molecules encoding the acetyl-CoA:acetoacetate-CoA transferase is atoA and/or atoD.

35. The recombinant microorganism of claim 32, wherein the microorganism is Clostridium acetobutylicum.

36. The recombinant microorganism of claim 32, wherein the one or more nucleic acid molecules encoding the acetate:acetoacetyl-CoA hydrolase is ctfA and/or ctfB.

37. The recombinant microorganism of any one of claims 1-7, wherein the acetyl-CoA:acetoacetate-CoA transferase or acetate:acetoacetyl-CoA hydrolase comprises an amino acid sequence selected from the group consisting of SEQ ID NOs: 43, 46, 97, 99, 101 and 103.

38. The recombinant microorganism of any one of claims 1-7, wherein the acetyl-CoA:acetoacetate-CoA transferase or acetate:acetoacetyl-CoA hydrolase is encoded by a nucleic acid sequence selected from the group consisting of SEQ ID NOs: 41, 42, 44, 45, 96, 98, 100 and 102.

39. The recombinant microorganism of any one of claims 1-7, wherein the acetoacetate decarboxylase is encoded by one or more nucleic acid molecules obtained from a microorganism selected from the group consisting of Clostridium sp., Bacillus sp., Chromobacterium sp. and Pseudomonas sp.

40. The recombinant microorganism of claim 39, wherein the microorganism is selected from the group consisting of Clostridium acetobutylicum, Clostridium beijerinckii, Clostridium cellulolyticum, Bacillus polymyxa, Chromobacterium violaceum and Pseudomonas putida.

41. The recombinant microorganism of claim 39, wherein the one or more nucleic acid molecules encoding the acetoacetate decarboxylase is adc.

42. The recombinant microorganism of any one of claims 1-7, wherein the acetoacetate decarboxylase comprises an amino acid sequence selected from the group consisting of SEQ ID NOs: 49 and 52.

43. The recombinant microorganism of any one of claims 1-7, wherein the acetoacetate decarboxylase is encoded by a nucleic acid sequence selected from the group consisting of SEQ ID NOs: 47, 48, 50 and 51.

44. The recombinant microorganism of any one of claims 1-7, wherein the 3-hydroxyisovalerate synthase is encoded by one or more nucleic acid molecules obtained from a microorganism selected from the group consisting of Mus sp., Saccharomyces sp., Lactobacillus sp. and Polaromonas sp.

45. The recombinant microorganism of claim 44, wherein the microorganism is selected from the group consisting of Mus musculus, Saccharomyces cerevisiae, Lactobacillus crispatus and Polaromonas naphthalenivorans.

46. The recombinant microorganism of claim 44, wherein the one or more nucleic acid molecules encoding the 3-hydroxyisovalerate synthase is selected from the group consisting of Hmgcs1, ERG13, PksG and/or Pnap_0477.

47. The recombinant microorganism of any one of claims 1-7, wherein the one or more nucleic acid molecules encoding the 3-hydroxyisovalerate synthase comprises an amino acid sequence selected from the group consisting of SEQ ID NOs: 105, 107, 109 and 111.

48. The recombinant microorganism of any one of claims 1-7, wherein the one or more nucleic acid molecules encoding the 3-hydroxyisovalerate synthase is encoded by a nucleic acid sequence selected from the group consisting of SEQ ID NOs: 104, 106, 108 and 110.

49. The recombinant microorganism of any one of claims 1-7, wherein the hydroxymethylglutaryl-CoA synthase is encoded by one or more nucleic acid molecules obtained from Saccharomyces sp.

50. The recombinant microorganism of claim 49, wherein the microorganism is from Saccharomyces cerevisiae.

51. The recombinant microorganism of claim 49, wherein the one or more nucleic acid molecules encoding the hydroxymethylglutaryl-CoA synthase is HmgS.

52. The recombinant microorganism of any one of claims 1-7, wherein the one or more nucleic acid molecules encoding the hydroxymethylglutaryl-CoA synthase comprises an amino acid sequence set forth in SEQ ID NO: 123.

53. The recombinant microorganism of any one of claims 1-7, wherein the one or more nucleic acid molecules encoding the hydroxymethylglutaryl-CoA synthase is encoded by a nucleic acid sequence set forth in SEQ ID NO: 122.

54. The recombinant microorganism of any one of claims 1-7, wherein the methylglutaconyl-CoA hydratase is encoded by one or more nucleic acid molecules obtained from Pseudomonas sp.

55. The recombinant microorganism of claim 54, wherein the microorganism is from Pseudomonas putida.

56. The recombinant microorganism of claim 54, wherein the one or more nucleic acid molecules encoding the methylglutaconyl-CoA hydratase is liuC.

57. The recombinant microorganism of any one of claims 1-7, wherein the one or more nucleic acid molecules encoding the methylglutaconyl-CoA hydratase comprises an amino acid sequence set forth in SEQ ID NO: 125.

58. The recombinant microorganism of any one of claims 1-7, wherein the one or more nucleic acid molecules encoding the methylglutaconyl-CoA hydratase is encoded by a nucleic acid sequence set forth in SEQ ID NO: 124.

59. The recombinant microorganism of any one of claims 1-7, wherein the methylcrotonyl-CoA carboxylase is encoded by one or more nucleic acid molecules obtained from Pseudomonas sp.

60. The recombinant microorganism of claim 59, wherein the microorganism is from Pseudomonas aeruginosa.

61. The recombinant microorganism of claim 59, wherein the one or more nucleic acid molecules encoding the methylcrotonyl-CoA carboxylase is liuB and/or liuD.

62. The recombinant microorganism of any one of claims 1-7, wherein the one or more nucleic acid molecules encoding the methylcrotonyl-CoA carboxylase comprises an amino acid sequence selected from SEQ ID NOs: 127 and 129.

63. The recombinant microorganism of any one of claims 1-7, wherein the one or more nucleic acid molecules encoding the methylcrotonyl-CoA carboxylase is encoded by a nucleic acid sequence selected from SEQ ID NOs: 126 and 128.

64. The recombinant microorganism of any one of claims 1-7, wherein the methylcrotonyl-CoA hydratase is a 3-ketoacyl-CoA thiolase.

65. The recombinant microorganism of any one of claims 1-7, wherein the methylcrotonyl-CoA hydratase is an enoyl-CoA hydratase.

66. The recombinant microorganism of any one of claims 1-7, wherein the methylcrotonyl-CoA hydratase is encoded by one or more nucleic acid molecules obtained from E. coli.

67. The recombinant microorganism of claim 66, wherein the one or more nucleic acid molecules encoding the methylcrotonyl-CoA hydratase is fadA and/or fadB.

68. The recombinant microorganism of any one of claims 1-7, wherein the one or more nucleic acid molecules encoding the methylcrotonyl-CoA hydratase comprises an amino acid sequence selected from SEQ ID NOs: 131 and 133.

69. The recombinant microorganism of any one of claims 1-7, wherein the one or more nucleic acid molecules encoding the methylcrotonyl-CoA hydratase is encoded by a nucleic acid sequence selected from SEQ ID NOs: 130 and 132.

70. The recombinant microorganism of any one of claims 1-7, wherein the 3-hydroxyisovaleryl-CoA thioesterase is encoded by one or more nucleic acid molecules obtained from E. coli.

71. The recombinant microorganism of claim 70, wherein the one or more nucleic acid molecules encoding the 3-hydroxyisovaleryl-CoA thioesterase is tesB.

72. The recombinant microorganism of any one of claims 1-7, wherein the one or more nucleic acid molecules encoding the 3-hydroxyisovaleryl-CoA thioesterase comprises an amino acid sequence set forth in SEQ ID NO: 135.

73. The recombinant microorganism of any one of claims 1-7, wherein the one or more nucleic acid molecules encoding the 3-hydroxyisovaleryl-CoA thioesterase is encoded by a nucleic acid sequence set forth in SEQ ID NO: 134.

74. The recombinant microorganism of any one of claims 1-7, wherein the 3HIV kinase is encoded by one or more nucleic acid molecules obtained from a microorganism selected from the group consisting of Thermoplasma sp. and Picrophilus sp.

75. The recombinant microorganism of claim 74, wherein the microorganism is selected from the group consisting of Thermoplasma acidophilum and Picrophilus torridus.

76. The recombinant microorganism of claim 74, wherein the one or more nucleic acid molecules encoding the 3HIV kinase is TA1305 and/or PTO1356.

77. The recombinant microorganism of claim 76, wherein the TA1305 comprises a L200E mutation.

78. The recombinant microorganism of any one of claims 1-7, wherein the one or more nucleic acid molecules encoding the 3HIV kinase comprises an amino acid sequence selected from the group consisting of SEQ ID NOs: 113, 115 and 117.

79. The recombinant microorganism of any one of claims 1-7, wherein the one or more nucleic acid molecules encoding the 3HIV kinase is encoded by a nucleic acid sequence selected from the group consisting of SEQ ID NOs: 112, 114 and 116.

80. The recombinant microorganism of any one of claims 1-7, wherein the 3HIV-3-phosphate decarboxylase is encoded by one or more nucleic acid molecules obtained from Streptococcus sp.

81. The recombinant microorganism of claim 80, wherein the microorganism is selected from Streptococcus mitis and/or Streptococcus gordonii.

82. The recombinant microorganism of claim 80, wherein the one or more nucleic acid molecules encoding the 3HIV-3-phosphate decarboxylase comprises smi_1746 and/or mvaD.

83. The recombinant microorganism of any one of claims 1-7, wherein the one or more nucleic acid molecules encoding the 3HIV-3-phosphate decarboxylase comprises an amino acid sequence selected from SEQ ID NOs: 119 and 121.

84. The recombinant microorganism of any one of claims 1-7, wherein the one or more nucleic acid molecules encoding the 3HIV-3-phosphate decarboxylase is encoded by a nucleic acid sequence selected from SEQ ID NOs: 118 and 120.

85. The recombinant microorganism of any one of claims 1-7, wherein the 3HIV decarboxylase is encoded by one or more nucleic acid molecules obtained from a microorganism selected from the group consisting of Streptococcus sp., Thermoplasma sp. and Picrophilus sp.

86. The recombinant microorganism of claim 85, wherein the microorganism is selected from the group consisting of Streptococcus gordonii, Thermoplasma acidophilum and Picrophilus torridus.

87. The recombinant microorganism of claim 85, wherein the one or more nucleic acid molecules encoding the 3HIV decarboxylase comprises mvaD, TA1305 and/or PTO1356.

88. The recombinant microorganism of any one of claims 1-7, wherein the one or more nucleic acid molecules encoding the 3HIV decarboxylase comprises an amino acid sequence selected from the group consisting of SEQ ID NOs: 113, 117 and 121.

89. The recombinant microorganism of any one of claims 1-7, wherein the one or more nucleic acid molecules encoding the 3HIV decarboxylase is encoded by a nucleic acid sequence selected from the group consisting of SEQ ID NOs: 112, 116 and 120.

90. The recombinant microorganism of claim 2, wherein the D-xylulose 1-kinase is encoded by one or more nucleic acid molecules obtained from Homo sapiens.

91. The recombinant microorganism of claim 90, wherein the one or more nucleic acid molecules encoding the D-xylulose 1-kinase is ketohexokinase C (khk-C).

92. The recombinant microorganism of claim 2, wherein the one or more nucleic acid molecules encoding the D-xylulose 1-kinase comprises an amino acid sequence set forth in SEQ ID NO: 55.

93. The recombinant microorganism of claim 2, wherein the one or more nucleic acid molecules encoding the D-xylulose 1-kinase is encoded by a nucleic acid sequence selected from the group consisting of SEQ ID NOs: 53 and 54.

94. The recombinant microorganism of claim 2, wherein the D-xylulose-1-phosphate aldolase is encoded by one or more nucleic acid molecules obtained from Homo sapiens.

95. The recombinant microorganism of claim 94, wherein the one or more nucleic acid molecules encoding the D-xylulose-1-phosphate aldolase is aldolase B (ALDOB).

96. The recombinant microorganism of claim 2, wherein the one or more nucleic acid molecules encoding the D-xylulose-1-phosphate aldolase comprises an amino acid sequence set forth in SEQ ID NO: 58.

97. The recombinant microorganism of claim 2, wherein the one or more nucleic acid molecules encoding the D-xylulose-1-phosphate aldolase is encoded by a nucleic acid sequence selected from the group consisting of SEQ ID NOs: 56 and 57.

98. The recombinant microorganism of claim 5, wherein the xylose reductase or aldose reductase is encoded by one or more nucleic acid molecules obtained from a microorganism selected from the group consisting of Hypocrea sp., Scheffersomyces sp., Saccharomyces sp., Pachysolen sp., Pichia sp., Candida sp., Aspergillus sp., Neurospora sp., and Cryptococcus sp.

99. The recombinant microorganism of claim 98, wherein the microorganism is selected from the group consisting of Hypocrea jecorina, Scheffersomyces stipitis, Saccharomyces cerevisiae, Pachysolen tannophilus, Pichia stipitis, Pichia quercuum, Candida shehatae, Candida tenuis, Candida tropicalis, Aspergillus niger, Neurospora crassa and Cryptococcus lactativorus.

100. The recombinant microorganism of claim 98, wherein the one or more nucleic acid molecules encoding the xylose reductase or aldose reductase is xyl1 and/or GRE3.

101. The recombinant microorganism of claim 5, wherein the one or more nucleic acid molecules encoding the xylose reductase or aldose reductase comprises an amino acid sequence selected from the group consisting of SEQ ID NOs: 84 and 87.

102. The recombinant microorganism of claim 5, wherein the one or more nucleic acid molecules encoding the xylose reductase or aldose reductase is encoded by a nucleic acid sequence selected from the group consisting of SEQ ID NOs: 82, 83, 85 and 86.

103. The recombinant microorganism of claim 5, wherein the xylitol dehydrogenase is encoded by one or more nucleic acid molecules obtained from a microorganism selected from the group consisting of Scheffersomyces sp., Trichoderma sp., Pichia sp., Saccharomyces sp., Gluconobacter sp., Galactocandida sp., Neurospora sp., and Serratia sp.

104. The recombinant microorganism of claim 103, wherein the microorganism is selected from the group consisting of Scheffersomyces stipitis, Trichoderma reesei, Pichia stipitis, Saccharomyces cerevisiae, Gluconobacter oxydans, Galactocandida mastotermitis, Neurospora crassa and Serratia marcescens.

105. The recombinant microorganism of claim 103, wherein the one or more nucleic acid molecules encoding the xylitol dehydrogenase is xyl2 and/or xdh1.

106. The recombinant microorganism of claim 5, wherein the one or more nucleic acid molecules encoding the xylitol dehydrogenase comprises an amino acid sequence selected from the group consisting of SEQ ID NOs: 90 and 92.

107. The recombinant microorganism of claim 5, wherein the one or more nucleic acid molecules encoding the xylitol dehydrogenase is encoded by a nucleic acid sequence selected from the group consisting of SEQ ID NOs: 88, 89 and 91.

108. The recombinant microorganism of claim 4, wherein the xylose isomerase is encoded by one or more nucleic acid molecules obtained from Pyromyces sp.

109. The recombinant microorganism of claim 108, wherein the one or more nucleic acid molecules encoding the xylose isomerase is xylA.

110. The recombinant microorganism of claim 4, wherein the one or more nucleic acid molecules encoding the xylose isomerase comprises an amino acid sequence set forth in SEQ ID NO: 95.

111. The recombinant microorganism of claim 4, wherein the one or more nucleic acid molecules encoding the xylose isomerase is encoded by a nucleic acid sequence selected from the group consisting of SEQ ID NOs: 93 and 94.

112. The recombinant microorganism of claim 6, wherein the xylose dehydrogenase is encoded by one or more nucleic acid molecules obtained from a microorganism selected from the group consisting of Caulobacter sp., Haloarcula sp., Haloferax sp., Halorubrum sp. and Trichoderma sp.

113. The recombinant microorganism of claim 112, wherein the microorganism is selected from the group consisting of Caulobacter crescentus, Haloarcula marismortui, Haloferax volcanii, Halorubrum lacusprofundi and Trichoderma reesei.

114. The recombinant microorganism of claim 112, wherein the one or more nucleic acid molecules encoding the xylose dehydrogenase is selected from xylB, xdh1 (HVO_B0028) and/or xyd1.

115. The recombinant microorganism of claim 6, wherein the one or more nucleic acid molecules encoding the xylose dehydrogenase comprises an amino acid sequence selected from the group consisting of SEQ ID NOs: 61, 63 and 65.

116. The recombinant microorganism of claim 6, wherein the one or more nucleic acid molecules encoding the xylose dehydrogenase is encoded by a nucleic acid sequence selected from the group consisting of SEQ ID NOs: 59, 60, 62 and 64.

117. The recombinant microorganism of claim 6, wherein the xylonolactonase is encoded by one or more nucleic acid molecules obtained from a microorganism selected from Caulobacter sp. and Haloferax sp.

118. The recombinant microorganism of claim 117, wherein the microorganism is selected from the group consisting of Caulobacter crescentus, Haloferax volcanii and Haloferax gibbonsii.

119. The recombinant microorganism of claim 117, wherein the one or more nucleic acid molecules encoding the xylonolactonase is xylC.

120. The recombinant microorganism of claim 6, wherein the one or more nucleic acid molecules encoding the xylonolactonase comprises an amino acid sequence set forth in SEQ ID NO: 67.

121. The recombinant microorganism of claim 6, wherein the one or more nucleic acid molecules encoding the xylonolactonase is encoded by a nucleic acid sequence set forth in SEQ ID NO: 66.

122. The recombinant microorganism of claim 6, wherein the xylonate dehydratase is encoded by one or more nucleic acid molecules obtained from a microorganism selected from the group consisting of Caulobacter sp., Sulfolobus sp. and E. coli.

123. The recombinant microorganism of claim 122, wherein the microorganism is selected from the group consisting of Caulobacter crescentus, Sulfolobus solfataricus and E. coli.

124. The recombinant microorganism of claim 122, wherein the one or more nucleic acid molecules encoding the xylonate dehydratase is selected from xylD, yjhG and/or yagF.

125. The recombinant microorganism of claim 6, wherein the one or more nucleic acid molecules encoding the xylonate dehydratase comprises an amino acid sequence selected from the group consisting of SEQ ID NOs: 69, 72 and 75.

126. The recombinant microorganism of claim 6, wherein the one or more nucleic acid molecules encoding the xylonate dehydratase is encoded by a nucleic acid sequence selected from the group consisting of SEQ ID NOs: 68, 70, 71, 73 and 74.

127. The recombinant microorganism of claim 6, wherein the 2-keto-3-deoxy-D-pentonate aldolase is encoded by one or more nucleic acid molecules obtained from a microorganism selected from Pseudomonas sp. and E. coli.

128. The recombinant microorganism of claim 127, wherein the microorganism is E. coli.

129. The recombinant microorganism of claim 127, wherein the one or more nucleic acid molecules encoding the 2-keto-3-deoxy-D-pentonate aldolase is selected from yjhH and/or yagE.

130. The recombinant microorganism of claim 6, wherein the one or more nucleic acid molecules encoding the 2-keto-3-deoxy-D-pentonate aldolase comprises an amino acid sequence selected from the group consisting of SEQ ID NOs: 78 and 81.

131. The recombinant microorganism of claim 6, wherein the one or more nucleic acid molecules encoding the 2-keto-3-deoxy-D-pentonate aldolase is encoded by a nucleic acid sequence selected from the group consisting of SEQ ID NOs: 76, 77, 79 and 80.

132. The recombinant microorganism of any one of claims 1-131, wherein MEG is produced through the conversion of glycolaldehyde in a C-2 branch pathway and isobutene is produced through the conversion of DHAP or pyruvate in a C-3 branch pathway.

133. The recombinant microorganism of claim 132, wherein at least a portion of the excess NADH produced in the C-3 branch pathway is used as a source of reducing equivalents in the C-2 branch pathway.

134. The recombinant microorganism of claim 132, wherein at least a portion of the excess NADH produced in the C-3 branch pathway is used to produce ATP.

135. The recombinant microorganism of any one of claims 1-131, wherein excess biomass formation is minimized and production of MEG and isobutene is maximized.

136. A method of producing MEG and isobutene using a recombinant microorganism of any of the preceding claims, wherein the method comprises cultivating the recombinant microorganism in a culture medium containing a feedstock providing a carbon source until the MEG and isobutene are produced.

137. A method of producing a recombinant microorganism that produces or accumulates MEG and isobutene from exogenous D-xylose, comprising introducing into the recombinant microorganism and/or overexpressing one or more of the following from (a) to (d):
(a) at least one endogenous or exogenous nucleic acid molecule encoding a D-tagatose 3-epimerase that catalyzes the conversion of D-xylulose to D-ribulose;
(b) at least one endogenous or exogenous nucleic acid molecule encoding a D-ribulokinase that catalyzes the conversion of D-ribulose from (a) to D-ribulose-1-phosphate,
(c) at least one endogenous or exogenous nucleic acid molecule encoding a D-ribulose-1-phosphate aldolase that catalyzes the conversion of D-ribulose-1-phosphate from (b) to glycolaldehyde and dihydroxyacetonephosphate (DHAP);
(d) at least one endogenous or exogenous nucleic acid molecule encoding a glycolaldehyde reductase that catalyzes the conversion of glycolaldehyde from (c) to MEG;
wherein the method further comprises introducing into and/or overexpressing in the recombinant microorganism one or more of the following from (e) to (h):
(e) at least one endogenous or exogenous nucleic acid molecule encoding a thiolase or acetyl coenzyme A acetyltransferase that catalyzes the conversion of acetyl-CoA to acetoacetyl-CoA;
(f) at least one endogenous or exogenous nucleic acid molecule encoding an acetyl-CoA:acetoacetate-CoA transferase or acetate:acetoacetyl-CoA hydrolase that catalyzes the conversion of acetoacetyl-CoA from (e) to acetoacetate;
(g) at least one endogenous or exogenous nucleic acid molecule encoding an acetoacetate decarboxylase that catalyzes the conversion of acetoacetate from (f) to acetone;
(h) at least one endogenous or exogenous nucleic acid molecule encoding a hydroxymethylglutaryl-CoA synthase that catalyzes the conversion of acetone from (g) and acetyl-CoA to 3-hydroxyisovalerate (3HIV);
or
wherein the method comprises introducing into and/or overexpressing in the recombinant microorganism one or more of the nucleic acid molecule from (a) to (d) above and further comprises introducing into and/or overexpressing in the recombinant microorganism one or more of the following from (i) to (n):
(i) at least one endogenous or exogenous nucleic acid molecule encoding an acetyl coenzyme A acetyltransferase that catalyzes the conversion of acetyl-CoA to acetoacetyl-CoA;
(j) at least one endogenous or exogenous nucleic acid molecule encoding a hydroxymethylglutaryl-CoA synthase that catalyzes the conversion of acetoacetyl-CoA from (i) and acetyl-CoA to 3-hydroxy-3-methylglutaryl-CoA (HMG-CoA);
(k) at least one endogenous or exogenous nucleic acid molecule encoding a methylglutaconyl-CoA hydratase that catalyzes the conversion of HMG-CoA from (j) to 3-methylglutaconyl-CoA;
(l) at least one endogenous or exogenous nucleic acid molecule encoding a methylcrotonyl-CoA carboxylase that catalyzes the conversion of 3-methylglutaconyl-CoA from (k) to 3-methylcrotonyl-CoA;
(m) at least one endogenous or exogenous nucleic acid molecule encoding a methylcrotonyl-CoA hydratase that catalyzes the conversion of 3-methylcrotonyl-CoA from (l) to 3-hydroxyisovaleryl-CoA,
(n) at least one endogenous or exogenous nucleic acid molecule encoding a 3-hydroxyisovaleryl-CoA thioesterase that catalyzes the conversion of 3-hydroxyisovaleryl-CoA from (m) to 3HIV;
wherein the method further comprises introducing into and/or overexpressing in the recombinant microorganism (a1) and (a2), and/or (b1) selected from:
(a1) at least one endogenous or exogenous nucleic acid molecule encoding a 3HIV kinase that catalyzes the conversion of 3HIV from (h) or (n) to 3HIV-3-phosphate;
(a2) at least one endogenous or exogenous nucleic acid molecule encoding a 3HIV-3-phosphate decarboxylase that catalyzes the conversion of 3HIV-3phosphate from (a1) to isobutene;
(b1) at least one endogenous or exogenous nucleic acid molecule encoding a 3HIV decarboxylase that catalyzes the conversion of 3HIV from (h) or (n) to isobutene;
and wherein the produced intermediate DHAP is converted to acetyl-CoA through the endogenous glycolysis pathway in the microorganism, and wherein MEG and isobutene are co-produced.

138. A method of producing a recombinant microorganism that produces or accumulates MEG and isobutene from exogenous D-xylose, comprising introducing into and/or overexpressing in the recombinant microorganism one or more of the following from (a) to (c):
(a) at least one endogenous or exogenous nucleic acid molecule encoding a D-xylulose 1-kinase that catalyzes the conversion of D-xylulose to D-xylulose-1-phosphate;
(b) at least one endogenous or exogenous nucleic acid molecule encoding a D-xylulose-1-phosphate aldolase that catalyzes the conversion of D-xylulose-1-phosphate from (a) to glycolaldehyde and dihydroxyacetonephosphate (DHAP);
(c) at least one endogenous or exogenous nucleic acid molecule encoding a glycolaldehyde reductase that catalyzes the conversion of glycolaldehyde from (b) to MEG;
wherein the method further comprises introducing into and/or overexpressing in the recombinant microorganism one or more of the following from (d) to (g):
(d) at least one endogenous or exogenous nucleic acid molecule encoding a thiolase or acetyl coenzyme A acetyltransferase that catalyzes the conversion of acetyl-CoA to acetoacetyl-CoA;
(e) at least one endogenous or exogenous nucleic acid molecule encoding an acetyl-CoA:acetoacetate-CoA transferase or acetate:acetoacetyl-CoA hydrolase that catalyzes the conversion of acetoacetyl-CoA from (d) to acetoacetate;
(f) at least one endogenous or exogenous nucleic acid molecule encoding an acetoacetate decarboxylase that catalyzes the conversion of acetoacetate from (e) to acetone;
(g) at least one endogenous or exogenous nucleic acid molecule encoding a 3-hydroxyisovalerate synthase that catalyzes the conversion of acetone from (f) and acetyl-CoA to 3-hydroxyisovalerate (3HIV);
or
wherein the method comprises introducing into and/or overexpressing in the recombinant microorganism one or more of the nucleic acid molecule from (a) to (c) above and further comprises introducing into and/or overexpressing in the recombinant microorganism one or more of the following from (h) to (m):
(h) at least one endogenous or exogenous nucleic acid molecule encoding an acetyl coenzyme A acetyltransferase that catalyzes the conversion of acetyl-CoA to acetoacetyl-CoA;
(i) at least one endogenous or exogenous nucleic acid molecule encoding a hydroxymethylglutaryl-CoA synthase that catalyzes the conversion of acetoacetyl-CoA from (h) and acetyl-CoA to 3-hydroxy-3-methylglutaryl-CoA (HMG-CoA);
(j) at least one endogenous or exogenous nucleic acid molecule encoding a methylglutaconyl-CoA hydratase that catalyzes the conversion of HMG-CoA from (i) to 3-methylglutaconyl-CoA;
(k) at least one endogenous or exogenous nucleic acid molecule encoding a methylcrotonyl-CoA carboxylase that catalyzes the conversion of 3-methylglutaconyl-CoA from (j) to 3-methylcrotonyl-CoA;
(l) at least one endogenous or exogenous nucleic acid molecule encoding a methylcrotonyl-CoA hydratase that catalyzes the conversion of 3-methylcrotonyl-CoA from (k) to 3-hydroxyisovaleryl-CoA;
(m) at least one endogenous or exogenous nucleic acid molecule encoding a 3-hydroxyisovaleryl-CoA thioesterase that catalyzes the conversion of 3-hydroxyisovaleryl-CoA from (l) to 3HIV;
wherein the method further comprises introducing into and/or overexpressing in the recombinant microorganism (a1) and (a2), and/or (b1) selected from:
(a1) at least one endogenous or exogenous nucleic acid molecule encoding a 3HIV kinase that catalyzes the conversion of 3HIV from (g) or (m) to 3HIV-3-phosphate;
(a2) at least one endogenous or exogenous nucleic acid molecule encoding a 3HIV-3-phosphate decarboxylase that catalyzes the conversion of 3HIV-3phosphate from (a1) to isobutene;
(b1) at least one endogenous or exogenous nucleic acid molecule encoding a 3HIV decarboxylase that catalyzes the conversion of 3HIV from (g) or (m) to isobutene;
and wherein the produced intermediate DHAP is converted to acetyl-CoA through the endogenous glycolysis pathway in the microorganism, and wherein MEG and isobutene are co-produced.

139. The method of claim 137 or claim 138, wherein the method further comprises introducing into the recombinant microorganism one or more modifications selected from the group consisting of:
(a) a deletion, insertion, or loss of function mutation in a gene encoding a D-xylulose-5-kinase that catalyzes the conversion of D-xylulose to D-xylulose-5-phosphate;
(b) a deletion, insertion, or loss of function mutation in a gene encoding a glycolaldehyde dehydrogenase that catalyzes the conversion of glycolaldehyde to glycolic acid; and
(c) a deletion, insertion, or loss of function mutation in a gene encoding a lactate dehydrogenase that catalyzes the conversion of pyruvate to lactate.

140. The method of any one of claims 137-139, wherein an endogenous or exogenous xylose isomerase catalyzes the conversion of D-xylose to D-xylulose.

141. The method of any one of claims 137-139, wherein the method further comprises introducing into and/or overexpressing in the recombinant microorganism at least one exogenous nucleic acid molecule encoding a xylose reductase or aldose reductase that catalyzes the conversion of D-xylose to xylitol and at least one exogenous nucleic acid molecule encoding a xylitol dehydrogenase that catalyzes the conversion of xylitol to D-xylulose.

142. A method of producing a recombinant microorganism that produces or accumulates MEG and isobutene from exogenous D-xylose, comprising introducing into and/or overexpressing in the recombinant microorganism one or more of the following from (a) to (c):
(a) at least one endogenous or exogenous nucleic acid molecule encoding a xylose dehydrogenase that catalyzes the conversion of D-xylose to D-xylonolactone; and
(b) at least one endogenous or exogenous nucleic acid molecule encoding a xylonolactonase that catalyzes the conversion of D-xylonolactone from (a) to D-xylonate;
(c) at least one endogenous or exogenous nucleic acid molecule encoding a xylose dehydrogenase that catalyzes the conversion of D-xylose to D-xylonate;
wherein the method further comprises introducing into and/or overexpressing in the recombinant microorganism one or more of the following from (d) to (f):
(d) at least one endogenous or exogenous nucleic acid molecule encoding a xylonate dehydratase that catalyzes the conversion of D-xylonate from (b) or (c) to 2-keto-3-deoxy-xylonate;
(e) at least one endogenous or exogenous nucleic acid molecule encoding a 2-keto-3-deoxy-D-pentonate aldolase that catalyzes the conversion of 2-keto-3-deoxy-xylonate from (d) to glycolaldehyde and pyruvate;
(f) at least one endogenous or exogenous nucleic acid molecule encoding a glycolaldehyde reductase that catalyzes the conversion of glycolaldehyde from (e) to MEG;
wherein the method further comprises introducing into and/or overexpressing in the recombinant microorganism one or more of the following from (g) to (j):
(g) at least one exogenous nucleic acid molecule encoding a thiolase or acetyl coenzyme A acetyltransferase that catalyzes the conversion of acetyl-CoA to acetoacetyl-CoA;
(h) at least one endogenous or exogenous nucleic acid molecule encoding an acetyl-CoA:acetoacetate-CoA transferase or acetate:acetoacetyl-CoA hydrolase that catalyzes the conversion of acetoacetyl-CoA from (g) to acetoacetate;
(i) at least one exogenous nucleic acid molecule encoding an acetoacetate decarboxylase that catalyzes the conversion of acetoacetate from (h) to acetone;
(j) at least one endogenous or exogenous nucleic acid molecule encoding a 3-hydroxyisovalerate synthase that catalyzes the conversion of acetone from (i) and acetyl-CoA to 3-hydroxy-isovalerate (3HIV);
or
wherein the method comprises introducing into and/or overexpressing in the recombinant microorganism one or more of the nucleic acid molecule from (a) to (c) above and one or more of the nucleic acid molecule from (d) to (f) above, and further comprises introducing into and/or overexpressing in the recombinant microorganism one or more of the following from (k) to (p):
(k) at least one endogenous or exogenous nucleic acid molecule encoding an acetyl coenzyme A acetyltransferase that catalyzes the conversion of acetyl-CoA to acetoacetyl-CoA;
(l) at least one endogenous or exogenous nucleic acid molecule encoding a hydroxymethylglutaryl-CoA synthase that catalyzes the conversion of acetoacetyl-CoA from (k) and acetyl-CoA to 3-hydroxy-3-methylglutaryl-CoA (HMG-CoA);
(m) at least one endogenous or exogenous nucleic acid molecule encoding a methylglutaconyl-CoA hydratase that catalyzes the conversion of HMG-CoA from (l) to 3-methylglutaconyl-CoA;
(n) at least one endogenous or exogenous nucleic acid molecule encoding a methylcrotonyl-CoA carboxylase that catalyzes the conversion of 3-methylglutaconyl-CoA from (m) to 3-methylcrotonyl-CoA;
(o) at least one endogenous or exogenous nucleic acid molecule encoding a methylcrotonyl-CoA hydratase that catalyzes the conversion of 3-methylcrotonyl-CoA from (n) to 3-hydroxyisovaleryl-CoA;
(p) at least one endogenous or exogenous nucleic acid molecule encoding a 3-hydroxyisovaleryl-CoA thioesterase that catalyzes the conversion of 3-hydroxyisovaleryl-CoA from (o) to 3HIV;
wherein the method further comprises introducing into and/or overexpressing in the recombinant microorganism (a1) and (a2), and/or (b1) selected from:
(a1) at least one endogenous or exogenous nucleic acid molecule encoding a 3HIV kinase that catalyzes the conversion of 3HIV from (j) or (p) to 3HIV-3-phosphate;
(a2) at least one endogenous or exogenous nucleic acid molecule encoding a 3HIV-3-phosphate decarboxylase that catalyzes the conversion of 3HIV-3phosphate from (a1) to isobutene;
(b1) at least one endogenous or exogenous nucleic acid molecule encoding a 3HIV decarboxylase that catalyzes the conversion of 3HIV from (j) or (p) to isobutene;
and wherein the produced intermediate pyruvate is converted to acetyl-CoA through the endogenous glycolysis pathway in the microorganism, and wherein MEG and isobutene are co-produced.

143. The method of claim 142, wherein the method further comprises introducing into the recombinant microorganism one or more modifications selected from the group consisting of:
(a) a deletion, insertion, or loss of function mutation in a gene encoding a D-xylose isomerase that catalyzes the conversion of D-xylose to D-xylulose;
(b) a deletion, insertion, or loss of function mutation in a gene encoding a glycolaldehyde dehydrogenase that catalyzes the conversion of glycolaldehyde to glycolic acid; and
(c) a deletion, insertion, or loss of function mutation in a gene encoding a lactate dehydrogenase that catalyzes the conversion of pyruvate to lactate.

144. The method of claim 137, wherein the D-tagatose 3-epimerase is encoded by one or more nucleic acid molecules obtained from a microorganism selected from the group consisting of Pseudomonas sp., Mesorhizobium sp. and Rhodobacter sp.

145. The method of claim 144, wherein the microorganism is selected from the group consisting of Pseudomonas cichorii, Pseudomonas sp. ST-24, Mesorhizobium loti and Rhodobacter sphaeroides.

146. The method of claim 144, wherein the one or more nucleic acid molecules is dte and/or FJ851309.1.

147. The method of claim 137, wherein the D-tagatose 3-epimerase comprises an amino acid sequence selected from the group consisting of SEQ ID NOs: 3 and 5.

148. The method of claim 137, wherein the D-tagatose 3-epimerase is encoded by a nucleic acid sequence selected from the group consisting of SEQ ID NOs: 1, 2 and 4.

149. The method of claim 137, wherein the D-ribulokinase is encoded by one or more nucleic acid molecules obtained from E. coli.

150. The method of claim 149, wherein the one or more nucleic acid molecules is fucK.

151. The method of claim 137, wherein the D-ribulokinase comprises an amino acid sequence set forth in SEQ ID NO: 8.

152. The method of claim 137, wherein the D-ribulokinase is encoded by a nucleic acid sequence selected from the group consisting of SEQ ID NOs: 6 and 7.

153. The method of claim 137, wherein the D-ribulose-1-phosphate aldolase is encoded by one or more nucleic acid molecules obtained from E. coli.

154. The method of claim 153, wherein the one or more nucleic acid molecules is fucA.

155. The method of claim 137, wherein the D-ribulose-1-phosphate aldolase comprises an amino acid sequence set forth in SEQ ID NO: 11.

156. The method of claim 137, wherein the D-ribulose-1-phosphate aldolase is encoded by a nucleic acid sequence selected from the group consisting of SEQ ID NOs: 9 and 10.

157. The method of any one of claims 137-143, wherein the glycolaldehyde reductase is encoded by one or more nucleic acid molecules obtained from a microorganism selected from E. coli and S. cerevisiae.

158. The method of claim 157, wherein the one or more nucleic acid molecules is selected from gldA, GRE2, GRE3, yqhD, ydjG, fucO, yafB (dkgB), and/or yqhE (dkgA).

159. The method of claim 158, wherein the one or more nucleic acid molecules is yqhD.

160. The method of claim 159, wherein the yqhD comprises a G149E mutation.

161. The method of any one of claims 137-143, wherein the glycolaldehyde reductase comprises an amino acid sequence selected from the group consisting of SEQ ID NOs: 13, 15, 17, 20, 23, 25, 28, 30 and 32.

162. The method of any one of claims 137-143, wherein the glycolaldehyde reductase is encoded by a nucleic acid sequence selected from the group consisting of SEQ ID NOs: 12, 14, 16, 18, 19, 21, 22, 24, 26, 27, 29 and 31.

163. The method of any one of claims 137-143, wherein the thiolase or acetyl coenzyme A acetyltransferase is encoded by one or more nucleic acid molecules obtained from a microorganism selected from the group consisting of Clostridium sp., Bacillus sp., E. coli, Saccharomyces sp. and Marinobacter sp.

164. The method of claim 163, wherein the microorganism is selected from the group consisting of Clostridium acetobutylicum, Clostridium thermosaccharolyticum, Bacillus cereus, E. coli, Saccharomyces cerevisiae and Marinobacter hydrocarbonoclasticus.

165. The method of claim 163, wherein the one or more nucleic acid molecules is thIA, atoB and/or ERG10.

166. The method of any one of claims 137-143, wherein the thiolase or acetyl coenzyme A acetyltransferase comprises an amino acid sequence selected from the group consisting of SEQ ID NOs: 35, 37 and 40.

167. The method of any one of claims 137-143, wherein the thiolase or acetyl coenzyme A acetyltransferase is encoded by a nucleic acid sequence selected from the group consisting of SEQ ID NOs: 33, 34, 36, 38 and 39.

168. The method of any one of claims 137-143, wherein the acetyl-CoA:acetoacetate-CoA transferase or acetate:acetoacetyl-CoA hydrolase is encoded by one or more nucleic acid molecules obtained from a microorganism selected from Clostridium sp. and E. coli.

169. The method of claim 168, wherein the microorganism is E. coli.

170. The method of claim 168, wherein the one or more nucleic acid molecules encoding the acetyl-CoA:acetoacetate-CoA transferase is atoA and/or atoD.

171. The method of claim 168, wherein the microorganism is Clostridium acetobutylicum.

172. The method of claim 168, wherein the one or more nucleic acid molecules encoding the acetate:acetoacetyl-CoA hydrolase is ctfA and/or ctfB.

173. The method of any one of claims 137-143, wherein the acetyl-CoA:acetoacetate-CoA transferase or acetate:acetoacetyl-CoA hydrolase comprises an amino acid sequence selected from the group consisting of SEQ ID NOs: 43, 46, 97, 99, 101 and 103.

174. The method of any one of claims 137-143, wherein the acetyl-CoA:acetoacetate-CoA transferase or acetate:acetoacetyl-CoA hydrolase is encoded by a nucleic acid sequence selected from the group consisting of SEQ ID NOs: 41, 42, 44, 45, 96, 98, 100 and 102.

175. The method of any one of claims 137-143, wherein the acetoacetate decarboxylase is encoded by one or more nucleic acid molecules obtained from a microorganism selected from the group consisting of Clostridium sp., Bacillus sp., Chromobacterium sp. and Pseudomonas sp.

176. The method of claim 175, wherein the microorganism is selected from the group consisting of Clostridium acetobutylicum, Clostridium beijerinckii, Clostridium cellulolyticum, Bacillus polymyxa, Chromobacterium violaceum and Pseudomonas putida.

177. The method of claim 175, wherein the one or more nucleic acid molecules encoding the acetoacetate decarboxylase is adc.

178. The method of any one of claims 137-143, wherein the acetoacetate decarboxylase comprises an amino acid sequence selected from the group consisting of SEQ ID NOs: 49 and 52.

179. The method of any one of claims 137-143, wherein the acetoacetate decarboxylase is encoded by a nucleic acid sequence selected from the group consisting of SEQ ID NOs: 47, 48, 50 and 51.

180. The method of any one of claims 137-143, wherein the 3-hydroxyisovalerate synthase is encoded by one or more nucleic acid molecules obtained from a microorganism selected from Mus sp., Saccharomyces sp., Lactobacillus sp. and Polaromonas sp.

181. The method of claim 180, wherein the microorganism is selected from Mus musculus, Saccharomyces cerevisiae, Lactobacillus crispatus and Polaromonas naphthalenivorans.

182. The method of claim 180, wherein the one or more nucleic acid molecules encoding the 3-hydroxyisovalerate synthase is selected from Hmgcs1, ERG13, PksG and/or Pnap_0477.

183. The method of any one of claims 137-143, wherein the one or more nucleic acid molecules encoding the 3-hydroxyisovalerate synthase comprises an amino acid sequence selected from the group consisting of SEQ ID NOs: 105, 107, 109 and 111.

184. The method of any one of claims 137-143, wherein the one or more nucleic acid molecules encoding the 3-hydroxyisovalerate synthase is encoded by a nucleic acid sequence selected from the group consisting of SEQ ID NOs: 104, 106, 108 and 110.

185. The method of any one of claims 137-143, wherein the hydroxymethylglutaryl-CoA synthase is encoded by one or more nucleic acid molecules obtained from Saccharomyces sp.

186. The method of claim 185, wherein the microorganism is from Saccharomyces cerevisiae.

187. The method of claim 185, wherein the one or more nucleic acid molecules encoding the hydroxymethylglutaryl-CoA synthase is HmgS.

188. The method of any one of claims 137-143, wherein the one or more nucleic acid molecules encoding the hydroxymethylglutaryl-CoA synthase comprises an amino acid sequence set forth in SEQ ID NO: 123.

189. The method of any one of claims 137-143, wherein the one or more nucleic acid molecules encoding the hydroxymethylglutaryl-CoA synthase is encoded by a nucleic acid sequence set forth in SEQ ID NO: 122.

190. The method of any one of claims 137-143, wherein the methylglutaconyl-CoA hydratase is encoded by one or more nucleic acid molecules obtained from Pseudomonas sp.

191. The method of claim 190, wherein the microorganism is from Pseudomonas putida.

192. The method of claim 190, wherein the one or more nucleic acid molecules encoding the methylglutaconyl-CoA hydratase is liuC.

193. The method of any one of claims 137-143, wherein the one or more nucleic acid molecules encoding the methylglutaconyl-CoA hydratase comprises an amino acid sequence set forth in SEQ ID NO: 125.

194. The method of any one of claims 137-143, wherein the one or more nucleic acid molecules encoding the methylglutaconyl-CoA hydratase is encoded by a nucleic acid sequence set forth in SEQ ID NO: 124.

195. The method of any one of claims 137-143, wherein the methylcrotonyl-CoA carboxylase is encoded by one or more nucleic acid molecules obtained from Pseudomonas sp.

196. The method of claim 195, wherein the microorganism is from Pseudomonas aeruginosa.

197. The method of claim 195, wherein the one or more nucleic acid molecules encoding the methylcrotonyl-CoA carboxylase is liuB and/or liuD.

198. The method of any one of claims 137-143, wherein the one or more nucleic acid molecules encoding the methylcrotonyl-CoA carboxylase comprises an amino acid sequence selected from SEQ ID NOs: 127 and 129.

199. The method of any one of claims 137-143, wherein the one or more nucleic acid molecules encoding the methylcrotonyl-CoA carboxylase is encoded by a nucleic acid sequence selected from SEQ ID NOs: 126 and 128.

200. The method of any one of claims 137-143, wherein the methylcrotonyl-CoA hydratase is a 3-ketoacyl-CoA thiolase.

201. The method of any one of claims 137-143, wherein the methylcrotonyl-CoA hydratase is an enoyl-CoA hydratase.

202. The method of any one of claims 137-143, wherein the methylcrotonyl-CoA hydratase is encoded by one or more nucleic acid molecules obtained from E. coli.

203. The method of claim 202, wherein the one or more nucleic acid molecules encoding the methylcrotonyl-CoA hydratase is fadA and/or fad B.

204. The method of any one of claims 137-143, wherein the one or more nucleic acid molecules encoding the methylcrotonyl-CoA hydratase comprises an amino acid sequence selected from SEQ ID NOs: 131 and 133.

205. The method of any one of claims 137-143, wherein the one or more nucleic acid molecules encoding the methylcrotonyl-CoA hydratase is encoded by a nucleic acid sequence selected from SEQ ID NOs: 130 and 132.

206. The method of any one of claims 137-143, wherein the 3-hydroxyisovaleryl-CoA thioesterase is encoded by one or more nucleic acid molecules obtained from E. coli.

207. The method of claim 206, wherein the one or more nucleic acid molecules encoding the 3-hydroxyisovaleryl-CoA thioesterase is tesB.

208. The method of any one of claims 137-143, wherein the one or more nucleic acid molecules encoding the 3-hydroxyisovaleryl-CoA thioesterase comprises an amino acid sequence set forth in SEQ ID NO: 135.

209. The method of any one of claims 137-143, wherein the one or more nucleic acid molecules encoding the 3-hydroxyisovaleryl-CoA thioesterase is encoded by a nucleic acid sequence set forth in SEQ ID NO: 134.

210. The method of any one of claims 137-143, wherein the 3HIV kinase is encoded by one or more nucleic acid molecules obtained from Thermoplasma sp. and Picrophilus sp.

211. The method of claim 210, wherein the microorganism is Thermoplasma acidophilum and Picrophilus torridus.

212. The method of claim 210, wherein the one or more nucleic acid molecules encoding the 3HIV kinase is TA1305 and/or PTO1356.

213. The method of claim 212, wherein the TA1305 comprises a L200E mutation.

214. The method of any one of claims 137-143, wherein the one or more nucleic acid molecules encoding the 3HIV kinase comprises an amino acid sequence selected from the group consisting of SEQ ID NOs: 113, 115 and 117.

215. The method of any one of claims 137-143, wherein the one or more nucleic acid molecules encoding the 3HIV kinase is encoded by a nucleic acid sequence selected from the group consisting of SEQ ID NOs: 112, 114 and 116.

216. The method of any one of claims 137-143, wherein the 3HIV-3-phosphate decarboxylase is encoded by one or more nucleic acid molecules obtained from Streptococcus sp.

217. The method of claim 216, wherein the microorganism is selected from Streptococcus mitis and/or Streptococcus gordonii.

218. The method of claim 216, wherein the one or more nucleic acid molecules encoding the 3HIV-3-phosphate decarboxylase comprises smi_1746 and/or mvaD.

219. The method of any one of claims 137-143, wherein the one or more nucleic acid molecules encoding the 3HIV-3-phosphate decarboxylase comprises an amino acid sequence selected from SEQ ID NOs: 119 and 121.

220. The method of any one of claims 137-143, wherein the one or more nucleic acid molecules encoding the 3HIV-3-phosphate decarboxylase is encoded by a nucleic acid sequence selected from SEQ ID NOs: 118 and 120.

221. The method of any one of claims 137-143, wherein the 3HIV decarboxylase is encoded by one or more nucleic acid molecules obtained from Streptococcus sp., Thermoplasma sp. and Picrophilus sp.

222. The method of claim 221, wherein the microorganism is Streptococcus gordonii, Thermoplasma acidophilum and Picrophilus torridus.

223. The method of claim 221, wherein the one or more nucleic acid molecules encoding the 3HIV decarboxylase comprises mvaD, TA1305 and/or PTO1356.

224. The method of any one of claims 137-143, wherein the one or more nucleic acid molecules encoding the 3HIV decarboxylase comprises an amino acid sequence selected from the group consisting of SEQ ID NOs: 113, 117and 121.

225. The method of any one of claims 137-143, wherein the one or more nucleic acid molecules encoding the mevalonate diphosphate decarboxylase is encoded by a nucleic acid sequence selected from the group consisting of SEQ ID NOs: 112, 116and 120.

226. The method of claim 138, wherein the D-xylulose 1-kinase is encoded by one or more nucleic acid molecules obtained from Homo sapiens.

227. The method of claim 226, wherein the one or more nucleic acid molecules encoding the D-xylulose 1-kinase is ketohexokinase C (khk-C).

228. The method of claim 138, wherein the one or more nucleic acid molecules encoding the D-xylulose 1-kinase comprises an amino acid sequence set forth in SEQ ID NO: 55.

229. The method of any claim 138, wherein the one or more nucleic acid molecules encoding the D-xylulose 1-kinase is encoded by a nucleic acid sequence selected from the group consisting of SEQ ID NOs: 53 and 54.

230. The method of claim 138, wherein the D-xylulose-1-phosphate aldolase is encoded by one or more nucleic acid molecules obtained from Homo sapiens.

231. The method of claim 230, wherein the one or more nucleic acid molecules encoding the D-xylulose-1-phosphate aldolase is aldolase B (ALDOB).

232. The method of claim 138, wherein the one or more nucleic acid molecules encoding the D-xylulose-1-phosphate aldolase comprises an amino acid sequence set forth in SEQ ID NO: 58.

233. The method of any claim 138, wherein the one or more nucleic acid molecules encoding the D-xylulose-1-phosphate aldolase is encoded by a nucleic acid sequence selected from the group consisting of SEQ ID NOs: 56 and 57.

234. The method of claim 141, wherein the xylose reductase or aldose reductase is encoded by one or more nucleic acid molecules obtained from a microorganism selected from the group consisting of Hypocrea sp., Scheffersomyces sp., Saccharomyces sp., Pachysolen sp., Pichia sp., Candida sp., Aspergillus sp., Neurospora sp., and Cryptococcus sp.

235. The method of claim 234, wherein the microorganism is selected from the group consisting of Hypocrea jecorina, Scheffersomyces stipitis, Saccharomyces cerevisiae, Pachysolen tannophilus, Pichia stipitis, Pichia quercuum, Candida shehatae, Candida tenuis, Candida tropicalis, Aspergillus niger, Neurospora crassa and Cryptococcus lactativorus.

236. The method of claim 234, wherein the one or more nucleic acid molecules encoding the xylose reductase or aldose reductase is xyl1 and/or GRE3.

237. The method of claim 141, wherein the one or more nucleic acid molecules encoding the xylose reductase or aldose reductase comprises an amino acid sequence selected from the group consisting of SEQ ID NOs: 84 and 87.

238. The method of claim 141, wherein the one or more nucleic acid molecules encoding the xylose reductase or aldose reductase is encoded by a nucleic acid sequence selected from the group consisting of SEQ ID NOs: 82, 83, 85 and 86.

239. The method of claim 141, wherein the xylitol dehydrogenase is encoded by one or more nucleic acid molecules obtained from a microorganism selected from the group consisting of Scheffersomyces sp., Trichoderma sp., Pichia sp., Saccharomyces sp., Gluconobacter sp., Galactocandida sp., Neurospora sp., and Serratia sp.

240. The method of claim 239, wherein the microorganism is selected from the group consisting of Scheffersomyces stipitis, Trichoderma reesei, Pichia stipitis, Saccharomyces cerevisiae, Gluconobacter oxydans, Galactocandida mastotermitis, Neurospora crassa and Serratia marcescens.

241. The method of claim 239, wherein the one or more nucleic acid molecules encoding the xylitol dehydrogenase is xyl2 and/or xdh1.

242. The method of claim 141, wherein the one or more nucleic acid molecules encoding the xylitol dehydrogenase comprises an amino acid sequence selected from the group consisting of SEQ ID NOs: 90 and 92.

243. The method of claim 141, wherein the one or more nucleic acid molecules encoding the xylitol dehydrogenase is encoded by a nucleic acid sequence selected from the group consisting of SEQ ID NOs: 88, 89 and 91.

244. The method of claim 140, wherein the xylose isomerase is encoded by one or more nucleic acid molecules obtained from Pyromyces sp.

245. The method of claim 244, wherein the one or more nucleic acid molecules encoding the xylose isomerase is xylA.

246. The method of claim 140, wherein the one or more nucleic acid molecules encoding the xylose isomerase comprises an amino acid sequence set forth in SEQ ID NO: 95.

247. The method of claim 140, wherein the one or more nucleic acid molecules encoding the xylose isomerase is encoded by a nucleic acid sequence selected from the group consisting of SEQ ID NOs: 93 and 94.

248. The method of claim 142, wherein the xylose dehydrogenase is encoded by one or more nucleic acid molecules obtained from a microorganism selected from the group consisting of Caulobacter sp., Haloarcula sp., Haloferax sp., Halorubrum sp. and Trichoderma sp.

249. The method of claim 248, wherein the microorganism is selected from the group consisting of Caulobacter crescentus, Haloarcula marismortui, Haloferax volcanii, Halorubrum lacusprofundi and Trichoderma reesei.

250. The method of claim 248, wherein the one or more nucleic acid molecules encoding the xylose dehydrogenase is selected from xylB, xdh1 (HVO_B0028) and/or xyd1.

251. The method of claim 142, wherein the one or more nucleic acid molecules encoding the xylose dehydrogenase comprises an amino acid sequence selected from the group consisting of SEQ ID NOs: 61, 63 and 65.

252. The method of claim 142, wherein the one or more nucleic acid molecules encoding the xylose dehydrogenase is encoded by a nucleic acid sequence selected from the group consisting of SEQ ID NOs: 59, 60, 62 and 64.

253. The method of claim 142, wherein the xylonolactonase is encoded by one or more nucleic acid molecules obtained from a microorganism selected from Caulobacter sp. and Haloferax sp.

254. The method of claim 253, wherein the microorganism is selected from the group consisting of Caulobacter crescentus, Haloferax volcanii and Haloferax gibbonsii.

255. The method of claim 253, wherein the one or more nucleic acid molecules encoding the xylonolactonase is xylC.

256. The method of claim 142, wherein the one or more nucleic acid molecules encoding the xylonolactonase comprises an amino acid sequence set forth in SEQ ID NO: 67.

257. The method of claim 142, wherein the one or more nucleic acid molecules encoding the xylonolactonase is encoded by a nucleic acid sequence set forth in SEQ ID NO: 66.

258. The method of claim 142, wherein the xylonate dehydratase is encoded by one or more nucleic acid molecules obtained from a microorganism selected from the group consisting of Caulobacter sp., Sulfolobus sp. and E. coli.

259. The method of claim 258, wherein the microorganism is selected from the group consisting of Caulobacter crescentus, Sulfolobus soffataricus and E. coli.

260. The method of claim 258, wherein the one or more nucleic acid molecules encoding the xylonate dehydratase is selected from xylD, yjhG and/or yagF.

261. The method of claim 142, wherein the one or more nucleic acid molecules encoding the xylonate dehydratase comprises an amino acid sequence selected from the group consisting of SEQ ID NOs: 69, 72 and 75.

262. The method of claim 142, wherein the one or more nucleic acid molecules encoding the xylonate dehydratase is encoded by a nucleic acid sequence selected from the group consisting of SEQ ID NOs: 68, 70, 71, 73 and 74.

263. The method of claim 142, wherein the 2-keto-3-deoxy-D-pentonate aldolase is encoded by one or more nucleic acid molecules obtained from a microorganism selected from Pseudomonas sp. and E. coli.

264. The method of claim 263, wherein the microorganism is E. coli.

265. The method of claim 263, wherein the one or more nucleic acid molecules encoding the 2-keto-3-deoxy-D-pentonate aldolase is selected from yjhH and/or yagE.

266. The method of claim 142, wherein the one or more nucleic acid molecules encoding the 2-keto-3-deoxy-D-pentonate aldolase comprises an amino acid sequence selected from the group consisting of SEQ ID NOs: 78 and 81.

267. The method of claim 142, wherein the one or more nucleic acid molecules encoding the 2-keto-3-deoxy-D-pentonate aldolase is encoded by a nucleic acid sequence selected from the group consisting of SEQ ID NOs: 76, 77, 79 and 80.

268. A recombinant microorganism co-producing monoethylene glycol (MEG) and isobutene.

269. The recombinant microorganism of claim 268, wherein MEG and isobutene are co-produced from xylose.

270. The recombinant microorganism of claim 268, wherein the recombinant microorganism comprises a deletion, insertion, or loss of function mutation in a gene encoding a D-xylulose-5-kinase and/or in a gene encoding a glycoaldehyde dehydrogenase.

271. The recombinant microorganism of claim 270, wherein the gene encoding the D-xylulose-5-kinase is xylB.

272. The recombinant microorganism of claim 270, wherein the gene encoding the glycoaldehyde dehydrogenase is aldA.

273. The recombinant microorganism of claim 268, wherein isobutene is synthesized via the intermediate 3-hydroxyisovalerate.

274. The recombinant microorganism of any one of claims 268-273, wherein MEG is produced through the conversion of glycolaldehyde in a C-2 branch pathway and isobutene is produced through the conversion of DHAP or pyruvate in a C-3 branch pathway.

275. The recombinant microorganism of claim 274, wherein at least a portion of the excess NADH produced in the C-3 branch pathway is used as a source of reducing equivalents in the C-2 branch pathway.

276. The recombinant microorganism of claim 274, wherein at least a portion of the excess NADH produced in the C-3 branch pathway is used to produce ATP.

277. The recombinant microorganism of any one of claims 268-273, wherein excess biomass formation is minimized and production of MEG and isobutene is maximized.
